(19) United States
(12) Patent Application Publication
(10) Pub. No.: US 2024/0092799 A1
(43) Pub. Date: Mar. 21, 2024

(54) MNK INHIBITORS

(71) Applicant: Hepagene Therapeutics (HK) Limited, Wan Chai, Hong Kong (CN)

(72) Inventors: Cheng MO, Shanghai (CN); Xiaodong Xu, Boston (CN)

(21) Appl. No.: 18/021,768

(22) PCT Filed: Aug. 20, 2021
(86) PCT No.: PCT/IB2021/057657

§ 371 Date: Feb. 16, 2023

Related U.S. Application Data
(60) Provisional application No. 63/068,268, filed on Aug. 20, 2020.
(60) Provisional application No. 63/157,126, filed on Mar. 5, 2021.

(51) Int. Cl. C07D 495/04; C07D 495/20; C07D 519/00; C07D 471/04; A61P 35/00

(52) U.S. Cl. CPC C07D 495/04; C07D 495/20; C07D 519/00; C07D 471/04; A61P 35/00

(57) ABSTRACT

The present technology is directed to compounds, compositions, and methods related to modulation of MNK. In particular, the present compounds and compositions may be used to treat MNK-mediated disorders and conditions, including, e.g., various solid and hematological cancers.

### CROSS REFERENCE TO RELATED APPLICATIONS

This application claims the benefit of U.S. Provisional Patent Application No. 63/068,268, filed on Aug. 20, 2020, and U.S. Provisional Patent Application No. 63/157,126, filed on Mar. 5, 2021, the entire disclosures of which are hereby incorporated by reference for any and all purposes.

### FIELD

The present technology is directed to compounds, compositions, and methods related to inhibitors of MAPK interacting kinase (MNK). In particular, the present compounds and compositions may be used to treat MNK-mediated disorders and conditions, including, e.g., various types of cancer as disclosed herein.

### BACKGROUND

Translation is a tightly controlled process for a select set of mRNAs, and dysregulation of this process drives aberrant proliferation, angiogenesis, survival, and alterations in immune function, all hallmarks of cancer. A key player in translational control is eIF4E, the mRNA 5′ cap-binding protein. Aberrant expression of eIF4E promotes tumorigenesis and has been implicated in cancer development and progression. Regulation of eIF4E is partly achieved through phosphorylation. This modification has been shown to be essential for eIF4E's role in tumorigenesis but not for normal development and cell homeostasis.

MNK, a Ser/Thr kinase, is the only kinase known to phosphorylated eIF4E at serine 209 which is its only phosphorylation site. MNK1/2 double knockout studies in mice further demonstrated that these kinases are not required for normal growth and development. Several selective MNK1/2 inhibitors, such as eFT508, BAY1143269 and ETC-206, show antitumor efficacy in various CDX models. These results show that blocking the eIF4E phosphorylation by selectively inhibiting MNK1/2 can be an effective therapeutic strategy to treat related diseases.

### SUMMARY

The present technology provides compounds, compositions, and methods related to modulation of MNK and treatment of MNK-mediated disorders and conditions. In one aspect, the present technology provides a compound according to Formula I or Formula II:

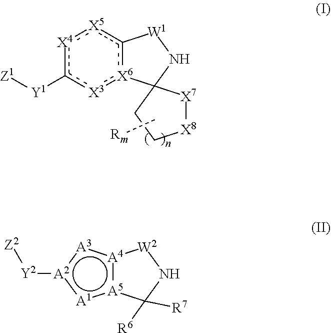

stereoisomers, tautomers, and/or pharmaceutically acceptable salts thereof; wherein
W¹ and W² are independently C(═NR⁹), C(═O), C(═S), S(═O), or S(═O)₂,
X¹ is N or CR^{2a};
X² is N or CR^{2b};
X³ is N, N(O), C(═O) or CR^{2c};
X⁴ is N or CR⁴;
X⁵ is N or CR⁵;
X⁶ is C or N, wherein when X⁶ is C, the dotted lines in Formula I indicate aromatic bonds, and when X⁶ is N, then X³ is C(═O) and the dotted lines in Formula I indicate single or double bonds;
X⁷ and X⁸ are independently O, NH, N(O), NR¹⁰, NC(O)R¹¹, NC(O)OR¹¹, S, S(═O), S(═O)₂, CHR¹³, and C(═O), provided that X⁷ and X⁸ are not both O;
X⁹ and X¹⁰ are independently N or CR^{2a};
Y¹ and Y² are independently absent, NH, NR¹⁰, O, CHR¹⁴, C(═O) S(═O) S(O)₂, cyclopropyl, or a 5-member heteroarylene ring;
Z¹ and Z² are independently a heteroaryl moiety selected from Formulas III, IV, V.

VI, VII, VIII, IX, X, or XI:

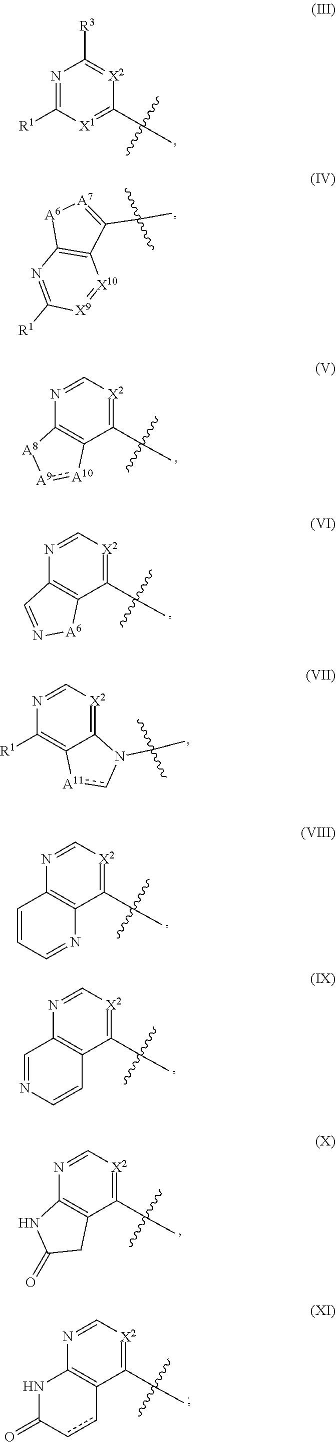

wherein the dotted lines in Formulas V, VII, and XI indicate a single or double bond;
A¹ is CR^{2e}, N, NR⁸, O or S, provided that the ring of which it is a member is a heteroaryl ring;
A² is C or N, provided that the ring of which it is a member is a heteroaryl ring;
A³ is CR^{2f}, N, NR⁸, O or S, provided that the ring of which it is a member is a heteroaryl ring;
A⁴ and A⁵ are each C or one is C and the other N, such that the ring of which they are members is a heteroaryl ring;
A⁶ is NR⁸, O, S, or S(═O);
A⁷ is CR^{2g} or N;
A⁸ is NR⁸, NHC(O), O, S, S(═O);
A⁹ is CH, CH₂, C(O), CR¹⁵, CR¹⁸, or N, provided that when A⁹ is CR¹⁵, A¹⁰ is CR¹⁶;
A¹⁰ is CH, CH₂, CR¹⁶, CR¹⁹, N, NH, or S, provided that when A¹⁰ is CR¹⁶, A⁹ is CR¹⁵;
A¹¹ is CH, CH₂ or N;
R is independently at each occurrence halo, NO₂, NR⁸R¹⁰, OR¹¹, SR¹², CN, COOR¹³, or a substituted or unsubstituted C₁₋₆ alkyl, C₃₋₇ cycloalkyl, or C₂₋₆ alkenyl group; or when m is at least 2, the two R moieties together form a C₁₋₄ alkylene bridge between non-adjacent ring members;
R¹, R^{2a}, R^{2b}, R^{2c}, R^{2d}, R^{2e}, R^{2f}, R^{2g}, and R³ are independently at each occurrence H, halo, NO₂, NR⁸R¹⁰, OR¹¹, SR¹², CN, COOR¹³, or a substituted or unsubstituted C₁₋₆ alkyl, C₃₋₇ cycloalkyl, or, C₂₋₆ alkenyl group;
R⁴ and R⁵ are independently H, halo, CN, OH, SR¹², NO₂, NR⁸R¹⁰, or a substituted or unsubstituted C₁₋₆ alkyl, C₁₋₆ alkoxy, or C₂₋₆ alkene; or R⁴ and R⁵ when present, together with the carbon atoms to which they are attached, form a fused phenyl or a 5- or 6-membered cycloalkenyl, heterocyclyl or heteroaryl ring;
R⁶ and R⁷ are independently H, NHR¹⁰, or a substituted or unsubstituted C₁₋₈-alkyl, C₂₋₈-alkenyl, cycloalkyl, cycloalkylalkyl, aryl, aralkyl, heterocyclyl, heterocyclylalkyl, heteroaryl, or heteroarylalkyl group; or R⁶ and R⁷ together with the carbon to which they are attached form a substituted or unsubstituted cycloalkyl or heterocyclyl ring;
R⁸ and R¹⁰ are independently at each occurrence H, an amino protecting group, or a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁₋₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group; or R⁸ and R¹⁰ together with the nitrogen to which they are attached form a substituted or unsubstituted heterocyclyl ring;
R⁹ is at independently each occurrence H or substituted or unsubstituted alkyl group;
R¹¹ is independently at each occurrence H, a hydroxyl protecting group, or a substituted or unsubstituted alkyl, cycloalkyl, alkenyl, heterocyclyl, heteroaryl, aryl or aralkyl group;
R¹² is independently at each occurrence H, a thiol protecting group, or a substituted or unsubstituted alkyl, cycloalkyl, alkenyl, aryl or aralkyl group;
R¹³ is independently at each occurrence H or a substituted or unsubstituted alkyl, cycloalkyl, alkenyl, aryl or aralkyl group; and
R¹⁴ is H, OH, or a substituted or unsubstituted alkyl group;
R¹⁵ and R¹⁶, together with the carbons to which they are attached, form a cyclohexenyl ring, optionally substituted with C(O)R¹⁷, C(O)OR¹⁷, or C(O)NR⁸R¹⁰;
R¹⁷ is independently at each occurrence H or a substituted or unsubstituted alkyl, cycloalkyl, or alkenyl group;
R¹⁸ and R¹⁹ are independently selected from CN, C(O)R¹⁷, C(O)OR¹⁷, C(O)NR⁸R¹⁰, or a substituted or unsubstituted alkyl, cycloalkyl, or alkenyl group;
m is 0, 1, 2 or 3; and
n is 1 or 2.

In any embodiments, when X⁴ is CH, at least one of X⁷ and X⁸ may be a heteroatom, or n may be at least 2 and the two R moieties together form a C₁₋₄ alkylene bridge between non-adjacent ring members, or W¹ may be S(═O)₂, or X³ may be N(O).

In a related aspect, a composition is provided that includes any one of the compounds of Formulas I and II disclosed herein (or any other compounds disclosed herein) and a pharmaceutically acceptable carrier.

In another aspect, a pharmaceutical composition is provided, the pharmaceutical composition including an effective amount any one of the compounds disclosed herein for treating an MNK-mediated disorder or condition, and optionally one or more of a pharmaceutically acceptable carrier and/or excipient(s).

In another aspect, a method of treatment is provided that includes administering an effective amount of a compound of any aspect or embodiment described herein, or administering a pharmaceutical composition including an effective amount of such a compound, to a subject suffering from an MNK-mediated disorder or condition.

In another aspect, a method is provided for inhibiting the activity of Mnk in at least one cell overexpressing Mnk, by contacting MINK with an effective amount of any one of the compounds of Formulas I, II or aspects or embodiments thereof as described herein.

### DETAILED DESCRIPTION

In various aspects, the present technology provides compounds and methods for inhibiting MNK activity and the treatment of MNK-mediated disorders and conditions. The compounds provided herein may be used in the disclosed methods. Also provided is the use of the compounds in preparing pharmaceutical formulations and medicaments for use in the disclosed methods.

The following terms are used throughout as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of the present technology. Procedures for inserting such labels into the compounds of the present technology will be readily apparent to those skilled in the art based on the disclosure herein.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); haloalkyl (e.g., CF₃); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; amines; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocyclyl and heteroaryl groups also include rings and ring systems in which a bond to a hydrogen atom is replaced with a bond to a carbon atom. Therefore, substituted cycloalkyl, aryl, heterocyclyl and heteroaryl groups may also be substituted with substituted or unsubstituted alkyl, alkenyl, and alkynyl groups as defined below.

Alkyl groups include straight chain and branched chain alkyl groups having from 1 to 12 carbon atoms, and typically from 1 to 10 carbons or, in some embodiments, from 1 to 8, 1 to 6, or 1 to 4 carbon atoms. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. In some embodiments, cycloalkylalkyl groups have from 4 to 16 carbon atoms, 4 to 12 carbon atoms, and typically 4 to 10 carbon atoms.

Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Alkenyl groups include straight and branched chain alkyl groups as defined above, except that at least one double bond exists between two carbon atoms. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Cycloalkenyl groups include cycloalkyl groups as defined above, having at least one double bond between two carbon atoms. In some embodiments the cycloalkenyl group may have one, two or three double bonds but does not include aromatic compounds. Cycloalkenyl groups have from 4 to 14 carbon atoms, or, in some embodiments, 5 to 14 carbon atoms, 5 to 10 carbon atoms, or even 5, 6, 7, or 8 carbon atoms. Examples of cycloalkenyl groups include cyclohexenyl, cyclopentenyl, cyclohexadienyl, cyclobutadienyl, and cyclopentadienyl.

Cycloalkenylalkyl groups are alkyl groups as defined above in which a hydrogen or carbon bond of the alkyl group is replaced with a bond to a cycloalkenyl group as defined above. Substituted cycloalkenylalkyl groups may be substituted at the alkyl, the cycloalkenyl or both the alkyl and cycloalkenyl portions of the group. Representative substituted cycloalkenylalkyl groups may be substituted one or more times with substituents such as those listed above.

Alkynyl groups include straight and branched chain alkyl groups as defined above, except that at least one triple bond exists between two carbon atoms. Alkynyl groups have from 2 to 12 carbon atoms, and typically from 2 to 10 carbons or, in some embodiments, from 2 to 8, 2 to 6, or 2 to 4 carbon atoms. In some embodiments, the alkynyl group has one, two, or three carbon-carbon triple bonds. Examples include, but are not limited to —C≡H, —C≡CCH₃, —CH₂C≡CH₃, —C≡CCH₂CH(CH₂CH₃)₂, among others. Representative substituted alkynyl groups may be mono-substituted or substituted more than once, such as, but not limited to, mono-, di- or tri-substituted with substituents such as those listed above.

Aryl groups are cyclic aromatic hydrocarbons that do not contain heteroatoms. Aryl groups herein include monocyclic, bicyclic and tricyclic ring systems. Thus, aryl groups include, but are not limited to, phenyl, azulenyl, heptalenyl, biphenyl, fluorenyl, phenanthrenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. In some embodiments, the aryl groups are phenyl or naphthyl. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once, e.g., 2, 3, 4, or 5 times. Monosubstituted aryl groups include, but are not limited to, 2-, 3-, 4-, 5-, or 6-substituted phenyl or naphthyl groups, which may be substituted with substituents such as those listed above.

Aralkyl groups are alkyl groups as defined above in which a hydrogen or carbon bond of an alkyl group is replaced with a bond to an aryl group as defined above. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heterocyclyl groups include non-aromatic ring compounds containing 3 or more ring members, of which one or more is a heteroatom such as, but not limited to, N, O, and S. In some embodiments, the heterocyclyl group contains 1, 2, 3 or 4 heteroatoms. In some embodiments, heterocyclyl groups include mono-, bi- and tricyclic rings having 3 to 16 ring members, whereas other such groups have 3 to 6, 3 to 10, 3 to 12, or 3 to 14 ring members. Heterocyclyl groups encompass partially unsaturated and saturated ring systems, such as, for example, imidazolinyl and imidazolidinyl groups. The phrase “heterocyclyl group” includes fused ring species comprising fused non-aromatic groups. The phrase also includes bridged polycyclic ring systems containing a heteroatom such as, but not limited to, quinuclidyl. However, the phrase does not include heterocyclyl groups that have other groups, such as alkyl, oxo or halo groups, bonded to one of the ring members. Rather, these are referred to as “substituted heterocyclyl groups”. Heterocyclyl groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, pyrrolinyl, imidazolinyl, pyrazolinyl, oxadiazolonyl (including 1,2,4-oxazol-5 (4H)-one-3-yl), thiazolinyl, piperidyl, piperazinyl, morpholinyl, thiomorpholinyl, tetrahydropyranyl, tetrahydrothiopyranyl, oxathiane, dioxyl, dithianyl, pyranyl, dihydropyridyl, dihydrodithiinyl, homopiperazinyl, quinuclidyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, indolizinyl, azaindolyl (pyrrolopyridinyl), indazolyl, indolinylbenzimidazolyl, imidazopyridinyl (azabenzimidazolyl), dihydroindolyl, dihydrobenzodioxinyl, pyrazolopyridinyl, triazolopyridinyl, benzotriazolyl, benzoxazolyl, benzothiazolyl, benzothiadiazolyl, dihydrobenzothiazinyl, dihydrobenzofuranyl, imidazopyridinyl, isoxazolopyridinyl, thianaphthyl, purinyl, xanthinyl, phthalazinyl, adeninyl, guaninyl, quinolinyl, isoquinolinyl, tetrahydroquinolinyl, cinnolinyl, quinoxalinyl, and quinazolinyl groups. Heteroaryl groups include fused ring compounds in which all rings are aromatic such as indolyl groups and include fused ring compounds in which only one of the rings is aromatic, such as 2,3-dihydro indolyl groups. Although the phrase “heteroaryl groups” includes fused ring compounds, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Heterocyclylalkyl groups are alkyl groups as defined above in which a hydrogen or carbon bond of an alkyl group is replaced with a bond to a heterocyclyl group as defined above. Substituted heterocyclylalkyl groups may be substituted at the alkyl, the heterocyclyl or both the alkyl and heterocyclyl portions of the group. Representative heterocyclyl alkyl groups include, but are not limited to, morpholin-4-yl-ethyl, furan-2-yl-methyl, imidazol-4-yl-methyl, pyridin-3-yl-methyl, tetrahydrofuran-2-yl-ethyl, and indol-2-yl-propyl. Representative substituted heterocyclylalkyl groups may be substituted one or more times with substituents such as those listed above.

Heteroaralkyl groups are alkyl groups as defined above in which a hydrogen or carbon bond of an alkyl group is replaced with a bond to a heteroaryl group as defined above. Substituted heteroaralkyl groups may be substituted at the alkyl, the heteroaryl or both the alkyl and heteroaryl portions of the group. Representative substituted heteroaralkyl groups may be substituted one or more times with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are heteroarylene groups, and so forth. Substituted groups having a single point of attachment to the compound of the present technology are not referred to using the “ene” designation. Thus, e.g., chloroethyl is not referred to herein as chloroethylene.

Alkoxy groups are hydroxyl groups (—OH) in which the bond to the hydrogen atom is replaced by a bond to a carbon atom of a substituted or unsubstituted alkyl group as defined above. Examples of linear alkoxy groups include but are not limited to methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include but are not limited to cyclopropyloxy, cyclobutyloxy, cyclopentyloxy, cyclohexyloxy, and the like. Representative substituted alkoxy groups may be substituted one or more times with substituents such as those listed above.

The terms “alkanoyl” and “alkanoyloxy” as used herein can refer, respectively, to —C(O)-alkyl groups and —O—C(O)-alkyl groups, each containing 2-5 carbon atoms. Similarly, “aryloyl” and “aryloyloxy” refer to —C(O)-aryl groups and —O—C(O)-aryl groups.

The terms “aryloxy” and “arylalkoxy” refer to, respectively, a substituted or unsubstituted aryl group bonded to an oxygen atom and a substituted or unsubstituted aralkyl group bonded to the oxygen atom at the alkyl. Examples include but are not limited to phenoxy, naphthyloxy, and benzyloxy. Representative substituted aryloxy and arylalkoxy groups may be substituted one or more times with substituents such as those listed above.

The term “carboxylate” as used herein refers to a —COOH group.

The term “ester” as used herein refers to —COOR⁷⁰ and —C(O)O-G groups. R⁷⁰ is a substituted or unsubstituted alkyl, cycloalkyl, alkenyl, alkynyl, aryl, aralkyl, heterocyclylalkyl or heterocyclyl group as defined herein. G is a carboxylate protecting group. Carboxylate protecting groups are well known to one of ordinary skill in the art. An extensive list of protecting groups for the carboxylate group functionality may be found in Protective Groups in Organic Synthesis, Greene, T. W.; Wuts, P. G. M., John Wiley & Sons, New York, NY, (3rd Edition, 1999) which can be added or removed using the procedures set forth therein and which is hereby incorporated by reference in its entirety and for any and all purposes as if fully set forth herein.

The term “amide” (or “amido”) includes C- and N-amide groups, i.e., —C(O)NR⁷¹R⁷², and —NR⁷¹C(O)R⁷² groups, respectively. R⁷¹ and R⁷² are independently hydrogen, or a substituted or unsubstituted alkyl, alkenyl, alkynyl, cycloalkyl, aryl, aralkyl, heterocyclylalkyl or heterocyclyl group as defined herein. Amido groups therefore include but are not limited to carbamoyl groups (—C(O)NH₂) and formamide groups (—NHC(O)H). In some embodiments, the amide is —NR⁷¹C(O)—(C₁₋₅ alkyl) and the group is termed “carbonylamino,” and in others the amide is —NHC(O)-alkyl and the group is termed “alkanoylamino.”

The term “nitrile” or “cyano” as used herein refers to the —CN group.

Urethane groups include N- and O-urethane groups, i.e., —NR⁷³C(O)OR⁷⁴ and —OC(O)NR⁷³R⁷⁴ groups, respectively. R⁷³ and R⁷⁴ are independently a substituted or unsubstituted alkyl, alkenyl, alkynyl, cycloalkyl, aryl, aralkyl, heterocyclylalkyl, or heterocyclyl group as defined herein. R⁷³ may also be H.

The term “amine” (or “amino”) as used herein refers to —NR⁷⁵R⁷⁶ groups, wherein R⁷⁵ and R⁷⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In other embodiments, the amine is NH₂, methylamino, dimethylamino, ethylamino, diethylamino, propylamino, isopropylamino, phenylamino, or benzylamino.

As used herein, the term “protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 3rd Ed. (John Wiley & Sons, Inc., New York), which is hereby incorporated by reference in its entirety and for any and all purposes as if fully set forth herein. Hydroxyl protecting groups include ethers, esters, and carbonates, among others. Hydroxyl protecting groups include but art not limited to: methoxymethyl ethers (MOM), methoxyethoxymethyl ethers (MEM), benzyloxymethyl ethers (BOM), tetrahydropyranyl ethers (THP), benzyl ethers (Bn), p-methoxybenzyl ethers, trimethylsilyl ethers (TMS), triethylsilyl ethers (TES), triisopropylsilyl ethers (TIPS), t-butyldimethylsilyl ethers (TBDMS), t-butyldiphenylsilyl ethers (TBDPS), o-nitrobenzyl ethers, p-nitrobenzyl ethers, trityl ethers, acetate, chloroacetate, dichloroacetate, trichloroacetate, trifluoroacetate, benzoate (Bz), methyl carbonate, allyl carbonate (alloc), dimethylthiocarbamate (DMTC), benzyl carbonate (Cbz), t-butyl carbonate (Boc), and 9-(fluorenylmethyl) carbonate (Fmoc). Amino protecting groups include, but are not limited to, urethanes, sulfonyl groups, silyl groups, and others. For example, amino protecting groups include mesitylenesulfonyl (Mts), benzyloxycarbonyl (Cbz or Z), t-butyloxycarbonyl (Boc), t-butyldimethylsilyl (TBS or TBDMS), 9-fluorenylmethyloxycarbonyl (Fmoc), allyloxycarbonyl (Alloc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, α,α-dimethyldimethoxybenzyloxycarbonyl (DDZ), 5-bromo-7-nitroindolinyl, and the like. Amino protecting groups susceptible to acid-mediated removal include but are not limited to Boc and TBDMS. Amino protecting groups resistant to acid-mediated removal and susceptible to hydrogen-mediated removal include but are not limited to Alloc, Cbz, nitro, and 2-chlorobenzyloxycarbonyl. Amino groups susceptible to base-mediated removal, but resistant to acid-mediated removal include Fmoc. Thiol protecting groups include but are not limited to thioethers (e.g., t-butyl, benzyl, substituted benzyl groups), acylated thiols, e.g., thioacetyl, and sulfenyl groups. Thioether protecting groups may be generally be prepared by reaction of the thiol under basic conditions with a halide, and may be often be removed by exposure to appropriate acids.

The term “sulfonamido” includes S- and N-sulfonamide groups, i.e., —SO₂NR⁷⁸R⁷⁹ and —NR⁷⁸SO₂R⁷⁹ groups, respectively. R⁷⁸ and R⁷⁹ are independently hydrogen, or a substituted or unsubstituted alkyl, alkenyl, alkynyl, cycloalkyl, aryl, aralkyl, heterocyclylalkyl, or heterocyclyl group as defined herein. Sulfonamido groups therefore include but are not limited to sulfamoyl groups (—SO₂NH₂). In some embodiments herein, the sulfonamido is —NHSO₂-alkyl and is referred to as the “alkylsulfonylamino” group.

The term “thiol” refers to —SH groups, while “sulfides” include —SR⁸⁰ groups, “sulfoxides” include —S(O)R⁸¹ groups, “sulfones” include —SO₂R⁸² groups, and “sulfonyls” include —SO₂OR⁸³. R⁸⁰, R⁸¹, R⁸² and R⁸³ are each independently a substituted or unsubstituted alkyl, cycloalkyl, alkenyl, alkynyl, aryl aralkyl, heterocyclyl or heterocyclylalkyl group as defined herein. In some embodiments the sulfide is an alkylthio group, —S-alkyl.

The term “urea” refers to NR⁸⁴—C(O)—NR⁸⁵R⁸⁶ groups. R⁸⁴, R⁸⁵, and R⁸⁶ groups are independently hydrogen, or a substituted or unsubstituted alkyl, alkenyl, alkynyl, cycloalkyl, aryl, aralkyl, heterocyclyl, or heterocyclylalkyl group as defined herein.

The term “amidine” refers to —C(NR⁸⁷)NR⁸⁸R⁸⁹ and —NR⁸⁷C(NR⁸⁸)R⁸⁹, wherein R⁸⁷, R⁸⁸, and R⁸⁹ are each independently hydrogen, or a substituted or unsubstituted alkyl, cycloalkyl, alkenyl, alkynyl, aryl aralkyl, heterocyclyl or heterocyclylalkyl group as defined herein.

The term “guanidine” refers to —NR⁹⁰C(NR⁹¹)NR⁹²R⁹³, wherein R⁹⁰, R⁹¹, R⁹² and R⁹³ are each independently hydrogen, or a substituted or unsubstituted alkyl, cycloalkyl, alkenyl, alkynyl, aryl aralkyl, heterocyclyl or heterocyclylalkyl group as defined herein.

The term “enamine” refers to —C(R⁹⁴)═C(R⁹⁵)NR⁹⁶R⁹⁷ and —NR⁹⁴C(R⁹⁵)═C(R⁹⁶)R⁹⁷, wherein R⁹⁴, R⁹⁵, R⁹⁶ and R⁹⁷ are each independently hydrogen, a substituted or unsubstituted alkyl, cycloalkyl, alkenyl, alkynyl, aryl aralkyl, heterocyclyl or heterocyclylalkyl group as defined herein.

The term “halogen” or “halo” as used herein refers to bromine, chlorine, fluorine, or iodine. In some embodiments, the halogen is fluorine. In other embodiments, the halogen is chlorine or bromine.

The term “hydroxyl” as used herein can refer to —OH or its ionized form, —O⁻. A “hydroxyalkyl” group is a hydroxyl-substituted alkyl group, such as HO—CH₂—.

The term “imide” refers to —C(O)NR⁹⁸C(O)R⁹⁹, wherein R⁹⁸ and R⁹⁹ are each independently hydrogen, or a substituted or unsubstituted alkyl, cycloalkyl, alkenyl, alkynyl, aryl aralkyl, heterocyclyl or heterocyclylalkyl group as defined herein.

The term “imine” refers to —CR¹⁰⁰(NR¹⁰¹) and —N(CR¹⁰⁰R¹⁰¹) groups, wherein R¹⁰⁰ and R¹⁰¹ are each independently hydrogen or a substituted or unsubstituted alkyl, cycloalkyl, alkenyl, alkynyl, aryl aralkyl, heterocyclyl or heterocyclylalkyl group as defined herein, with the proviso that R¹⁰⁰ and R¹⁰¹ are not both simultaneously hydrogen.

The term “nitro” as used herein refers to an —NO₂ group.

The term “trifluoromethyl” as used herein refers to —CF₃.

The term “trifluoromethoxy” as used herein refers to —OCF₃.

The term “azido” refers to —N₃.

The term “trialkyl ammonium” refers to a —N(alkyl)₃ group. A trialkylammonium group is positively charged and thus typically has an associated anion, such as halogen anion.

The term “isocyano” refers to —NC.

The term “isothiocyano” refers to —NCS.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or atoms, and so forth.

Pharmaceutically acceptable salts of compounds described herein are within the scope of the present technology and include acid or base addition salts which retain the desired pharmacological activity and is not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g. alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g. Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g. arginine, lysine and ornithine). Such salts can be prepared in situ during isolation and purification of the compounds or by separately reacting the purified compound in its free base or free acid form with a suitable acid or base, respectively, and isolating the salt thus formed.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereochemical or geometric isomeric forms, it should be understood that the present technology encompasses any tautomeric, conformational isomeric, stereochemical and/or geometric isomeric forms of the compounds having one or more of the utilities described herein, as well as mixtures of these various different forms.

“Tautomers” refers to isomeric forms of a compound that are in equilibrium with each other. The presence and concentrations of the isomeric forms will depend on the environment the compound is found in and may be different depending upon, for example, whether the compound is a solid or is in an organic or aqueous solution. Typically, tautomers differ by the position of a proton within the molecule. For example, in aqueous solution, ketone and enol tautomers may be present.

Because of the limits of representing compounds by structural formulas, it is to be understood that all chemical formulas of the compounds described herein represent all tautomeric forms of compounds and are within the scope of the present technology.

Stereoisomers of compounds (also known as optical isomers) include all chiral, diastereomeric, and racemic forms of a structure, unless the specific stereochemistry is expressly indicated. Thus, compounds used in the present technology include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

In one aspect, the present technology provides heterocyclic derivatives that are useful for inhibiting MNK, treating a MNK-mediated disorder or condition, and intermediates for making such compounds. Thus, there are provided compounds having the structure of Formula I or Formula II:

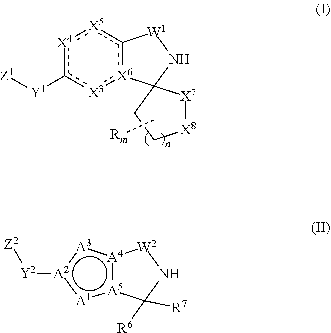

stereoisomers, tautomers, and/or pharmaceutically acceptable salts thereof;
wherein
W¹ and W² are independently C(═NR⁹), C(═O), C(═S), S(═O), or S(═O)₂;
X¹ is N or CR^{2a};
X² is N or CR^{2b};
X³ is N, N(O), C(═O) or CR^{2c};
X⁴ is N or CR⁴;
X⁵ is N or CR⁵;
X⁶ is C or N, wherein when X⁶ is C, the dotted lines in Formula I indicate aromatic bonds, and when X⁶ is N, then X³ is C(═O) and the dotted lines in Formula I indicate single or double bonds;
X⁷ and X⁸ are independently O, NH, N(O), NR¹⁰, NC(O)R¹¹, NC(O)OR¹¹, S, S(═O), S(═O)₂, CHR¹³, and C(═O), provided that X⁷ and X⁸ are not both O;
X⁹ and X¹⁰ are independently N or CR^{2d};
Y¹ and Y² are independently absent, NH, NR¹⁰, O, CHR¹⁴, C(═O), S(═O), S(O)₂, cyclopropyl, or a 5-member heteroarylene ring;
Z¹ and Z² are independently a heteroaryl moiety selected from Formulas III, IV, V. VI, VII, VIII, IX, X, or XI:

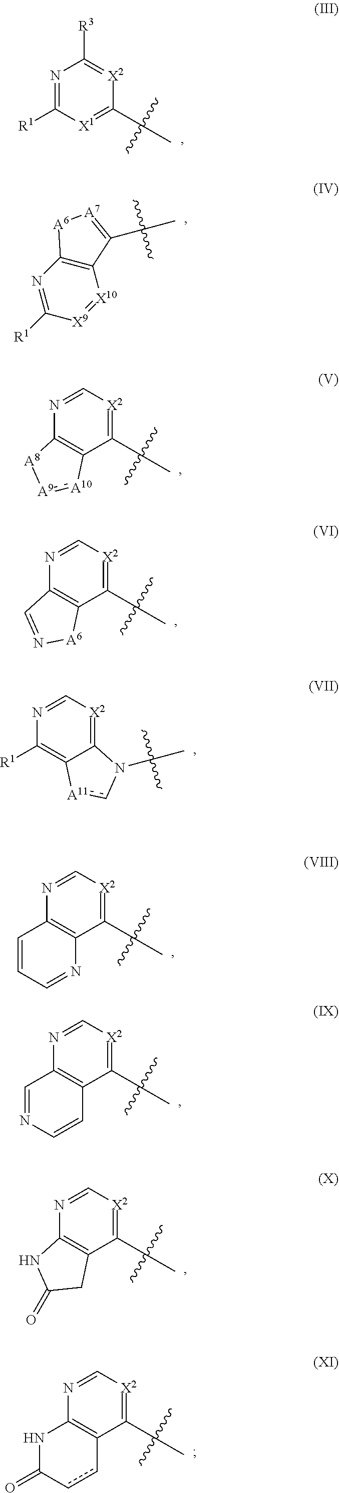

wherein the dotted lines in Formulas V, VII, and XI indicate a single or double bond;
A¹ is CR^{2e}, N, NR⁸, O or S, provided that the ring of which it is a member is a heteroaryl ring;
A² is C or N, provided that the ring of which it is a member is a heteroaryl ring;
A³ is CR²f N, NR⁸, O or S, provided that the ring of which it is a member is a heteroaryl ring;
A⁴ and A⁵ are each C or one is C and the other N, such that the ring of which they are members is a heteroaryl ring;
A⁶ is NR⁸, O, S, or S(═O);
A⁷ is CR^{2g} or N;
A⁸ is NR⁸, NHC(O), O, S, S(═O);
A⁹ is CH, CH₂, C(O), CR¹⁵, CR¹⁸, or N, provided that when A⁹ is CR¹⁵, A¹⁰ is CR¹⁶;
A¹⁰ is CH, CH₂, CR¹⁶, CR¹⁹, N, NH, or S, provided that when A¹⁰ is CR¹⁶, A⁹ is CR¹⁵;
A¹¹ is CH, CH₂ or N;
R is independently at each occurrence halo, NO₂, NR⁸R¹⁰, OR¹¹, SR¹², CN, COOR¹³, or a substituted or unsubstituted C₁₋₆ alkyl, C₃₋₇ cycloalkyl, or C₂₋₆ alkenyl group; or when m is at least 2, the two R moieties together form a C₁₋₄ alkylene bridge between non-adjacent ring members;
R¹, R^{2a}, R^{2b}, R^{2c}, R^{2d}, R^{2e}, R^{2f}, R^{2g}, and R³ are independently at each occurrence H, halo, NO₂, NR⁸R¹⁰, OR¹¹, SR¹², CN, COOR¹³, or a substituted or unsubstituted C₁₋₆ alkyl, C₃₋₇ cycloalkyl, or, C₂₋₆ alkenyl group;
R⁴ and R⁵ are independently H, halo, CN, OH, SR¹², NO₂, NR⁸R¹⁰, or a substituted or unsubstituted C₁₋₆ alkyl, C₁₋₆ alkoxy, or C₂₋₆ alkene; or R⁴ and R⁵ when present, together with the carbon atoms to which they are attached, form a fused phenyl or a 5- or 6-membered cycloalkenyl, heterocyclyl or heteroaryl ring;
R⁶ and R⁷ are independently H, NHR¹⁰, or a substituted or unsubstituted C₁₋₈-alkyl, C₂₋₈-alkenyl, cycloalkyl, cycloalkylalkyl, aryl, aralkyl, heterocyclyl, heterocyclylalkyl, heteroaryl, or heteroarylalkyl group; or R⁶ and R⁷ together with the carbon to which they are attached form a substituted or unsubstituted cycloalkyl or heterocyclyl ring;
R⁸ and R¹⁰ are independently at each occurrence H, an amino protecting group, or a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group; or R⁸ and R¹⁰ together with the nitrogen to which they are attached form a substituted or unsubstituted heterocyclyl ring;
R⁹ is at independently each occurrence H or substituted or unsubstituted alkyl group;
R¹¹ is independently at each occurrence H, a hydroxyl protecting group, or a substituted or unsubstituted alkyl, cycloalkyl, alkenyl, heterocyclyl, heteroaryl, aryl or aralkyl group;
R¹² is independently at each occurrence H, a thiol protecting group, or a substituted or unsubstituted alkyl, cycloalkyl, alkenyl, aryl or aralkyl group;
R¹³ is independently at each occurrence H or a substituted or unsubstituted alkyl, cycloalkyl, alkenyl, aryl or aralkyl group; and
R¹⁴ is H, OH, or a substituted or unsubstituted alkyl group;
R¹⁵ and R¹⁶, together with the carbons to which they are attached, form a cyclohexenyl ring, optionally substituted with C(O)R¹⁷, C(O)OR¹⁷, or C(O)NR⁸R¹⁰;
R¹⁷ is independently at each occurrence H or a substituted or unsubstituted alkyl, cycloalkyl, or alkenyl group;
R¹⁸ and R¹⁹ are independently selected from CN, C(O)R¹⁷, C(O)OR¹⁷, C(O)NR⁸R¹⁰, or a substituted or unsubstituted alkyl, cycloalkyl, or alkenyl group;
m is 0, 1, 2 or 3; and n is 1 or 2.

In any embodiments of the compounds of Formulas I or II, when X⁴ is CH, at least one of X⁷ and X⁸ may be a heteroatom, or n may be at least 2 and the two R moieties together form a C₁₋₄ alkylene bridge between non-adjacent ring members, or W¹ may be S(═O)₂, or X³ may be N(O).

In any embodiments of the compounds of Formulas I or II (including those where Z¹ and Z² are independently a moiety of Formula III, IV, V. VI, VII, VIII, IX, X, or XI), X¹ may be CR²a, e.g., CH or C—OCH₃. In any embodiments, X¹ may be N. In any embodiments, X² may be N. In any embodiments, X² may be CR^{2b}, e.g., CH. In any embodiments, X¹ may be CH or C—OCH₃ and X² may be N. In any embodiments, X¹ may be CH and X² may be N. In any embodiments, X¹ may be C—OCH₃ and X² may be N.

In any embodiments of the compounds, R¹ may be NH₂, NHC(O)-alkyl, or NHC(O)-cycloalkyl. In any embodiments, R¹ may be NH₂. In any embodiments, R¹ may be NR⁸R¹⁰ e.g., NHR¹⁰. In any embodiments, R¹ may be NHCH₃. In any embodiments, R¹⁰ may be a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group. In any embodiments, R¹⁰ may be a substituted or unsubstituted cycloalkyl, cycloalkylalkyl, aryl, aralkyl, heterocyclylalkyl, heterocyclyl, heteroaryl, or heteroarylene-heterocyclyl group. In any embodiments, R¹⁰ may be a substituted or unsubstituted alkyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group. For example, R¹⁰ may be substituted or unsubstituted C(O)—C₅-C₆ cycloalkyl, C(O)-phenyl, C(O)-pyrrolidinyl, C(O)-furan including tetrahydrofuran, C₁-C₃ alkyl-O—CH₃, C₁-C₃ alkyl-OH, (CH₂)₀₋₁—C₄-C₆ cycloalkyl, oxazolinyl, phenyl, furan including tetrahydrofuran, pyran including tetrahydropyran, imidazolyl, piperidinyl, pyrazolyl, pyrrolidinyl, pyridonyl, pyridinyl, pyridinyl-morpholinyl, pyridinyl-piperidinyl, or pyridinyl-piperazinyl group. In any embodiments, R¹⁰ may be phenyl, 3-methyl-pyridinyl, N-methyl-imidazolyl, or N-methyl-5-(4-methylpiperazin-1-yl)pyridinyl. In any embodiments, R¹⁰ may be selected from the group consisting of:

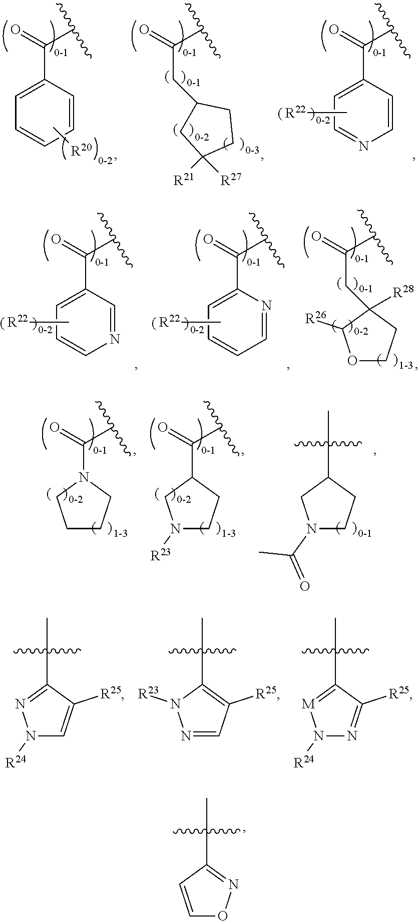

—CH₃, —(CH₂)₁₋₃OH, —(CH₂)₁₋₃OCH₃, —C(O)N(CH₃)₂, and —C(O)NHCH₃, wherein R²⁰ is halo, or N(CH₃)C(O)CH₃; R²¹ is H, OH, or OCH₃; R²² is CH₃, CH₂OH, —C(O)NHCH₃, —N(CH₃)C(O)CH₃,

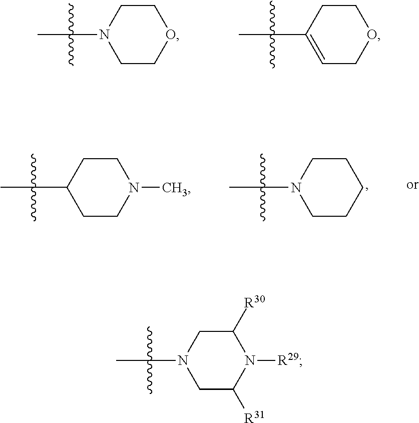

R²³ is H or C(O)CH₃; R²⁴ is H, CH₃, CF₃, (CH₂)₁₋₂OH, or CH(CH₃)₂; R²⁵ is H or CH₃; R²⁶ at each location is independently H or OH; R²⁷ is H or CH₃; R²⁸ is H or C(O)OCH₃; R²⁹ is H, CH₃, CH₂CH₃, or CH₂CH₂OH; R³⁰ and R³¹ are each independently H or CH₃; R³² is H or

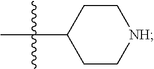 [SMILES: *C1CCNCC1]

and M is CH or N. In other embodiments, R⁸ and R¹⁰ together with the nitrogen to which they are attached form a substituted or unsubstituted heterocyclyl ring. In some such embodiments, R⁸ and R¹⁰ together with the nitrogen to which they are attached may be selected from the group consisting of:

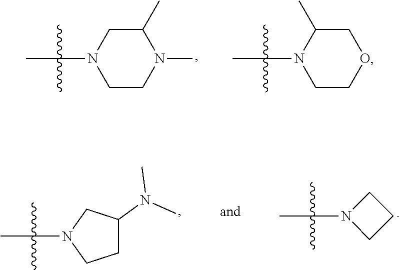

In any embodiments of compounds of Formulas I or II, Y¹ may be absent. In any embodiments, Y¹ may be NH. In any embodiments, Y² may be NH. In any embodiments, Y² may be absent. In any embodiments, Y¹ or Y² may be a 5-member heteroaryl group. For example, Y¹ or Y² may be an oxazole, isoxazole, thiazole, imidazole, oxadiazole, dioxazole, or isothiazole.

In any embodiments of the compounds herein, the compound may be a compound of Formula I. In any embodiments, X³ may be N, or X³ may be N(O) (i.e., an N-oxide). In any embodiments, X³ may be CR^{2c}. In any embodiments, X³ may be CH. In any embodiments, X³ may be C(O) and X⁶ may be N. In any embodiments, X⁴ may be CR⁴. In any embodiments, X⁴ may be CH. In any embodiments, X⁵ may be CR⁵. In any embodiments, R⁵ may be C₁-C₄ alkyl or C₂-C₃ alkenyl, e.g., methyl, ethyl, propyl, allyl. In any embodiments, X⁴ and X⁵ together form a fused phenyl, pyrrolinyl, or pyrrolyl ring.

In any embodiments, W¹ may be C(═O). In any embodiments, W¹ may be S(═O)₂.

As indicated by Formula I, the spirocyclic ring of compounds of Formula I may be a cycloalkyl or a heterocyclyl ring, including a bridged cycloalkyl or a bridged heterocyclyl ring. In any embodiments, X⁷ may be N(O) (i.e., the N-oxide), or X⁷ may be O or NH. In any embodiments, X⁷ may be S, SO, or SO₂. In any embodiments, X⁸ may be CH₂. In any embodiments X⁸ may be C(═O). In any embodiments, X⁸ may be NH. Thus, in any embodiments, X⁷ and X⁸ may together form for example NH—CH₂, O—CH₂, S(═O)₂—CH₂, C(═O)—NH, NH—C(═O), O—C(═O), S(═O)₂—NH, and the like. In any embodiments, m may be 0. In any embodiments, m may be 2 and the two R moieties together form a C₁₋₄ alkylene bridge between non adjacent ring members. For example, the two R moieties may form a methylene or ethylene bridge to provide a bridged cycloalkyl or heterocyclyl spirocycle. It will be understood by those of skill in the art that because the C₁₋₄ alkylene bridge may attach to any non-adjacent ring members, they may attach to one of X⁷ and X⁸, valence permitting. For example, if X⁷ is NH, the H may be substituted with an R group, and that R group may be one end of a C₁₋₄ alkylene bridge.

In any embodiments of the compounds herein, the compound of Formula I may be a compound of:

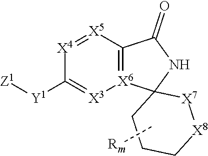

or a stereoisomer, tautomer, and/or pharmaceutically acceptable salt thereof.

In any embodiments of the compounds herein, the compound of Formula I may be a compound of Formula IA:

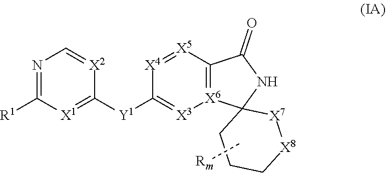

or a stereoisomer, tautomer, and/or pharmaceutically acceptable salt thereof.

Examples of compounds of Formula IA include, Formulas IAA-IAG:

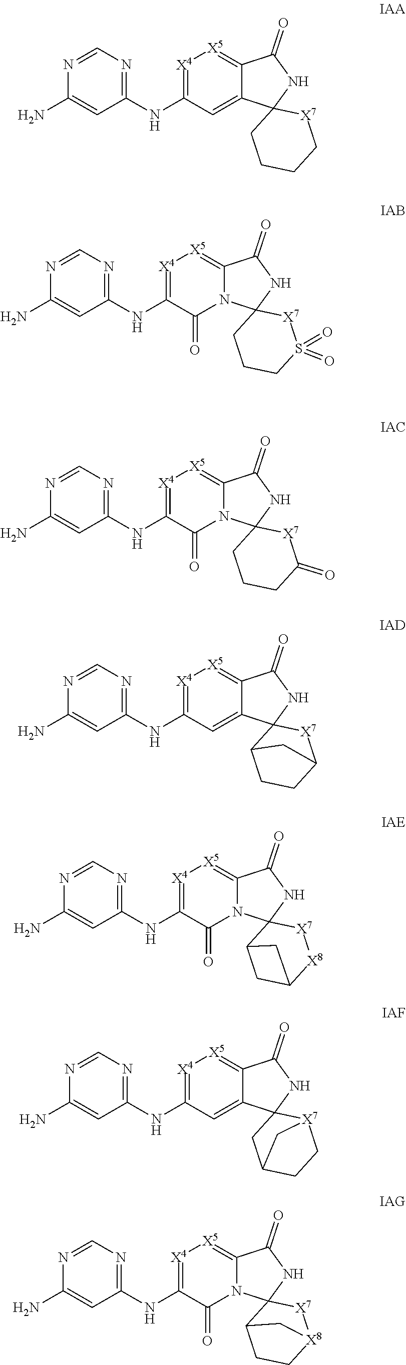

In any embodiments of the compounds herein, the compound may have the structure of Formula IB:

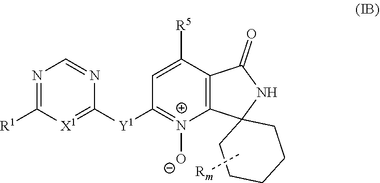

or a stereoisomer, tautomer, and/or pharmaceutically acceptable salt thereof.

In any embodiments of the compounds herein, the compound may have the structure of Formula IB-1:

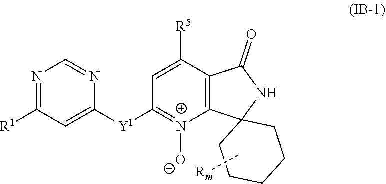

or a stereoisomer, tautomer, and/or pharmaceutically acceptable salt thereof.

In any embodiments of the compounds herein, the compound may have the structure of Formula II. In any embodiments, A¹ may be CH. In any embodiments A¹ may be N or NH. In any embodiments A¹ may be O. In any embodiments A¹ may be S. In any embodiments A² may be C. In any embodiments A² may be N. In any embodiments A³ may be S. In any embodiments, A¹ may be CH and A³ may be S. In any embodiments A³ may be N or NH. In any embodiments A³ may be CH. In any embodiments A³ may be O. In any embodiments A¹ may be N and A³ may be S. In any embodiments A⁴ may be C. In any embodiments A⁴ may be N. In any embodiments A¹ may be N and A⁴ may be N. In any embodiments A⁵ may be C. In any embodiments A⁵ may be N. In any embodiments, A¹ may be N and A⁵ may be N.

In any embodiments of compounds of Formula II, W² may be C(═O). In any embodiments, W² may be S(═O)₂.

In any embodiments of compounds of Formula III, Z¹ and/or Z² may be a moiety of Formula III, where R¹, R³, X¹ and X² may have any of the values disclosed herein and in any combination. In some embodiments, X² may be N. In some embodiments, X² may be CH. In any embodiments the Z¹ and/or Z² moiety of Formula III may be, e.g., 4-amino-1,3-pyrimidin-6-yl. In any embodiments where the Z¹ and/or Z² moiety of Formula III (e.g., 1,3-pyrimidin-6-yl), R¹ may be NR⁸R¹⁰ having any of the values disclosed herein. In any embodiments, Z¹ and/or Z² may be a moiety of Formula IV, where R¹, A⁶, A⁷, X⁹ and X¹⁰ may have any of the values disclosed herein and in any combination. In some such embodiments, A⁶ may be NH, and/or A⁷ may be N and/or X⁹ may be N. For example, the Z¹ and/or Z² moiety of Formula IV may be 6-amino-pyrazolo-pyrimidin-3-yl. In any embodiments, Z may be a moiety of Formula V where X², A⁸, A⁹ and A¹⁰ may have any of the values disclosed herein, and in any combination. In some such embodiments, X² may be N, and/or A⁸ may be NR⁸, and/or A⁹ may be CH or CR¹⁸, and/or A¹⁰ may be N, NH, CH, or CH₂. For example, Z¹ and/or Z² of Formula V may be a purine such as 9H-purine or 7H-pyrrolo[2,3-d]pyrimidine. In any embodiments where Z¹ and/or Z² is a moiety of Formula V, X² may be N, and/or A⁸ may be S, and/or A⁹ may be CH or CR¹⁸, and/or A¹⁰ may be N, NH, CH, or CH₂. In any embodiment, R¹⁸ may have any value disclosed herein, and in any combination. In any embodiment, R¹⁸ may be a substituted or unsubstituted alkyl or cycloalkyl group. In any embodiments where Z¹ and/or Z² is a moiety of Formula V, X² may be N, and/or A⁸ may be NR⁸, and/or A⁹ may be N, and/or A¹⁰ may be N, NH, CH, or CH₂. In any embodiments where Z¹ and/or Z² is a moiety of Formula V, X² may be N, and/or A⁸ may be S, and/or A⁹ may be N, and/or A¹⁰ may be N, NH, CH, or CH₂. In any embodiments where Z¹ and/or Z² is a moiety of Formula V, A⁹ and A¹⁰ may be, respectively, R¹⁵ and R¹⁶, such that together with the carbons to which they are attached, form an optionally substituted cyclohexenyl ring (i.e., and Z¹ and/or Z² is tricyclic (optionally substituted)). In any embodiment, the cyclohexenyl ring may be substituted with C(O)R¹⁷, C(O)OR¹⁷, or C(O)NR⁸R¹⁰. In any embodiment, the cyclohexenyl ring may be substituted with C(O)NR⁸R¹⁰, wherein R⁸ and R¹⁰ may have any of the values disclosed herein, and in any combination. In any embodiments, Z¹ and/or Z² may be a moiety of Formula VI where X² and A⁶ may have any of the values disclosed herein and in any combination. In some such embodiments X² may be N and A⁶ may be CH₂ or may be NH. In any embodiments, Z¹ and/or Z² may be a moiety of Formula VII, where R¹, X², and A¹¹ may have any of the values disclosed herein and in any combination. For example, in some such embodiments, R¹ may be NH₂ and/or X² may be N, and/or A¹¹ may be N or NH. In any embodiments, Z¹ and/or Z² may be a moiety of Formula VIII, where X² may have any of the values disclosed herein. In any embodiments, Z¹ and/or Z² may be a moiety of Formula IX, where X² may have any of the values disclosed herein and in any combination. In any embodiment, X² may be N. In any embodiments, Z¹ and/or Z² may be a moiety of Formula X, where X² may have any of the values disclosed herein and in any combination. In any embodiment, X² may be N. In any embodiments, Z¹ and/or Z² may be a moiety of Formula XI, where X² may have any of the values disclosed herein and in any combination. In any embodiment, X² may be N.

In any embodiments of compounds of Formula II, R⁶ and R⁷ are independently H or a substituted or unsubstituted C₁₋₈ alkyl, C₂₋₈ alkenyl, cycloalkyl, cycloalkylalkyl, aryl, aralkyl, heterocyclyl, heterocyclylalkyl, heteroaryl, or heteroarylalkyl group. In any embodiments, R⁶ and R⁷ are independently H or a substituted or unsubstituted C₁₋₈ alkyl, C₂₋₈ alkenyl, cycloalkyl, cycloalkylalkyl, arylalkyl, heterocyclylalkyl, or heteroarylalkyl group. In any embodiments, one of R⁶ and R⁷ is H and the other is a substituted or unsubstituted C₁₋₈ alkyl, C₂₋₈ alkenyl, cycloalkyl, cycloalkylalkyl, aryl, aralkyl, heterocyclyl, heterocyclylalkyl, heteroaryl, or heteroarylalkyl group. In any embodiments R⁶ and R⁷ are independently a substituted or unsubstituted C₁₋₈ alkyl. In any embodiments, one of R⁶ and R⁷ is H and the other is a substituted or unsubstituted C₁₋₈ alkyl. In any embodiments R⁶ and R⁷ are both H.

In any embodiments of compounds of Formula II, R⁶ and R⁷ together with the carbon to which they are attached form a 5- or 6-membered substituted or unsubstituted cycloalkyl or heterocyclyl ring. In any embodiments, the heterocyclyl ring may have 1 or two heteroatoms selected from N, O and S. In any embodiments, R⁶ and R⁷ together with the carbon to which they are attached form a substituted or unsubstituted cycloalkyl group. In any such embodiments, the cycloalkyl group may be unsubstituted and/or may be a bridged bicyclic cycloalkyl group. In any such embodiments, the cycloalkyl group may be a cyclohexyl or a bicyclo[2.2.1]heptanyl group. In other embodiments, R⁶ and R⁷ together may form a heterocyclyl group such as a pyranyl, thiopyranyl or oxides thereof, or piperidinyl group. In any embodiments, the cycloalkyl or heterocyclyl ring is substituted with 1, 2 or 3 substituents as defined herein.

In any embodiments, the compounds of Formula II may have a structure selected from the group consisting of Formula IIA, Formula IIB, Formula IIC, Formula IID, Formula IIE, and Formula IIF:

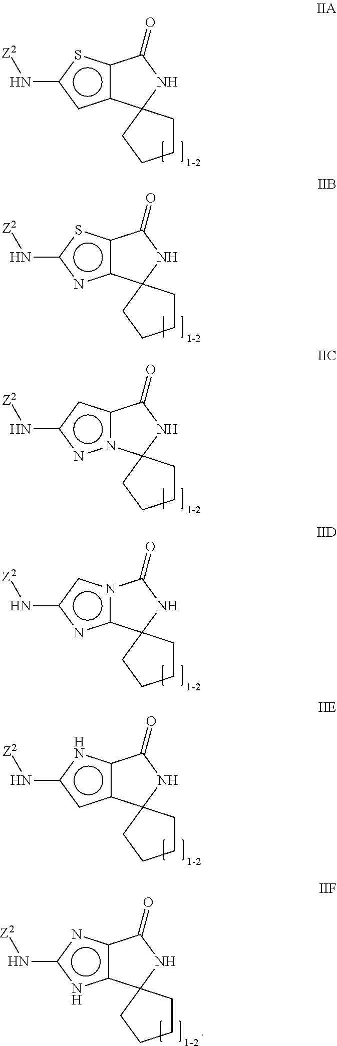

The compounds of Formulas IIA-IIF thus include a spirocyclic cyclopentyl or a spirocyclic cyclohexyl group, as well as any of the Z² moieties described herein.

In an aspect of the present technology, a composition is provided that includes any one of the aspects and embodiments of compounds disclosed herein (e.g., compounds of Formula I, IA, IAA-IAG, II, and IIA-IIF) and a pharmaceutically acceptable carrier. In a related aspect, a pharmaceutical composition is provided which includes an effective amount of the compound of any one of the aspects and embodiments of compounds described herein for treating an MNK-mediated disorder or condition (optionally including a pharmaceutically acceptable carrier and/or excipient(s)). The MNK-mediated disorder or condition may be selected from the group consisting of colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome.

In a further related aspect, a method of treatment is provided that includes administering an effective amount of a compound of any one of the aspects and embodiments described herein or administering a pharmaceutical composition comprising an effective amount of a compound of any one of the aspects and embodiments described herein to a subject suffering from an MNK-mediated disorder or condition. The MNK-mediated disorder or condition may be selected from the group consisting of colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome.

“Effective amount” refers to the amount of a compound or composition required to produce a desired effect. One example of an effective amount includes amounts or dosages that yield acceptable toxicity and bioavailability levels for therapeutic (pharmaceutical) use including, but not limited to, the treatment of hyperlipidemia. Another example of an effective amount includes amounts or dosages that are capable of reducing symptoms associated with metabolic syndrome, such as, for example, obesity and/or metabolic syndrome. The effective amount of the compound may selectively modulate MNK. As used herein, a “subject” or “patient” is a mammal, such as a cat, dog, rodent or primate. Typically the subject is a human, and, preferably, a human suffering from or suspected of suffering from an MNK-mediated disorder or condition. The term “subject” and “patient” can be used interchangeably.

In still another aspect, the present technology provides methods for inhibiting the activity of Mnk in at least one cell overexpressing Mnk, comprising contacting the at least one cell with an effective amount of any compound as described herein, including but not limited to a compound of Formula I, IA, IAA-IAG, II, or IIA-IIF. In some embodiments, the contacting takes place in vitro, e.g., as part of an assay.

Thus, the instant present technology provides pharmaceutical compositions and medicaments comprising any of the compounds disclosed herein (e.g., compounds of Formula I, IA, IAA-IAG, II, or IIA-IIF) and a pharmaceutically acceptable carrier or one or more excipients or fillers. The compositions may be used in the methods and treatments described herein. Such compositions and medicaments include a therapeutically effective amount of any compound as described herein, including but not limited to a compound of Formula I, IA, IAA-IAG, II, or IIA-IIF. The pharmaceutical composition may be packaged in unit dosage form.

The pharmaceutical compositions and medicaments may be prepared by mixing one or more compounds of the present technology, stereoisomers thereof, and/or pharmaceutically acceptable salts thereof, with pharmaceutically acceptable carriers, excipients, binders, diluents or the like to prevent and treat disorders associated with the effects of increased plasma and/or hepatic lipid levels. The compounds and compositions described herein may be used to prepare formulations and medicaments that prevent or treat a variety of disorders associated with or mediated by MNK, including, e.g., colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome. Such compositions can be in the form of, for example, granules, powders, tablets, capsules, syrup, suppositories, injections, emulsions, elixirs, suspensions or solutions. The instant compositions can be formulated for various routes of administration, for example, by oral, parenteral, topical, rectal, nasal, vaginal administration, or via implanted reservoir. Parenteral or systemic administration includes, but is not limited to, subcutaneous, intravenous, intraperitoneal, and intramuscular, injections. The following dosage forms are given by way of example and should not be construed as limiting the instant present technology.

For oral, buccal, and sublingual administration, powders, suspensions, granules, tablets, pills, capsules, gelcaps, and caplets are acceptable as solid dosage forms. These can be prepared, for example, by mixing one or more compounds of the instant present technology, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Compounds of the present technology may be administered to the lungs by inhalation through the nose or mouth. Suitable pharmaceutical formulations for inhalation include solutions, sprays, dry powders, or aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aqueous and nonaqueous (e.g., in a fluorocarbon propellant) aerosols are typically used for delivery of compounds of the present technology by inhalation.

Dosage forms for the topical (including buccal and sublingual) or transdermal administration of compounds of the present technology include powders, sprays, ointments, pastes, creams, lotions, gels, solutions, and patches. The active component may be mixed under sterile conditions with a pharmaceutically-acceptable carrier or excipient, and with any preservatives, or buffers, which may be required. Powders and sprays can be prepared, for example, with excipients such as lactose, talc, silicic acid, aluminum hydroxide, calcium silicates and polyamide powder, or mixtures of these substances. The ointments, pastes, creams and gels may also contain excipients such as animal and vegetable fats, oils, waxes, paraffins, starch, tragacanth, cellulose derivatives, polyethylene glycols, silicones, bentonites, silicic acid, talc and zinc oxide, or mixtures thereof. Absorption enhancers can also be used to increase the flux of the compounds of the present technology across the skin. The rate of such flux can be controlled by either providing a rate controlling membrane (e.g., as part of a transdermal patch) or dispersing the compound in a polymer matrix or gel.

Besides those representative dosage forms described above, pharmaceutically acceptable excipients and carriers are generally known to those skilled in the art and are thus included in the instant present technology. Such excipients and carriers are described, for example, in “Remingtons Pharmaceutical Sciences” Mack Pub. Co., New Jersey (1991), which is incorporated herein by reference.

The formulations of the present technology may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant present technology.

Those skilled in the art are readily able to determine an effective amount by simply administering a compound of the present technology to a patient in increasing amounts until for example, (for metabolic syndrome and/or obesity) the elevated plasma or elevated white blood cell count or hepatic cholesterol or triglycerides or progression of the disease state is reduced or stopped. For metabolic syndrome and/or obesity, the progression of the disease state can be assessed using in vivo imaging, as described, or by taking a tissue sample from a patient and observing the target of interest therein.

The compounds of the present technology can be administered to a patient at dosage levels in the range of about 0.1 to about 1,000 mg per day. For a normal human adult having a body weight of about 70 kg, a dosage in the range of about 0.01 to about 100 mg per kg of body weight per day is sufficient. The specific dosage used, however, can vary or may be adjusted as considered appropriate by those of ordinary skill in the art. For example, the dosage can depend on a number of factors including the requirements of the patient, the severity of the condition being treated and the pharmacological activity of the compound being used. The determination of optimum dosages for a particular patient is well known to those skilled in the art.

Various assays and model systems can be readily employed to determine the therapeutic effectiveness of the treatment according to the present technology.

Effectiveness of the compositions and methods of the present technology may also be demonstrated by a decrease in the symptoms of cancer, such as, for example, a decrease in tumor growth rate, inhibition of tumor growth, or shrinkage of a tumor. Effectiveness of the compositions and methods of the present technology may also be demonstrated by a decrease in the signs and symptoms of various cancers listed herein.

For each of the indicated conditions described herein, test subjects will exhibit a 10%, 20%, 30%, 50% or greater reduction, up to a 75-90%, or 95% or greater, reduction, in one or more symptom(s) caused by, or associated with, the disorder in the subject, compared to placebo-treated or other suitable control subjects.

The compounds of the present technology can also be administered to a patient along with other conventional therapeutic agents that may be useful in the treatment of a MNK-mediated disorder or condition such as colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome. The administration may include oral administration, parenteral administration, or nasal administration. In any of these embodiments, the administration may include subcutaneous injections, intravenous injections, intraperitoneal injections, or intramuscular injections. In any of these embodiments, the administration may include oral administration. The methods of the present technology can also comprise administering, either sequentially or in combination with one or more compounds of the present technology, a conventional therapeutic agent in an amount that can potentially be effective for the treatment of any one or more of the foregoing MNK-mediated disorders or conditions.

In one aspect, a compound of the present technology is administered to a patient in an amount or dosage suitable for therapeutic use. Generally, a unit dosage comprising a compound of the present technology will vary depending on patient considerations. Such considerations include, for example, age, protocol, condition, sex, extent of disease, contraindications, concomitant therapies and the like. An exemplary unit dosage based on these considerations can also be adjusted or modified by a physician skilled in the art. For example, a unit dosage for a patient comprising a compound of the present technology can vary from 1×10⁻⁴ g/kg to 1 g/kg, or from 1×10⁻³ g/kg to 1 g/kg. Dosage of a compound of the present technology can also vary from 0.01 mg/kg to 10 or 50 or 100 mg/kg or from 0.1 mg/kg to 10 mg/kg.

The examples herein are provided to illustrate advantages of the present technology and to further assist a person of ordinary skill in the art with preparing or using the compounds of the present technology or salts, pharmaceutical compositions, derivatives, solvates, metabolites, prodrugs, racemic mixtures or tautomeric forms thereof. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### EXAMPLES

### General Synthetic and Analytical Details

All reagents and materials are or were purchased from commercial vendors. A list of abbreviations for reagents used may be found in Table 1, below.

TABLE 1

| Abbreviation | Reagent or Moiety |
|---|---|
| AIBN | azobisisobutyronitrile |
| Ad2PBu | butyldi-1-adamantylphosphine |
| t-Bu | tert-butyl |
| BPO | benzoyl peroxide |
| BrettPhos | 2-(dicyclohexylphosphino)3,6-dimethoxy- |
| | 2′,4′,6′-triisopropyl-1,1′-biphenyl |
| CDI | 1,1′-carbonyldiimidazole |
| m-CPBA | m-chloroperoxybenzoic acid |
| DCM | dichloromethane |
| DIAD | diisopropyl azodicarboxylate |
| DMF | dimethylformamide |
| DMA | dimethylacetamide |
| DMSO | dimethyl sulfoxide |
| EtOH | ethyl alcohol |
| Et | ethyl |
| EtOAc | ethyl acetate |
| Me | methyl |
| MeOH | methanol |
| NCS | N-chlorosuccinimide |
| NBS | N-bromosuccinimide |
| NIS | N-iodosuccinimide |
| Pd₂(dba)₃ | tris(dibenzylideneacetone)dipalladium |
| Pd(dppf)Cl₂ | [1,1′-bis(diphenylphosphino)ferrocene] |
| | palladium(II) dichloride |
| Ph | phenyl |
| PMBCl | 4-methoxybenzyl chloride |
| TEA | triethylamine |
| TFA | trifluoroacetic acid |
| TFAA | trifluoroacetic anhydride |
| THF | tetrahydrofuran |
| Xantphos | 4,5-bis(diphenylphosphino)-9,9-dimethylxanthene |

### Representative General Synthetic Schemes

The following compounds were or can be prepared as indicated in the following synthetic schemes using procedures known to those of ordinary skill in the art.

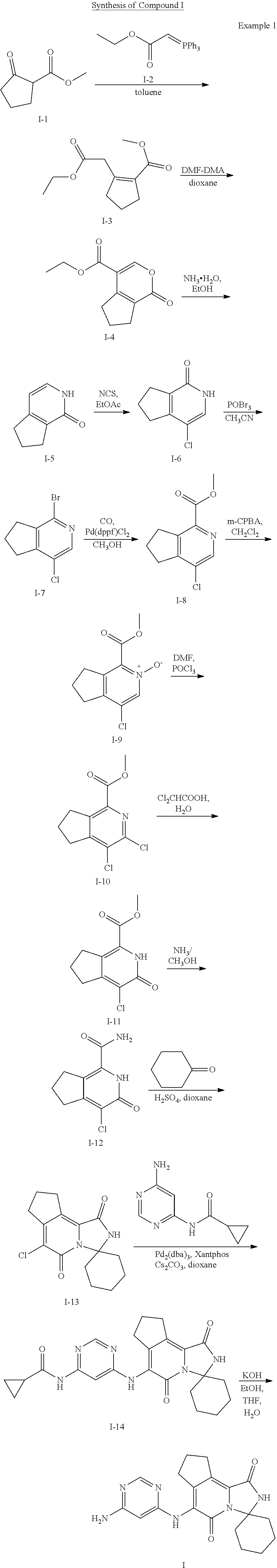

Methyl 2-(2-ethoxy-2-oxoethyl)cyclopent-1-ene-1-carboxylate (Compound I-3): To a solution of I-1 (30.0 g, 211.04 mmol) in toluene (300.0 mL) was added I-2 (81.6 g, 234.23 mmol) The resulting mixture was stirred at 110° C. for 48 h. After the reaction was complete, the mixture was cooled to room temperature. The resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (20/1, v/v) to afford the title compound (26.7 g, 60%) as a yellow oil. LCMS (ESI, m/z): [M+H]⁺=213.1.

Ethyl 1-oxo-5H,6H,7H-cyclopenta[c]pyran-4-carboxylate (Compound I-4): To a solution of I-3 (26.7 g, 125.80 mmol) in dioxane (240.0 mL) was added DMF-DMA (180.0 mL, 1344.36 mmol). The resulting mixture was stirred at 120° C. for 12 h. After the reaction was complete, the resulting mixture was cooled to room temperature and concentrated under reduced pressure. The residue was purified by flash column chromatography with petroleum ether/ethyl acetate (10/1, v/v) to afford the title compound (18.6 g, 71%) as a white solid. LCMS (ESI, m/z): [M+H]⁺=209.1.

2H,5H,6H,7H-Cyclopenta[c]pyridin-1-one (Compound I-5): To a solution of I-4 (18.6 g, 89.33 mmol) in EtOH (240.0 mL) was added NH₃·H₂O (120.0 mL). The resulting mixture was stirred at 78° C. for 2 h. After the reaction was complete, the resulting mixture was cooled to room temperature and concentrated under reduced pressure. The residue was purified by flash column chromatography with CH₂Cl₂/MeOH (10/1, v/v) to afford the title compound (3.4 g, 28%) as a yellow solid. LCMS (ESI, m/z): [M+H]⁺=136.1.

4-Chloro-2H,5H,6H,7H-cyclopenta[c]pyridin-1-one (Compound I-6): To a solution of I-5 (3.4 g, 25.18 mmol) in ethyl acetate (200.0 mL) was added NCS (3.3 g, 24.81 mmol). The resulting mixture was stirred at room temperature for 16 h. After the reaction was complete, the resulting mixture was concentrated under reduced pressure. The residue was purified by flash column chromatography with CH₂Cl₂/MeOH (40/1, v/v) to afford the title compound (2.5 g, 59%) as a pink solid. LCMS (ESI, m/z): [M+H]⁺=170.0.

1-Bromo-4-chloro-5H,6H,7H-cyclopenta[c]pyridine (Compound I-7): To a solution of I-6 (2.5 g, 14.74 mmol) in CH₃CN (200.0 mL) was added phosphoryl bromide (42.5 g, 148.25 mmol). The resulting mixture was stirred at 80° C. for 24 h. After the reaction was complete, the resulting mixture was cooled to room temperature and quenched with water at 0° C. The mixture was extracted with ethyl acetate. The combined organic layer was washed with brine, dried over anhydrous sodium sulfate and filtered. The filtrate was concentrated under vacuum. The residue was purified by reverse phase flash chromatography with CH₃CN/H₂O (50/50, v/v) to afford the title compound (793.8 mg, 23%) as a black solid. LCMS (ESI, m/z): [M+H]⁺=231.9.

Methyl 4-chloro-5H,6H,7H-cyclopenta[c]pyridine-1-carboxylate (Compound I-8): To a solution of I-7 (793.8 mg, 3.41 mmol) in CH₃OH (30.0 mL) was added Pd(dppf)Cl₂ (503.1 mg, 0.69 mmol) and TEA (1561.9 mg, 15.44 mmol). The resulting mixture was stirred at room temperature for 16 h under CO. After the reaction was complete, the resulting mixture was concentrated under reduced pressure. The residue was purified by flash column chromatography with Petroleum ether/ethyl acetate (80/20, v/v) to afford methyl the title compound (610.5 mg, 80%) as a white solid. LCMS (ESI, m/z): [M+H]⁺=212.0.

4-Chloro-1-(methoxycarbonyl)-5H,6H,7H-cyclopenta[c]pyridin-2-ium-2-olate (Compound I-9): To a solution of I-8 (130.0 mg, 0.61 mmol) in CH₂Cl₂ (10.0 mL) was added m-CPBA (330.7 mg, 1.92 mmol). The resulting mixture was stirred at room temperature for 16 h. After the reaction was complete, the resulting mixture was quenched with TMSCHN₂. The mixture was concentrated under reduced pressure. The residue was purified by flash column chromatography with petroleum ether/ethyl acetate (40/60, v/v) to afford the title compound (136.3 mg, 97%) as a white solid. LCMS (ESI, m/z): [M+H]⁺=228.0.

Methyl 3,4-dichloro-6,7-dihydro-5H-cyclopenta[c]pyridine-1-carboxylate (Compound I-10): To a solution of I-9 (190.0 mg, 0.84 mmol) in DMF (5.0 mL) was added POCl₃ (256.0 mg, 1.67 mmol). The resulting mixture was stirred at 0° C. for 1 h and then stirred at room temperature for 16 h. After the reaction was complete, the reaction was diluted with H₂O and filtered. The solid was washed with H₂O and dried to afford the title compound (167.4 mg, crude) as a white solid. LCMS (ESI, m/z): [M+H]⁺=246.0.

Methyl 4-chloro-3-oxo-2H,5H,6H,7H-cyclopenta[c]pyridine-1-carboxylate (Compound I-11): To a solution of I-10 (157.4 mg, 0.64 mmol) in water (0.5 mL) was added Cl₂CHCOOH (5.0 mL). The resulting mixture was stirred at 120° C. for 16 h. After the reaction was complete, the mixture was cooled to room temperature. The pH value of the mixture was adjusted to 7 with saturated NaHCO₃ (aq.). The resulting mixture was extracted with CH₂Cl₂. The combined organic layer was washed with brine, dried over anhydrous Na₂SO₄ and filtered. The filtrate was concentrated under vacuum. The residue was purified by flash column chromatography with CH₂Cl₂/MeOH (90/10, v/v) to afford the title compound (220.0 mg, crude) as a white solid. LCMS (ESI, m/z): [M+H]⁺=228.0.

4-Chloro-3-oxo-2H,5H,6H,7H-cyclopenta[c]pyridine-1-carboxamide (Compound I-12): The solution of I-11 (190.0 mg, 0.84 mmol) in NH₃/MeOH (12.0 mL, 7 mol/L) was stirred at 60° C. for 16 h. After the reaction was completed, the resulting mixture cooled to room temperature. The resulting mixture was concentrated under reduced pressure to afford the title compound (171.1 mg, crude) as a yellow solid. LCMS (ESI, m/z): [M+H]⁺=213.0.

6′-Chloro-8′,9′-dihydrospiro[cyclohexane-1,3′-cyclopenta[c]imidazo[1,5-a]pyridine]-1′,5′(2′H,7′H)-dione (Compound I-13): To a solution of I-12 (152.3 mg, 0.72 mmol) in 1,4-dioxane (10.0 mL) was added cyclohexanone (647.5 mg, 6.60 mmol) and H₂SO₄ (30.2 mg, 0.31 mmol). The resulting mixture was stirred at 100° C. for 16 h. After the reaction was complete, the resulting mixture was cooled to room temperature and concentrated under reduced pressure. The residue was purified by reverse phase flash chromatography with MeCN/H₂O (50/50, v/v) to afford the title compound (158.0 mg, 22%) as a yellow solid. LCMS (ESI, m/z): [M+H]⁺=293.1.

N-(6-((1′,5′-Dioxo-1′,2′,5′,7′,8′,9′-hexahydrospiro[cyclohexane-1,3′-cyclopenta[c]imidazo[1,5-a]pyridin]-6′-yl)amino)pyrimidin-4-yl)cyclopropanecarboxamide (Compound I-14): To a solution of I-13 (181.6 mg, 0.62 mmol) in dioxane (20.0 mL) was added tert-butyl N-(6-aminopyrimidin-4-yl)carbamate (202.6 mg, 0.96 mmol), Pd₂(dba)₃ (102.5 mg, 0.11 mmol), XantPhos (108.0 mg, 0.19 mmol) and Cs₂CO₃ (703.0 mg, 2.63 mmol). The resulting mixture was stirred at 100° C. for 16 h under N₂. After the reaction was completed, the reaction mixture was cooled to room temperature and then filtered. The filtrate was concentrated under reduced pressure. The residue was purified by flash column chromatography with CH₂Cl₂/MeOH (90/10, v/v) and then purified by reverse phase flash chromatography with MeOH/H₂O (50/50, v/v) to afford the title compound (37.1 mg, 8%) as a yellow solid. LCMS (ESI, m/z): [M+H]⁺=435.2.

6′-((6-Aminopyrimidin-4-yl)amino)-8′,9′-dihydrospiro[cyclohexane-1,3′-cyclopenta[c]imidazo[1,5-a]pyridine]-1′,5′(2′H,7′H)-dione (Compound I): To a solution of I-14 (37.1 mg, 0.09 mmol) in EtOH (2.0 mL), H₂O (1.0 mL) and THF (1.0 mL) was added KOH (28.7 mg, 0.51 mmol). The resulting mixture was stirred at 30° C. for 16 h. After the reaction was complete the reaction, the resulting mixture was concentrated under reduced pressure. The residue was purified by Prep-HPLC with the following conditions: Column: XBridge Prep OBD C18 Column, 19×250 mm, 5 um; Mobile Phase A: Water (10 mmol/L NH₄HCO₃), Mobile Phase B: MeOH-HPLC; Flow rate: 25 mL/min; Gradient: 38% B to 55% B in 7 min; 254 nm; to afford the title compound (8.9 mg, 28%) as a white solid. LCMS (ESI, m/z): [M+H]⁺=367.2. ¹H NMR (300 MHz, DMSO-d₆): δ 10.14 (s, 1H), 8.23 (s, 1H), 7.94 (s, 1H), 6.30 (s, 2H), 5.62 (s, 1H), 3.00-2.95 (m, 4H), 2.62-2.58 (m, 2H), 1.98-1.93 (m, 2H), 1.72-1.59 (m, 5H), 1.45-1.41 (m, 2H), 1.24-1.15 (m, 1H).

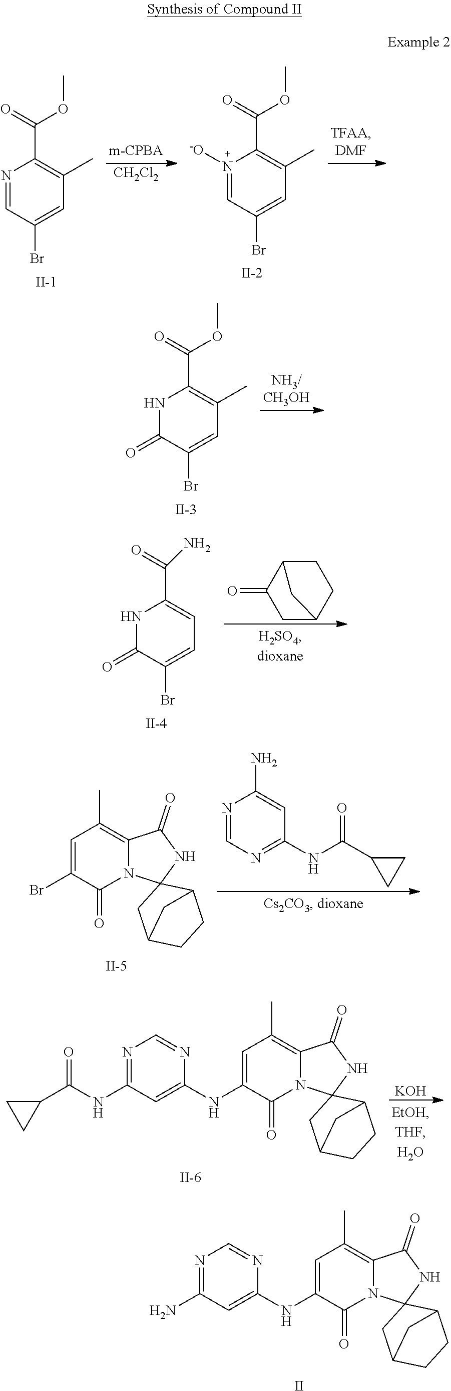

5-Bromo-2-(methoxycarbonyl)-3-methylpyridin-1-ium-1-olate (Compound II-2): To a stirred solution of II-1 (5.9 g, 25.64 mmol) in DCM (150.0 mL) was added m-CPBA (13.3 g, 76.94 mmol). The resulting mixture was stirred at room temperature for 16 h. The resulting mixture was concentrated under reduced pressure. The residue was purified by flash column chromatography with petroleum ether/EtOAc to afford the title compound (4.3 g, 66%) as a yellow solid. LCMS (ESI, m/z): [M+H]⁺=246.0.

Methyl 5-bromo-3-methyl-6-oxo-1H-pyridine-2-carboxylate (Compound II-3): To a solution of II-2 (4.3 g, 17.47 mmol) in DMF (50.0 mL) was added and TFAA (40.4 g, 192.23 mmol). The resulting mixture was stirred at room temperature for 16 h. After the reaction was completed, the reaction mixture was purified by reverse phase flash chromatography with CH₃CN/water (80/20, v/v) to afford the title compound (2.0 g, 46%) as a light yellow solid. LCMS (ESI, m/z): [M+H]⁺=246.0.

5-Bromo-3-methyl-6-oxo-1H-pyridine-2-carboxamide (Compound II-4): The solution of II-3 (2.0 g, 8.05 mmol) in NH₃/MeOH (50.0 mL, 7 mol/L) was stirred at 60° C. for 16 h. The resulting mixture was concentrated under reduced pressure. The residue was washed with Et₂O and filtered. The solid was collected and dried to afford the title compound (1.9 g, crude) as a light yellow solid. LCMS (ESI, m/z): [M+H]⁺=231.0.

6′-Bromo-8′-methyl-2′H-spiro[bicyclo[2.2.1]heptane-2,3′-imidazo[1,5-a]pyridine]-1′,5′-dione (Compound II-5): To a stirred solution of II-4 (1.0 g, 4.54 mmol) in 1,4-dioxane was added H₂SO₄ (0.1 mL, 0.47 mmol). The resulting mixture was stirred at 100° C. for 16 h. The resulting mixture was concentrated under reduced pressure. The residue was purified by flash column chromatography with petroleum ether/EtOAc to afford the title compound (428.3 mg, 29%) as a light yellow solid. LCMS (ESI, m/z): [M+H]⁺=323.0.

N-(6-((8′-Methyl-1′,5′-dioxo-1′,5′-dihydro-2′H-Spiro[bicyclo[2.2.1]heptane-2,3′-imidazo[1,5-a]pyridin]-6′-yl)amino)pyrimidin-4-yl)cyclopropanecarboxamide (Compound II-6): A mixture of II-5 (409.3 mg, 1.27 mmol), N-(6-aminopyrimidin-4-yl)cyclopropanecarboxamide (248.2 mg, 1.39 mmol), Pd₂(dba)₃ (150.7 mg, 0.16 mmol), XantPhos (190.5 mg, 0.33 mmol) and Cs₂CO₃ (916.0 mg, 2.81 mmol) in dioxane (10.0 mL) was stirred at 100° C. for 16 h. After the reaction was completed, the resulting mixture was filtered. The filtrate was concentrated under reduced pressure. The residue was purified by flash column chromatography with petroleum ether/EtOAc to afford the title compound (547.9 mg, 98%) as a light yellow solid. LCMS (ESI, m/z): [M+H]⁺=421.0.

(1R,2R,4S)-6′-((6-Aminopyrimidin-4-yl)amino)-8′-methyl-2′H-spiro[bicyclo[2.2.1]heptane-2,3′-imidazo[1,5-a]pyridine]-1′,5′-dione & (1R,2S,4S)-6′-((6-aminopyrimidin-4-yl)amino)-8′-methyl-2′H-spiro[bicyclo[2.2.1]heptane-2,3′-imidazo[1,5-a]pyridine]-1′,5′-dione (Compound II): To a stirred solution of II-6 (547.9 mg, 1.30 mmol) in THF (5.0 mL) and EtOH (5.0 mL) was added a solution of KOH (177.7 mg, 3.17 mmol) in H₂O (5.0 mL). The resulting mixture was stirred at room temperature for 16 h. The resulting mixture was concentrated under reduced pressure. The residue was purified by reverse phase flash column chromatography with CH₃CN/H₂O (60/40, v/v) and then purified by Prep-HPLC with the following conditions: Column: XBridge Prep Phenyl OBD Column, 5 um, 19×250 mm; Mobile Phase A: Water (10 mmol/L NH₄HCO_{3+0.1}% NH₃·H₂O), Mobile Phase B: MeOH-HPLC; Flow rate: 25 mL/min; Gradient: 45% B to 61% B in 12 min; 254/220 nm; RT1:10.9/11.53 min to afford two diastereomeric compounds: Compound IIa: (17.9 mg, 4%) as a white solid. Compound IIb: (6.3 mg, 2%) as a white solid.

Compound IIIa: LCMS (ESI, m/z): [M+H]⁺=353.2. ¹H NMR (400 MHz, DMSO-d₆): δ9.51 (s, 1H), 8.63 (s, 1H), 8.37 (s, 1H), 8.17 (s, 1H), 6.52 (s, 2H), 6.17 (d, J=0.4 Hz, 1H), 3.51-3.48 (m, 1H), 3.10-3.07 (m, 1H), 2.45-2.43 (m, 4H), 2.34-2.30 (m, 1H), 1.76-1.74 (m, 1H), 1.58-1.55 (m, 4H), 1.50-1.49 (m, 1H).

Compound IIb: LCMS (ESI, m/z): [M+H]⁺=353.2. ¹H NMR (300 MHz, DMSO-d₆): δ 9.61 (s, 1H), 8.67 (s, 1H), 8.40 (s, 1H), 8.17 (s, 1H), 6.52 (s, 2H), 6.22 (s, 1H), 3.19-3.15 (m, 1H), 2.43-2.40 (m, 4H), 2.28-2.23 (m, 1H), 1.94-1.83 (m, 3H), 1.63-1.27 (m, 4H).

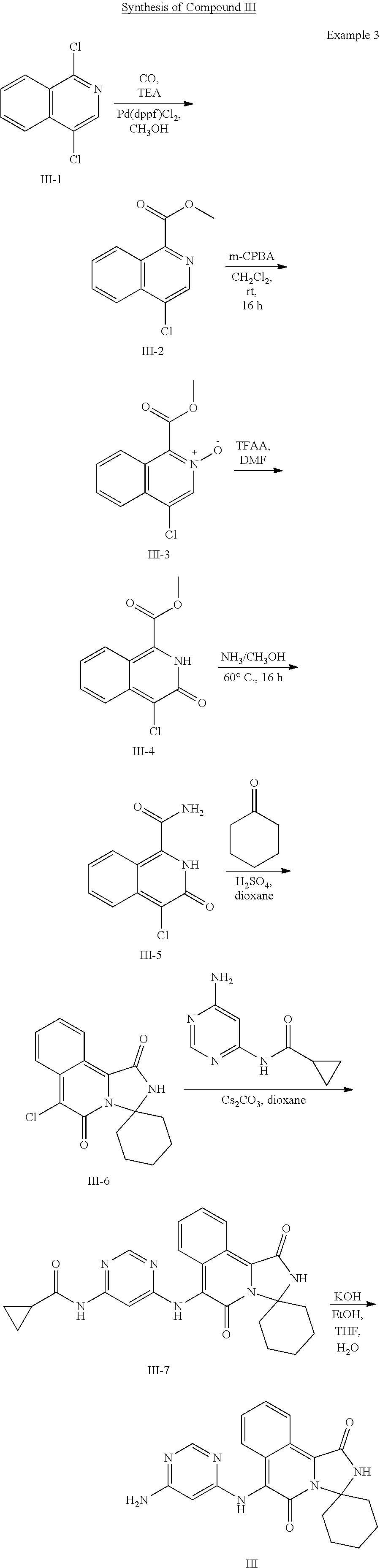

Methyl 4-chloroisoquinoline-1-carboxylate (Compound III-2): To a solution of III-1 (1.1 g, 5.35 mmol) in CH₃OH (30.0 mL) was added Pd(dppf)Cl₂ (681.0 mg, 0.93 mmol) and TEA (2.3 g, 22.73 mmol). The resulting mixture was stirred at room temperature for 16 h under CO. After the reaction was complete, the mixture was concentrated under reduced pressure. The residue was purified by flash column chromatography with Petroleum ether/EtOAc (80/20, v/v) to afford the title compound (940.0 mg, 79%) as a white solid. LCMS (ESI, m/z): [M+H]⁺=222.0.

4-Chloro-1-(methoxycarbonyl)isoquinolin-2-ium-2-olate (Compound III-3): To a solution of III-2 (940.0 mg, 4.24 mmol) in CH₂Cl₂ (30.0 mL) was added m-CPBA (2.3 g, 13.50 mmol). The reaction mixture was stirred at room temperature for 16 h. After the reaction was complete, the mixture was quenched with TMSCHN₂ and then concentrated under reduced pressure. The residue was purified by flash column chromatography with Petroleum ether/EtOAc (40/60, v/v) to afford the title compound (480.0 mg, 48%) as a light yellow solid. LCMS (ESI, m/z): [M+H]⁺=238.0.

Methyl 4-chloro-3-oxo-2H-isoquinoline-1-carboxylate (Compound III-4): To a solution of III-3 (480.0 mg, 2.02 mmol) in DMF (8.00 mL) was added TFAA (4.51 g, 21.47 mmol). The reaction mixture was stirred at room temperature for 16 h. After the reaction was complete, the residue was purified by reverse phase flash column chromatography with ACN/H₂O (80/20, v/v) to afford the title compound (417.0 mg, 87%) as a yellow solid. LCMS (ESI, m/z): [M+H]⁺=238.0.

4-Chloro-3-oxo-2H-isoquinoline-1-carboxamide (Compound III-5): The solution of III-4 (443.0 mg, 1.86 mmol) in NH₃/MeOH (20.0 mL, 7 mol/L) was stirred at 60° C. for 16 h. After the reaction was complete, the mixture was concentrated under vacuum. The residue was washed with Et₂O and filtered. The solid was collected and dried to afford the title compound (410.6 mg, crude) as a yellow solid. LCMS (ESI, m/z): [M+H]⁺=223.0.

6′-Chloro-2′H-spiro[cyclohexane-1,3′-imidazo[4,3-a]isoquinoline]-1′,5′-dione (Compound III-6): To a solution of III-5 (290.0 mg, 1.30 mmol) in dioxane (8.0 mL) was added cyclohexanone (1.1 g, 11.22 mmol) and H₂SO₄ (23.7 mg, 0.24 mmol). The reaction mixture was stirred at 100° C. for 16 h. After the reaction was complete, the reaction mixture concentrated under vacuum. The residue was washed with Et₂O and filtered. The solid was collected and dried to afford the title compound (399.0 mg, crude) as a yellow solid. LCMS (ESI, m/z): [M+H]⁺=303.1.

N-(6-[1′,5′-Dioxo-2′H-spiro[cyclohexane-1,3′-imidazo[4,3-a]isoquinolin]-6′-ylamino]pyrimidin-4-yl)cyclopropanecarboxamide (Compound III-7): To a mixture of III-6 (359.0 mg, 1.18 mmol) dioxane (20.0 mL) was added N-(6-aminopyrimidin-4-yl)cyclopropanecarboxamide (387.0 mg, 2.17 mmol), Pd₂(dba)₃ (190.7 mg, 0.21 mmol), XantPhos (208.8 mg, 0.36 mmol) and Cs₂CO₃ (1.3 g, 4.11 mmol). The reaction mixture was stirred at 110° C. for 16 h. After the reaction was complete, the mixture was filtered. The filtrate was concentrated under vacuum. The residue was purified by flash column chromatography with CH₂Cl₂/MeOH (94/6, v/v) and then purified by reverse phase flash column chromatography CH₃OH/H₂O (70/30, v/v) to afford the title compound (321.0 mg, 61%) as a yellow solid. LCMS (ESI, m/z): [M+H]⁺=445.2.

6′-((6-Aminopyrimidin-4-yl)amino)-2′H-spiro[cyclohexane-1,3′-imidazo[5,1-a]isoquinoline]-1′,5′-dione (Compound III): To a solution of III-7 (300.0 mg, 0.67 mmol) in THF (6.0 mL), EtOH (12.0 mL) and H₂O (6.0 mL) was added KOH (227.2 mg, 4.00 mmol). The reaction mixture was stirred at room temperature for 16 h. After the reaction was complete, the resulting mixture was concentrated under vacuum. The residue was purified by flash chromatography with CH₃OH/H₂O (70/30, v/v) and then purified by Prep-HPLC with the following conditions: Column: Column: XBridge Prep OBD C18 Column, 19×250 mm, 5 um; Mobile Phase A: Water (10 mmol/L NH₄HCO₃), Mobile Phase B: MeOH-HPLC; Flow rate: 25 mL/min; Gradient: 32% B to 56% B in 7 min; 254 nm; to afford the title compound (38.3 mg, 15%) as a yellow solid. LCMS (ESI, m/z): [M+H]⁺=377.2. ¹H NMR (300 MHz, DMSO-d₆): δ 10.71 (s, 1H), 8.80 (d, J=9.0 Hz, 1H), 8.34 (d, J=7.8 Hz, 1H), 7.88 (d, J=0.6 Hz, 1H), 7.38-7.31 (m, 2H), 7.16-7.10 (m, 1H), 6.46 (s, 2H), 5.44 (s, 1H), 3.32-3.17 (m, 2H), 1.89-1.60 (m, 5H), 1.56-1.45 (m, 2H), 1.32-1.22 (m, 1H).

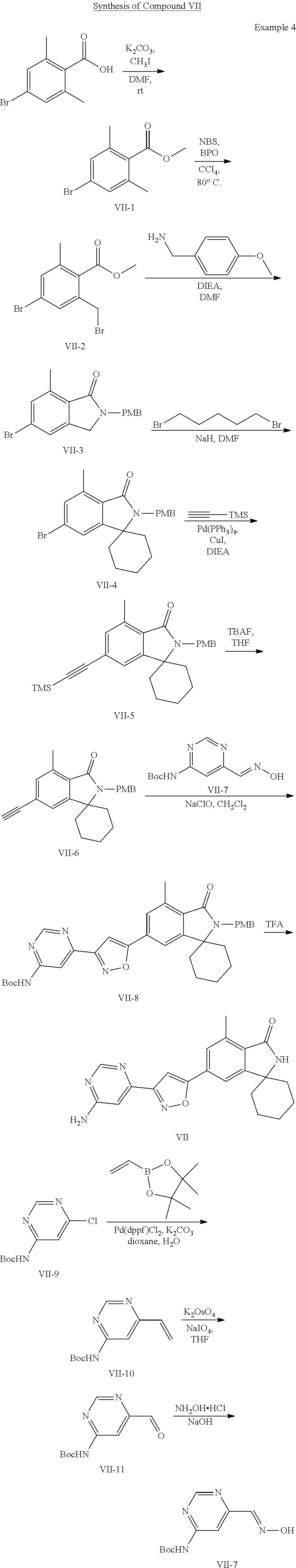

tert-Butyl N-(6-ethenylpyrimidin-4-yl)carbamate (Compound VII-10): To a solution of tert-butyl (6-chloropyrimidin-4-yl)carbamate (2.0 g, 8.75 mmol) in H₂O (5.0 mL) and dioxane (50.0 mL) was added 2-ethenyl-4,4,5,5-tetramethyl-1,3,2-dioxaborolane (1.5 g, 9.62 mmol), K₂CO₃ (3.6 g, 26.24 mmol) and Pd(dppf)Cl₂ (0.6 g, 0.88 mmol). The reaction mixture was stirred at 100° C. for 16 h under N₂. After the reaction was completed, the reaction mixture was cooled to room temperature and filtered. The filtrate was concentrated under reduced pressure. The residue was purified by flash column chromatography with petroleum ether/ethyl acetate (75/25, v/v) to afford tert-butyl N-(6-ethenylpyrimidin-4-yl)carbamate (1.4 g, 70%) as a white solid. LCMS (ESI, m/z): [M+H]⁺=222.1.

tert-Butyl N-(6-formylpyrimidin-4-yl)carbamate (Compound VII-11): To a mixture of tert-butyl N-(6-ethenylpyrimidin-4-yl)carbamate (1.2 g, 5.42 mmol) and K₂OsO₄ (7.5 mg, 0.02 mmol) in H₂O (10.0 mL) and THF (10.0 mL) was added NaIO₄ (5.8 g, 27.12 mmol). The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (60/40, v/v) to afford tert-butyl N-(6-formylpyrimidin-4-yl)carbamate (540.0 mg, 45%) as a white solid. LCMS (ESI, m/z): [M+H]⁺=224.1.

tert-Butyl (E)-(6-((hydroxyimino)methyl)pyrimidin-4-yl)carbamate (Compound VII-7): To a solution of tert-butyl N-(6-formylpyrimidin-4-yl)carbamate (540.0 mg, 2.42 mmol) in EtOH (15.0 mL) was added hydroxylammonium chloride (185.0 mg, 2.66 mmol) and NaOH (256.4 mg, 2.42 mmol). The resulting mixture was stirred at room temperature for 16 h. After the reaction was completed, the mixture was filtered. The solid was collected and dried to afford tert-butyl (E)-(6-((hydroxyimino)methyl)pyrimidin-4-yl)carbamate (507.0 mg, crude) as a white solid. LCMS (ESI, m/z): [M+H]⁺=239.1.

Methyl 4-bromo-2,6-dimethylbenzoate (Compound VII-1): To a solution of 4-bromo-2,6-dimethylbenzoic acid (5.0 g, 21.83 mmol) in DMF (60.0 mL) was added K₂CO₃ (4.5 g, 32.74 mmol) and CH₃I (3.4 g, 24.01 mmol). The mixture was stirred at room temperature for 16 h. After the reaction was completed, the mixture was dilute with water and filtered. The solid was collected and dried to afford methyl 4-bromo-2,6-dimethylbenzoate (5.2 g, crude) as a yellow solid.

Methyl 4-bromo-2-(bromomethyl)-6-methylbenzoate (Compound VII-2): To a solution of methyl 4-bromo-2,6-dimethylbenzoate (5.2 g, 21.23 mmol) in CCl₄ (100.0 mL) was added NBS (4.2 g, 23.35 mmol) and BPO (0.27 g, 1.06 mmol). The reaction mixture was stirred at 80° C. for 16 h. After the reaction was completed, the reaction mixture was cooled to room temperature and filtered. The filtrate was concentrated under reduced pressure to afford methyl 4-bromo-2-(bromomethyl)-6-methylbenzoate (6.5 g, crude) as a yellow oil.

5-Bromo-2-(4-methoxybenzyl)-7-methylisoindolin-1-one (Compound VII-3): To a solution of methyl 4-bromo-2-(bromomethyl)-6-methylbenzoate (6.5 g, 20.19 mmol) in DMF (100.0 mL) was added DIEA (7.8 g, 60.56 mmol) and (4-methoxyphenyl)methanamine (4.5 g, 30.28 mmol) at 0° C. The resulting mixture was stirred at room temperature for 16 h. After the reaction was completed, the mixture was diluted with H₂O and filtered. The solid was purified by flash column chromatography with petroleum ether/EtOAc (70/30, v/v) to afford 5-bromo-2-(4-methoxybenzyl)-7-methylisoindolin-1-one (4.0 g, 57%) as a white solid. LCMS (ESI, m/z): [M+H]⁺=346.0.

6′-Bromo-2′-(4-methoxybenzyl)-4′-methylspiro[cyclohexane-1,1′-isoindolin]-3′-one (Compound VII-4): To a solution of 5-bromo-2-(4-methoxybenzyl)-7-methylisoindolin-1-one (2.6 g, 7.51 mmol) in DMF (30.0 mL) was added NaH (0.8 g, 60%) at 0° C. under N₂. The mixture was stirred at 0° C. for 30 min. Then 1,5-dibromopentane (2.2 g, 9.77 mmol) was added dropwise to the mixture at 0° C. The reaction mixture was stirred at room temperature for additional 16 h. After the reaction was completed, the reaction mixture was diluted with water and extracted with EtOAc. The combined organic layer was washed with brine, dried over anhydrous Na₂SO₄ and filtered. The filtrate was concentrated under vacuum. The residue was purified by flash column chromatography with petroleum ether/EtOAc (80/20, v/v) to afford 6′-bromo-2′-(4-methoxybenzyl)-4′-methylspiro[cyclohexane-1,1′-isoindolin]-3′-one (1.1 g, 37%) as a yellow solid. LCMS (ESI, m/z): [M+H]⁺=414.1.

2′-(4-Methoxybenzyl)-4′-methyl-6′-((trimethylsilyl)ethynyl)spiro [cyclohexane-1,1′-isoindolin]-3′-one (Compound VII-5): To a solution of 6′-bromo-2′-(4-methoxybenzyl)-4′-methylspiro[cyclohexane-1,1′-isoindolin]-3′-one (1.0 g, 2.41 mmol) in THF (25.0 mL) was added DIEA (467.9 mg, 3.62 mmol), ethynyltrimethylsilane (284.5 mg, 2.90 mmol), CuI (46.0 mg, 0.24 mmol) and Pd(PPh₃)₄ (139.4 mg, 0.12 mmol). The reaction mixture was stirred at 50° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/EtOAc (80/20, v/v) to afford compound VII-5 (1.0 g, 78%) as a light yellow solid. LCMS (ESI, m/z): [M+H]⁺=432.2.

6′-Ethynyl-2′-(4-methoxybenzyl)-4′-methylspiro[cyclohexane-1,1′-isoindolin]-3′-one (Compound VII-6): To a solution of 2′-(4-methoxybenzyl)-4′-methyl-6′-((trimethylsilyl)ethynyl)spiro[cyclohexane-1,1′-isoindolin]-3′-one (1.0 g, 7.31 mmol) in THF (20.0 mL) was added TBAF (4.0 mL, 1 mol/L). The reaction mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/EtOAc (40/60, v/v) to afford 6′-ethynyl-2′-(4-methoxybenzyl)-4′-methylspiro[cyclohexane-1,1′-isoindolin]-3′-one (457.3 mg, 53%) as a yellow solid. LCMS (ESI, m/z): [M+H]⁺=360.2.

tert-Butyl (6-(5-(2′-(4-methoxybenzyl)-4′-methyl-3′-oxospiro[cyclohexane-1,1′-isoindolin]-6′-yl)isoxazol-3-yl)pyrimidin-4-yl)carbamate (Compound VII-8): To a solution of 6′-ethynyl-2′-(4-methoxybenzyl)-4′-methylspiro[cyclohexane-1,1′-isoindolin]-3′-one (457.3 mg, 1.27 mmol) in CH₂Cl₂ (20.0 mL) was added tert-butyl (E)-(6-((hydroxyimino)methyl)pyrimidin-4-yl)carbamate (454.3 mg, 1.91 mmol) and NaClO (3.2 g, 12.71 mmol). The reaction mixture was stirred at room temperature for 16 h. After the reaction was completed, the resulting mixture was diluted with water and extracted with CH₂Cl₂. The combined organic layer was washed with brine, dried over anhydrous sodium sulfate and filtered. The filtrate was concentrated under vacuum. The residue was purified by flash column chromatography with petroleum ether/EtOAc (40/60, v/v) to afford tert-butyl (6-(5-(2′-(4-methoxybenzyl)-4′-methyl-3′-oxospiro[cyclohexane-1,1′-isoindolin]-6′-yl)isoxazol-3-yl)pyrimidin-4-yl)carbamate (261.0 mg, 38%) as a yellow solid. LCMS (ESI, m/z): [M+H]⁺=596.3.

6′-(3-(6-Aminopyrimidin-4-yl)isoxazol-5-yl)-4′-methylspiro[cyclohexane-1,1′-isoindolin]-3′-one (Compound VII): A solution of tert-butyl (6-(5-(2′-(4-methoxybenzyl)-4′-methyl-3′-oxospiro[cyclohexane-1,1′-isoindolin]-6′-yl)isoxazol-3-yl)pyrimidin-4-yl)carbamate (261.0 mg, 0.44 mmol) in TFA (5.0 mL) was stirred at 50° C. for 4 h. After the reaction was completed, the pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: Column: XBridge Prep C18 OBD Column, 19×150 mm, 5 um; Mobile Phase A: Water (10 mmol/L NH₄HCO₃), Mobile Phase B: ACN; Flow rate: 20 mL/min; Gradient: 55% B to 80% B in 4.3 min; 210/254 nm; RT1:4.13 min to afford 6′-(3-(6-aminopyrimidin-4-yl)isoxazol-5-yl)-4′-methylspiro[cyclohexane-1,1′-isoindolin]-3′-one (52.3 mg, 37%) as a light yellow solid. LCMS (ESI, m/z): [M+H]⁺=376.1. ¹H NMR (300 MHz, DMSO-d₆): δ 9.24 (s, 1H), 8.52 (s, 1H), 8.09 (s, 1H), 7.81 (s, 1H), 7.66 (s, 1H), 7.20 (s, 2H), 7.09 (d, J=1.2 Hz, 1H), 2.67 (s, 3H), 2.07-1.91 (m, 2H), 1.79-1.63 (m, 5H), 1.40-1.33 (m, 3H).

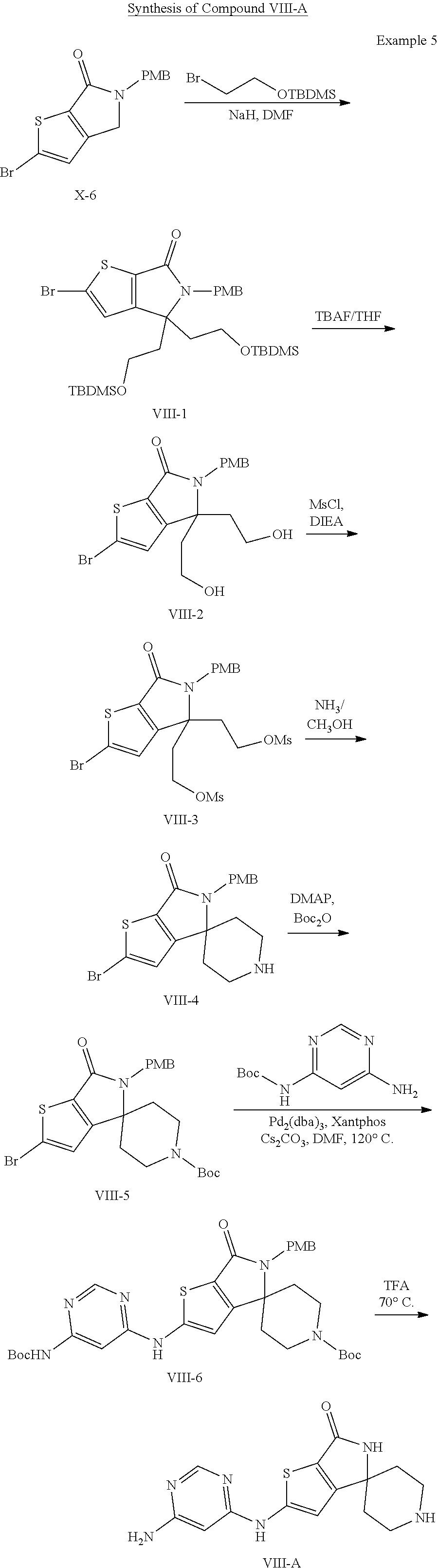

2-bromo-4,4-bis(2-((tert-butyldimethylsilyl)oxy)ethyl)-5-(4-methoxybenzyl)-4,5-dihydro-6H-thieno[2,3-c]pyrrol-6-one (Compound VIII-1): To a solution of 2-bromo-5-[(4-methoxyphenyl)methyl]-4H-thieno[2,3-c]pyrrol-6-one (1000.0 mg, 2.95 mmol) in DMF (20.0 mL) was added NaH (354.7 mg, 60%) at 0° C. under N₂. The mixture was stirred at 0° C. for 30 min. Then (2-bromoethoxy)(tert-butyl)dimethylsilane (2829.2 mg, 11.82 mmol) was added to the mixture at 0° C. The reaction mixture was stirred at room temperature for 16 h. After the reaction was completed, the resulting mixture was diluted with water and extracted with ethyl acetate. The combined organic layer was washed with brine, dried over anhydrous sodium sulfate and filtered. The filtrate was concentrated under vacuum. The residue was purified by flash chromatography with petroleum ether/ethyl acetate (80/20, v/v) to afford the title compound (399.0 mg, 20%) as a yellow oil. LCMS (ESI, m/z): [M+H]⁺=654.2.

2-bromo-4,4-bis(2-hydroxyethyl)-5-(4-methoxybenzyl)-4,5-dihydro-6H-thieno[2,3-c]pyrrol-6-one (Compound VIII-2): To a solution of 2-bromo-4,4-bis([2-[(tert-butyldimethylsilyl)oxy]ethyl])-5-[(4-methoxyphenyl)methyl]thieno[2,3-c]pyrrol-6-one (399.0 mg, 0.60 mmol) in THF (10.0 mL) was added TBAF (637.2 mg, 2.43 mmol). The mixture was stirred at room temperature for 16 h. After the reaction was completed, the resulting mixture was concentrated under vacuum. The residue was purified by flash column chromatography with petroleum ether/ethyl acetate (60/40, v/v) to afford the title compound (200.0 mg, 76%) as a white solid. LCMS (ESI, m/z): [M+H]⁺=426.0.

(2-bromo-5-(4-methoxybenzyl)-6-oxo-5,6-dihydro-4H-thieno[2,3-c]pyrrole-4,4-diyl)bis(ethane-2,1-diyl) dimethanesulfonate (Compound VIII-3): To a solution of 2-bromo-4,4-bis(2-hydroxyethyl)-5-[(4-methoxyphenyl)methyl]thieno[2,3-c]pyrrol-6-one (576.0 mg, 1.35 mmol) in THF (10.0 mL) was added MsCl (325.0 mg, 2.83 mmol) and DIEA (523.8 mg, 4.05 mmol) at 0° C. under N₂. The reaction mixture was stirred at room temperature for 16 h. After the reaction was completed, the resulting mixture was diluted with water and extracted with CH₂Cl₂. The combined organic layer was washed with brine, dried over anhydrous sodium sulfate and filtered. The filtrate was concentrated under vacuum. The residue was purified by flash column chromatography with CH₂Cl₂/MeOH (85/15, v/v) to afford the title compound (590.0 mg, 74%) as an off-white solid. LCMS (ESI, m/z): [M+H]⁺=582.0.

2′-bromo-5′-(4-methoxybenzyl)spiro[piperidine-4,4′-thieno[2,3-c]pyrrol]-6′(5′H)-one (Compound VIII-4): To a solution of (2-bromo-5-(4-methoxybenzyl)-6-oxo-5,6-dihydro-4H-thieno[2,3-c]pyrrole-4,4-diyl)bis(ethane-2,1-diyl) dimethanesulfonate (200.0 mg, 0.34 mmol) in CH₃OH (5.0 mL) was added NH₃/MeOH (3.3 mL, 7.0 mol/L) at 0° C. under N₂. The reaction mixture was stirred at 80° C. for 16 h. After the reaction was completed, the resulting mixture was concentrated under vacuum. The residue was purified by flash column chromatography with CH₂Cl₂/MeOH (85/15, v/v) to afford the title compound (101.0 mg, 72%) as a white solid. LCMS (ESI, m/z): [M+H]⁺=407.0.

tert-butyl 2′-bromo-5′-(4-methoxybenzyl)-6′-oxo-5′,6′-dihydrospiro[piperidine-4,4′-thieno[2,3-c]pyrrole]-1-carboxylate (Compound VIII-5): To a solution of 2′-bromo-5′-(4-methoxybenzyl)spiro[piperidine-4,4′-thieno[2,3-c]pyrrol]-6′(5′H)-one (100.0 mg, 0.24 mmol) in THF (10.0 mL) was added DMAP (6.0 mg, 0.04 mmol) and Boc₂O (58.9 mg, 0.27 mmol). The reaction mixture was stirred at room temperature for 16 h. After the reaction was completed, the resulting mixture was concentrated under vacuum. The residue was purified by flash column chromatography with petroleum ether/ethyl acetate (50/50, v/v) to afford the title compound (80.0 mg, 64%) as a white solid. LCMS (ESI, m/z): [M+H]⁺=507.1.

tert-butyl 2′-((6-((tert-butoxycarbonyl)amino)pyrimidin-4-yl)amino)-5′-(4-methoxybenzyl)-6′-oxo-5′,6′-dihydrospiro[piperidine-4,4′-thieno[2,3-c]pyrrole]-1-carboxylate (Compound VIII-6): To a solution of tert-butyl 2′-bromo-5′-(4-methoxybenzyl)-6′-oxo-5′,6′-dihydrospiro[piperidine-4,4′-thieno[2,3-c]pyrrole]-1-carboxylate (140.0 mg, 0.27 mmol) in DMF (5.0 mL) was added tert-butyl N-(6-aminopyrimidin-4-yl)carbamate (63.8 mg, 0.30 mmol), Cs₂CO₃ (269.6 mg, 0.82 mmol), XantPhos (63.8 mg, 0.11 mmol) and Pd₂(dba)₃ (50.5 mg, 0.05 mmol) at room temperature. The reaction mixture was irradiated with microwave radiation at 120° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (95/5, v/v) to afford the title compound (60.0 mg, 34%) as a white solid. LCMS (ESI, m/z): [M+H]⁺=637.3.

2′-((6-aminopyrimidin-4-yl)amino)spiro[piperidine-4,4′-thieno[2,3-c]pyrrol]-6′(5′H)-one (Compound VIII-A): A solution of tert-butyl 2′-((6-((tert-butoxycarbonyl)amino)pyrimidin-4-yl)amino)-5′-(4-methoxybenzyl)-6′-oxo-5′,6′-dihydrospiro[piperidine-4,4′-thieno[2,3-c]pyrrole]-1-carboxylate (60.0 mg, 0.09 mmol) in TFA (5.0 mL) was stirred at 70° C. for 16 h. After the reaction was completed, the resulting mixture was cooled to room temperature. The pH value of the mixture was adjusted to 7.0 with saturated NaHCO₃ (aq.). The mixture was extracted with ethyl acetate. The combined organic layer was washed with brine, dried over anhydrous sodium sulfate and filtered. The filtrate was concentrated under vacuum. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 2% B to 28% B in 7 min; 254 nm; to afford the title compound (7.1 mg, 23%) as a yellow solid. LCMS (ESI, m/z): [M+H]⁺=317.1. ¹H NMR (400 MHz, DMSO-d₆): δ 10.45 (s, 1H), 8.43 (s, 1H), 8.16 (s, 1H), 6.58-6.56 (m, 3H), 5.78 (s, 1H), 2.89-2.84 (m, 4H), 1.83-1.73 (m, 2H), 1.46-1.40 (m, 2H).

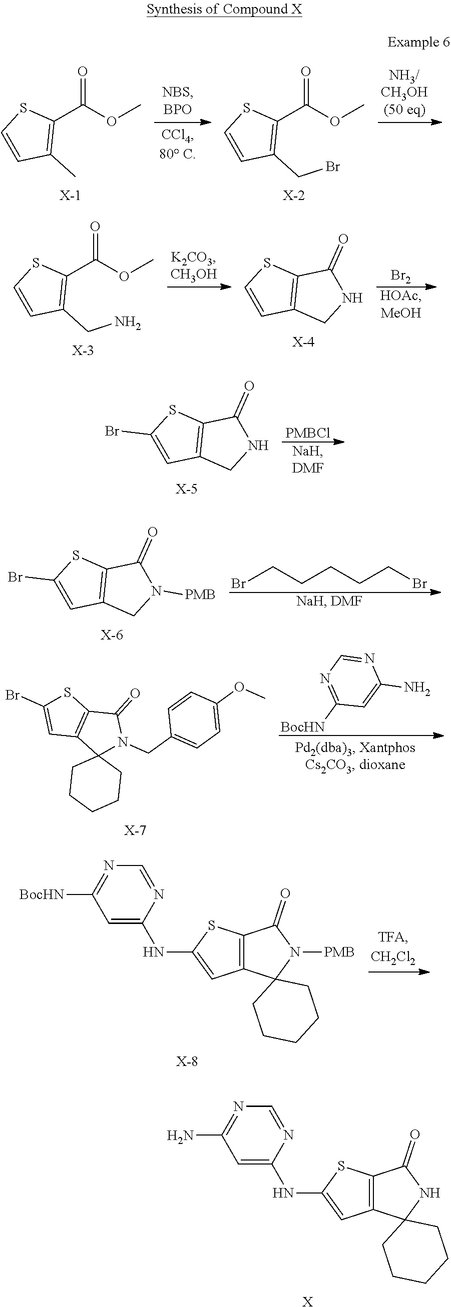

Methyl 3-(bromomethyl)thiophene-2-carboxylate (Compound X-2): To a solution of methyl 3-methylthiophene-2-carboxylate (20.0 g, 128.04 mmol) in CCl₄ (380.0 mL) was added NBS (25.1 g, 140.84 mmol) and BPO (1.6 g, 6.40 mmol). The reaction mixture was stirred at 85° C. for 2 h. After the reaction was completed, the reaction mixture was cooled to room temperature and filtered. The filtrate was concentrated under vacuum. The residue was purified by flash column chromatography with dichloromethane/petroleum ether (30/70, v/v) to afford methyl 3-(bromomethyl)thiophene-2-carboxylate (23.1 g, 76%) as a yellow solid. LCMS (ESI, m/z): [M+H]⁺=235.0.

Methyl 3-(aminomethyl)thiophene-2-carboxylate (Compound X-3): To a solution of methyl 3-(bromomethyl)thiophene-2-carboxylate (23.1 g, 98.25 mmol) in methanol (200.0 mL) was added NH₃/CH₃OH (693.0 mL, 7.0 mol/L) at 0° C. The reaction mixture was stirred at room temperature for 2 h. After the reaction was completed, the resulting mixture was concentrated under vacuum. The residue was purified by flash column chromatography with dichloromethane/methanol (82/18, v/v) to afford methyl 3-(aminomethyl)thiophene-2-carboxylate (14.1 g, 83%) as a yellow solid. LCMS (ESI, m/z): [M+H]⁺=172.0.

4,5-Dihydro-6H-thieno[2,3-c]pyrrol-6-one (Compound X-4): To a solution of methyl 3-(aminomethyl)thiophene-2-carboxylate (14.1 g, 82.35 mmol) in CH₃OH (500.0 mL) was added K₂CO₃ (11.4 g, 82.35 mmol). The reaction mixture was stirred at 70° C. for 16 h. After the reaction was completed, the resulting mixture was cooled to room temperature and concentrated under vacuum. The residue was purified by flash column chromatography with dichloromethane/methanol (96/4, v/v) to afford of 4,5-dihydro-6H-thieno[2,3-c]pyrrol-6-one (6.5 g 56%) as a white solid. LCMS (ESI, m/z): [M+H]⁺=140.0.

2-Bromo-4,5-dihydro-6H-thieno[2,3-c]pyrrol-6-one (Compound X-5): To a solution of 4H,5H-thieno[2,3-c]pyrrol-6-one (6.5 g, 46.70 mmol) in HOAc (48.0 mL) and MeOH (32.0 mL) was added Br₂ (16.0 g, 100.12 mmol) at 0° C. The reaction mixture was stirred at room temperature for 16 h. After the reaction was completed, the resulting mixture was diluted with H₂O and extracted with ethyl acetate. The combined organic layer was washed with 5% Na₂SO₃, saturated NaHCO₃, brine, dried over anhydrous sodium sulfate and filtered. The filtrate was concentrated under vacuum. The residue was purified by flash column chromatography with dichloromethane/methanol (96/4, v/v) to afford 2-bromo-4,5-dihydro-6H-thieno[2,3-c]pyrrol-6-one (7.1 g, 69%) as a white solid. LCMS (ESI, m/z): [M+H]⁺=218.0.

2-Bromo-5-(4-methoxybenzyl)-4,5-dihydro-6H-thieno[2,3-c]pyrrol-6-one (Compound X-6): To a solution of 2-bromo-4H,5H-thieno[2,3-c]pyrrol-6-one (4.0 g, 18.34 mmol) in DMF (40.0 mL) was added NaH (0.6 g, 60%) at 0° C. under N₂. The mixture was stirred at 0° C. for 30 min. Then 4-methoxybenzyl chloride (2.5 g, 16.50 mmol) was added to the mixture at 0° C. The reaction mixture was stirred at room temperature for another 16 h. After the reaction was completed, the resulting mixture was quenched with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (96/4, v/v) to afford 2-bromo-5-[(4-methoxyphenyl)methyl]-4H-thieno[2,3-c]pyrrol-6-one (300.0 mg, 38%) as a yellow solid. LCMS (ESI, m/z): [M+H]⁺=338.0.

2′-Bromo-5′-(4-methoxybenzyl)spiro[cyclohexane-1,4′-thieno[2,3-c]pyrrol]-6′(5′H)-one (Compound X-7): To a solution of 2-bromo-5-[(4-methoxyphenyl)methyl]-4H-thieno[2,3-c]pyrrol-6-one (5.0 g, 14.78 mmol) in DMF (50.0 mL) was added NaH (0.9 g, 60%) at 0° C. under N₂. The mixture was stirred at 0° C. for 30 min. Then 1,5-dibromopentane (4.4 g, 19.21 mmol) was added to the mixture at 0° C. under N₂. The reaction mixture was stirred at room temperature for 16 h. After the reaction was completed, the resulting mixture was quenched with H₂O and extracted with ethyl acetate. The combined organic layer was washed with brine, dried over anhydrous sodium sulfate and filtered. The filtrate was concentrated under vacuum. The residue was purified by flash column chromatography with dichloromethane/methanol (93/7, v/v) to afford 2′-bromo-5′-[(4-methoxyphenyl)methyl]spiro[cyclohexane-1,4′-thieno[2,3-c]pyrrol]-6′-one (3.1 g, 51%) as a yellow solid. LCMS (ESI, m/z): [M+H]⁺=406.0.

tert-Butyl (6-((5′-(4-methoxybenzyl)-6′-oxo-5′,6′-dihydrospiro[cyclohexane-1,4′-thieno[2,3-c]pyrrol]-2′-yl)amino)pyrimidin-4-yl)carbamate (Compound X-8): To a solution of 2′-bromo-5′-(4-methoxybenzyl)spiro[cyclohexane-1,4′-thieno[2,3-c]pyrrol]-6′(5′H)-one (220.0 mg, 0.54 mmol) in dioxane (10.0 mL) was added Cs₂CO₃ (612.1 mg, 1.88 mmol), tert-butyl N-(6-aminopyrimidin-4-yl)carbamate (208.3 mg, 0.99 mmol), Pd₂(dba)₃ (89.2 mg, 0.097 mmol) and Xantphos (93.9 mg, 0.16 mmol). The reaction mixture was stirred for at 100° C. for 16 h under N₂. After the reaction was completed, the mixture was cooled to room temperature and filtered. The filtrate was concentrated under vacuum. The residue was purified by flash column chromatography with dichloromethane/methanol (92/8, v/v) to afford tert-butyl N-(6-[5′-[(4-methoxyphenyl)methyl]-6′-oxospiro[cyclohexane-1,4′-thieno[2,3-c]pyrrol]-2′-ylamino]pyrimidin-4-yl)carbamate (80.0 mg, 27%) as a light yellow solid. LCMS (ESI, m/z): [M+H]⁺=536.2.

2′-((6-Aminopyrimidin-4-yl)amino)spiro[cyclohexane-1,4′-thieno[2,3-c]pyrrol]-6′(5′H)-one (Compound X): A solution of tert-butyl N-(6-[5′-[(4-methoxyphenyl)methyl]-6′-oxospiro[cyclohexane-1,4′-thieno[2,3-c]pyrrol]-2′-ylamino]pyrimidin-4-yl)carbamate (76.0 mg, 0.14 mmol) in TFA (3.0 mL) was stirred at 50° C. for 16 h. After the reaction was completed, the pH value of the mixture was adjusted to 8 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 15% B to 35% B in 8 min; 254 nm; to afford 2′-((6-aminopyrimidin-4-yl)amino)spiro[cyclohexane-1,4′-thieno[2,3-c]pyrrol]-6′(5′H)-one (13.7 mg, 30%) as a white solid. LCMS (ESI, m/z): [M+H]⁺=316.1. ¹H NMR (300 MHz, DMSO-d₆): δ 10.43 (s, 1H), 8.35 (s, 1H), 8.16 (s, 1H), 6.57-6.55 (m, 3H), 5.78 (d, J=0.9 Hz, 1H), 1.72-1.67 (m, 6H), 1.52-1.48 (m, 4H).

Following the procedure described above for Example 6 and substituting the appropriate reagents, starting materials and purification methods known to those skilled in the art, the compounds listed below were prepared.

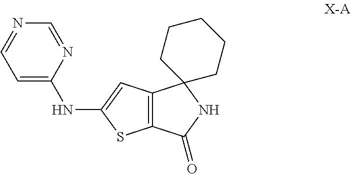 [SMILES: O=C1NC2(CCCCC2)c2cc(Nc3ccncn3)sc21]

2′-(pyrimidin-4-ylamino)spiro[cyclohexane-1,4′-thieno[2,3-c]pyrrol]-6′(5′H)-one (Compound X-A): LCMS (ESI, m/z): [M+H]⁺=301.1. ¹H NMR (300 MHz, DMSO-d₆): δ 11.11 (s, 1H), 8.78 (d, J=0.9 Hz, 1H), 8.48 (s, 1H), 8.37 (d, J=6.0 Hz, 1H), 6.89-6.86 (m, 1H), 6.78 (s, 1H), 1.80-1.48 (m, 10H).

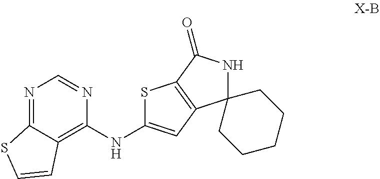 [SMILES: O=C1NC2(CCCCC2)c2cc(Nc3ncnc4sccc34)sc21]

2′-(thieno[2,3-d]pyrimidin-4-ylamino)spiro[cyclohexane-1,4′-thieno[2,3-c]pyrrol]-6′(5′H)-one (Compound X-B): LCMS (ESI, m/z): [M+H]⁺=357.1. ¹H NMR (300 MHz, DMSO-d₆): δ 11.21 (br, 1H), 8.68 (s, 1H), 8.48 (s, 1H), 7.85-7.79 (m, 2H), 6.99 (s, 1H), 1.83-1.63 (m, 6H), 1.59-1.45 (m, 4H).

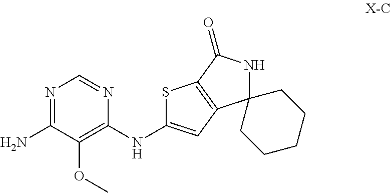 [SMILES: COc1c(N)ncnc1Nc1cc2c(s1)C(=O)NC21CCCCC1]

2′-((6-amino-5-methoxypyrimidin-4-yl)amino)spiro[cyclohexane-1,4′-thieno[2,3-c]pyrrol]-6′(5′H)-one (Compound X-C): The title compound was synthesized by using tert-butyl N-(6-amino-5-methoxypyrimidin-4-yl)-N-(tert-butoxycarbonyl)carbamate as the starting material of which the synthesis was shown below. LCMS (ESI, m/z): [M+H]⁺=346.1. ¹H NMR (300 MHz, DMSO-d₆): δ 10.12 (s, 1H), 8.31 (s, 1H), 7.97 (s, 1H), 6.93 (s, 1H), 6.57 (s, 2H), 3.65 (s, 3H), 1.69-1.47 (m, 10H).

The synthesis of tert-butyl N-(6-amino-5-methoxypyrimidin-4-yl)-N-(tert-butoxycarbonyl)carbamate: To a solution of tert-butyl-(tert-butoxycarbonyl)-N-(6-cyclopropaneamido-5-methoxypyrimidin-4-yl)carbamate (3.0 g, 7.34 mmol) (prepared analogously to compound XX-3) in EtOH (30.0 mL) was added a solution of KOH (1.2 g, 22.03 mmol) in H₂O (15.0 mL). The resulting mixture was stirred at room temperature for 16 h. After the reaction was completed, the resulting mixture was concentrated under vacuum. The residue was purified by flash column chromatography with petroleum ether/ethyl acetate (53/47, v/v) to afford tert-butyl N-(6-amino-5-methoxypyrimidin-4-yl)-N-(tert-butoxycarbonyl)carbamate (816.4 mg, 32%) as a yellow solid. LCMS (ESI, m/z): [M+H]⁺=341.3.

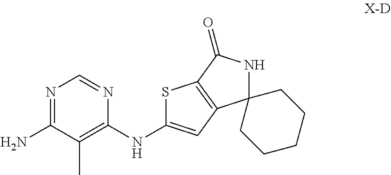 [SMILES: Cc1c(N)ncnc1Nc1cc2c(s1)C(=O)NC21CCCCC1]

2′-((6-amino-5-methylpyrimidin-4-yl)amino)spiro[cyclohexane-1,4′-thieno[2,3-c]pyrrol]-6′(5′H)-one (Compound VII-D):

The title compounds was synthesized according to the synthetic procedure of Compound X-C with the corresponding starting material. LCMS (ESI, m/z): [M+H]⁺=330.2. ¹H NMR (400 MHz, DMSO-d₆): δ 9.58 (s, 1H), 8.26 (s, 1H), 8.07 (s, 1H), 6.85 (s, 1H), 6.35 (s, 2H), 1.98 (s, 3H), 1.67-1.66 (m, 6H), 1.53-1.49 (m, 4H).

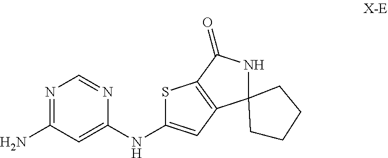 [SMILES: Nc1cc(Nc2cc3c(s2)C(=O)NC32CCCC2)ncn1]

2′-((6-aminopyrimidin-4-yl)amino)spiro[cyclopentane-1,4′-thieno[2,3-c]pyrrol]-6′(5′H)-one (Compound X-E): LCMS (ESI, m/z): [M+H]⁺=302.0. ¹H NMR (300 MHz, DMSO-d₆): δ 10.44 (s, 1H), 8.19-8.16 (m, 2H), 6.55 (s, 2H), 6.50 (s, 1H), 5.78 (d, J=0.6 Hz, 1H), 1.96-1.75 (m, 8H).

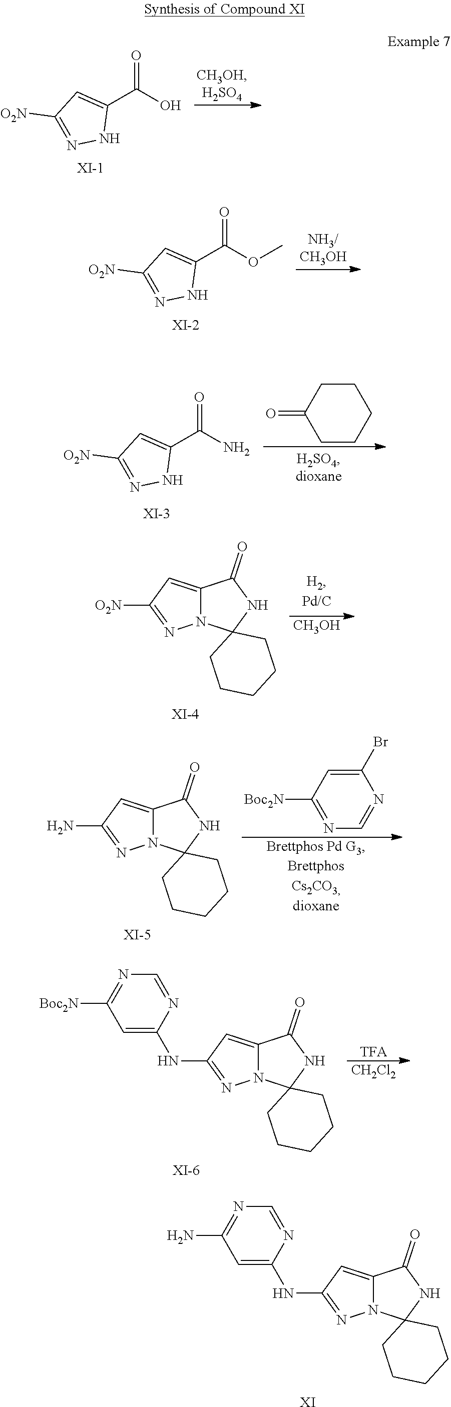

Methyl 3-nitro-1H-pyrazole-5-carboxylate (Compound XI-2): To a solution of 5-nitro-2H-pyrazole-3-carboxylic acid (5.0 g, 31.83 mmol) in methanol (60.0 mL) was added sulfuric acid (10.0 mL). The resulting mixture was stirred at 68° C. for 3 h. After the reaction was completed, the resulting mixture was concentrated under reduced pressure. The pH value of the residue was adjusted to 7 with saturated NaHCO₃ (aq.). The resulting mixture was diluted with water and extracted with EtOAc. The combined organic layer was washed with brine, dried over anhydrous Na₂SO₄ and filtered. The filtrate was concentrated under reduced pressure to afford methyl 3-nitro-1H-pyrazole-5-carboxylate (3.7 g, crude) as a white solid. LCMS (ESI, m/z): [M+H]⁺=172.0.

3-Nitro-1H-pyrazole-5-carboxamide (Compound XI-3): The solution of methyl 3-nitro-1H-pyrazole-5-carboxylate (3.7 g, 21.62 mmol) in NH₃/MeOH (72.0 mL, 7.0 mol/L) was stirred at 60° C. for 16 h. After the reaction was completed, the resulting mixture was concentrated under reduced pressure. The residue was purified by flash column chromatography with CH₂Cl₂/MeOH (80/20, v/v) to afford 3-nitro-1H-pyrazole-5-carboxamide (1.1 g, 33%) as a white solid. LCMS (ESI, m/z): [M+H]⁺=157.0

2′-Nitrospiro[cyclohexane-1,6′-imidazo[1,5-b]pyrazol]-4′(5′H)-one (Compound XI-4): To a solution of 3-nitro-1H-pyrazole-5-carboxamide (1.3 g, 8.01 mmol) in dioxane (50.0 mL) was added cyclohexanone (7.9 g, 79.98 mmol) and H₂SO₄ (393.0 mg, 4.01 mmol). The resulting mixture was stirred at 100° C. for 16 h. After the reaction was completed, the resulting mixture was concentrated under reduced pressure. The residue was purified by flash column chromatography with CH₂Cl₂/MeOH (80/20, v/v) to afford 2′-nitrospiro[cyclohexane-1,6′-imidazo[1,5-b]pyrazol]-4′(5′H)-one (1.2 g, 61%) as a yellow oil. LCMS (ESI, m/z): [M+H]⁺=237.1

2′-Aminospiro[cyclohexane-1,6′-imidazo[1,5-b]pyrazol]-4′(5′H)-one (Compound XI-5): To a solution of 2′-nitrospiro[cyclohexane-1,6′-imidazo[1,5-b]pyrazol]-4′(5′H)-one (1.2 g, 4.91 mmol) in CH₃OH (30.0 ml) was added Pd/C (390.0 mg, dry). The resulting mixture was stirred at room temperature for 16 h under H₂. After the reaction was completed, the resulting mixture was filtered. The filtrate was concentrated under reduced pressure to afford 2′-aminospiro[cyclohexane-1,6′-imidazo[1,5-b]pyrazol]-4′(5′H)-one (130.0 mg, crude) as a yellow solid. LCMS (ESI, m/z): [M+H]⁺=207.1.

tert-Butyl (tert-butoxycarbonyl)(6-((4′-oxo-4′,5′-dihydrospiro[cyclohexane-1,6′-imidazo[1,5-b]pyrazol]-2′-yl)amino)pyrimidin-4-yl)carbamate (Compound XI-6): To a solution of 2′-aminospiro[cyclohexane-1,6′-imidazo[1,5-b]pyrazol]-4′(5′H)-one (158.0 mg, 0.77 mmol) in dioxane (20.0 mL) was added tert-butyl N-(6-bromopyrimidin-4-yl)-N-(tert-butoxycarbonyl)carbamate (286.0 mg, 0.76 mmol), BrettPhos (41.0 mg, 0.08 mmol), BrettPhos Pd G3 (140.0 mg, 0.15 mmol) and Cs₂CO₃ (500.0 mg, 1.54 mmol). The resulting mixture was stirred at 100° C. for 16 h under N₂. After the reaction was completed, the resulting mixture was diluted with water and extracted with EtOAc. The combined organic layer was washed with brine, dried over anhydrous Na₂SO₄ and filtered. The filtrate was concentrated under reduced pressure. The residue was purified by reverse phase flash chromatography with ACN/H₂O (50/50, v/v) to afford tert-butyl (tert-butoxycarbonyl)(6-((4′-oxo-4′,5′-dihydrospiro[cyclohexane-1,6′-imidazo[1,5-b]pyrazol]-2′-yl)amino)pyrimidin-4-yl)carbamate (61.0 mg, 16%) as a white solid. LCMS (ESI, m/z): [M+H]⁺=500.3.

2′-((6-Aminopyrimidin-4-yl)amino)spiro[cyclohexane-1,6′-imidazo[1,5-b]pyrazol]-4′(5′H)-one (Compound XI): To a solution of tert-butyl (tert-butoxycarbonyl)(6-((4′-oxo-4′,5′-dihydrospiro[cyclohexane-1,6′-imidazo[1,5-b]pyrazol]-2′-yl)amino)pyrimidin-4-yl)carbamate (61.0 mg, 0.12 mmol) in CH₂Cl₂ (10.0 mL) was added TFA (2.0 mL). The resulting mixture was stirred at room temperature for 1 h. After the reaction was completed, the resulting mixture was concentrated under reduced pressure. The pH value of the residue was adjusted to 7 with saturated NaHCO₃ (aq.). The resulting mixture was extracted with CH₂Cl₂. The combined organic layer was washed with brine, dried over anhydrous Na₂SO₄ and filtered. The filtrate was concentrated under reduced pressure. The residue was purified by Prep-HPLC with the following conditions: Column: YMC-Actus Triart C18, 20×250 mm, 5 um, 12 nm; Mobile Phase A: Water (10 mmol/L NH₄HCO₃), Mobile Phase B: ACN; Flow rate: 60 mL/min; Gradient: 10% B to 40% B in 7 min; 254 nm; to afford 2′-((6-aminopyrimidin-4-yl)amino)spiro[cyclohexane-1,6′-imidazo[1,5-b]pyrazol]-4′(5′H)-one (9.5 mg, 26%) as a white solid, LCMS (ESI, m/z): [M+H]⁺=300.1. ¹H NMR (300 MHz, DMSO-d₆): δ 9.77 (s, 1H), 9.48 (s, 1H), 8.02 (s, 1H), 6.54 (s, 1H), 6.38 (s, 2H), 6.15 (s, 1H), 2.08-1.82 (m, 4H), 1.79-1.66 (m, 5H), 1.44-1.39 (m, 1H).

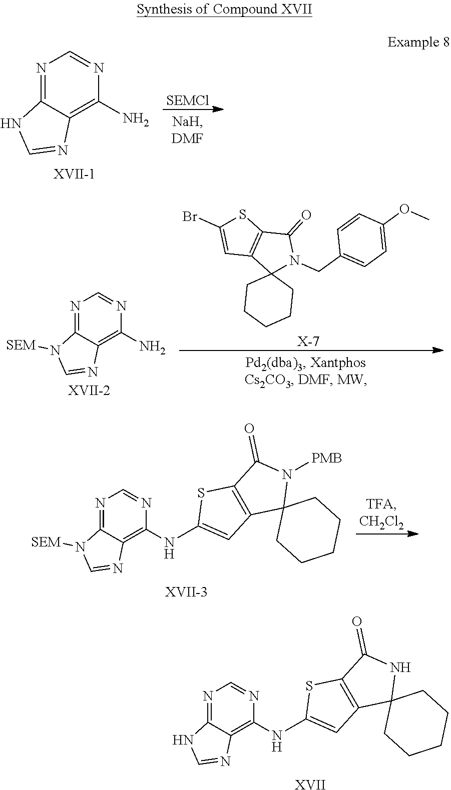

9-((2-(Trimethylsilyl)ethoxy)methyl)-9H-purin-6-amine (Compound XVII-2): To a solution of 9H-purin-6-amine (2.0 g, 14.80 mmol) in DMF (30.0 mL) was added NaH (0.43 g, 60%) at 0° C. under N₂. The mixture was stirred at 0° C. for 20 min. Then SEM-Cl (2.71 g, 16.28 mmol) was added to the mixture. The resulting mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (50/50, v/v) to afford compound XVII-2 (885.6 mg, 22%) as a white solid. LCMS (ESI, m/z): [M+H]⁺=266.1.

5′-(4-Methoxybenzyl)-2′-((9-((2-(trimethylsilyl)ethoxy)methyl)-9H-purin-6-yl)amino)spiro[cyclohexane-1,4′-thieno[2,3-c]pyrrol]-6′(5′H)-one (Compound XVII-3): To a solution of 2′-bromo-5′-(4-methoxybenzyl)spiro[cyclohexane-1,4′-thieno[2,3-c]pyrrol]-6′(5′H)-one (300.0 mg, 0.74 mmol) in DMF (10.0 mL) was added 9-((2-(trimethylsilyl)ethoxy)methyl)-9H-purin-6-amine (391.9 mg, 1.48 mmol), Pd₂(dba)₃ (135.2 mg, 0.15 mmol), XantPhos (170.9 mg, 0.30 mmol) and Cs₂CO₃ (360.8 mg, 1.10 mmol). The reaction mixture was irradiated with microwave radiation at 120° C. for 3 h under N₂. After the reaction was completed, the mixture was cooled to room temperature and filtered. The filtrate was diluted with of H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (92/8, v/v) to afford compound XVII-3 (406.0 mg. 47%) as a yellow solid. LCMS (ESI, m/z): [M+H]⁺=591.2.

2′-((9H-Purin-6-yl)amino)spiro[cyclohexane-1,4′-thieno[2,3-c]pyrrol]-6′(5′H)-one (Compound XVII): A solution of 5′-(4-methoxybenzyl)-2′-((9-((2-(trimethylsilyl)ethoxy)methyl)-9H-purin-6-yl)amino)spiro[cyclohexane-1,4′-thieno[2,3-c]pyrrol]-6′(5′H)-one (406.0 mg, 0.68 mmol) in TFA (10.0 mL) was stirred at 80° C. for 16 h. After the reaction was complete, the mixture was cooled to room temperature and then basified to pH=8 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by flash column chromatography with CH₂Cl₂/MeOH (92/8, v/v) and then purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 24% B to 34% B in 7 min; 254/220 nm; RT1:6.57 min to afford compound XVII (5.5 mg, 3%) as a white solid. LCMS (ESI, m/z): [M+H]⁺=341.2. ¹H NMR (300 MHz, DMSO-d₆): δ 8.53 (s, 1H), 8.45 (s, 1H), 8.35 (s, 1H), 7.05 (s, 1H), 1.79-1.69 (m, 6H), 1.60-1.36 (m, 4H).

Following the procedure described above for compound XVII and substituting the appropriate reagents, starting materials and purification methods known to those skilled in the art, the compounds listed below were prepared.

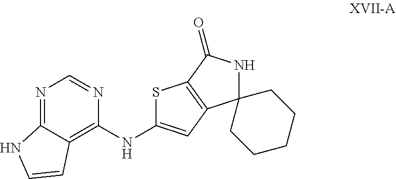 [SMILES: O=C1NC2(CCCCC2)c2cc(Nc3ncnc4[nH]ccc34)sc21]

2′-((7H-pyrrolo[2,3-d]pyrimidin-4-yl)amino)spiro[cyclohexane-1,4′-thieno[2,3-c]pyrrol]-6′(5′H)-one (Compound XVII-A): LCMS (ESI, m/z): [M+H]⁺=340.0. ¹H NMR (400 MHz, DMSO-d₆): δ 11.95 (s, 1H), 11.00 (s, 1H), 8.42 (s, 1H), 8.40 (s, 1H), 7.33-7.32 (m, 1H), 6.88 (s, 1H), 6.77-6.76 (m, 1H), 1.75-1.61 (m, 6H), 1.56-1.53 (m, 4H).

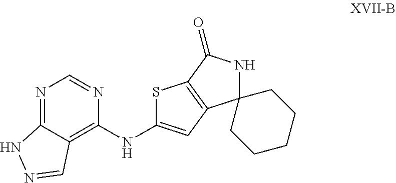 [SMILES: O=C1NC2(CCCCC2)c2cc(Nc3ncnc4[nH]ncc34)sc21]

2′-((1H-pyrazolo[3,4-d]pyrimidin-4-yl)amino)spiro[cyclohexane-1,4′-thieno[2,3-c]pyrrol]-6′(5′H)-one (Compound XVII-B): LCMS (ESI, m/z): [M+H]⁺=341.0. ¹H NMR (300 MHz, DMSO-d₆): δ 13.85 (s, 1H), 11.74 (s, 1H), 8.57 (s, 1H), 8.55 (s, 1H), 8.33 (s, 1H), 6.96 (s, 1H), 1.73-1.68 (m, 6H), 1.65-1.53 (m, 4H).

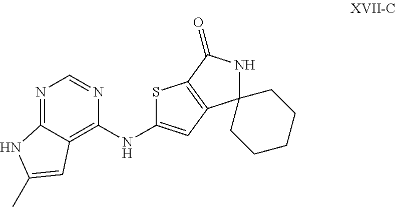 [SMILES: Cc1cc2c(Nc3cc4c(s3)C(=O)NC43CCCCC3)ncnc2[nH]1]

2′-((6-methyl-7H-pyrrolo[2,3-d]pyrimidin-4-yl)amino)spiro[cyclohexane-1,4′-thieno[2,3-c]pyrrol]-6′(5′H)-one (Compound XVII-C): LCMS (ESI, m/z): [M+H]⁺=354.1. ¹H NMR (300 MHz, DMSO-d₆): δ 11.78 (s, 1H), 10.83 (s, 1H), 8.37 (s, 1H), 8.33 (s, 1H), 6.83 (s, 1H), 6.42 (s, 1H), 2.37 (s, 3H), 1.73-1.68 (m, 6H), 1.65-1.51 (m, 4H).

Following the synthetic route described above for compound XVII and substituting the appropriate reagents, starting materials (including, e.g., the corresponding 6,5 and 6,6 bicyclic amino-heterocycle) and purification methods known to those skilled in the art, the compounds listed below may be synthesized.

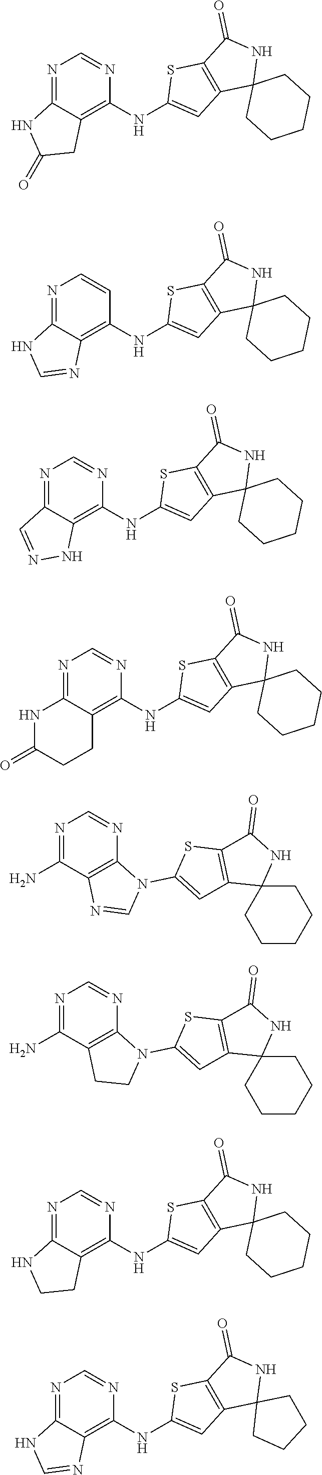

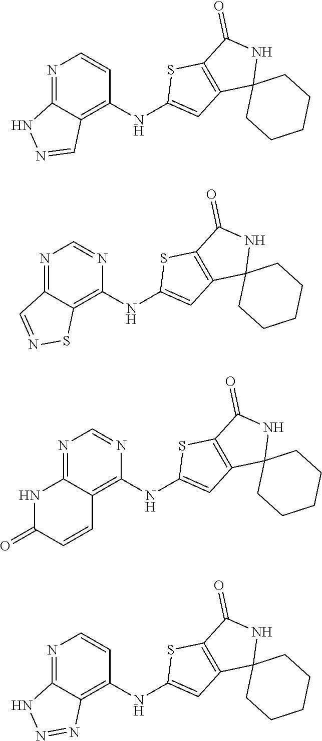 [SMILES: O=C1NC2(CCCCC2)c2cc(Nc3ccnc4[nH]ncc34)sc21 ; O=C1NC2(CCCCC2)c2cc(Nc3ncnc4cnsc34)sc21 ; O=C1NC2(CCCCC2)c2cc(Nc3ncnc4[nH]c(=O)ccc34)sc21 ; O=C1NC2(CCCCC2)c2cc(Nc3ccnc4[nH]nnc34)sc21]

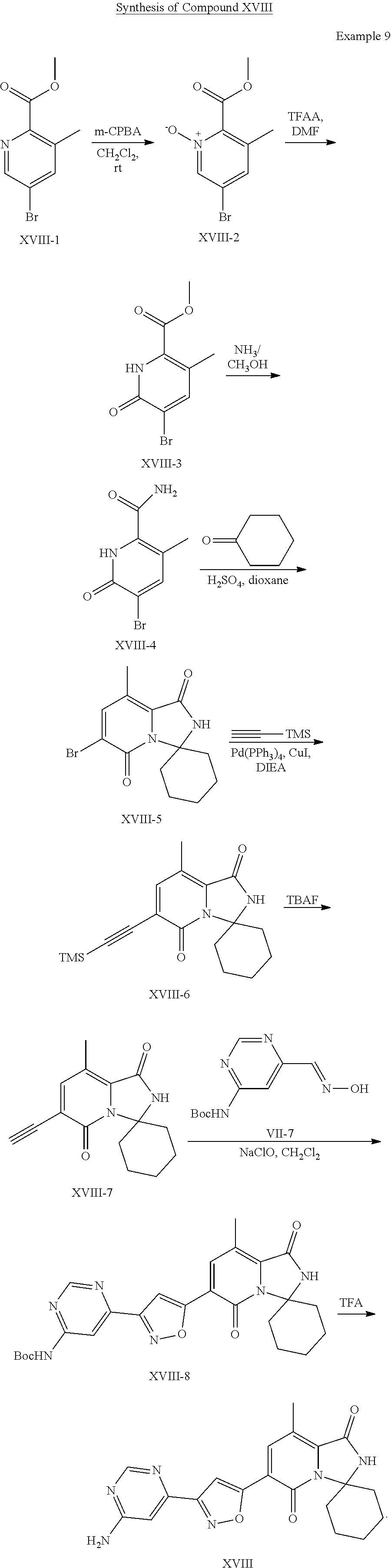

5-Bromo-2-(methoxycarbonyl)-3-methylpyridine 1-oxide (Compound XVIII-2): To a solution of methyl 5-bromo-3-methylpicolinate (6.0 g, 26.08 mmol) in CH₂Cl₂ (200.0 mL) was added m-CPBA (13.5 g, 78.23 mmol). The mixture was stirred at room temperature for 16 h. After the reaction was completed, the mixture was concentrated under reduced pressure. The residue was purified by flash column chromatography with petroleum ether/ethyl acetate (80/20, v/v) to afford compound XVIII-2 (5.5 g, 86%) as a light yellow solid. LCMS (ESI, m/z): [M+H]⁺=246.0.

Methyl 5-bromo-3-methyl-6-oxo-1,6-dihydropyridine-2-carboxylate (Compound XVIII-3): To a solution of 5-bromo-2-(methoxycarbonyl)-3-methylpyridine 1-oxide (5.5 g, 22.43 mmol) in DMF (80.0 mL) was added TFAA (51.8 g, 246.77 mmol). The reaction mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (60/40, v/v) to afford compound XVIII-3 (1.6 g, 28%) as a yellow solid. LCMS (ESI, m/z): [M+H]⁺=246.0.

5-Bromo-3-methyl-6-oxo-1,6-dihydropyridine-2-carboxamide (Compound XVIII-4): A solution of methyl 5-bromo-3-methyl-6-oxo-1,6-dihydropyridine-2-carboxylate (1.6 g, 6.38 mmol) in NH₃/CH₃OH (80.0 mL, 7.0 mol/L) was stirred at 60° C. for 16 h. After the reaction was completed, the mixture was concentrated under vacuum to afford compound XVIII-4 (1.7 g, crude) as a yellow solid. LCMS (ESI, m/z): [M+H]⁺=231.0.

6′-Bromo-8′-methyl-2′H-spiro[cyclohexane-1,3′-imidazo[1,5-a]pyridine]-1′,5′-dione (Compound XVIII-5): To a solution of 5-bromo-3-methyl-6-oxo-1,6-dihydropyridine-2-carboxamide (1.7 g, 7.31 mmol) in dioxane (50.0 mL) was added cyclohexanone (2.9 g, 29.26 mmol) and H₂SO₄ (0.14 g, 1.46 mmol). The reaction mixture was stirred at 100° C. for 16 h. After the reaction was completed, the reaction mixture was concentrated under vacuum. The residue was washed with Et₂O and filtered. The solid was collected and dried to afford compound XVIII-5 (1.7 g, crude) as a yellow solid. LCMS (ESI, m/z): [M+H]⁺=311.0.

8′-Methyl-6′-((trimethylsilyl)ethynyl)-2′H-spiro[cyclohexane-1,3′-imidazo[1,5-a]pyridine]-1′,5′-dione (Compound XVIII-6): To a solution of 6′-bromo-8′-methyl-2′H-spiro[cyclohexane-1,3′-imidazo[1,5-a]pyridine]-1′,5′-dione (650.0 mg, 2.09 mmol) in THF (25.0 mL) was added DIEA (404.9 mg, 3.13 mmol), ethynyltrimethylsilane (246.2 mg, 2.51 mmol), CuI (39.8 mg, 0.21 mmol), and Pd(PPh₃)₄ (120.7 mg, 0.10 mmol). The reaction mixture was stirred at 50° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (93/7, v/v) to afford compound XVIII-6 (249.0 mg, 26%) as a light yellow solid. LCMS (ESI, m/z): [M+H]⁺=329.2.

6′-Ethynyl-8′-methyl-2′H-spiro[cyclohexane-1,3′-imidazo[1,5-a]pyridine]-1′,5′-dione (Compound XVIII-7): To a solution of 8′-methyl-6′-((trimethylsilyl)ethynyl)-2′H-spiro[cyclohexane-1,3′-imidazo[1,5-a]pyridine]-1′, 5′-dione (249.0 mg, 0.76 mmol) in THF (10.0 mL) was added TBAF (1.0 mL, 1 mol/L in THF). The reaction mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (93/7, v/v) to afford compound XVIII-7 (170.0 mg, 87%) as a yellow solid. LCMS (ESI, m/z): [M+H]⁺=257.1.

tert-Butyl (6-(5-(8′-methyl-1′,5′-dioxo-1′,5′-dihydro-2′H-spiro[cyclohexane-1,3′-imidazo[1,5-a]pyridin]-6′-yl)isoxazol-3-yl)pyrimidin-4-yl)carbamate (Compound XVIII-8): To a stirred solution of 6′-ethynyl-8′-methyl-2′H-spiro[cyclohexane-1,3′-imidazo[1,5-a]pyridine]-1′,5′-dione (170.0 mg, 0.66 mmol) in CH₂Cl₂ (15.0 mL) was added tert-butyl (E)-(6-((hydroxyimino)methyl)pyrimidin-4-yl)carbamate (237.0 mg, 0.99 mmol) and NaClO (493.7 mg, 6.63 mmol). The reaction mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (70/30, v/v) to afford compound XVIII-8 (185.6 mg, 38%) as a yellow solid. LCMS (ESI, m/z): [M+H]⁺=493.2.

6′-(3-(6-Aminopyrimidin-4-yl)isoxazol-5-yl)-8′-methyl-2′H-spiro[cyclohexane-1,3′-imidazo[1,5-a]pyridine]-1′,5′-dione (Compound XVIII): To a solution of tert-butyl (6-(5-(8′-methyl-1′,5′-dioxo-1′,5′-dihydro-2′H-spiro[cyclohexane-1,3′-imidazo[1,5-a]pyridin]-6′-yl)isoxazol-3-yl)pyrimidin-4-yl)carbamate (185.6 mg, 0.38 mmol) in CH₂Cl₂ (10.0 mL) was added TFA (2.0 mL). The reaction mixture was stirred at room temperature for 4 h. After the reaction was completed, the pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: Column: XBridge Prep OBD C18 Column, 19×250 mm, 5 um; Mobile Phase A: Water (10 mmol/L NH₄HCO₃), Mobile Phase B: MeOH-HPLC; Flow rate: 25 mL/min; Gradient: 47% B to 70% B in 7 min; 254 nm; to afford compound XVIII (6.7 mg, 4%) as a yellow solid. LCMS (ESI, m/z): [M+H]⁺=393.1. ¹H NMR (300 MHz, DMSO-d₆): δ 10.54 (s, 1H), 8.51 (s, 1H), 8.24 (s, 1H), 7.62 (s, 1H), 7.17 (s, 2H), 7.08 (s, 1H), 3.09-3.01 (m, 2H), 1.80-1.63 (m, 5H), 1.53-1.49 (m, 2H), 1.28-1.24 (m, 2H).

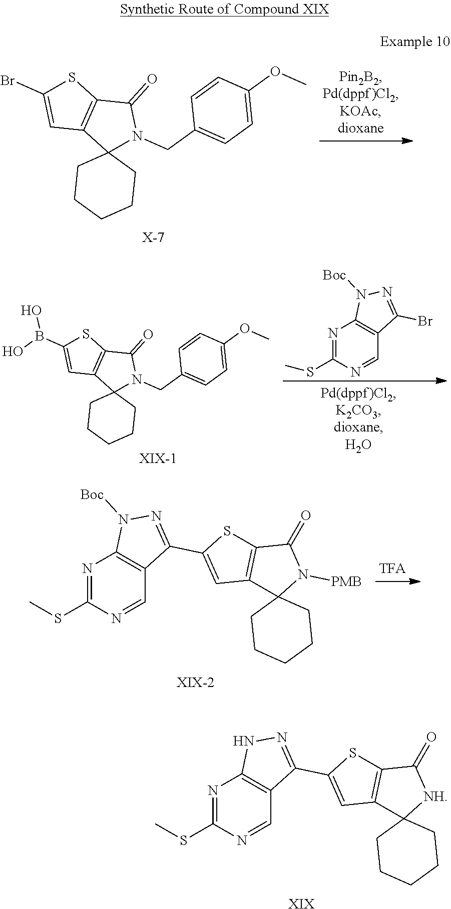

(5′-(4-Methoxybenzyl)-6′-oxo-5′,6′-dihydrospiro[cyclohexane-1,4′-thieno[2,3-c]pyrrol]-2′-yl)boronic acid (Compound XIX-1): To a solution of 2′-bromo-5′-(4-methoxybenzyl)spiro[cyclohexane-1,4′-thieno[2,3-c]pyrrol]-6′(5′H)-one (500.0 mg, 1.23 mmol) in dioxane (10.0 mL) was added bis(pinacolato)diboron (468.7 mg, 1.84 mmol), KOAc (362.9 mg, 3.69 mmol) and Pd(dppf)Cl₂ (90.0 mg, 0.12 mmol). The reaction mixture was stirred at 80° C. for 16 h under N₂. After the reaction was completed, the mixture was filtered. The filtrate was concentrated under vacuum. The residue was purified by reverse phase flash column chromatography with ACN/H₂O (50/50, v/v) to afford compound XIX-1 (150.0 mg, 32%) as a white solid. LCMS (ESI, m/z): [M+H]⁺=372.1.

tert-Butyl 3-(5′-(4-methoxybenzyl)-6′-oxo-5′,6′-dihydrospiro[cyclohexane-1,4′-thieno[2,3-c]pyrrol]-2′-yl)-6-(methylthio)-1H-pyrazolo[3,4-d]pyrimidine-1-carboxylate (Compound XIX-2): To a solution of (5′-(4-methoxybenzyl)-6′-oxo-5′,6′-dihydrospiro[cyclohexane-1,4′-thieno[2,3-c]pyrrol]-2′-yl)boronic acid (140.0 mg, 0.37 mmol) in dioxane (5.0 mL) and H₂O (0.5 mL) was added tert-butyl 3-bromo-6-(methyl sulfanyl)pyrazolo[3,4-d]pyrimidine-1-carboxylate (130.1 mg, 0.37 mmol), K₂CO₃ (131.2 mg, 0.94 mmol) and Pd(dppf)Cl₂ (55.1 mg, 0.07 mmol). The reaction mixture was stirred at 100° C. for 16 h under N₂. After the reaction was completed, the mixture was filtered. The filtrate was concentrated under vacuum. The residue was purified by flash column chromatography with ethyl acetate/petroleum ether (30/70, v/v) to afford compound XIX-2 (29.0 mg, 13%) as an off-white solid. LCMS (ESI, m/z): [M+H]⁺=592.2.

2′-(6-(Methylthio)-1H-pyrazolo[3,4-d]pyrimidin-3-yl)spiro[cyclohexane-1,4′-thieno[2,3-c]pyrrol]-6′(5′H)-one (Compound XIX): A solution of tert-butyl 3-(5′-(4-methoxybenzyl)-6′-oxo-5′,6′-dihydrospiro[cyclohexane-1,4′-thieno[2,3-c]pyrrol]-2′-yl)-6-(methylthio)-1H-pyrazolo[3,4-d]pyrimidine-1-carboxylate (62.0 mg, 0.10 mmol) in TFA (10.0 mL) was stirred at 50° C. for 16 h. After the reaction was completed, the mixture was cooled to room temperature. The pH value of the mixture was adjusted to 8 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: Column: Xselect CSH OBD Column 30×150 mm, 5 um; Mobile Phase A: Water (0.1% FA), Mobile Phase B: ACN; Flow rate: 60 mL/min; Gradient: 32% B to 50% B in 7 min; 254/220 nm; to afford compound XIX (8.8 mg, 22%) as a white solid. LCMS (ESI, m/z): [M+H]⁺=372.1. ¹H NMR (400 MHz, DMSO-d₆): δ 14.14 (s, 1H), 9.61 (s, 1H), 9.00 (s, 1H), 8.10 (s, 1H), 2.60 (s, 3H), 1.94-1.88 (m, 2H), 1.78-1.61 (m, 7H), 1.46-1.35 (s, 1H).

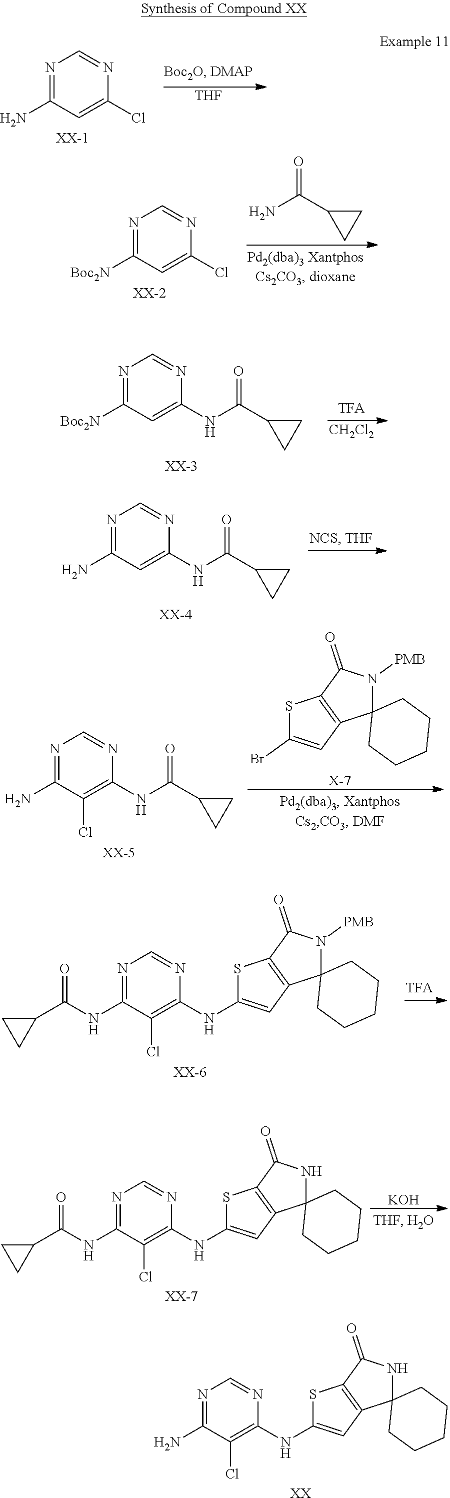

tert-Butyl N-(tert-butoxycarbonyl)-N-(6-chloropyrimidin-4-yl)carbamate (Compound XX-2): To a solution of 6-chloropyrimidin-4-amine (20.0 g, 154.38 mmol) in THF (400.0 mL) was added Boc₂O (75.0 g, 343.65 mmol) and DMAP (1.9 g, 15.47 mmol). The resulting mixture was stirred at room temperature for 16 h. After the reaction was completed, the resulting mixture was concentrated under reduced pressure. The residue was purified by flash column chromatography with CH₂Cl₂/MeOH (90/10, v/v) to afford compound XX-2 (30.0 g, 59%) as a white solid. LCMS (ESI, m/z): [M+H]⁺=330.1.

tert-Butyl N-(tert-butoxycarbonyl)-N-(6-cyclopropaneamidopyrimidin-4-yl)carbamate (Compound XX-3): To a solution of tert-butyl N-(tert-butoxycarbonyl)-N-(6-chloropyrimidin-4-yl)carbamate (27.5 g, 83.39 mmol) in dioxane (500.0 mL) was added cyclopropanecarboxamide (9.2 g, 108.57 mmol), Pd₂(dba)₃ (7.6 g, 8.30 mmol), XantPhos (9.7 g, 16.76 mmol) and Cs₂CO₃ (81.5 g, 250.14 mmol). The resulting mixture was stirred at 90° C. for 16 h under N₂. After the reaction was completed, the resulting mixture was cooled to room temperature and filtered. The filtrate was concentrated under reduced pressure. The residue was purified by flash column chromatography with ethyl acetate/petroleum ether (20/80, v/v) to afford compound XX-3 (19.0 g, 76%) as a yellow solid. LCMS (ESI, m/z): [M+H]⁺=379.2.

N-(6-Aminopyrimidin-4-yl)cyclopropanecarboxamide (Compound XX-4): To a solution of tert-butyl N-(tert-butoxycarbonyl)-N-(6-cyclopropaneamidopyrimidin-4-yl)carbamate (8.6 g, 22.73 mmol) in CH₂Cl₂ (100.0 mL) was added TFA (40.0 mL). The resulting mixture was stirred at room temperature for 1 h. After the reaction was completed, the resulting mixture was concentrated under reduced pressure. The pH value of the residue was adjusted to 7 with saturated NaHCO₃ (aq.). The mixture was extracted with ethyl acetate. The combined organic layer was washed with brine, dried over anhydrous sodium sulfate and filtered. The filtrate was concentrated under reduced pressure. The residue was purified by reverse phase flash chromatography with MeOH/H₂O (50/50, v/v) to afford compound XX-4 (3.5 g, 87%) as a white solid. LCMS (ESI, m/z): [M+H]⁺=179.1.

N-(6-Amino-5-chloropyrimidin-4-yl)cyclopropanecarboxamide (Compound XX-5): To a solution of N-(6-aminopyrimidin-4-yl)cyclopropanecarboxamide (3.0 g, 16.84 mmol) in THF (80.0 mL) was added NCS (2.2 g, 16.78 mmol). The resulting mixture was stirred at room temperature for 16 h.

After the reaction was completed, the resulting mixture was concentrated under reduced pressure. The residue was purified by flash column chromatography with CH₂Cl₂/MeOH (90/10, V/V) to afford N-(6-amino-5-chloropyrimidin-4-yl)cyclopropanecarboxamide (3.0 g, 84%) as a yellow solid. LCMS (ESI, m/z): [M+H]⁺=213.0.

N-(5-Chloro-6-((5′-(4-methoxybenzyl)-6′-oxo-5′,6′-dihydrospiro[cyclohexane-1,4′-thieno[2,3-c]pyrrol]-2′-yl)amino)pyrimidin-4-yl)cyclopropanecarboxamide (Compound XX-6): To a solution of N-(6-amino-5-chloropyrimidin-4-yl)cyclopropanecarboxamide (200.0 mg, 0.94 mmol) in DMF (20.0 mL) was added 2′-bromo-5′-(4-methoxybenzyl)spiro[cyclohexane-1,4′-thieno[2,3-c]pyrrol]-6′(5′H)-one (420.0 mg, 1.03 mmol), Pd₂(dba)₃ (260.0 mg, 0.28 mmol), XantPhos (327.0 mg, 0.57 mmol) and Cs₂CO₃ (920.0 mg, 2.82 mmol). The reaction mixture was irradiated with microwave radiation at 120° C. for 3 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash chromatography with CH₂Cl₂/MeOH (90/10, v/v) to afford compound XX-6 (321.0 mg, 63%) as a yellow solid. LCMS (ESI, m/z): [M+H]⁺=538.1.

N-(5-Chloro-6-((6′-oxo-5′,6′-dihydrospiro[cyclohexane-1,4′-thieno[2,3-c]pyrrol]-2′-yl)amino)pyrimidin-4-yl)cyclopropanecarboxamide (Compound XX-7): A solution of N-(5-chloro-6-((5′-(4-methoxybenzyl)-6′-oxo-5′,6′-dihydrospiro[cyclohexane-1,4′-thieno[2,3-c]pyrrol]-2′-yl)amino)pyrimidin-4-yl)cyclopropanecarboxamide (80.0 mg, 0.15 mmol) in TFA (5.0 mL) was stirred at 60° C. for 16 h. After the reaction was completed, the resulting mixture was cooled to room temperature. The pH value of the mixture was adjusted to 8 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 32% B to 41% B in 8 min; 254/220 nm to afford compound XX-7 (13.8 mg, 21%) as a yellow solid. LCMS (ESI, m/z): [M+H]⁺=418.1. ¹H NMR (300 MHz, DMSO-d₆): δ 10.67-10.58 (m, 2H), 8.57 (s, 1H), 8.48 (s, 1H), 7.19 (s, 1H), 2.08-1.96 (m, 1H), 1.71-1.66 (m, 6H), 1.61-1.52 (m, 4H), 0.87-0.85 (m, 4H).

2′-((6-Amino-5-chloropyrimidin-4-yl)amino)spiro[cyclohexane-1,4′-thieno[2,3-c]pyrrol]-6′(5′H)-one (Compound XX): To a solution of N-(5-chloro-6-((6′-oxo-5′,6′-dihydrospiro[cyclohexane-1,4′-thieno[2,3-c]pyrrol]-2′-yl)amino)pyrimidin-4-yl)cyclopropanecarboxamide (164.0 mg, 0.39 mmol) in THE (5.0 mL) and EtOH (10.0 mL) was added a solution of KOH (128.0 mg, 2.28 mmol) in H₂O (5.0 mL). The resulting mixture was stirred at 60° C. for 16 h. After the reaction was completed, the resulting mixture was cooled to room temperature and concentrated under reduced pressure. The residue was purified by Prep-HPLC with the following conditions: YMC-Actus Triart C18, 30×250 mm, 5 um; Mobile Phase A: Water (10 mmol/L NH₄HCO₃), Mobile Phase B: ACN; Flow rate: 60 mL/min; Gradient: 29% B to 38% B in 8 min; 254/220 nm to afford compound XX (7.5 mg, 5%) as a white solid. LCMS (ESI, m/z): [M+H]⁺=350.2. ¹H NMR (300 MHz, CD₃OD): δ 8.14 (s, 1H), 7.00 (s, 1H), 1.90-1.59 (m, 10H).

Following the procedure described above for Example 11 and substituting the appropriate reagents, starting materials and purification methods known to those skilled in the art, the compounds listed below were synthesized

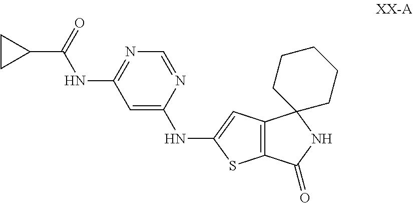 [SMILES: O=C1NC2(CCCCC2)c2cc(Nc3cc(NC(=O)C4CC4)ncn3)sc21]

N-(6-((6′-Oxo-5′,6′-dihydrospiro[cyclohexane-1,4′-thieno[2,3-c]pyrrol]-2′-yl)amino)pyrimidin-4-yl)cyclopropanecarboxamide (Compound XX-A): LCMS (ESI, m/z): [M+H]⁺=384.1. ¹H NMR (300 MHz, DMSO-d₆): δ 11.07-10.96 (m, 2H), 8.55 (s, 1H), 8.43 (s, 1H), 7.59 (s, 1H), 6.69 (s, 1H), 2.06-2.02 (m, 1H), 1.81-1.65 (m, 6H), 1.52-1.48 (m, 4H), 0.87-0.84 (m, 4H).

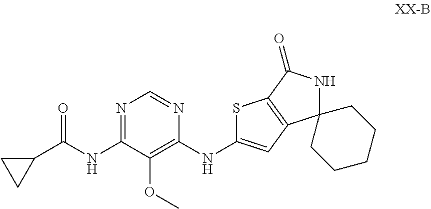 [SMILES: COc1c(NC(=O)C2CC2)ncnc1Nc1cc2c(s1)C(=O)NC21CCCCC1]

N-(5-Methoxy-6-((6′-oxo-5′,6′-dihydrospiro[cyclohexane-1,4′-thieno[2,3-c]pyrrol]-2′-yl)amino)pyrimidin-4-yl)cyclopropanecarboxamide (Compound XX-B): LCMS (ESI, m/z): [M+H]⁺=414.1. ¹H NMR (300 MHz, DMSO-d₆): δ 10.67 (s, 1H), 10.31 (s, 1H), 8.42 (s, 1H), 8.37 (s, 1H), 7.07 (s, 1H), 3.71 (s, 3H), 2.10-2.06 (m, 1H), 1.72-1.52 (m, 10H), 0.85-0.83 (m, 4H).

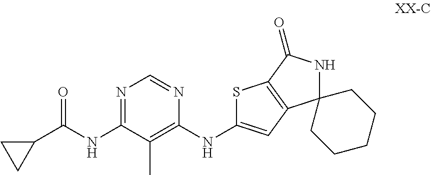 [SMILES: Cc1c(NC(=O)C2CC2)ncnc1Nc1cc2c(s1)C(=O)NC21CCCCC1]

N-(5-Methyl-6-((6′-oxo-5′,6′-dihydrospiro[cyclohexane-1,4′-thieno[2,3-c]pyrrol]-2′-yl)amino)pyrimidin-4-yl)cyclopropanecarboxamide (Compound XX-C): LCMS (ESI, m/z): [M+H]⁺=398.2. ¹H NMR (300 MHz, DMSO-d₆): δ 10.45 (s, 1H), 10.13 (s, 1H), 8.52 (s, 1H), 8.41 (s, 1H), 7.03 (s, 1H), 1.99 (s, 3H), 1.94-1.90 (m, 1H), 1.73-1.62 (m, 6H), 1.59-1.48 (m, 4H), 0.85-0.79 (m, 4H).

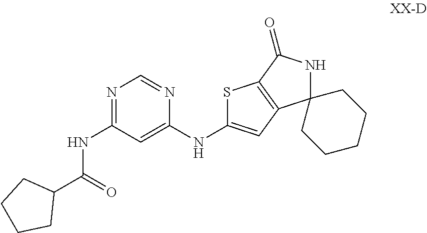 [SMILES: O=C1NC2(CCCCC2)c2cc(Nc3cc(NC(=O)C4CCCC4)ncn3)sc21]

N-(6-((6′-Oxo-5′,6′-dihydrospiro[cyclohexane-1,4′-thieno[2,3-c]pyrrol]-2′-yl)amino)pyrimidin-4-yl)cyclopentanecarboxamide (Compound XX-D): LCMS (ESI, m/z): [M+H]⁺=412.1. ¹H NMR (300 MHz, DMSO-d₆): δ 11.08 (s, 1H), 10.60 (s, 1H), 8.51 (d, J=0.9 Hz, 1H), 8.43 (s, 1H), 7.61 (d, J=1.2 Hz, 1H), 6.67 (s, 1H), 2.92-2.70 (m, 1H), 1.88-1.77 (m, 2H), 1.72-1.64 (m, 10H), 1.63-1.46 (m, 6H).

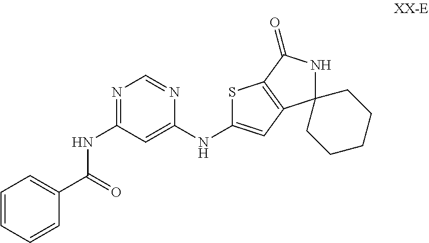 [SMILES: O=C(Nc1cc(Nc2cc3c(s2)C(=O)NC32CCCCC2)ncn1)c1ccccc1]

N-(6-((6′-oxo-5′,6′-dihydrospiro[cyclohexane-1,4′-thieno[2,3-c]pyrrol]-2′-yl)amino)pyrimidin-4-yl)benzamide (Compound XX-E): LCMS (ESI, m/z): [M+H]⁺=420.1. ¹H NMR (400 MHz, DMSO-d₆): δ 11.21 (s, 1H), 10.99 (s, 1H), 8.63 (s, 1H), 8.46 (s, 1H), 8.03-8.01 (m, 2H), 7.81 (s, 1H), 7.64-7.60 (m, 1H), 7.54-7.51 (m, 2H), 6.73 (s, 1H), 1.79-1.69 (m, 6H), 1.68-1.51 (m, 4H).

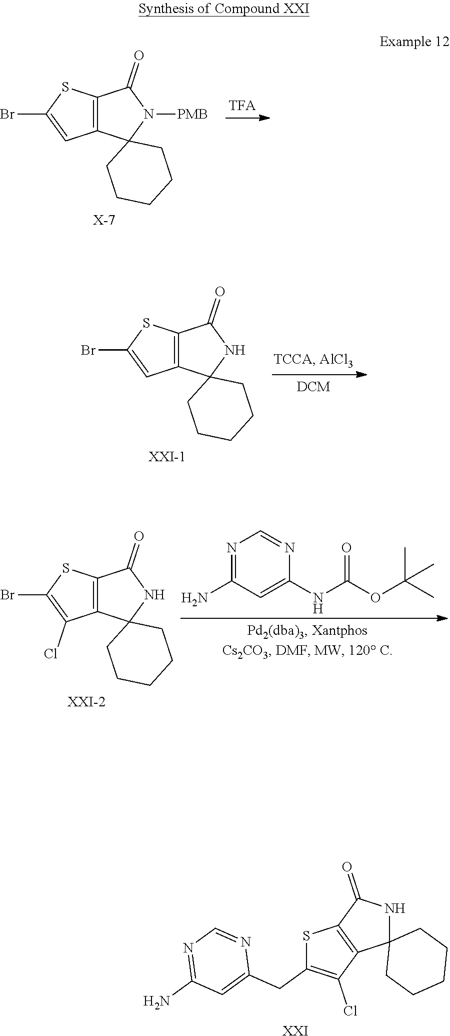

2′-Bromospiro[cyclohexane-1,4′-thieno[2,3-c]pyrrol]-6′(5′H)-one (Compound XXI-1): A solution of 2′-bromo-5′-(4-methoxybenzyl)spiro[cyclohexane-1,4′-thieno[2,3-c]pyrrol]-6′(5′H)-one (750.0 mg, 1.85 mmol) in TFA (10.0 mL) was stirred at 80° C. for 16 h. After the reaction was completed, the resulting mixture was concentrated under reduced pressure. The residue was purified by flash column chromatography with petroleum ether/ethyl acetate (40/60, v/v) to afford compound XXI-1 (531.7 mg, 98%) as a grey solid. LCMS (ESI, m/z): [M+H]⁺=286.0.

2′-Bromo-3′-chlorospiro[cyclohexane-1,4′-thieno[2,3-c]pyrrol]-6′(5′II)-one (Compound XXI-2): To a solution of 2′-bromospiro[cyclohexane-1,4′-thieno[2,3-c]pyrrol]-6′(5′H)-one (531.7 mg, 1.86 mmol) in CH₂Cl₂ (15.0 mL) was added trichloroisocyanuric acid (431.2 mg. 1.86 mmol) and AlCl₃ (1.2 g, 9.29 mmol) at 10° C. The resulting mixture was stirred at 10° C. for 2 h. After the reaction was completed, the mixture was concentrated under reduced pressure. The residue was purified by flash column chromatography with petroleum ether/ethyl acetate (70/30, v/v) to afford compound XXI-2 (496.0 mg. 83%) as a white solid. LCMS (ESI, m/z): [M+H]⁺=320.0.

2′-((6-Aminopyrimidin-4-yl)amino)-3′-chlorospiro[cyclohexane-1,4′-thieno[2,3-c]pyrrol]-6′(5′H)-one (Compound XXI): To a solution of 2′-bromo-3′-chlorospiro[cyclohexane-1,4′-thieno[2,3-c]pyrrol]-6′(5′H)-one (200.0 mg, 0.62 mmol) in DMF (15.0 mL) was added tert-butyl (6-aminopyrimidin-4-yl)carbamate (157.0 mg, 0.75 mmol), Pd₂(dba)₃ (57.0 mg, 0.06 mmol), XantPhos (72.2 mg, 0.12 mmol) and Cs₂CO₃ (609.7 mg, 1.87 mmol). The reaction mixture was irradiated with microwave radiation at 120° C. for 3 h under N₂. After the reaction was completed, the mixture was filtered. The filtrate was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (85/15, v/v) and then purified by Prep-HPLC with the following conditions: Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 31% B to 40% B in 8 min; 254/220 nm; to afford compound XXI (4.4 mg, 2%) as a white solid. LCMS (ESI, m/z): [M+H]⁺=350.0. ¹H NMR (400 MHz, DMSO-d₆): δ 9.80 (s, 1H), 8.82 (s, 1H), 8.17 (d, J=0.8 Hz, 1H), 6.66 (s, 2H), 6.14 (d, J=0.8 Hz, 1H), 2.13-2.06 (m, 2H), 1.72-1.59 (m, 5H), 1.42-1.38 (m, 2H), 1.32-1.21 (m, 1H).

Following the procedure described above for Example 12 and substituting the appropriate reagents, starting materials and purification methods known to those skilled in the art, the compounds listed below were synthesized.

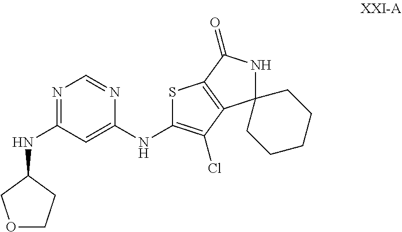 [SMILES: O=C1NC2(CCCCC2)c2c1sc(Nc1cc(N[C@H]3CCOC3)ncn1)c2Cl]

(S)-3′-chloro-2′-((6-((tetrahydrofuran-3-yl)amino)pyrimidin-4-yl)amino)spiro[cyclohexane-1,4′-thieno[2,3-c]pyrrol]-6′(5′H)-one (Compound XXI-A): LCMS (ESI, m/z): [M+H]⁺=420.3. ¹H NMR (400 MHz, DMSO-d₆): δ 9.82 (s, 1H), 8.81 (s, 1H), 8.26 (s, 1H), 7.40 (d, J=6.0 Hz, 1H), 6.19 (s, 1H), 4.33 (s, 1H), 3.88-3.81 (m, 2H), 3.75-3.70 (m, 1H), 3.57-3.54 (m, 1H), 2.20-2.08 (m, 3H), 1.87-1.83 (m, 1H), 1.79-1.63 (m, 5H), 1.43-1.39 (m, 2H), 1.29-1.23 (m, 1H).

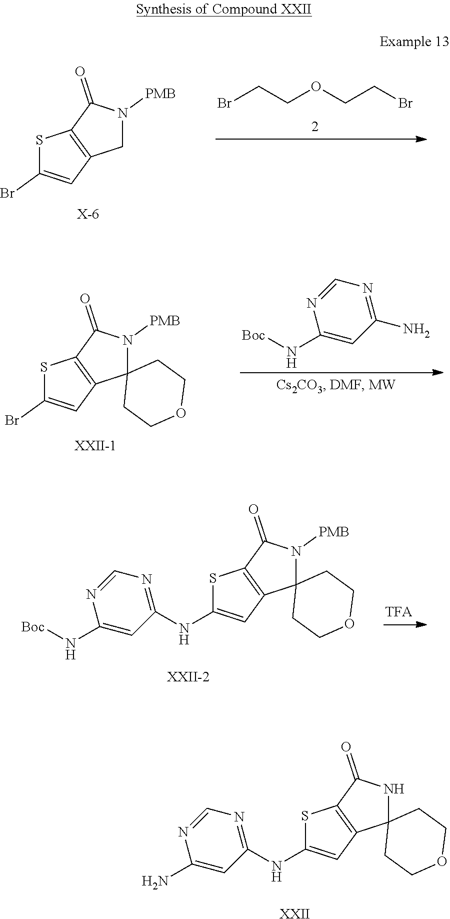

2′-Bromo-5′-(4-methoxybenzyl)-2,3,5,6-tetrahydrospiro[pyran-4,4′-thieno[2,3-c]pyrrol]-6′(5′H)-one (Compound XXII-1): To a solution of 2-bromo-5-[(4-methoxyphenyl)methyl]-4H-thieno[2,3-c]pyrrol-6-one (500.0 mg, 1.47 mmo) in DMF (10.0 mL) was added NaH (395.9 mg, 60%) at 0° C. under N₂. The mixture was stirred at 0° C. for 30 min. Then 1-bromo-2-(2-bromoethoxy)ethane (342.8 mg, 1.47 mmol) was added to the mixture. The reaction mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (50/50, v/v) to afforded compound XXII-1 (260.0 mg, 43%) as a red solid. LCMS (ESI, m/z): [M+H]⁺=408.0.

tert-Butyl (6-((5′-(4-methoxybenzyl)-6′-oxo-2,3,5,5′,6,6′-hexahydrospiro[pyran-4,4′-thieno[2,3-c]pyrrol]-2′-yl)amino)pyrimidin-4-yl)carbamate (Compound XXII-2): To a solution of 2′-bromo-5′-(4-methoxybenzyl)-2,3,5,6-tetrahydrospiro[pyran-4,4′-thieno[2,3-c]pyrrol]-6′(5′H)-one (200.0 mg, 0.49 mmol) in DMF (10.0 mL) was added tert-butyl N-(6-aminopyrimidin-4-yl)carbamate (102.9 mg, 0.49 mmol), Cs₂CO₃ (478.7 mg, 1.47 mmol), BrettPhos (105.1 mg, 0.19 mmol) and BrettPhos Pd G3 (88.8 mg, 0.09 mmol). The reaction mixture was stirred at 120° C. for 16 h. After the reaction was completed, the mixture was cooled to room temperature and diluted with H₂O. The mixture was extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (30/70, v/v) to afforded compound XXII-2 (56.0 mg, 21%) as a white solid. LCMS (ESI, m/z): [M+H]⁺=538.2.

2′-((6-Aminopyrimidin-4-yl)amino)-2,3,5,6-tetrahydrospiro[pyran-4,4′-thieno[2,3-c]pyrrol]-6′(5′H)-one (Compound XXII): A solution of tert-butyl (6-((5′-(4-methoxybenzyl)-6′-oxo-2,3,5,5′,6,6′-hexahydrospiro[pyran-4,4′-thieno[2,3-c]pyrrol]-2′-yl)amino)pyrimidin-4-yl)carbamate (50.0 mg, 0.09 mmol) in TFA (5.0 mL) was stirred at 50° C. for 16 h. After the reaction was completed, the pH value of the mixture was adjusted to 8 with saturated NaHCO₃ (aq.) and then extracted with ethyl acetate. The combined organic layer was washed with brine, dried over anhydrous sodium sulfate and filtered. The filtrate was concentrated under vacuum. The residue was purified by Prep-HPLC with the following conditions: Column: YMC-Actus Triart C18, 30×250 mm, 5 um; Mobile Phase A: Water (10 mmol/L NH₄HCO₃), Mobile Phase B: ACN; Flow rate: 60 mL/min; Gradient: 12% B to 21% B in 10 min; 254/220 nm to afford compound XXII (4.7 mg, 15%) as a white solid. LCMS (ESI, m/z): [M+H]⁺=318.0. ¹H NMR (400 MHz, DMSO-d₆): δ 10.50 (s, 1H), 8.61 (s, 1H), 8.16 (d, J=0.8 Hz, 1H), 6.62 (s, 1H), 6.55 (s, 2H), 5.79 (d, J=1.2 Hz, 1H), 3.78-3.72 (m, 4H), 1.99-1.92 (m, 2H), 1.52-1.47 (m, 2H).

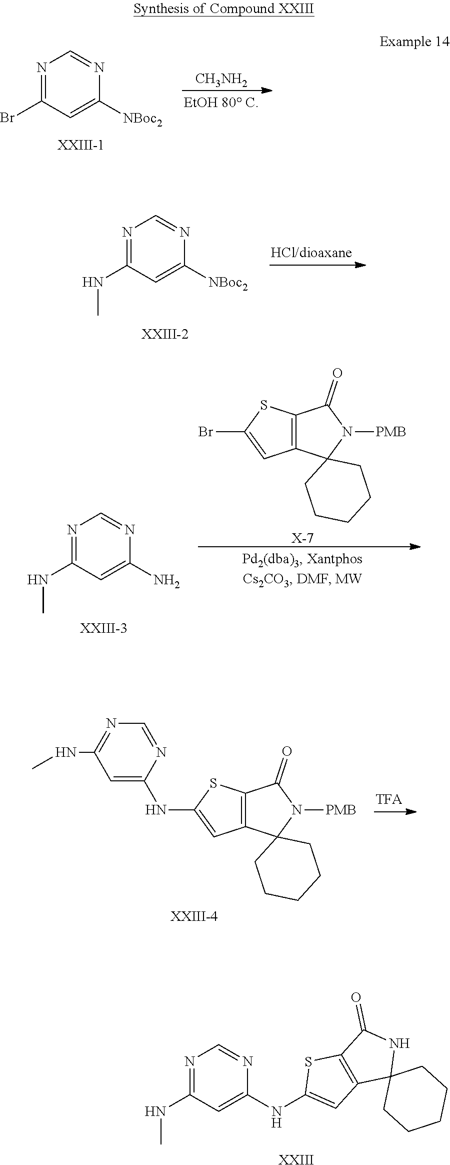

tert-Butyl N-(tert-butoxycarbonyl)-N-[6-(methylamino)pyrimidin-4-yl]carbamate (Compound XXIII-2): To a solution of tert-butyl N-(6-bromopyrimidin-4-yl)-N-(tert-butoxycarbonyl)carbamate (1.0 g, 2.67 mmol) in EtOH (10.0 mL) was added CH₃NH₂ (1.4 g, 30%). The resulting mixture was stirring at 80° C. for 16 h. After the reaction was completed, the resulting mixture was cooled to room temperature and concentrated under reduced pressure. The residue was purified by flash column chromatography with CH₂Cl₂/MeOH (80/20, v/v) to afford compound XXIII-2 (1.2 g, 95%) as a white solid. LCMS (ESI, m/z): [M+H]⁺=325.2.

N4-Methylpyrimidine-4,6-diamine (Compound XXIII-3): A solution of tert-butyl N-(tert-butoxycarbonyl)-N-[6-(methylamino)pyrimidin-4-yl]carbamate (1.2 g, 3.64 mmol) in HCl/dioxane (20.0 mL, 4.0 mol/L) was stirred at room temperature for 16 h. After the reaction was completed, the pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by flash column chromatography with CH₂Cl₂/MeOH (80/20, v/v) to afford compound XXIII-3 (560.0 mg, 99%) as a white solid. LCMS (ESI, m/z): [M+H]⁺=125.1.

5′-(4-Methoxybenzyl)-2′-((6-(methylamino)pyrimidin-4-yl)amino)spiro[cyclohexane-1,4′-thieno[2,3-c]pyrrol]-6′(5′H)-one (Compound XXIII-4): To a solution of 2′-bromo-5′-(4-methoxybenzyl)spiro[cyclohexane-1,4′-thieno[2,3-c]pyrrol]-6′(5′H)-one (200.0 mg, 0.49 mmol) in DMF (12.0 mL) was added N4-methylpyrimidine-4,6-diamine (183.3 mg, 1.48 mmol), Pd₂(dba)₃ (135.2 mg, 0.15 mmol), XantPhos (170.9 mg, 0.30 mmol) and Cs₂CO₃ (481.1 mg, 1.48 mmol). The resulting mixture was stirred at 130° C. for 2 h under N₂. After the reaction was completed, the resulting mixture was cooled to room temperature and filtered. The filtrate was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (90/10, v/v) to afford compound XXIII-4 (77.0 mg, 34%) as a yellow solid. LCMS (ESI, m/z): [M+H]⁺=450.2.

2′-((6-(Methylamino)pyrimidin-4-yl)amino)spiro[cyclohexane-1,4′-thieno[2,3-c]pyrrol]-6′(5′H)-one (Compound XXIII): A solution of 5′-(4-methoxybenzyl)-2′-((6-(methylamino)pyrimidin-4-yl)amino)spiro[cyclohexane-1,4′-thieno[2,3-c]pyrrol]-6′(5′H)-one (57.0 mg, 0.13 mmol) in TFA (8.0 mL) was stirred at 75° C. for 4 h. After the reaction was completed, the resulting mixture was cooled to room temperature. The pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 25% B to 55% B in 7 min; 254 nm to afford compound XXIII (4.5 mg, 10%) as a white solid. LCMS (ESI, m/z): [M+H]⁺=330.1. ¹H NMR (300 MHz, DMSO-d₆): δ 10.47 (s, 1H), 8.36 (s, 1H), 8.22 (s, 1H), 7.09-6.99 (m, 1H), 6.57 (s, 1H), 5.72 (s, 1H), 2.76 (d, J=4.5 Hz, 3H), 1.73-1.48 (m, 10H).

Following the procedure described above for Example 14 and substituting the appropriate reagents, starting materials and purification methods known to those skilled in the art, the compounds listed below were prepared.

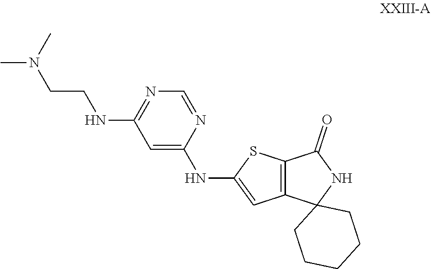 [SMILES: CN(C)CCNc1cc(Nc2cc3c(s2)C(=O)NC32CCCCC2)ncn1]

2′-((6-((2-(Dimethylamino)ethyl)amino)pyrimidin-4-yl)amino)spiro [cyclohexane-1,4′-thieno[2,3-c]pyrrol]-6′(5′H)-one (Compound XXIII-A: LCMS (ESI, m/z): [M+H]⁺=387.1. ¹H NMR (300 MHz, DMSO-d₆): δ 10.45 (s, 1H), 8.36 (s, 1H), 8.21 (s, 1H), 6.98 (s, 1H), 6.57 (s, 1H), 5.80 (s, 1H), 2.40-2.36 (m, 2H), 2.18 (s, 6H), 1.72-1.65 (m, 6H), 1.57-1.48 (m, 4H).

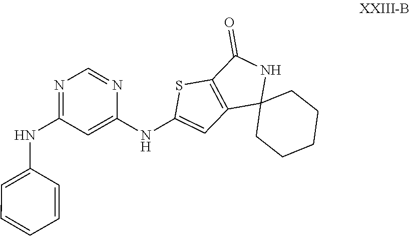 [SMILES: O=C1NC2(CCCCC2)c2cc(Nc3cc(Nc4ccccc4)ncn3)sc21]

2′-((6-(Phenylamino)pyrimidin-4-yl)amino)spiro[cyclohexane-1,4′-thieno[2,3-c]pyrrol]-6′(5′H)-one (Compound XXIII-B): LCMS (ESI, m/z): [M+H]⁺=392.2. ¹H NMR (300 MHz, DMSO-d₆): δ 10.69 (s, 1H), 9.33 (s, 1H), 8.41 (s, 2H), 7.57-7.54 (m, 2H), 7.36-7.30 (m, 2H), 7.04-6.98 (m, 1H), 6.64 (s, 1H), 6.19 (d, J=0.6 Hz, 1H), 1.73-1.49 (m, 10H).

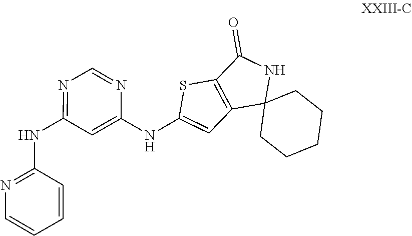 [SMILES: O=C1NC2(CCCCC2)c2cc(Nc3cc(Nc4ccccn4)ncn3)sc21]

2′-((6-(Pyridin-2-ylamino)pyrimidin-4-yl)amino)spiro[cyclohexane-1,4′-thieno[2,3-c]pyrrol]-6′(5′H)-one (Compound XXIII-C): The title compound was synthesized by using tert-butyl N-(tert-butoxycarbonyl)-N-[6-(pyridin-2-ylamino)pyrimidin-4-yl]carbamate as the starting material of which the synthesis was shown below. LCMS (ESI, m/z): [M+H]⁺=393.2. ¹H NMR (300 MHz, DMSO-d₆): δ 10.95 (s, 1H), 9.99 (s, 1H), 8.44-8.39 (m, 2H), 8.29-8.27 (m, 1H), 7.72-7.66 (m, 1H), 7.52 (s, 1H), 7.41 (d, J=8.4 Hz, 1H), 6.97-6.93 (m, 1H), 6.66 (s, 1H), 1.72-1.47 (m, 10H).

The synthesis of tert-butyl N-(tert-butoxycarbonyl)-N-[6-(pyridin-2-ylamino)pyrimidin-4-yl]carbamate: To a solution of tert-butyl N-(tert-butoxycarbonyl)-N-(6-chloropyrimidin-4-yl)carbamate (1000.0 mg, 3.03 mmol) in dioxane (20.0 mL) was added pyridin-2-amine (856.18 mg, 9.10 mmol), Pd₂(dba)₃ (833.0 mg, 0.91 mmol), XantPhos (1052.7 mg, 1.82 mmol) and Cs₂CO₃ (2963.9 mg, 9.20 mmol). The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (90/10, v/v) to afford tert-butyl N-(tert-butoxycarbonyl)-N-[6-(pyridin-2-ylamino)pyrimidin-4-yl]carbamate (600.0 mg, 51%) as a white solid. LCMS (ESI, m/z): [M+H]⁺=388.2.

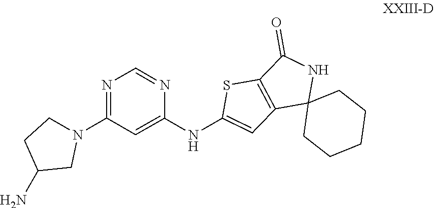 [SMILES: NC1CCN(c2cc(Nc3cc4c(s3)C(=O)NC43CCCCC3)ncn2)C1]

2′-((6-(3-Aminopyrrolidin-1-yl)pyrimidin-4-yl)amino)spiro[cyclohexane-1,4′-thieno[2,3-c]pyrrol]-6′(5′H)-one (Compound XXIII-D): The title compound was synthesized by using tert-butyl (1-(6-aminopyrimidin-4-yl)pyrrolidin-3-yl)carbamate as the starting material of which the synthesis was shown below.

LCMS (ESI, m/z): [M+H]⁺=385.2. ¹H NMR (300 MHz, DMSO-d₆): δ 10.53 (s, 1H), 8.36 (s, 1H), 8.26 (s, 1H), 6.59 (s, 1H), 5.64 (s, 1H), 3.63-3.44 (m, 4H), 2.11-1.83 (m, 3H), 1.79-1.65 (m, 8H), 1.64-1.48 (m, 4H).

The synthesis of tert-butyl (1-(6-aminopyrimidin-4-yl)pyrrolidin-3-yl)carbamate: To a solution of 6-chloropyrimidin-4-amine (1.0 g, 7.72 mmol) in DMF (20.0 mL) was added tert-butyl pyrrolidin-3-ylcarbamate (2.88 g, 15.46 mmol) and Cs₂CO₃ (3.8 g, 11.58 mmol). The resulting mixture was stirred at 100° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (90/10, v/v) to afford tert-butyl (1-(6-aminopyrimidin-4-yl)pyrrolidin-3-yl)carbamate (600.0 g, 28%) as a white solid. LCMS (ESI, m/z): [M+H]⁺=280.3.

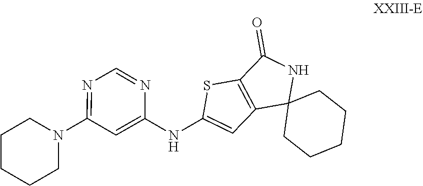 [SMILES: O=C1NC2(CCCCC2)c2cc(Nc3cc(N4CCCCC4)ncn3)sc21]

2′-((6-(Piperidin-1-yl)pyrimidin-4-yl)amino)spiro[cyclohexane-1,4′-thieno[2,3-c]pyrrol]-6′(5′H)-one (Compound XXIII-E): LCMS (ESI, m/z): [M+H]⁺=384.1. ¹H NMR (300 MHz, DMSO-d₆): δ 10.52 (s, 1H), 8.37 (s, 1H), 8.29 (s, 1H), 6.61 (s, 1H), 5.95 (s, 1H), 3.56-3.52 (m, 4H), 1.73-1.53 (m, 16H).

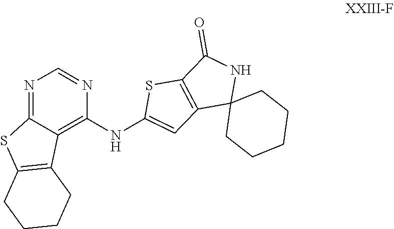 [SMILES: O=C1NC2(CCCCC2)c2cc(Nc3ncnc4sc5c(c34)CCCC5)sc21]

2′-((5,6,7,8-Tetrahydrobenzo[4,5]thieno[2,3-d]pyrimidin-4-yl)amino)spiro [cyclohexane-1,4′-thieno[2,3-c]pyrrol]-6′(5′H)-one (Compound XXIII-F): LCMS (ESI, m/z): [M+H]⁺=411.0. ¹H NMR (400 MHz, DMSO-d₆): δ 9.34 (s, 1H), 8.59 (s, 1H), 8.43 (s, 1H), 7.18 (s, 1H), 3.16 (s, 2H), 2.85 (s, 2H), 1.89-1.87 (m, 4H), 1.71-1.69 (m, 6H), 1.61-1.48 (m, 4H).

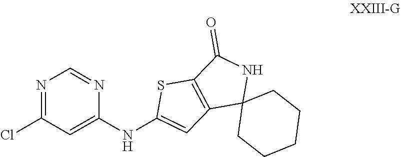 [SMILES: O=C1NC2(CCCCC2)c2cc(Nc3cc(Cl)ncn3)sc21]

2′-((6-Chloropyrimidin-4-yl)amino)spiro[cyclohexane-1,4′-thieno[2,3-c]pyrrol]-6′(5′H)-one (Compound XXIII-G): LCMS (ESI, m/z): [M+H]⁺=334.9. ¹H NMR (300 MHz, DMSO-d₆): δ 11.33 (s, 1H), 8.66 (d, J=0.6 Hz, 1H), 8.55 (s, 1H), 6.89 (d, J=0.6 Hz, 1H), 6.83 (s, 1H), 1.77-1.60 (m, 6H), 1.53-1.48 (m, 4H).

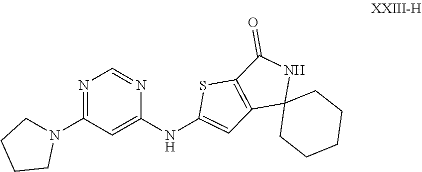 [SMILES: O=C1NC2(CCCCC2)c2cc(Nc3cc(N4CCCC4)ncn3)sc21]

2′-((6-(Pyrrolidin-1-yl)pyrimidin-4-yl)amino)spiro[cyclohexane-1,4′-thieno[2,3-c]pyrrol]-6′(5′H)-one (Compound XXIII-H): LCMS (ESI, m/z): [M+H]⁺=370.2. ¹H NMR (300 MHz, DMSO-d₆): δ 10.54 (s, 1H), 8.36 (s, 1H), 8.27 (s, 1H), 6.59 (s, 1H), 5.67 (s, 1H), 3.38-3.29 (m, 4H), 2.00-1.89 (m, 4H), 1.73-1.65 (m, 6H), 1.51-1.40 (m, 4H).

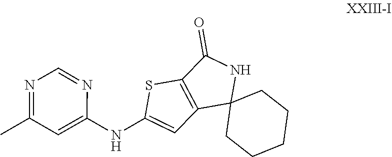 [SMILES: Cc1cc(Nc2cc3c(s2)C(=O)NC32CCCCC2)ncn1]

2′-((6-Methylpyrimidin-4-yl)amino)spiro[cyclohexane-1,4′-thieno[2,3-c]pyrrol]-6′(5′H)-one (Compound XXIII-I): LCMS (ESI, m/z): [M+H]⁺=315.0. ¹H NMR (300 MHz, DMSO-d₆): δ 11.71 (s, 1H), 8.88 (s, 1H), 8.59 (s, 1H), 6.88 (s, 1H), 6.81 (s, 1H), 2.41 (s, 3H), 1.79-1.65 (m, 6H), 1.64-1.33 (m, 4H).

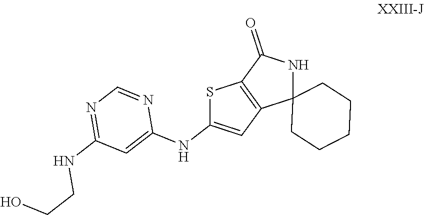 [SMILES: O=C1NC2(CCCCC2)c2cc(Nc3cc(NCCO)ncn3)sc21]

2′-((6-((2-Hydroxyethyl)amino)pyrimidin-4-yl)amino)spiro[cyclohexane-1,4′-thieno[2,3-c]pyrrol]-6′(5′H)-one (Compound XXIII-J): The title compound was synthesized by using N4-[2-[(tert-butyldimethylsilyl)oxy]ethyl]pyrimidine-4,6-diamine as the starting material of which the synthesis was shown below. LCMS (ESI, m/z): [M+H]⁺=360.2. ¹H NMR (300 MHz, DMSO-d₆): δ 10.45 (s, 1H), 8.35 (s, 1H), 8.21 (s, 1H), 7.09 (s, 1H), 6.57 (s, 1H), 5.82 (s, 1H), 4.77-4.73 (m, 1H), 3.54-3.48 (m, 2H), 3.30-3.25 (m, 2H), 1.72-1.65 (m, 6H), 1.60-1.46 (m, 4H).

### Synthesis of N4-[2-[(tert-butyldimethylsilyl)oxy]ethyl]pyrimidine-4,6-diamine

Step 1. To a solution of 6-chloropyrimidin-4-amine (10.0 g, 77.19 mmol) in EtOH (300.0 mL) was added 2-aminoethan-1-ol (18.9 g, 202.94 mmol). The mixture was stirred at 80° C. for 16 h. After the reaction was completed, the reaction mixture was evaporated in vacuo. The residue was purified by flash column chromatography with CH₂Cl₂/MeOH (90/10, v/v) to afford 2-((6-aminopyrimidin-4-yl)amino)ethan-1-ol (1.9 g, 14%) as a yellow solid. LCMS (ESI, m/z): [M+H]⁺=155.1.

Step 2. To a mixture of 2-[(6-aminopyrimidin-4-yl)amino]ethanol (500.0 mg, 3.24 mmol) and imidazole (221.0 mg, 3.25 mmol) in CH₂Cl₂ (30.0 mL) was added t-butyldimethylchlorosilane (487.0 mg, 3.23 mmol) at 0° C. The resulting mixture was stirred at room temperature for 16 h. After the reaction was completed, the pH value of the mixture was adjusted to 7.0 with saturated NaHCO₃ (aq). The resulting mixture was extracted with CH₂Cl₂. The combined organic layer was washed with brine, dried over anhydrous sodium sulfate and filtered. The filtrate was concentrated under reduced pressure to afford N4-[2-[(tert-butyldimethylsilyl)oxy]ethyl]pyrimidine-4,6-diamine (560.0 mg, crude) as a white solid. LCMS (ESI, m/z): [M+H]⁺=269.2.

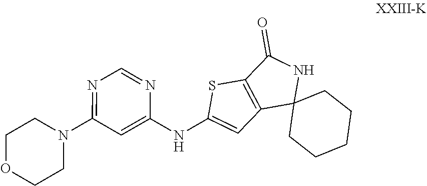 [SMILES: O=C1NC2(CCCCC2)c2cc(Nc3cc(N4CCOCC4)ncn3)sc21]

2′-((6-Morpholinopyrimidin-4-yl)amino)spiro[cyclohexane-1,4′-thieno[2,3-c]pyrrol]-6′(5′H)-one (Compound XXIII-K): LCMS (ESI, m/z): [M+H]⁺=386.2. ¹H NMR (300 MHz, DMSO-d₆): δ10.64 (s, 1H), 8.39-8.34 (m, 2H), 6.63 (s, 1H), 5.95 (s, 1H), 3.70-3.67 (m, 4H), 3.50-3.47 (m, 4H), 1.73-1.65 (m, 6H), 1.60-1.48 (m, 4H).

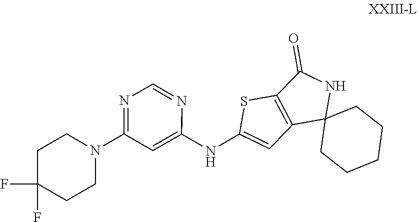 [SMILES: O=C1NC2(CCCCC2)c2cc(Nc3cc(N4CCC(F)(F)CC4)ncn3)sc21]

2′-((6-(4,4-Difluoropiperidin-1-yl)pyrimidin-4-yl)amino)spiro[cyclohexane-1,4′-thieno[2,3-c]pyrrol]-6′(5′H)-one (Compound XXIII-L): LCMS (ESI, m/z): [M+H]⁺=420.0. ¹H NMR (300 MHz, DMSO-d₆): δ 10.67 (s, 1H), 8.41 (s, 1H), 8.36 (s, 1H), 6.64 (s, 1H), 6.06 (s, 1H), 3.72-3.68 (m, 4H), 2.22-1.91 (m, 4H), 1.84-1.57 (m, 6H), 1.56-1.31 (m, 4H).

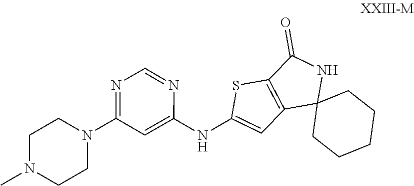 [SMILES: CN1CCN(c2cc(Nc3cc4c(s3)C(=O)NC43CCCCC3)ncn2)CC1]

2′-((6-(4-Methylpiperazin-1-yl)pyrimidin-4-yl)amino)spiro[cyclohexane-1,4′-thieno[2,3-c]pyrrol]-6′(5′H)-one (Compound XXIII-M): LCMS (ESI, m/z): [M+H]⁺=399.1. ¹H NMR (300 MHz, CD₃OD): δ 8.40 (d, J=0.6 Hz, 1H), 6.72 (s, 1H), 6.09 (d, J=0.9 Hz, 1H), 4.72-4.56 (m, 2H), 3.65-3.55 (m, 2H), 3.30-3.17 (m, 2H), 2.99 (s, 3H), 1.89-1.52 (m, 10H).

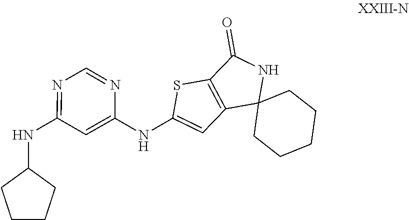 [SMILES: O=C1NC2(CCCCC2)c2cc(Nc3cc(NC4CCCC4)ncn3)sc21]

2′-((6-(Cyclopentylamino)pyrimidin-4-yl)amino)spiro[cyclohexane-1,4′-thieno[2,3-c]pyrrol]-6′(5′H)-one (Compound XXIII-N): LCMS (ESI, m/z): [M+H]⁺=384.2. ¹H NMR (300 MHz, DMSO-d₆): δ 10.42 (s, 1H), 8.33 (s, 1H), 8.18 (s, 1H), 7.06 (d, J=6.9 Hz, 1H), 6.55 (s, 1H), 5.73 (s, 1H), 4.15-3.89 (m, 1H), 1.91-1.85 (m, 2H), 1.70-1.29 (m, 16H).

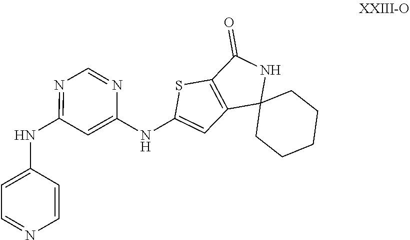 [SMILES: O=C1NC2(CCCCC2)c2cc(Nc3cc(Nc4ccncc4)ncn3)sc21]

2′-((6-(pyridin-4-ylamino)pyrimidin-4-yl)amino)spiro[cyclohexane-1,4′-thieno[2,3-c]pyrrol]-6′(5′H)-one (Compound XXIII-O): LCMS (ESI, m/z): [M+H]⁺=393.0. ¹H NMR (300 MHz, DMSO-d₆): δ 10.87 (s, 1H), 9.82 (s, 1H), 8.54 (s, 1H), 8.46 (s, 1H), 8.38-8.36 (m, 2H), 7.66-7.64 (m, 2H), 6.70 (s, 1H), 6.30 (d, J=0.6 Hz, 1H), 1.75-1.66 (m, 6H), 1.53-1.39 (m, 4H).

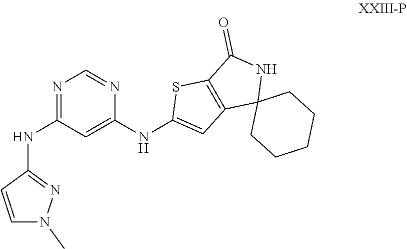 [SMILES: Cn1ccc(Nc2cc(Nc3cc4c(s3)C(=O)NC43CCCCC3)ncn2)n1]

2′-((6-((1-methyl-1H-pyrazol-3-yl)amino)pyrimidin-4-yl)amino)spiro[cyclohexane-1,4′-thieno[2,3-c]pyrrol]-6′(5′H)-one (Compound XXIII-P): LCMS (ESI, m/z): [M+H]⁺=396.0. ¹H NMR (400 MHz, DMSO-d₆): δ 10.76 (s, 1H), 9.56 (s, 1H), 8.37-8.34 (m, 2H), 7.57 (d, J=2.0 Hz, 1H), 6.80 (s, 1H), 6.63 (s, 1H), 6.12 (s, 1H), 3.78 (s, 3H), 1.72-1.65 (m, 6H), 1.51-1.35 (m, 4H).

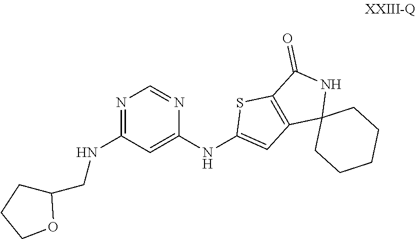 [SMILES: O=C1NC2(CCCCC2)c2cc(Nc3cc(NCC4CCCO4)ncn3)sc21]

2′-((6-(((tetrahydrofuran-2-yl)methyl)amino)pyrimidin-4-yl)amino)spiro[cyclohexane-1,4′-thieno[2,3-c]pyrrol]-6′(5′H)-one (Compound XXIII-Q): LCMS (ESI, m/z): [M+H]⁺=400.0. ¹H NMR (400 MHz, DMSO-d₆): δ 10.45 (s, 1H), 8.34 (s, 1H), 8.21 (s, 1H), 7.19 (s, 1H), 6.57 (s, 1H), 5.85 (s, 1H), 3.97-3.91 (m, 1H), 3.80-3.75 (m, 1H), 3.65-3.61 (m, 1H), 1.93-1.64 (m, 14H).

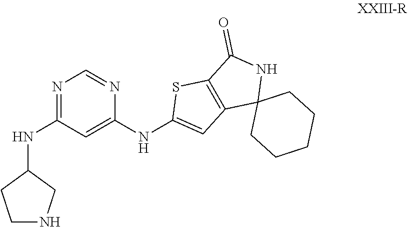 [SMILES: O=C1NC2(CCCCC2)c2cc(Nc3cc(NC4CCNC4)ncn3)sc21]

2′-((6-(pyrrolidin-3-ylamino)pyrimidin-4-yl)amino)spiro[cyclohexane-1,4′-thieno[2,3-c]pyrrol]-6′(5′H)-one (Compound XXIII-R): The title compound was synthesized by using tert-butyl 3-((6-aminopyrimidin-4-yl)amino)pyrrolidine-1-carboxylate as the starting material of which the synthesis was shown below. LCMS (ESI, m/z): [M+H]⁺=385.2. ¹H NMR (300 MHz, DMSO-d₆): δ 10.61 (s, 1H), 8.37 (s, 2H), 8.27 (s, 1H), 7.58 (s, 1H), 6.59 (s, 1H), 5.84 (s, 1H), 4.51-4.28 (m, 1H), 3.31-3.12 (m, 3H), 2.97-2.92 (m, 1H), 2.18-2.08 (m, 1H), 1.86-1.80 (m, 1H), 1.79-1.63 (m, 6H), 1.62-1.46 (m, 4H).

The synthesis of tert-butyl 3-((6-aminopyrimidin-4-yl)amino)pyrrolidine-1-carboxylate: To a solution of 6-chloropyrimidin-4-amine (2.0 g, 15.44 mmol) in DMF (100.0 mL) was added tert-butyl 3-aminopyrrolidine-1-carboxylate (5.8 g, 0.03 mmol) and Cs₂CO₃ (3.8 g, 11.66 mmol) at room temperature. The reaction mixture was irradiated with microwave radiation at 130° C. for 3 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by reverse phase flash chromatography with MeOH/H₂O (50/50, v/v) to afford tert-butyl 3-[(6-aminopyrimidin-4-yl)amino]pyrrolidine-1-carboxylate (600.0 mg, 14%) as a white solid. LCMS (ESI, m/z): [M+H]⁺=280.2.

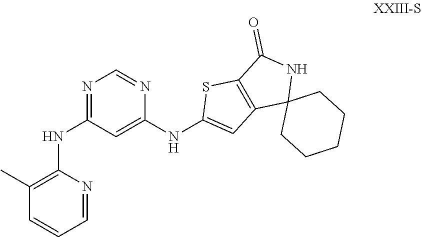 [SMILES: Cc1cccnc1Nc1cc(Nc2cc3c(s2)C(=O)NC32CCCCC2)ncn1]

2′-((6-((3-methylpyridin-2-yl)amino)pyrimidin-4-yl)amino)spiro [cyclohexane-1,4′-thieno[2,3-c]pyrrol]-6′(5′H)-one (Compound XXIII-S): LCMS (ESI, m/z): [M+H]⁺=407.2. ¹H NMR (300 MHz, DMSO-d₆): δ 10.91 (s, 1H), 8.69 (s, 1H), 8.45-8.42 (m, 2H), 8.22 (s, 1H), 7.62-7.59 (m, 1H), 7.43 (d, J=0.9 Hz, 1H), 7.04-7.01 (m, 1H), 6.66 (s, 1H), 2.32 (s, 3H), 1.79-1.65 (m, 6H), 1.64-1.42 (m, 4H).

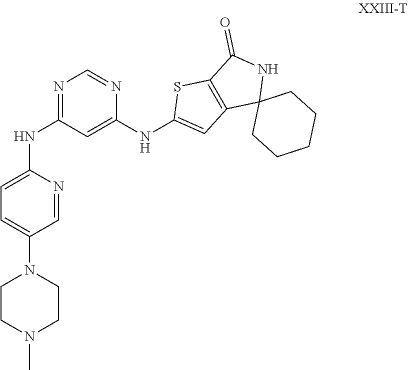 [SMILES: CN1CCN(c2ccc(Nc3cc(Nc4cc5c(s4)C(=O)NC54CCCCC4)ncn3)nc2)CC1]

2′-((6-((5-(4-methylpiperazin-1-yl)pyridin-2-yl)amino)pyrimidin-4-yl)amino)spiro[cyclohexane-1,4′-thieno[2,3-c]pyrrol]-6′(5′H)-one (Compound XXIII-T): LCMS (ESI, m/z): [M+H]⁺=491.3. ¹H NMR (300 MHz, DMSO-d₆): δ 10.88 (s, 1H), 9.81 (s, 1H), 8.48 (s, 2H), 7.97 (d, J=2.4 Hz, 1H), 7.50-7.41 (m, 2H), 7.24 (s, 1H), 6.64 (s, 1H), 3.31-3.16 (m, 8H), 2.72 (s, 3H), 1.72-1.63 (m, 6H), 1.60-1.31 (m, 4H).

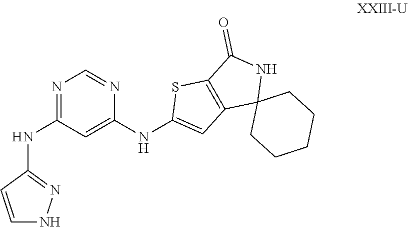 [SMILES: O=C1NC2(CCCCC2)c2cc(Nc3cc(Nc4cc[nH]n4)ncn3)sc21]

2′-((6-((1H-pyrazol-3-yl)amino)pyrimidin-4-yl)amino)spiro[cyclohexane-1,4′-thieno[2,3-c]pyrrol]-6′(5′H)-one (XXIII-U): the title compound was synthesized by using tert-butyl N-(tert-butoxycarbonyl)-N-[6-([1-[(4-methoxyphenyl)methyl]pyrazol-3-yl]amino)pyrimidin-4-yl]carbamate as the starting material of which the synthesis was shown below. LCMS (ESI, m/z): [M+H]⁺=382.2. ¹H NMR (400 MHz, DMSO-d₆): δ 12.26 (s, 1H), 10.79 (s, 1H), 9.55 (s, 1H), 8.36-8.34 (m, 2H), 7.63 (s, 1H), 6.90 (s, 1H), 6.62 (s, 1H), 6.14 (s, 1H), 1.75-1.60 (m, 6H), 1.51-1.38 (m, 4H).

The synthesis of tert-butyl N-(tert-butoxycarbonyl)-N-[6-([1-[(4-methoxyphenyl)methyl]pyrazol-3-yl]amino)pyrimidin-4-yl]carbamate: To a solution of tert-butyl N-(tert-butoxycarbonyl)-N-(6-chloropyrimidin-4-yl)carbamate (1.6 g, 4.85 mmol) in DMF (40.0 mL) was added 1-[(4-methoxyphenyl)methyl]pyrazol-3-amine (1.6 g, 7.88 mmol), Pd₂(dba)₃ (888.0 mg, 0.97 mmol), XantPhos (557.0 mg, 0.96 mmol) and Cs₂CO₃ (4.0 g, 12.12 mmol) at room temperature. The resulting mixture was stirred at 100° C. for 16 h under N₂. After the reaction was completed, the resulting mixture was cooled to room temperature and diluted with of H₂O. The mixture was extracted with ethyl acetate. The combined organic layer was washed with brine, dried over anhydrous sodium sulfate and filtered. The filtrate was concentrated under reduced pressure. The residue was purified by flash column chromatography with CH₂Cl₂/MeOH (90/10, v/v) to afford tert-butyl N-(tert-butoxycarbonyl)-N-[6-([1-[(4-methoxyphenyl)methyl]pyrazol-3-yl]amino)pyrimidin-4-yl]carbamate (1.0 g, 43%) as a white solid. LCMS (ESI, m/z): [M+H]⁺=497.2.

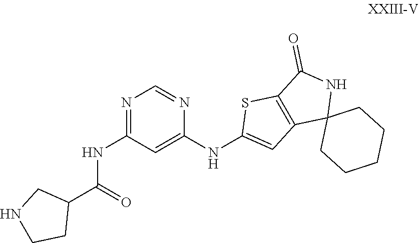 [SMILES: O=C1NC2(CCCCC2)c2cc(Nc3cc(NC(=O)C4CCNC4)ncn3)sc21]

N-(6-((6′-oxo-5′,6′-dihydrospiro[cyclohexane-1,4′-thieno[2,3-c]pyrrol]-2′-yl)amino)pyrimidin-4-yl)pyrrolidine-3-carboxamide (Compound XXIII-V): the title compound was synthesized by using benzyl 3-([6-[bis(tert-butoxycarbonyl)amino]pyrimidin-4-yl]carbamoyl)pyrrolidine-1-carboxylate as the starting material of which the synthesis was shown below. LCMS (ESI, m/z): [M+H]⁺=413.1. ¹H NMR (300 MHz, DMSO-d₆): δ 10.72 (s, 1H), 8.54 (d, J=0.9 Hz, 1H), 8.47 (s, 1H), 7.62 (d, J=0.9 Hz, 1H), 6.69 (s, 1H), 3.17-3.00 (m, 2H), 2.88-2.73 (m, 3H), 1.94-1.82 (m, 2H), 1.75-1.65 (m, 6H), 1.62-1.47 (m, 4H).

The synthesis of benzyl 3-([6-[bis(tert-butoxycarbonyl)amino]pyrimidin-4-yl]carbamoyl)pyrrolidine-1-carboxylate: To a mixture of benzyl 3-carbamoylpyrrolidine-1-carboxylate (1.0 g, crude) in 1,4-dioxane (30.0 mL) was added tert-butyl N-(tert-butoxycarbonyl)-N-(6-chloropyrimidin-4-yl)carbamate (1.3 g, 4.03 mmol), XantPhos (466.1 mg, 0.81 mmol), Cs₂CO₃ (2.0 g, 6.04 mmol) and Pd₂(dba)₃ (368.8 mg, 0.40 mmol) at room temperature. The mixture was stirred at 100° C. for 16 h under N₂. After the reaction was completed, the mixture was cooled to room temperature and filtered. The filtrate was diluted with water and extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (44/56, v/v) to afford the title compound (1.9 g, 88%) as a yellow solid. LCMS (ESI, m/z): [M+H]⁺=542.3.

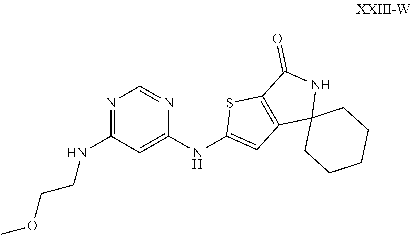 [SMILES: COCCNc1cc(Nc2cc3c(s2)C(=O)NC32CCCCC2)ncn1]

2′-((6-((2-methoxyethyl)amino)pyrimidin-4-yl)amino)spiro[cyclohexane-1,4′-thieno[2,3-c]pyrrol]-6′(5′H)-one (Compound XXIII-W): LCMS (ESI, m/z): [M+H]⁺=374.3. ¹HNMR (400 MHz, DMSO-d₆): δ 10.44 (s, 1H), 8.33 (s, 1H), 8.21 (s, 1H), 7.15 (s, 1H), 6.57 (s, 1H), 5.83 (s, 1H), 3.44-3.42 (m, 4H), 3.27 (s, 3H), 1.71-1.63 (m, 6H), 1.60-1.48 (m, 4H).

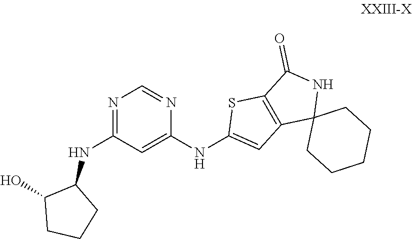 [SMILES: O=C1NC2(CCCCC2)c2cc(Nc3cc(N[C@H]4CCC[C@@H]4O)ncn3)sc21]

2′-((6-(((1S,2S)-2-hydroxycyclopentyl)amino)pyrimidin-4-yl)amino)spiro[cyclohexane-1,4′-thieno[2,3-c]pyrrol]-6′(5′H)-one (Compound XXIII-X): the title compound was synthesized by using N4-[(1S,2S)-2-[(tert-butyldimethylsilyl)oxy]cyclopentyl]pyrimidine-4,6-diamine as the starting material of which the synthesis was shown below. LCMS (ESI, m/z): [M+H]⁺=400.3. ¹H NMR (400 MHz, DMSO-d₆): δ 10.46 (s, 1H), 8.33 (s, 1H), 8.20 (s, 1H), 7.03 (d, J=4.8 Hz, 1H), 6.58 (s, 1H), 5.82 (s, 1H), 4.89 (d, J=3.2 Hz, 1H), 3.88-3.74 (m, 2H), 2.06-2.00 (m, 1H), 1.88-1.83 (m, 1H). 1.71-1.65 (m, 8H), 1.60-1.48 (m, 6H).

The synthesis of N4-[(1 S,2S)-2-[(tert-butyldimethylsilyl)oxy]cyclopentyl]pyrimidine-4,6-diamine:

Step 1. To a solution of tert-butyl N-(tert-butoxycarbonyl)-N-(6-chloropyrimidin-4-yl)-carbamate (10.0 g, 30.32 mmol) in isopropanol (250.0 mL) was added (1S,2S)-2-aminocyclopentan-1-ol (8.3 g, 81.56 mmol) and DIEA (19.6 g, 151.65 mmol) at room temperature. The resulting mixture was stirred at 100° C. for 16 h. After the reaction was completed, the resulting mixture was cooled to room temperature and filtered. The filtrate was concentrated under reduced pressure. The residue was purified by flash column chromatography with petroleum ether/ethyl acetate (10/90, v/v) to afford tert-butyl N-(tert-butoxycarbonyl)-N-(6-[[(1S,2S)-2-hydroxycyclopentyl]amino]pyrimidin-4-yl)carbamate (5.2 g, 43%) as a white solid. LCMS (ESI, m/z): [M+H]⁺=395.2.

Step 2. A solution of tert-butyl N-(tert-butoxycarbonyl)-N-(6-[[(1S,2S)-2-hydroxycyclopentyl]amino]pyrimidin-4-yl)carbamate (5.2 g, 13.18 mmol) in HCl/1,4-dioxane (30.0 mL, 4.0 mol/L) was stirred at room temperature for 16 h. After the reaction was completed, the resulting mixture was concentrated under reduced pressure. The pH value of the residue was adjusted to 7.0 with saturated NaHCO₃ (aq.). The mixture was extracted with ethyl acetate. The combined organic layer was washed with brine, dried over anhydrous sodium sulfate and filtered. The filtrate was concentrated under vacuum. The residue was purified by reverse phase flash chromatography with MeOH/H₂O (70/30, v/v) to afford (1S,2S)-2-[(6-aminopyrimidin-4-yl)amino]cyclopentan-1-ol (2.6 g, 99%) as a colorless oil. LCMS (ESI, m/z): [M+H]⁺=195.1.

Step 3. To a solution of (1S,2S)-2-[(6-aminopyrimidin-4-yl)amino]cyclopentan-1-ol (1.0 g, 5.15 mmol) in CH₂Cl₂ (50.0 mL) was added TBSCl (770.0 mg, 5.11 mmol) and imidazole (0.35 g, 5.14 mmol) at room temperature. The resulting mixture was stirred at room temperature for 16 h. After the reaction was completed, the resulting mixture was diluted with H₂O and extracted with CH₂Cl₂. The combined organic layer was washed with brine, dried over anhydrous sodium sulfate and filtered. The filtrate was concentrated under vacuum to afford the title compound (720.0 mg, crude) as a yellow oil. LCMS (ESI, m/z): [M+H]⁺=309.2.

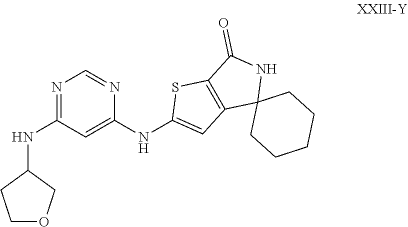 [SMILES: O=C1NC2(CCCCC2)c2cc(Nc3cc(NC4CCOC4)ncn3)sc21]

2′-((6-((tetrahydrofuran-3-yl)amino)pyrimidin-4-yl)amino)spiro[cyclohexane-1,4′-thieno[2,3-c]pyrrol]-6′(5′H)-one (Compound XXIII-Y): the title compound was synthesized by using N⁴-(tetrahydrofuran-3-yl)pyrimidine-4,6-diamine as the starting material of which the synthesis was shown below. LCMS (ESI, m/z): [M+H]⁺=386.2. ¹H NMR (300 MHz, DMSO-d₆): δ 10.50 (s, 1H), 8.32 (s, 1H), 8.22 (s, 1H), 7.29 (d, J=6.3 Hz, 1H), 6.56 (s, 1H), 5.79 (s, 1H), 4.42-4.31 (m, 1H), 3.85-3.73 (m, 2H), 3.71-3.68 (m, 1H), 3.66-3.52 (m, 1H), 2.26-2.08 (m, 1H), 1.83-1.65 (m, 7H), 1.60-1.31 (m, 4H).

The synthesis of N⁴-(tetrahydrofuran-3-yl)pyrimidine-4,6-diamine: To a solution of tert-butyl N-(tert-butoxycarbonyl)-N-(6-chloropyrimidin-4-yl)carbamate (500.0 mg, 1.52 mmol) in dioxane (10.0 mL) was added tetrahydrofuran-3-amine (396.3 mg, 4.55 mmol), Pd₂(dba)₃ (416.5 mg, 0.46 mmol), XantPhos (526.4 mg, 0.91 mmol) and Cs₂CO₃ (1481.9 mg, 4.55 mmol) at room temperature. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (90/10, v/v) to afford the title compound (100.0 mg, 17%) as a white solid. LCMS (ESI, m/z): [M+H]⁺=181.1.

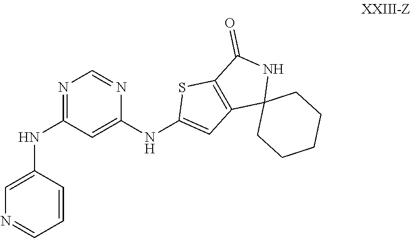 [SMILES: O=C1NC2(CCCCC2)c2cc(Nc3cc(Nc4cccnc4)ncn3)sc21]

2′-((6-((tetrahydrofuran-3-yl)amino)pyrimidin-4-yl)amino)spiro[cyclohexane-1,4′-thieno[2,3-c]pyrrol]-6′(5′H)-one (Compound XXIII-Y): the title compound was synthesized by using N⁴-(tetrahydrofuran-3-yl)pyrimidine-4,6-diamine as the starting material of which the synthesis was shown below. LCMS (ESI, m/z): [M+H]⁺=393.0. ¹H NMR (300 MHz, DMSO-d₆): δ 10.75 (s, 1H), 9.54 (s, 1H), 8.74 (d, J=2.7 Hz, 1H), 8.45-8.43 (m, 2H), 8.21-8.19 (m, 1H), 8.13-8.09 (m, 1H), 7.37-7.32 (m, 1H), 6.67 (s, 1H), 6.21 (d, J=0.6 Hz, 1H), 1.74-1.66 (m, 6H), 1.60-1.39 (m, 4H).

Following the procedure described above for Example 14 and substituting the appropriate reagents, starting materials and purification methods known to those skilled in the art, the compounds listed below may be prepared.

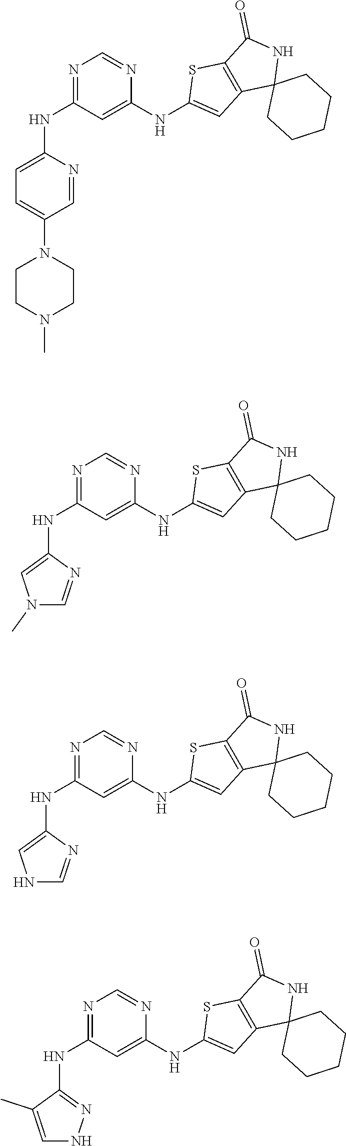 [SMILES: CN1CCN(c2ccc(Nc3cc(Nc4cc5c(s4)C(=O)NC54CCCCC4)ncn3)nc2)CC1 ; Cn1cnc(Nc2cc(Nc3cc4c(s3)C(=O)NC43CCCCC3)ncn2)c1 ; O=C1NC2(CCCCC2)c2cc(Nc3cc(Nc4c[nH]cn4)ncn3)sc21 ; Cc1c[nH]nc1Nc1cc(Nc2cc3c(s2)C(=O)NC32CCCCC2)ncn1]

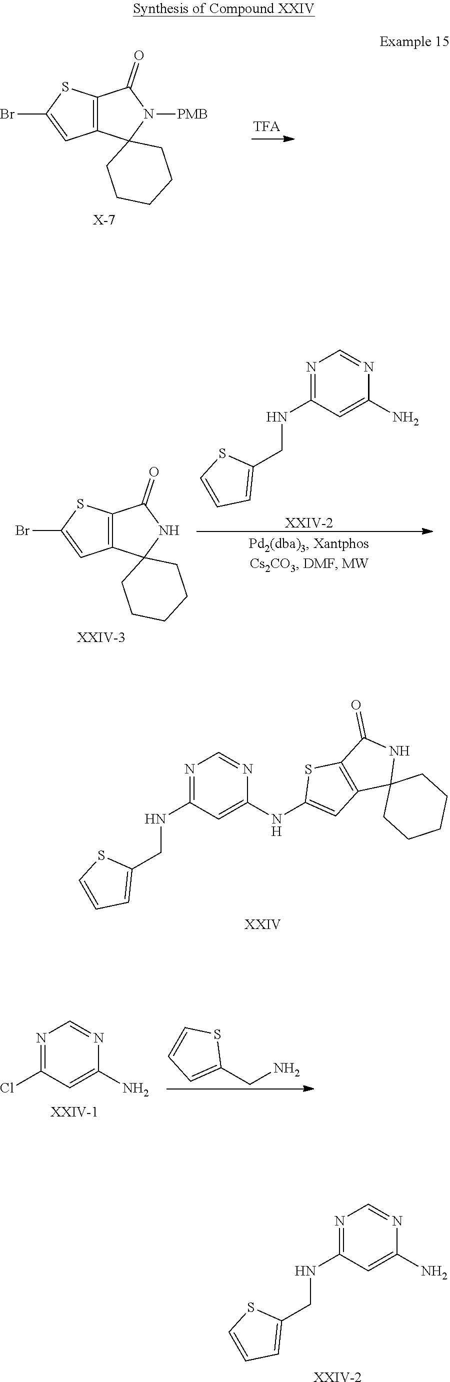

N4-(Thiophen-2-ylmethyl)pyrimidine-4,6-diamine (Compound XXIV-2): To a solution of 6-chloropyrimidin-4-amine (1.0 g, 7.72 mmol) in (MeOCH₂CH₂)₂₀ (30.0 mL) was added thiophen-2-ylmethanamine (4.4 g, 38.60 mmol). The resulting mixture was stirred at 160° C. for 5 h. After the reaction was completed, the resulting mixture was cooled to room temperature and diluted with H₂O. The mixture was extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (90/10, v/v) to afford compound XXIV-2 (922.5 mg, 57%) as a yellow solid. LCMS (ESI, m/z): [M+H]⁺=207.1.

2′-Bromospiro[cyclohexane-1,4′-thieno[2,3-c]pyrrol]-6′(5′H)-one (Compound XXIV-3): A solution of 2′-bromo-5′-(4-methoxybenzyl)spiro[cyclohexane-1,4′-thieno[2,3-c]pyrrol]-6′(5′H)-one (200.0 mg, 0.49 mmol) in TFA (2.0 mL) was stirred at 50° C. for 16 h. After the reaction was completed, the resulting mixture was cooled to room temperature. The pH value of the mixture was adjusted to 8.0 with saturated NaHCO₃ (aq.). The mixture was extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford compound XXIV-3 (128.0 mg, 36%) as a yellow solid. LCMS (ESI, m/z): [M+H]⁺=286.0.

2′-((6-((Thiophen-2-ylmethyl)amino)pyrimidin-4-yl)amino)spiro[cyclohexane-1,4′-thieno[2,3-c]pyrrol]-6′(5′H)-one (Compound XXIV): To a solution of 2′-bromospiro[cyclohexane-1,4′-thieno[2,3-c]pyrrol]-6′(5′H)-one (150.0 mg, 0.52 mmol) in DMF (10.0 mL) was added N4-(thiophen-2-ylmethyl)pyrimidine-4,6-diamine (162.2 mg, 0.79 mmol), Pd₂(dba)₃ (96.0 mg, 0.11 mmol), XantPhos (121.3 mg, 0.21 mmol) and Cs₂CO₃ (512.3 mg, 1.57 mmol). The final reaction mixture was irradiated with microwave radiation at 120° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (83/17, v/v) and then purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 34% to 44% in 9 min; 254/220 nm; to afford compound XXIV (14.4 mg, 6%) as a white solid. LCMS (ESI, m/z): [M+H]⁺=412.0. ¹H NMR (300 MHz, DMSO-d₆): δ 10.50 (s, 1H), 8.36 (s, 1H), 8.27 (s, 1H), 7.71-7.67 (m, 1H), 7.39-7.37 (m, 1H), 7.03-6.96 (m, 2H), 6.58 (s, 1H), 5.84 (s, 1H), 4.65 (d, J=6.0 Hz, 2H), 1.72-1.67 (m, 6H), 1.63-1.46 (m, 4H).

Following the procedure described above for Example 15 and substituting the appropriate reagents, starting materials and purification methods known to those skilled in the art, the compounds listed below were prepared.

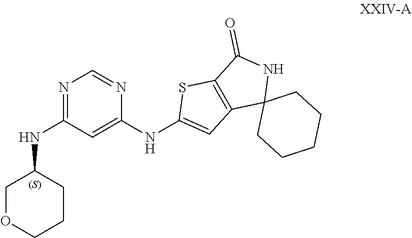

(S)-2′-((6-((tetrahydro-2H-pyran-3-yl)amino)pyrimidin-4-yl)amino)spiro[cyclohexane-1,4′-thieno[2,3-c]pyrrol]-6′(5′H)-one (Compound XXIV-A): LCMS (ESI, m/z): [M+H]⁺=400.1. ¹H NMR (300 MHz, DMSO-d₆): δ 10.46 (s, 1H), 8.37 (s, 1H), 8.23 (s, 1H), 7.07 (d, J=7.2 Hz, 1H), 6.57 (s, 1H), 5.84 (s, 1H), 3.86-3.70 (m, 3H), 3.38-3.32 (m, 1H), 3.14-3.07 (m, 1H), 1.96-1.92 (m, 1H), 1.72-1.47 (m, 13H).

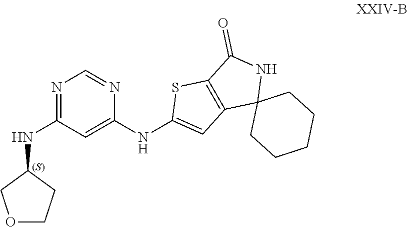

(S)-2′-((2-((tetrahydrofuran-3-yl)amino)pyridin-4-yl)amino)spiro[cyclohexane-1,4′-thieno[2,3-c]pyrrol]-6′(5′H)-one (Compound XXIV-B): LCMS (ESI, m/z): [M+H]⁺=385.1. ¹H NMR (400 MHz, DMSO-d₆): δ 9.40 (s, 1H), 8.58 (s, 1H), 7.76 (d, J=5.2 Hz, 1H), 6.75 (s, 1H), 6.57 (d, J=6.4 Hz, 1H), 6.21 (d, J=5.6 Hz, 2H), 4.33-4.29 (m, 1H), 3.86-3.79 (m, 2H), 3.72-3.69 (m, 1H), 3.51-3.48 (m, 1H), 2.16-2.08 (m, 1H), 1.80-1.73 (m, 3H), 1.72-1.64 (m, 4H), 1.60-1.49 (m, 4H).

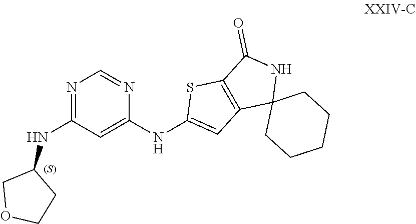

(S)-2′-((6-((tetrahydrofuran-3-yl)amino)pyrimidin-4-yl)amino)spiro[cyclohexane-1,4′-thieno[2,3-c]pyrrol]-6′(5′H)-one (Compound XXIV-C): The title compound was synthesized by using tert-butyl N-(tert-butoxycarbonyl)-N-[6-[(3 S)-oxolan-3-ylamino]pyrimidin-4-yl]carbamate as the starting material of which the synthesis was shown below. LCMS (ESI, m/z): [M+H]⁺=386.1. ¹H NMR (400 MHz, DMSO-d₆): δ 10.41 (s, 1H), 8.29 (s, 1H), 8.17 (s, 1H), 7.26 (d, J=6.4 Hz, 1H), 6.51 (s, 1H), 5.74 (s, 1H), 4.37 (s, 1H), 3.79-3.73 (m, 2H), 3.67-3.65 (m, 1H), 3.48-3.46 (m, 1H), 2.10-2.07 (m, 1H), 1.84-1.60 (m, 7H), 1.59-1.46 (m, 4H).

The synthesis of tert-butyl N-(tert-butoxycarbonyl)-N-[6-[(3 S)-oxolan-3-ylamino]pyrimidin-4-yl]carbamate: To a solution of (S)-tetrahydrofuran-3-amine hydrochloride (1.0 g, 8.09 mmol) in DMF (30.0 mL) was added tert-butyl N-(tert-butoxycarbonyl)-N-(6-chloropyrimidin-4-yl)carbamate (4.0 g, 12.14 mmol), Pd₂(dba)₃ (741.0 mg, 0.81 mmol), XantPhos (936.4 mg, 1.62 mmol) and Cs₂CO₃ (5.3 g, 16.18 mmol) at room temperature. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (84/16, v/v) to afford tert-butyl N-(tert-butoxycarbonyl)-N-[6-[(3 S)-oxolan-3-ylamino]pyrimidin-4-yl]carbamate (583.0 mg, 18%) as a yellow solid. LCMS (ESI, m/z): [M+H]⁺=381.2.

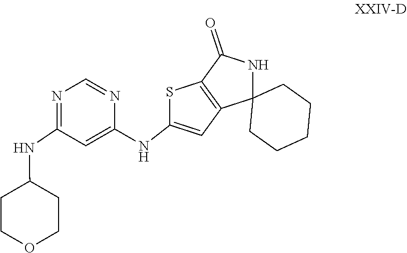 [SMILES: O=C1NC2(CCCCC2)c2cc(Nc3cc(NC4CCOCC4)ncn3)sc21]

2′-((6-((tetrahydro-2H-pyran-4-yl)amino)pyrimidin-4-yl)amino)spiro[cyclohexane-1,4′-thieno[2,3-c]pyrrol]-6′(5′H)-one (Compound XXIV-D): LCMS (ESI, m/z): [M+H]⁺=400.1. ¹H NMR (300 MHz, DMSO-d₆): δ 10.46 (s, 1H), 8.37 (s, 1H), 8.23 (s, 1H), 7.11 (d, J=7.5 Hz, 1H), 6.57 (s, 1H), 5.79 (s, 1H), 3.89-3.85 (m, 3H), 3.43-3.35 (m, 2H), 1.86-1.81 (m, 2H), 1.72-1.64 (m, 6H), 1.61-1.37 (m, 6H).

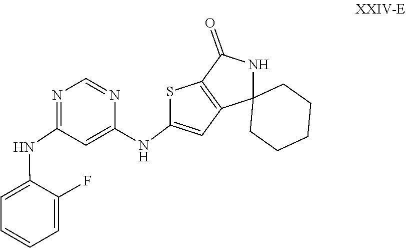 [SMILES: O=C1NC2(CCCCC2)c2cc(Nc3cc(Nc4ccccc4F)ncn3)sc21]

2′-((6-((2-fluorophenyl)amino)pyrimidin-4-yl)amino)spiro[cyclohexane-1,4′-thieno[2,3-c]pyrrol]-6′(5′H)-one (Compound XXIV-E): LCMS (ESI, m/z): [M+H]⁺=410.0. ¹H NMR (300 MHz, DMSO-d₆): δ 10.70 (s, 1H), 9.11 (s, 1H), 8.41-8.37 (m, 2H), 7.79-7.76 (m, 1H), 7.29-7.14 (m, 3H), 6.63 (s, 1H), 6.12 (s, 1H), 1.73-1.48 (m, 10H).

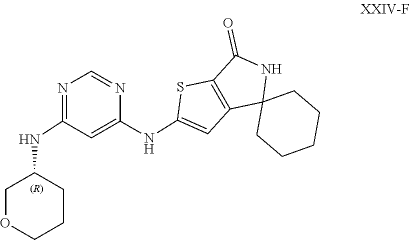

(R)-2′-((6-((tetrahydro-2H-pyran-3-yl)amino)pyrimidin-4-yl)amino)spiro[cyclohexane-1,4′-thieno[2,3-c]pyrrol]-6′(5′H)-one (Compound XXIV-F): LCMS (ESI, m/z): [M+H]⁺=400.1. ¹H NMR (400 MHz, DMSO-d₆): δ 10.45 (s, 1H), 8.35 (s, 1H), 8.23 (s, 1H), 7.06 (d, J=7.2 Hz, 1H), 6.58 (s, 1H), 5.84 (s, 1H), 3.86-3.83 (m, 2H), 3.74-3.71 (m, 1H), 3.30-3.25 (m, 1H), 3.14-3.09 (m, 1H), 1.95-1.92 (m, 1H), 1.72-1.65 (m, 7H). 1.60-1.48 (m, 6H).

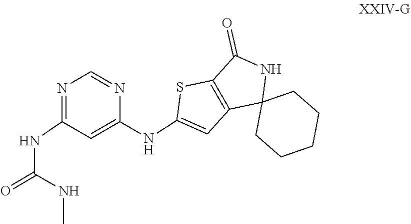 [SMILES: CNC(=O)Nc1cc(Nc2cc3c(s2)C(=O)NC32CCCCC2)ncn1]

1-methyl-3-(6-((6′-oxo-5′,6′-dihydrospiro[cyclohexane-1,4′-thieno[2,3-c]pyrrol]-2′-yl)amino)pyrimidin-4-yl)urea (Compound XXIV-G): the title compound was synthesized by using tert-butyl N-(tert-butoxycarbonyl)-N-[6-[(methylcarbamoyl)amino]pyrimidin-4-yl]carbamate as the starting material of which the synthesis was shown below, and BrettPhos and BrettPhos Pd G3 were used as ligand and catalyst for the last step. LCMS (ESI, m/z): [M+H]⁺=373.2. ¹H NMR (400 MHz, DMSO-d₆): δ 10.95 (s, 1H), 9.34 (s, 1H), 8.43-8.40 (m, 2H), 7.31 (s, 1H), 7.05 (s, 1H), 6.66 (s, 1H), 2.71-2.67 (m, 3H), 1.73-1.64 (m, 6H), 1.62-1.47 (m, 4H).

The synthesis of tert-butyl N-(tert-butoxycarbonyl)-N-[6-[(methylcarbamoyl)amino]pyrimidin-4-yl]carbamate: To a solution of 1-methylurea (3.0 g, 40.50 mmol) in DMF (45.0 mL) was added tert-butyl N-(tert-butoxycarbonyl)-N-(6-chloropyrimidin-4-yl)carbamate (7.9 g, 23.89 mmol), BrettPhos (4.4 g, 8.10 mmol), BrettPhos Pd G3 (3.7 g, 4.05 mmol) and Cs₂CO₃ (19.8 g, 60.74 mmol) at room temperature. The resulting mixture was stirred at 80° C. for 16 h under N₂. After the reaction was completed, the resulting mixture was cooled to room temperature and filtered. The filtrate was diluted with H₂O and extracted with ethyl acetate. The combined organic layer was washed with brine, dried over anhydrous sodium sulfate and filtered. The filtrate was evaporated in vacuo. The residue was purified by flash column chromatography with CH₂Cl₂/MeOH (92/8, v/v) and then purified by reverse phase flash chromatography with ACN/Water (53/47, v/v) to afford tert-butyl N-(tert-butoxycarbonyl)-N-[6-[(methylcarbamoyl)amino]pyrimidin-4-yl]carbamate (1.1 g, 7%) as a white solid. LCMS (ESI, m/z): [M+H]⁺=368.2.

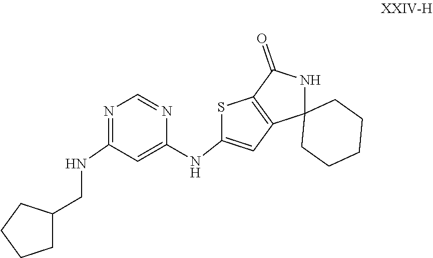 [SMILES: O=C1NC2(CCCCC2)c2cc(Nc3cc(NCC4CCCC4)ncn3)sc21]

2′-((6-((cyclopentylmethyl)amino)pyrimidin-4-yl)amino)spiro[cyclohexane-1,4′-thieno[2,3-c]pyrrol]-6′(5′H)-one (Compound XXIV-H): LCMS (ESI, m/z): [M+H]⁺=398.2. ¹H NMR (400 MHz, DMSO-d₆): δ 10.42 (s, 1H), 8.34 (s, 1H), 8.19 (s, 1H), 7.14 (s, 1H), 6.57 (s, 1H), 5.78 (s, 1H), 3.17-3.10 (m, 1H), 2.12-2.03 (m, 2H), 1.91-1.45 (m, 18H).

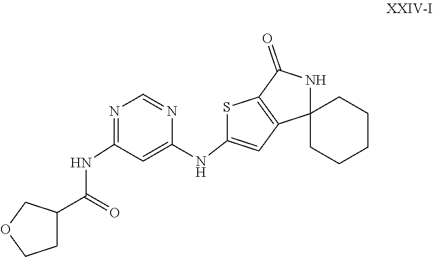 [SMILES: O=C1NC2(CCCCC2)c2cc(Nc3cc(NC(=O)C4CCOC4)ncn3)sc21]

N-(6-((6′-oxo-5′,6′-dihydrospiro[cyclohexane-1,4′-thieno[2,3-c]pyrrol]-2′-yl)amino)pyrimidin-4-yl)tetrahydrofuran-3-carboxamide (Compound XXIV-I): the title compound was synthesized by using tert-butyl (6-(tetrahydrofuran-3-carboxamido)pyrimidin-4-yl)carbamate as the starting material of which the synthesis was shown below. LCMS (ESI, m/z): [M+H]⁺=414.2. ¹HNMR (300 MHz, DMSO-d₆): δ 11.15 (s, 1H), 10.81 (s, 1H), 8.56 (s, 1H), 8.47 (s, 1H), 7.62 (d, J=0.9 Hz, 1H), 6.70 (s, 1H), 3.94-3.88 (m, 1H), 3.83-3.65 (m, 3H), 3.32-3.29 (m, 1H), 2.12-2.05 (m, 2H), 1.75-1.60 (m, 6H), 1.59-1.44 (m, 4H).

The synthesis of tert-butyl (6-(tetrahydrofuran-3-carboxamido)pyrimidin-4-yl)carbamate: To a solution of tetrahydrofuran-3-carboxamide (1.0 g, 8.68 mmol) in DMF (15.0 mL) was added tert-butyl (6-chloropyrimidin-4-yl)carbamate (1.9 g, 8.68 mmol), Cs₂CO₃ (8.4 g, 26.1 mmol), Brettphos (1.8 g, 3.47 mmol) and Brettphos Pd G3 (1.5 g, 1.73 mmol) at room temperature. The reaction mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (20/80, v/v) to afford the title compound (0.7 g, 26%) as an off-white solid. LCMS (ESI, m/z): [M+H]⁺=309.1

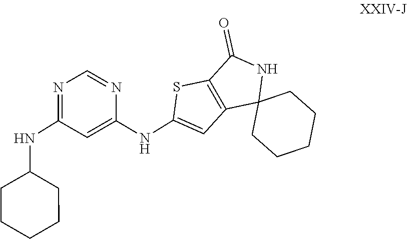 [SMILES: O=C1NC2(CCCCC2)c2cc(Nc3cc(NC4CCCCC4)ncn3)sc21]

2′-((6-(cyclohexylamino)pyrimidin-4-yl)amino)spiro[cyclohexane-1,4′-thieno[2,3-c]pyrrol]-6′(5′H)-one (Compound XXIV-J): LCMS (ESI, m/z): [M+H]⁺=398.2. ¹H NMR (400 MHz, DMSO-d₆): δ 10.41 (s, 1H), 8.34 (s, 1H), 8.20 (s, 1H), 6.97 (d, J=8.0 Hz, 1H), 6.56 (s, 1H), 5.76 (s, 1H), 3.82-3.65 (m, 1H), 1.89-1.86 (m, 2H), 1.73-1.47 (m, 13H), 1.35-1.14 (m, 5H).

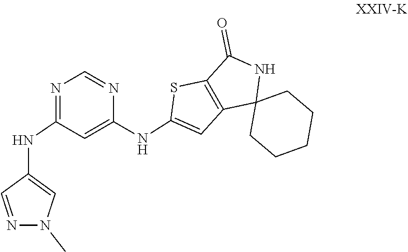 [SMILES: Cn1cc(Nc2cc(Nc3cc4c(s3)C(=O)NC43CCCCC3)ncn2)cn1]

2′-((6-((1-methyl-1H-pyrazol-4-yl)amino)pyrimidin-4-yl)amino)spiro[cyclohexane-1,4′-thieno[2,3-c]pyrrol]-6′(5′H)-one (Compound XXIV-K): LCMS (ESI, m/z): [M+H]⁺=396.2. ¹HNMR (400 MHz, DMSO-d₆): δ 10.54 (s, 1H), 9.00 (s, 1H), 8.35-8.32 (m, 2H), 7.85 (s, 1H), 7.41 (s, 1H), 6.59 (s, 1H), 5.97 (s, 1H), 3.81 (s, 3H), 1.75-1.59 (m, 6H), 1.57-1.46 (m, 4H).

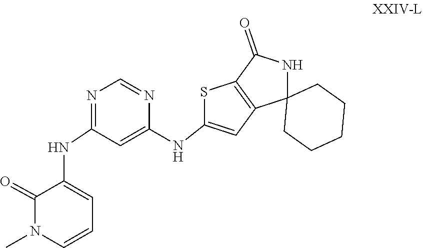 [SMILES: Cn1cccc(Nc2cc(Nc3cc4c(s3)C(=O)NC43CCCCC3)ncn2)c1=O]

2′-((6-((1-methyl-2-oxo-1,2-dihydropyridin-3-yl)amino)pyrimidin-4-yl)amino)spiro[cyclohexane-1,4′-thieno[2,3-c]pyrrol]-6′(5′H)-one (Compound XXIV-L): LCMS (ESI, m/z): [M+H]⁺=423.3. ¹H NMR (300 MHz, DMSO-d₆): δ 10.81 (br, 1H), 8.19 (s, 1H), 8.45-8.33 (m, 3H), 7.33-7.31 (m, 1H), 6.69 (s, 1H), 6.55 (s, 1H), 6.29-6.25 (m, 1H), 3.53 (s, 3H), 1.72-1.67 (m, 6H), 1.65-1.41 (m, 4H).

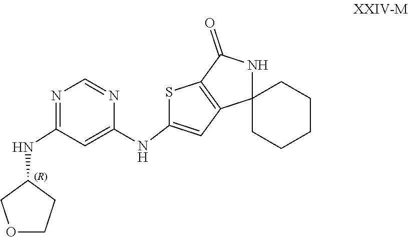

(R)-2′-((6-((tetrahydrofuran-3-yl)amino)pyrimidin-4-yl)amino)spiro[cyclohexane-1,4′-thieno[2,3-c]pyrrol]-6′(5′H)-one (Compound XXIV-M): LCMS (ESI, m/z): [M+H]⁺=386.1. ¹H NMR (300 MHz, CD₃OD): δ 8.25 (s, 1H), 6.67 (s, 1H), 5.85 (s, 1H), 4.45-4.39 (m, 1H), 4.01-3.93 (m, 2H), 3.89-3.82 (m, 1H), 3.71-3.56 (m, 1H), 2.36-2.24 (m, 1H), 1.97-1.87 (m, 1H), 1.84-1.54 (m, 10H).

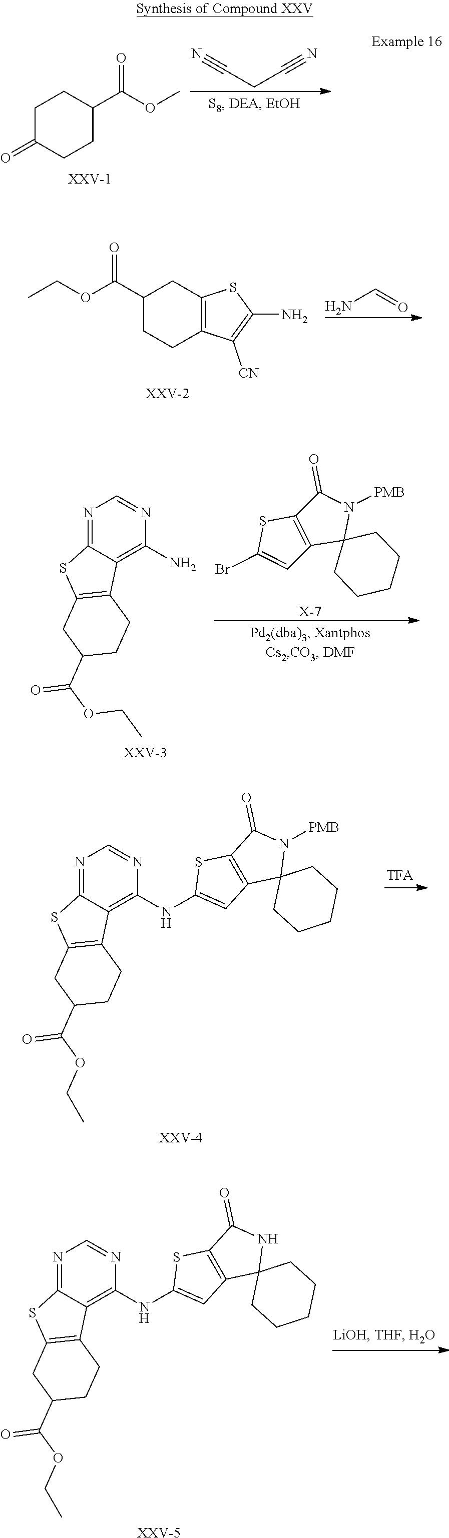

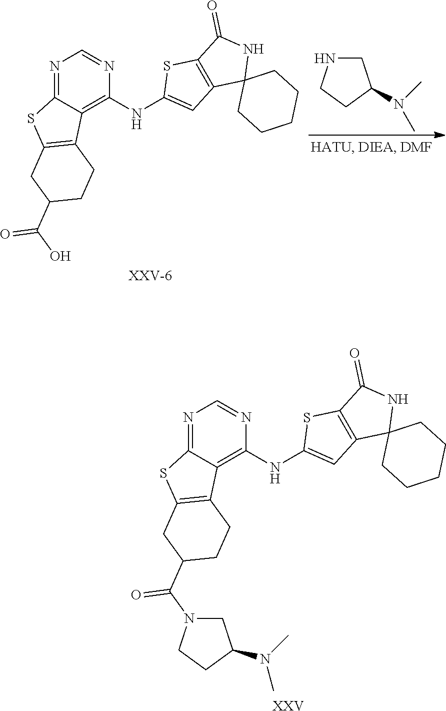

Ethyl 2-amino-3-cyano-4,5,6,7-tetrahydro-1-benzothiophene-6-carboxylate (Compound XXV-2): To a solution of ethyl 4-oxocyclohexane-1-carboxylate (10.0 g, 58.75 mmol) in EtOH (180.0 mL) was added malononitrile (3.9 g, 58.73 mmol), S₈ (4.7 g, 147.50 mmol) and DEA (4.6 g, 30.56 mmol). The mixture was stirred at 70° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by reverse phase flash chromatography with ACN/H₂O (90/10, v/v) to afford compound XXV-2 (4.5 g, 31%) as a black solid. LCMS (ESI, m/z): [M+H]⁺=251.1.

Ethyl 4-amino-5,6,7,8-tetrahydrobenzo[4,5]thieno[2,3-d]pyrimidine-7-carboxylate (Compound XXV-3): A solution of ethyl 2-amino-3-cyano-4,5,6,7-tetrahydro-1-benzothiophene-6-carboxylate (4.0 g, 15.98 mmol) in formamide (40.0 mL) was stirred at 150° C. for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford compound XXV-3 (2.2 g, 50%) as a yellow solid. LCMS (ESI, m/z): [M+H]⁺=278.1.

Ethyl 4-((5′-(4-methoxybenzyl)-6′-oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxylate (Compound XXV-4): To a solution of ethyl 4-amino-5,6,7,8-tetrahydrobenzo[4,5]thieno[2,3-d]pyrimidine-7-carboxylate (300.0 mg, 1.08 mmol) in DMF (22.0 mL) was added 2′-bromo-5′-(4-methoxybenzyl)spiro[cyclohexane-1,4′-thieno[2,3-c]pyrrol]-6′(5′H)-one (523.1 mg, 1.29 mmol), Pd₂(dba)₃ (297.2 mg, 0.33 mmol), XantPhos (375.5 mg, 0.65 mmol) and Cs₂CO₃ (1.1 g, 3.22 mmol). The final reaction mixture was irradiated with microwave radiation at 130° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (50/50, v/v) to afford compound XXV-4 (385.0 mg, 59%) as a yellow solid. LCMS (ESI, m/z): [M+H]⁺=603.2.

Ethyl 4-((6′-oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxylate (Compound XXV-5): A solution of ethyl 4-((5′-(4-methoxybenzyl)-6′-oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxylate (240.0 mg, 0.47 mmol) in TFA (15.0 mL) was stirred at 60° C. for 16 h. After the reaction was completed, the resulting mixture was cooled to room temperature. The pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions: (Column: Xselect CSH OBD Column 30×150 mm, 5 um; Mobile Phase A: Water (0.1% FA), Mobile Phase B: ACN; Flow rate: 60 mL/min; Gradient: 36% B to 46% B in 8 min; 254/220 nm to afford compound XXV-5 (32.1 mg, 17%) as a white solid. LCMS (ESI, m/z): [M+H]⁺=483.2. ¹H NMR (300 MHz, DMSO-d₆): δ 9.41 (s, 1H), 8.61 (s, 1H), 8.44 (s, 1H), 7.20 (s, 1H), 4.18-4.10 (m, 2H), 3.34-2.97 (m, 5H), 2.28-2.20 (m, 1H), 2.00-1.96 (m, 1H), 1.73-1.67 (m, 6H), 1.66-1.51 (m, 4H), 1.25-1.21 (m, 3H).

4-((6′-Oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxylic acid (Compound XXV-6): To a solution of ethyl 4-((6′-oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxylate (40.0 mg, 0.08 mmol) in THF (2.0 mL) and H₂O (2.0 mL) was added LiOH (8.0 mg, 0.33 mmol). The resulting mixture was at room temperature stirred for 16 h. After the reaction was completed, the resulting mixture was concentrated under reduced pressure. The pH value of the mixture was adjusted to 3 with HCl (aq.). The mixture was extracted with ethyl acetate. The combined organic layer was washed with brine, dried over anhydrous sodium sulfate and filtered. The filtrate was concentrated under vacuum. The residue was purified by reverse flash chromatography with ACN/H₂O (v/v, 50/50) and then purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 18% B to 27% B to afford compound XXV-6 (9.3 mg, 24%) as a yellow solid. LCMS (ESI, m/z): [M+H]⁺=455.2. ¹H NMR (300 MHz, DMSO-d₆): δ 12.45 (s, 1H), 9.35 (s, 1H), 8.55 (s, 1H), 8.36 (s, 1H), 7.10 (s, 1H), 3.16-3.14 (m, 2H), 3.12-3.08 (m, 1H), 3.05-2.96 (m, 2H), 2.89-2.83 (m, 1H), 2.21-2.18 (m, 1H), 1.96-1.91 (m, 1H), 1.70-1.56 (m, 9H).

2′-((7-((S)-3-(Dimethylamino)pyrrolidine-1-carbonyl)-5,6,7,8-tetrahydrobenzo[4,5]thieno[2,3-d]pyrimidin-4-yl)amino)spiro[cyclohexane-1,4′-thieno[2,3-c]pyrrol]-6′(5′H)-one (Compound XXV): To a solution of 4-((6′-oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxylic acid (75.0 mg, 0.17 mmol) in DMF (10.0 mL) was added (3S)—N,N-dimethylpyrrolidin-3-amine (38.0 mg, 0.33 mmol), HATU (78.0 mg, 0.21 mmol) and DIEA (107.0 mg, 0.83 mmol). The resulting mixture was stirred at 0° C. for 1 h under N₂. After the reaction was completed, the residue was purified by reverse phase flash chromatography with ACN/water (v/v, 50/50) and then purified by Prep-HPLC with the following conditions (Column: XBridge Prep OBD C18 Column, 30×150 mm, 5 um; Mobile Phase A: Water (10 mmol/L NH₄HCO₃), Mobile Phase B: ACN; Flow rate: 60 mL/min; Gradient: 35% B to 43% B in 9 min to afford compound XXV (22.6 mg, 25%) as a yellow solid. LCMS (ESI, m/z): [M+H]⁺=551.4. ¹H NMR (400 MHz, DMSO-d₆): δ 9.40 (s, 1H), 8.59 (s, 1H), 8.42 (s, 1H), 7.18 (s, 1H), 3.92-3.50 (m, 3H), 3.24-3.17 (m, 2H), 3.06-2.97 (m, 4H), 2.76-2.60 (m, 1H), 2.18 (s, 6H), 2.16-2.02 (m, 2H), 1.91-1.52 (m, 12H).

Following the procedure described above for Example 16 and substituting the appropriate reagents, starting materials and purification methods known to those skilled in the art, the compounds listed below were prepared.

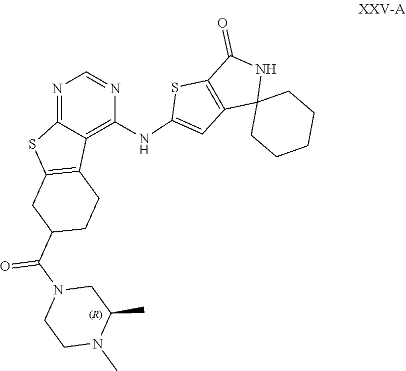

2′-((7-((R)-3,4-Dimethylpiperazine-1-carbonyl)-5,6,7,8-tetrahydrobenzo[4,5]thieno[2,3-d]pyrimidin-4-yl)amino)spiro[cyclohexane-1,4′-thieno[2,3-c]pyrrol]-6′(5′H)-one (Compound XXV-A): LCMS (ESI, m/z): [M+H]⁺=551.2. ¹H NMR (300 MHz, DMSO-d₆): δ 9.39 (s, 1H), 8.61 (s, 1H), 8.43 (s, 1H), 7.22 (s, 1H), 4.27-4.13 (m, 1H), 3.91-3.87 (m, 1H), 3.26-3.20 (m, 3H), 2.99-2.93 (m, 3H), 2.90-2.73 (m, 1H), 2.28 (s, 3H), 2.11-1.94 (m, 4H), 1.85-1.52 (m, 11H), 1.05-1.00 (m, 3H).

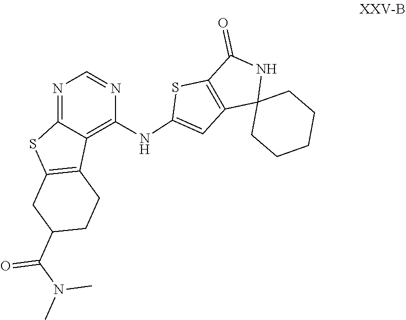 [SMILES: CN(C)C(=O)C1CCc2c(sc3ncnc(Nc4cc5c(s4)C(=O)NC54CCCCC4)c23)C1]

N,N-Dimethyl-4-((6′-oxo-5′,6′-dihydrospiro[cyclohexane-1,4′-thieno[2,3-c]pyrrol]-2′-yl)amino)-5,6,7,8-tetrahydrobenzo[4,5]thieno[2,3-d]pyrimidine-7-carboxamide (Compound XXV-B): LCMS (ESI, m/z): [M+H]⁺=482.2. ¹H NMR (300 MHz, DMSO-d₆): δ 9.38 (s, 1H), 8.61 (s, 1H), 8.43 (s, 1H), 7.23 (s, 1H), 3.25-3.16 (m, 2H), 3.10 (s, 3H), 2.97-2.94 (m, 2H), 2.88 (s, 3H), 2.10-2.06 (m, 1H), 1.86-1.55 (m, 12H).

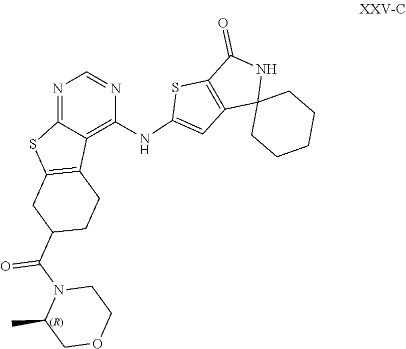

2′-((7-((R)-3-Methylmorpholine-4-carbonyl)-5,6,7,8-tetrahydrobenzo[4,5]thieno[2,3-d]pyrimidin-4-yl)amino)spiro[cyclohexane-1,4′-thieno[2,3-c]pyrrol]-6′(5′H)-one (Compound XXV-C): LCMS (ESI, m/z): [M+H]⁺=538.2. ¹H NMR (300 MHz, DMSO-d₆): δ 9.38 (s, 1H), 8.62 (s, 1H), 8.45 (s, 1H), 7.24 (d, J=3.6 Hz, 1H), 4.45-4.42 (m, 1H), 4.17-4.09 (m, 1H), 3.88-3.85 (m, 2H), 3.77-3.65 (m, 1H), 3.56-3.34 (m, 2H), 3.20-3.11 (m, 2H), 3.07-2.91 (m, 2H), 2.11-1.97 (m, 1H), 1.89-1.82 (m, 1H), 1.80-1.52 (m, 11H), 1.32-1.15 (m, 3H).

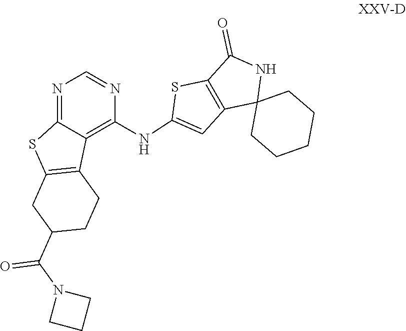 [SMILES: O=C1NC2(CCCCC2)c2cc(Nc3ncnc4sc5c(c34)CCC(C(=O)N3CCC3)C5)sc21]

2′-((7-(azetidine-1-carbonyl)-5,6,7,8-tetrahydrobenzo[4,5]thieno[2,3-d]pyrimidin-4-yl)amino)spiro[cyclohexane-1,4′-thieno[2,3-c]pyrrol]-6′(5′H)-one (Compound XXV-D): LCMS (ESI, m/z): [M+H]⁺=494.1. ¹H NMR (300 MHz, DMSO-d₆): δ 9.40 (s, 1H), 8.60 (s, 1H), 8.43 (s, 1H), 7.18 (s, 1H), 4.29-4.20 (m, 2H), 3.92-3.87 (m, 2H), 3.15-3.09 (m, 1H), 2.93-2.90 (m, 2H), 2.78-2.73 (m, 1H), 2.28-2.18 (m, 2H), 2.05-1.99 (m, 1H), 1.76-1.58 (m, 12H).

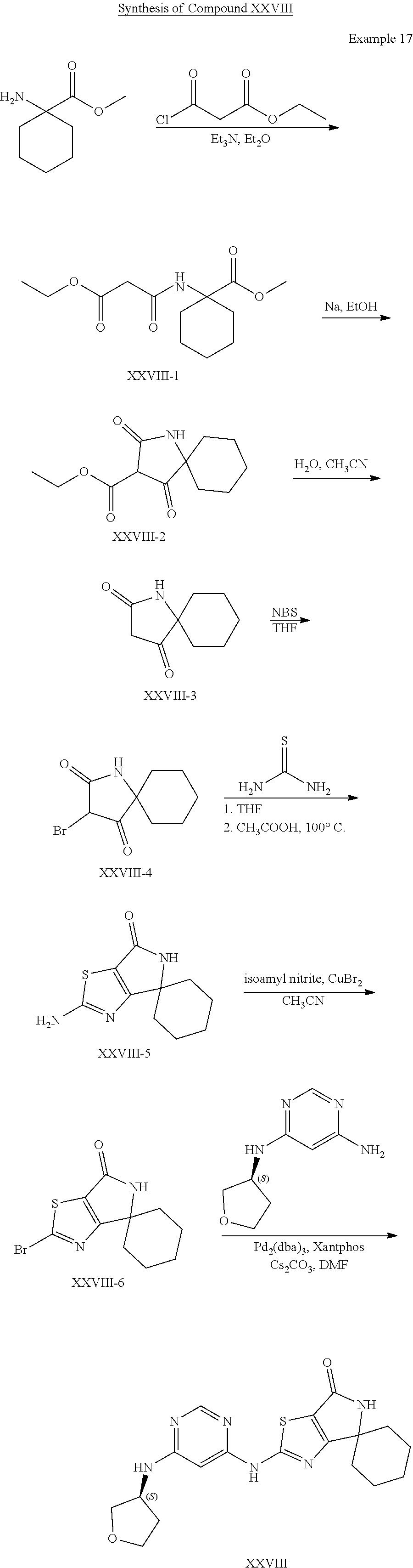

methyl 1-(3-ethoxy-3-oxopropanamido)cyclohexane-1-carboxylate (Compound XXVIII-1): To a solution of methyl 1-aminocyclohexane-1-carboxylate (5.0 g, 31.80 mmol) in Et₂O (80.0 mL) was added Et₃N (6.4 g, 63.61 mmol) and ethyl 3-chloro-3-oxopropanoate (4.8 g, 31.80 mmol) at 0° C. under N₂. The reaction mixture was stirred at room temperature for 16 h. After the reaction was completed, the resulting mixture was diluted with water and extracted with ethyl acetate. The combined organic layer was washed with brine, dried over anhydrous sodium sulfate and filtered. The filtrate was concentrated under reduced pressure to afford the title compound (7.2 g, crude) as a yellow oil. LCMS (ESI, m/z): [M+H]⁺=272.1.

ethyl 2,4-dioxo-1-azaspiro[4.5]decane-3-carboxylate (Compound XXVIII-2): A solution of Na (0.92 g, 39.81 mmol) in EtOH (50.0 mL) was stirred at room temperature for 30 min. Then methyl 1-(3-ethoxy-3-oxopropanamido)cyclohexane-1-carboxylate (7.2 g, 26.54 mmol) was added to the mixture. The reaction mixture was stirred at 100° C. for another 2 h. After the reaction was completed, the mixture was cooled to room temperature and quenched with HCl (2.0 mol/L). The mixture was extracted with CH₂Cl₂. The combined organic layer was washed with brine, dried over anhydrous sodium sulfate and filtered. The filtrate was concentrated under reduced pressure to afford the title compound (5.1 g, crude) as an off-white solid. LCMS (ESI, m/z): [M+H]⁺=240.1.

1-azaspiro[4.5]decane-2,4-dione (Compound XXVIII-3): A solution of ethyl 2,4-dioxo-1-azaspiro[4.5]decane-3-carboxylate (5.1 g, 21.32 mmol) in CH₃CN (50.0 mL) and water (10.0 mL) was stirred at 80° C. for 4 h. After the reaction was completed, the mixture was concentrated under vacuum. The residue was trituration with MTBE and hexane and then filtered. The solid was collected and dried to afford the title compound (3.2 g, crude) as an off-white solid. LCMS (ESI, m/z): [M+H]⁺=168.1.

3-bromo-1-azaspiro[4.5]decane-2,4-dione (Compound XXVIII-4): To a solution of 1-azaspiro[4.5]decane-2,4-dione (3.2 g, 19.14 mmol) in THF (50.0 mL) was added NBS (3.4 g, 19.14 mmol) at room temperature. The reaction mixture was stirred at room temperature for 16 h. After the reaction was completed, the mixture was concentrated under vacuum. The residue was purified by flash column chromatography with CH₂Cl₂/CH₃OH (92/8) to afford the title compound (3.5 g, 74%) as an off-white solid. LCMS (ESI, m/z): [M+H]⁺=246.0.

2′-aminospiro[cyclohexane-1,4′-pyrrolo[3,4-d]thiazol]-6′(5′H)-one (Compound XXVIII-5): To a solution of 3-bromo-1-azaspiro[4.5]decane-2,4-dione (3.5 g, 14.22 mmol) in THF (50.0 mL) was added thiourea (1.1 g, 14.22 mmol) at room temperature. The mixture was stirred at room temperature for 16 h. The mixture was concentrated under vacuum. Then CH₃COOH (50.0 mL) was added to the residue. The final reaction mixture was stirred at 100° C. for another 4 h. After the reaction was completed, the mixture was cooled to room temperature and filtered. The solid was collected and dried to afford the title compound (2.3 g, crude) as a white solid. [M+H]⁺=224.1.

2′-bromospiro[cyclohexane-1,4′-pyrrolo[3,4-d]thiazol]-6′(5′H)-one (Compound XXVIII-6): To a solution of 2′-aminospiro[cyclohexane-1,4′-pyrrolo[3,4-d]thiazol]-6′(5′H)-one (1.5 g, 6.72 mmol) in ACN (30.0 mL) was added CuBr₂ (1.2 g, 5.37 mmol) and isoamyl nitrite (1.6 g, 13.44 mmol). The reaction mixture was stirred at room temperature for 16 h. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (96/4, v/v) to afford the title compound (1.1 g, 58%) as a yellow solid. LCMS (ESI, m/z): [M+H]⁺=287.0.

(S)-2′-((6-((tetrahydrofuran-3-yl)amino)pyrimidin-4-yl)amino)spiro[cyclohexane-1,4′-pyrrolo[3,4-d]thiazol]-6′(5′H)-one (Compound XXVIII): To a solution of 2′-bromospiro[cyclohexane-1,4′-pyrrolo[3,4-d]thiazol]-6′(5′H)-one (100.0 mg, 0.35 mmol) in DMF (10.0 mL) was added (S)—N4-(tetrahydrofuran-3-yl)pyrimidine-4,6-diamine (62.8 mg, 0.35 mmol), XantPhos (40.3 mg, 0.07 mmol), Cs₂CO₃ (340.4 mg, 1.04 mmol) and Pd₂(dba)₃ (31.9 mg, 0.04 mmol) at room temperature. The resulting mixture was stirred at 100° C. for 16 h under N₂. After the reaction was completed, the mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/CH₃OH (94/6, v/v) and then purified by Prep-HPLC with the following conditions (Column: YMC-Actus Triart C18 ExRS, 30 mmx 150 mm, 5 um; Mobile Phase A: Water (10 mmol/L NH₄HCO₃), Mobile Phase B: ACN; Flow rate: 60 mL/min; Gradient: 25% B to 35% B in 8 min, 254 nm) to afford the title compound (25.7 mg, 19%) as a white solid. LCMS (ESI, m/z): [M+H]⁺=387.2. ¹H NMR (400 MHz, DMSO-d₆): δ 11.70 (s, 1H), 8.43 (s, 1H), 8.33 (s, 1H), 7.57 (d, J=6.0 Hz, 1H), 6.10 (s, 1H), 4.40 (s, 1H), 3.87-3.71 (m, 3H), 3.54-3.51 (m, 1H), 2.19-2.14 (m, 1H), 1.82-1.42 (m, 11H).

Following the procedure described above for Example 17 and substituting the appropriate reagents, starting materials and purification methods known to those skilled in the art, the compounds listed below were synthesized.

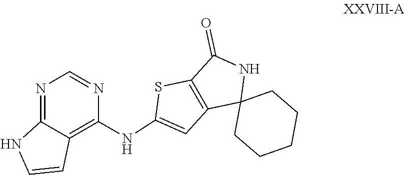 [SMILES: O=C1NC2(CCCCC2)c2cc(Nc3ncnc4[nH]ccc34)sc21]

2′-((7H-pyrrolo[2,3-d]pyrimidin-4-yl)amino)spiro[cyclohexane-1,4′-pyrrolo[3,4-d]thiazol]-6′(5′H)-one (Compound XXVIII-A): LCMS (ESI, m/z): [M+H]⁺=341.1. ¹H NMR (300 MHz, DMSO-d₆): δ 12.32-12.05 (m, 2H), 8.53 (s, 2H), 7.37 (d, J=3.6 Hz, 1H), 7.01 (d, J=3.3 Hz, 1H), 1.85-1.35 (m, 10H).

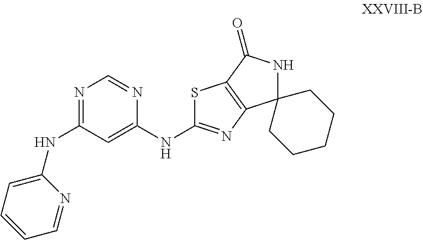 [SMILES: O=C1NC2(CCCCC2)c2nc(Nc3cc(Nc4ccccn4)ncn3)sc21]

2′-((6-(pyridin-2-ylamino)pyrimidin-4-yl)amino)spiro[cyclohexane-1,4′-pyrrolo[3,4-d]thiazol]-6′(5′H)-one (Compound XXVIII-B): LCMS (ESI, m/z): =394.1. ¹H NMR (300 MHz, DMSO-d₆): δ 12.06 (s, 1H), 10.12 (s, 1H), 8.55 (d, J=0.6 Hz, 1H), 8.49 (s, 1H), 8.30-8.28 (m, 1H), 7.75-7.69 (m, 2H), 7.49 (d, J=8.4 Hz, 1H), 7.01-6.97 (m, 1H), 1.81-1.65 (m, 6H), 1.64-1.46 (m, 4H).

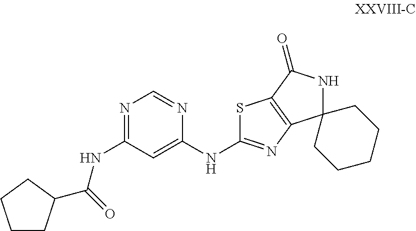 [SMILES: O=C1NC2(CCCCC2)c2nc(Nc3cc(NC(=O)C4CCCC4)ncn3)sc21]

N-(6-((6′-oxo-5′,6′-dihydrospiro[cyclohexane-1,4′-pyrrolo[3,4-d]thiazol]-2′-yl)amino)pyrimidin-4-yl)cyclopentanecarboxamide (Compound XXVIII-D): LC MS (ESI, m/z): [M+H]⁺=413.1. ¹H NMR (300 MHz, DMSO-d₆): δ 12.28 (s, 1H), 10.73 (s, 1H), 8.65 (d, J=0.9 Hz, 1H), 8.54 (s, 1H), 7.84 (s, 1H), 2.98-2.93 (m, 1H), 1.91-1.64 (m, 18H).

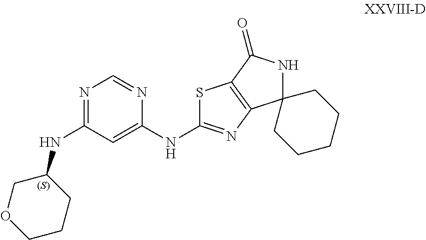

(S)-2′-((6-((tetrahydro-2H-pyran-3-yl)amino)pyrimidin-4-yl)amino)spiro[cyclohexane-1,4′-pyrrolo[3,4-d]thiazol]-6′(5′H)-one (Compound XXVIII-D): LC MS (ESI, m/z): [M+H]⁺=401.2. ¹H NMR (400 MHz, DMSO-d₆): δ 11.69 (s, 1H), 8.43 (s, 1H), 8.31 (s, 1H), 7.30 (d, J=6.0 Hz, 1H), 6.11 (s, 1H), 3.85-3.71 (m, 3H), 3.36-3.32 (m, 1H), 3.13-3.07 (m, 1H), 1.95-1.92 (m, 1H), 1.81-1.65 (m, 7H), 1.62-1.37 (m, 6H).

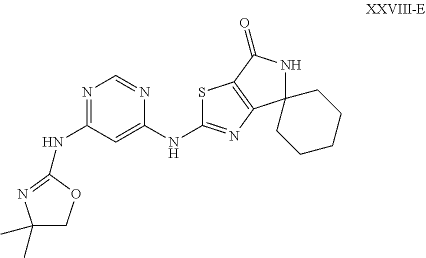 [SMILES: CC1(C)COC(Nc2cc(Nc3nc4c(s3)C(=O)NC43CCCCC3)ncn2)=N1]

2′-((6-((4,4-dimethyl-4,5-dihydrooxazol-2-yl)amino)pyrimidin-4-yl)amino)spiro[cyclohexane-1,4′-pyrrolo[3,4-d]thiazol]-6′(5′H)-one (Compound XXVIII-E): the title compound was synthesized by using tert-butyl (6-((4,4-dimethyl-4,5-dihydrooxazol-2-yl)amino)pyrimidin-4-yl)carbamate as the starting material of which the synthesis was shown below. LCMS (ESI, m/z): [M+H]⁺=414.0. ¹H NMR (300 MHz, DMSO-d₆): δ 11.95 (s, 1H), 9.17 (s, 1H), 8.58 (s, 1H), 8.46 (s, 1H), 6.41 (s, 1H), 4.09 (s, 2H), 1.82-1.50 (m, 10H), 1.35 (s, 6H).

The synthesis of tert-butyl (6-((4,4-dimethyl-4,5-dihydrooxazol-2-yl)amino)pyrimidin-4-yl)carbamate:

Step 1. To a solution of tert-butyl (6-aminopyrimidin-4-yl)carbamate (500.0 mg, 1.61 mmol) in CHCl₃ (20.0 mL) and in H₂O (10.0 mL) was added thiophosgene (185.2 mg, 1.61 mmol) and NaHCO₃ (676.7 mg, 8.06 mmol). The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum. The residue was purified by flash column chromatography with petroleum ether/ethyl acetate (40/60, v/v) to afford tert-butyl (6-isothiocyanatopyrimidin-4-yl)carbamate (382.0 mg, 94%) as a white solid. LCMS (ESI, m/z): [M+H]⁺=253.1.

Step 2. To a solution of tert-butyl (6-isothiocyanatopyrimidin-4-yl)carbamate (513.0 mg, 2.03 mmol) in THF (10.0 mL) was added 2-amino-2-methylpropan-1-ol (362.5 mg, 4.07 mmol). The resulting mixture was stirred at room temperature for 16 h. After the reaction was completed, the mixture was concentrated under vacuum. The residue was purified by flash column chromatography with CH₂Cl₂/MeOH (85/15, v/v) to afford tert-butyl (6-(3-(1-hydroxy-2-methylpropan-2-yl)thioureido)pyrimidin-4-yl)carbamate (307.0 mg, 44%) as a white solid, LCMS (ESI, m/z): [M+H]⁺=342.2.

Step 3. To a solution of tert-butyl (6-(3-(1-hydroxy-2-methylpropan-2-yl)thioureido)pyrimidin-4-yl)carbamate (307.0 mg, 0.89 mmol) in THF (5.0 mL) and H₂O (5.0 mL) was added p-toluenesulfonyl chloride (342.8 mg, 1.79 mmol) and NaOH (35.9 mg, 0.89 mmol). The resulting mixture was stirred at room temperature for 16 h. After the reaction was completed, the resulting mixture was concentrated under vacuum. The residue was purified by flash column chromatography with petroleum ether/ethyl acetate (75/25, v/v) to afford tert-butyl (6-((4,4-dimethyl-4,5-dihydrooxazol-2-yl)amino)pyrimidin-4-yl)carbamate (102.0 mg, 47%) as a light brown solid. LCMS (ESI, m/z): [M+H]⁺=308.2.

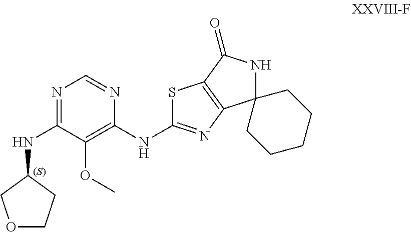

(S)-2′-((5-methoxy-6-((tetrahydrofuran-3-yl)amino)pyrimidin-4-yl)amino)spiro[cyclohexane-1,4′-pyrrolo[3,4-d]thiazol]-6′(5′H)-one (Compound XXVIII-F): LCMS (ESI, m/z): [M+H]⁺=417.2. ¹H NMR (400 MHz, DMSO-d₆): δ 11.38 (s, 1H), 8.51 (s, 1H), 8.17 (s, 1H), 7.08 (d, J=6.8 Hz, 1H), 4.60-4.55 (m, 1H), 3.91-3.85 (m, 2H), 3.75-3.70 (m, 1H), 3.64 (s, 3H), 3.60-3.57 (m, 1H), 2.19-2.13 (m, 1H), 2.01-1.95 (m, 1H), 1.83-1.78 (m, 4H), 1.70-1.52 (m, 5H), 1.39-1.32 (m, 1H).

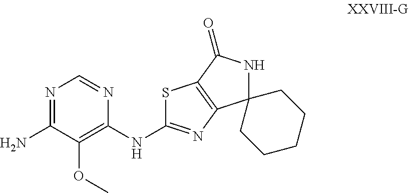 [SMILES: COc1c(N)ncnc1Nc1nc2c(s1)C(=O)NC21CCCCC1]

2′-((6-amino-5-methoxypyrimidin-4-yl)amino)spiro[cyclohexane-1,4′-pyrrolo[3,4-d]thiazol]-6′(5′H)-one (Compound XXVIII-G): LCMS (ESI, m/z): [M+H]⁺=347.3. ¹H NMR (400 MHz, DMSO-d₆): δ 11.33 (s, 1H), 8.50 (s, 1H), 8.05 (s, 1H), 6.75 (s, 2H), 3.64 (s, 3H), 1.83-1.78 (m, 4H), 1.70-1.51 (m, 5H), 1.42-1.33 (m, 1H).

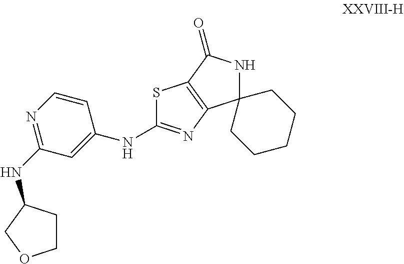 [SMILES: O=C1NC2(CCCCC2)c2nc(Nc3ccnc(N[C@H]4CCOC4)c3)sc21]

(S)-2′-((2-((tetrahydrofuran-3-yl)amino)pyridin-4-yl)amino)spiro[cyclohexane-1,4′-pyrrolo[3,4-d]thiazol]-6′(5′H)-one (Compound XXVIII-H): LCMS (ESI, m/z): [M+H]⁺=386.2. ¹H NMR (400 MHz, DMSO-d₆): δ 10.83 (s, 1H), 8.54 (s, 1H), 7.86 (d, J=5.6 Hz, 1H), 6.83 (s, 1H), 6.69 (d, J=5.6 Hz, 2H), 4.32-4.26 (m, 1H), 3.88-3.81 (m, 2H), 3.73-3.68 (m, 1H), 3.54-3.51 (m, 1H), 2.20-2.11 (m, 1H), 1.85-1.81 (m, 5H), 1.71-1.63 (m, 2H), 1.62-1.44 (m, 4H).

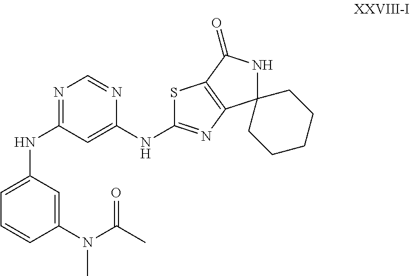 [SMILES: CC(=O)N(C)c1cccc(Nc2cc(Nc3nc4c(s3)C(=O)NC43CCCCC3)ncn2)c1]

N-methyl-N-(3-((6-((6′-oxo-5′,6′-dihydrospiro[cyclohexane-1,4′-pyrrolo[3,4-d]thiazol]-2′-yl)amino)pyrimidin-4-yl)amino)phenyl)acetamide (Compound XXVIII-I): LCMS (ESI, m/z): [M+H]⁺=464.1. ¹H NMR (300 MHz, DMSO-d₆): δ 11.99 (s, 1H), 9.72 (s, 1H), 8.55 (s, 1H), 8.50 (s, 1H), 7.63 (s, 1H), 7.54 (d, J=8.4 Hz, 1H), 7.41-7.36 (m, 1H), 6.97 (d, J=7.8 Hz, 1H), 6.48 (s, 1H), 3.18 (s, 3H), 1.85-1.46 (m, 13H).

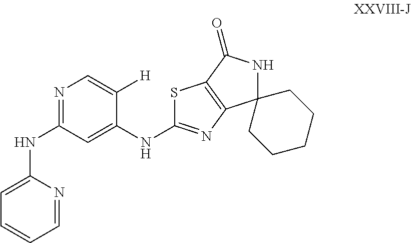 [SMILES: O=C1NC2(CCCCC2)c2nc(Nc3ccnc(Nc4ccccn4)c3)sc21]

2′-((2-(pyridin-2-ylamino)pyridin-4-yl)amino)spiro[cyclohexane-1,4′-pyrrolo[3,4-d]thiazol]-6′(5′H)-one (Compound XXVIII-J): LCMS (ESI, m/z): [M+H]⁺=393.2. ¹H NMR (400 MHz, DMSO-d₆): δ 9.66 (s, 1H), 8.59 (s, 1H), 8.22 (d, J=4.0 Hz, 1H), 8.13-8.09 (m, 2H), 7.67-7.59 (m, 2H), 7.19-7.17 (m, 1H), 6.88-6.85 (m, 1H), 1.90-1.80 (m, 4H), 1.68-1.62 (m, 2H), 1.56-1.53 (m, 3H), 1.44-1.42 (m, 1H).

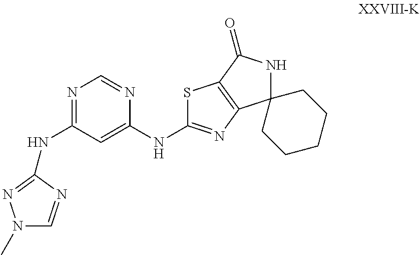 [SMILES: Cn1cnc(Nc2cc(Nc3nc4c(s3)C(=O)NC43CCCCC3)ncn2)n1]

2′-((6-((1-methyl-1H-1,2,4-triazol-3-yl)amino)pyrimidin-4-yl)amino)spiro[cyclohexane-1,4′-pyrrolo[3,4-d]thiazol]-6′(5′H)-one (Compound XXVIII-K): [M+H]⁺=398.1. ¹H NMR (400 MHz, DMSO-d₆): δ 12.03 (s, 1H), 10.14 (s, 1H), 8.48-8.42 (m, 2H), 8.34 (s, 1H), 7.52 (s, 1H), 3.83 (s, 3H), 1.84-1.46 (m, 10H).

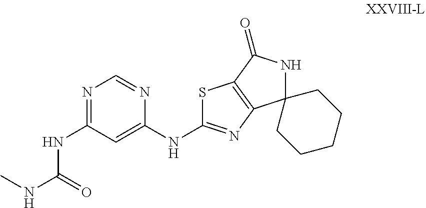 [SMILES: CNC(=O)Nc1cc(Nc2nc3c(s2)C(=O)NC32CCCCC2)ncn1]

1-methyl-3-(6-((6′-oxo-5′,6′-dihydrospiro[cyclohexane-1,4′-pyrrolo[3,4-d]thiazol]-2′-yl)amino)pyrimidin-4-yl)urea (Compound XXVIII-L): LCMS (ESI, m/z): [M+H]⁺=374.2. ¹HNMR (400 MHz, DMSO-d₆): δ 11.51 (s, 1H), 9.42 (s, 1H), 8.53 (s, 1H), 8.44 (s, 1H), 7.35-7.28 (m, 2H), 2.71 (d, J=4.4 Hz, 3H), 1.82-1.45 (m, 10H).

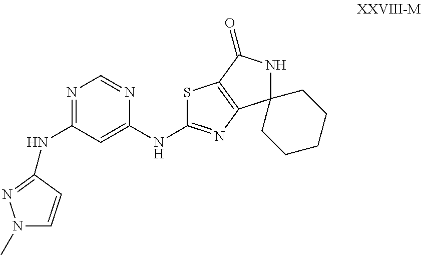 [SMILES: Cn1ccc(Nc2cc(Nc3nc4c(s3)C(=O)NC43CCCCC3)ncn2)n1]

2′-((6-((1-methyl-1H-pyrazol-3-yl)amino)pyrimidin-4-yl)amino)spiro[cyclohexane-1,4′-pyrrolo[3,4-d]thiazol]-6′(5′H)-one (Compound XXVIII-M): LCMS (ESI, m/z): [M+H]⁺=397.1. ¹H NMR (400 MHz, DMSO-d₆): δ 11.87 (s, 1H), 9.73 (s, 1H), 8.47-8.43 (m, 2H), 7.58 (s, 1H), 7.01 (s, 1H), 6.18 (s, 1H), 3.78 (s, 3H), 1.98-1.39 (m, 10H).

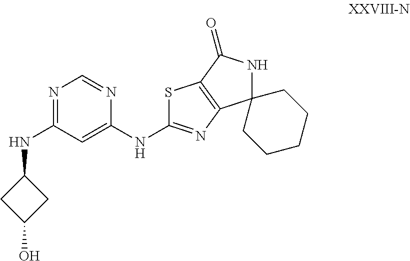 [SMILES: O=C1NC2(CCCCC2)c2nc(Nc3cc(N[C@H]4C[C@H](O)C4)ncn3)sc21]

Trans-2′-((6-(((1r,3r)-3-hydroxycyclobutyl)amino)pyrimidin-4-yl)amino)spiro[cyclohexane-1,4′-pyrrolo[3,4-d]thiazol]-6′(5′H)-one (Compound XXVIII-N): LCMS (ESI, m/z): [M+H]⁺=387.1. ¹H NMR (400 MHz, DMSO-d₆): δ 11.68 (s, 1H), 8.42 (s, 1H), 8.29 (s, 1H), 7.60-7.59 (m, 1H), 5.98 (s, 1H), 5.03 (s, 1H), 4.31-4.28 (m, 2H), 2.18-2.03 (m, 4H), 1.91-1.66 (m, 6H), 1.58-1.32 (m, 4H).

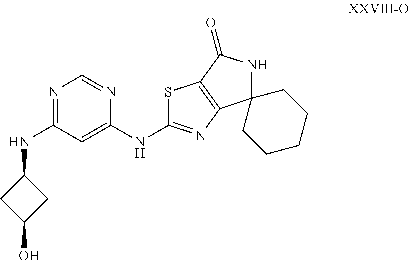 [SMILES: O=C1NC2(CCCCC2)c2nc(Nc3cc(N[C@H]4C[C@@H](O)C4)ncn3)sc21]

Cis-2′-((6-(((1s,3s)-3-hydroxycyclobutyl)amino)pyrimidin-4-yl)amino)spiro[cyclohexane-1,4′-pyrrolo[3,4-d]thiazol]-6′(5′H)-one (Compound XXVIII-O): LCMS (ESI, m/z): [M+H]⁺=387.3. ¹H NMR (400 MHz, DMSO-d₆): δ 11.65 (s, 1H), 8.43 (s, 1H), 8.27 (s, 1H), 7.55 (d, J=6.8 Hz, 1H), 6.00 (s, 1H), 5.08 (d, J=6.0 Hz, 1H), 3.87-3.82 (m, 2H), 2.67-2.54 (m, 2H), 1.81-1.64 (m, 8H), 1.62-1.45 (m, 4H).

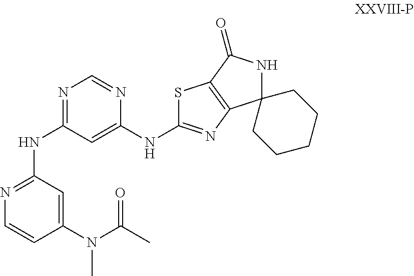 [SMILES: CC(=O)N(C)c1ccnc(Nc2cc(Nc3nc4c(s3)C(=O)NC43CCCCC3)ncn2)c1]

N-methyl-N-(2-((6-((6′-oxo-5′,6′-dihydrospiro[cyclohexane-1,4′-pyrrolo[3,4-d]thiazol]-2′-yl)amino)pyrimidin-4-yl)amino)pyridin-4-yl)acetamide (Compound XXVIII-P): LCMS (ESI, m/z): [M+H]⁺=465.1. ¹H NMR (400 MHz, DMSO-d₆): δ 12.09 (s, 1H), 10.17 (s, 1H), 8.57-8.48 (m, 2H), 8.30 (d, J=5.2 Hz, 1H), 7.65 (s, 1H), 7.48 (s, 1H), 7.03-7.01 (m, 1H), 3.22 (s, 3H), 2.05 (s, 3H), 1.95-1.66 (m, 10H).

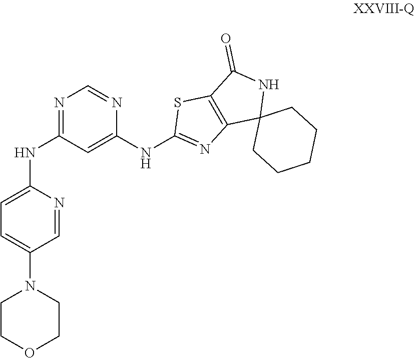 [SMILES: O=C1NC2(CCCCC2)c2nc(Nc3cc(Nc4ccc(N5CCOCC5)cn4)ncn3)sc21]

2′-((6-((5-morpholinopyridin-2-yl)amino)pyrimidin-4-yl)amino)spiro[cyclohexane-1,4′-pyrrolo[3,4-d]thiazol]-6′(5′H)-one (Compound XXVIII-Q): LCMS (ESI, m/z): [M+H]⁺=479.2. ¹H NMR (400 MHz, DMSO-d₆): δ 11.99 (s, 1H), 9.86 (s, 1H), 8.55-8.43 (m, 2H), 7.95 (s, 1H), 7.65-7.45 (m, 8H), 3.77-3.67 (m, 4H), 3.15-3.08 (m, 4H), 2.20-1.46 (m, 10H).

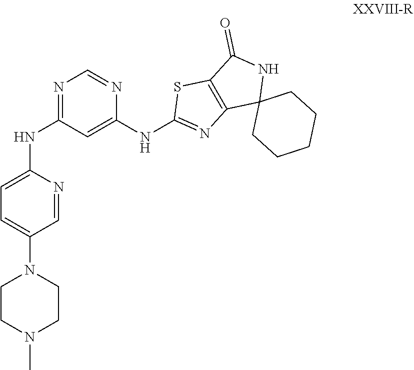 [SMILES: CN1CCN(c2ccc(Nc3cc(Nc4nc5c(s4)C(=O)NC54CCCCC4)ncn3)nc2)CC1]

2′-((6-((5-(4-methylpiperazin-1-yl)pyridin-2-yl)amino)pyrimidin-4-yl)amino)spiro [cyclohexane-1,4′-pyrrolo[3,4-d]thiazol]-6′(5′H)-one (Compound XXVIII-R): LCMS (ESI, m/z): [M+H]⁺=492.3. ¹H NMR (400 MHz, DMSO-d₆): δ 11.98 (s, 1H), 9.86 (s, 1H), 8.49-8.47 (m, 2H), 7.95 (s, 1H), 7.44 (s, 3H), 3.13-3.11 (m, 4H), 2.49-2.46 (m, 4H), 2.23 (s, 3H), 1.82-1.75 (m, 4H), 1.71-1.66 (m, 2H), 1.58-1.55 (m, 3H), 1.45-1.38 (m, 1H).

Following the synthetic route described above for Example 17 and substituting the appropriate reagents, starting materials and purification methods known to those skilled in the art, the compounds listed below may be synthesized.

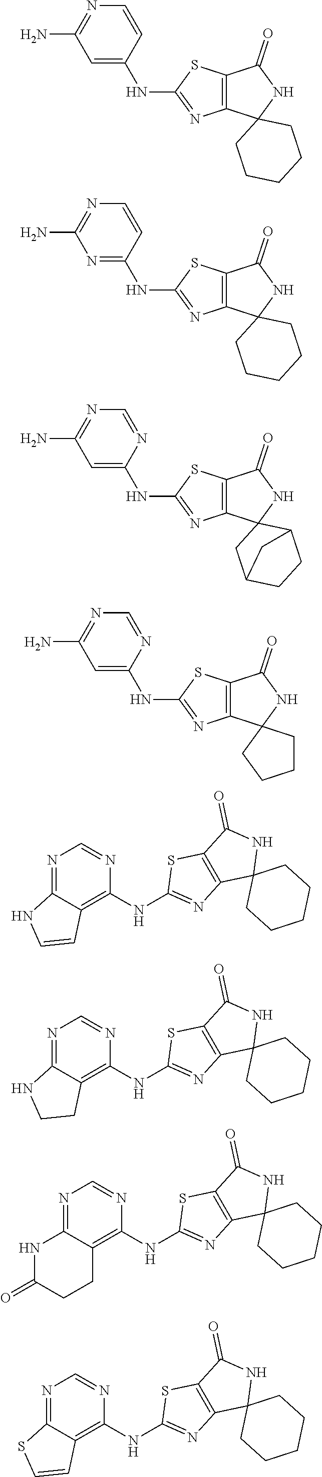

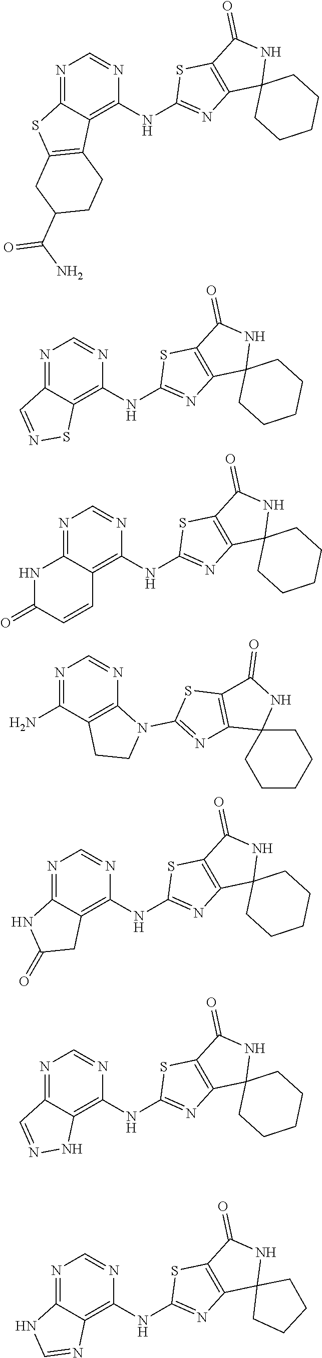

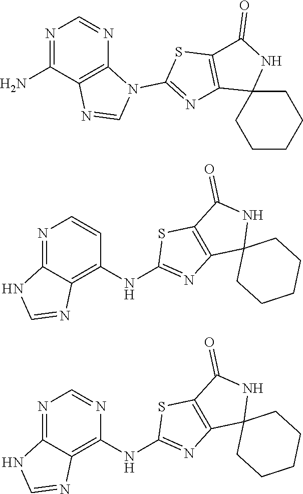 [SMILES: Nc1ncnc2c1ncn2-c1nc2c(s1)C(=O)NC21CCCCC1 ; O=C1NC2(CCCCC2)c2nc(Nc3ccnc4[nH]cnc34)sc21 ; O=C1NC2(CCCCC2)c2nc(Nc3ncnc4[nH]cnc34)sc21]

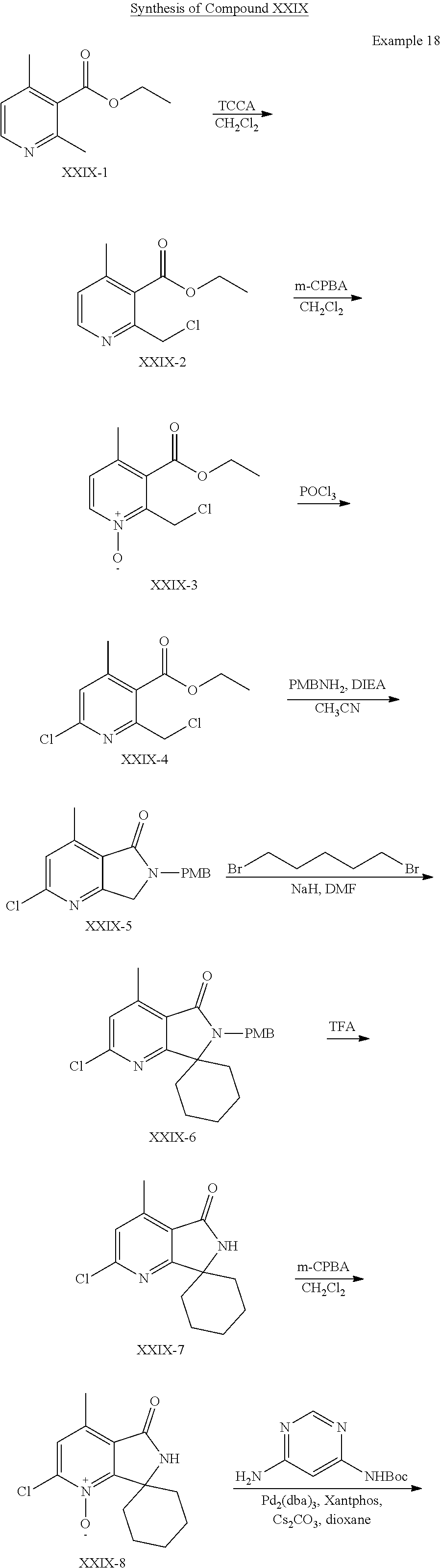

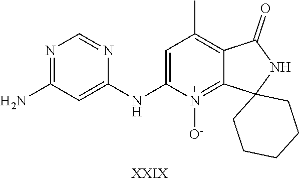

Ethyl 2-(chloromethyl)-4-methylnicotinate (Compound XXIX-2): To a solution of ethyl 2,4-dimethylnicotinate (10.0 g, 55.80 mmol) in CH₂Cl₂ (200.0 mL) was added trichloroisocyanuric acid (19.4 g, 83.70 mmol). The reaction mixture was stirred at room temperature for 16 h. After the reaction was completed, the pH value of the mixture was adjusted to 8 with saturated Na₂CO₃ (aq.). The mixture was extracted with CH₂Cl₂. The combined organic layer was washed with brine, dried over anhydrous sodium sulfate and filtered. The filtrate was concentrated under vacuum to afford compound XXIX-2 (11.5 g, crude) as a yellow oil. LCMS (ESI, m/z): [M+H]⁺=214.0.

2-(Chloromethyl)-3-(ethoxycarbonyl)-4-methylpyridine 1-oxide (Compound XXIX-3): To a solution of ethyl 2-(chloromethyl)-4-methylnicotinate (11.5 g, 53.82 mmol) in CH₂Cl₂ (250.0 mL) was added m-CPBA (23.2 g, 134.56 mmol) at 0° C. The resulting mixture was stirred at room temperature for 16 h. After the reaction was completed, the mixture was diluted with H₂O and extracted with CH₂Cl₂. The combined organic layer was washed with brine, dried over anhydrous sodium sulfate and filtered. The filtrate was concentrated under vacuum to afford compound XXIX-3 (13.4 g, crude) as a yellow oil. LCMS (ESI, m/z): [M+H]⁺=230.0.

Ethyl 6-chloro-2-(chloromethyl)-4-methylnicotinate (Compound XXIX-4): A solution of 2-(chloromethyl)-3-(ethoxycarbonyl)-4-methylpyridine 1-oxide (13.4 g, crude) in POCl₃ (80.0 mL) was stirred at 90° C. for 16 h. After the reaction was completed, the resulting mixture was cooled to room temperature and diluted with of H₂O. The mixture was extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (90/10, v/v) to afford compound XXIX-4 (6.5 g, 45%) as a yellow oil. LCMS (ESI, m/z): [M+H]⁺=248.0.

2-Chloro-6-(4-methoxybenzyl)-4-methyl-6,7-dihydro-5H-pyrrolo[3,4-b]pyridin-5-one (Compound XXIX-5): To a solution of ethyl 6-chloro-2-(chloromethyl)-4-methylnicotinate (6.5 g, 26.20 mmol) in ACN (100.0 mL) was added (4-methoxyphenyl)methanamine (3.0 g, 21.83 mmol) and DIEA (10.2 g, 78.60 mmol). The reaction mixture was stirred at room temperature for 16 h. After the reaction was completed, the resulting mixture was concentrated under reduced pressure. The residue was purified by flash column chromatography with ethyl acetate/petroleum ether (70/30, v/v) to afford compound XXIX-5 (6.1 g, 77%) as a light yellow solid. LCMS (ESI, m/z): [M+H]⁺=303.1.

2′-Chloro-6′-(4-methoxybenzyl)-4′-methylspiro[cyclohexane-1,7′-pyrrolo[3,4-b]pyridin]-5′(6′H)-one (Compound XXIX-6): To a mixture of 2-chloro-6-(4-methoxybenzyl)-4-methyl-6,7-dihydro-5H-pyrrolo[3,4-b]pyridin-5-one (1.6 g, 5.28 mmol) and 1,5-dibromopentane (1.5 g, 6.34 mmol) in DMF (30.0 mL) was added NaH (443.9 mg, 60%) at 0° C. under N₂. The resulting mixture was stirred at room temperature for 16 h. After the reaction was competed, the resulting mixture was diluted with of H₂O and extracted with ethyl acetate. The combined organic layer was washed with brine, dried over anhydrous sodium sulfate and filtered. The filtrate was concentrated under vacuum. The residue was purified by flash column chromatography with ethyl acetate/petroleum ether (83/17, v/v) to afford compound XXIX-6 (854.4 mg, 44%) as a yellow solid. LCMS (ESI, m/z): [M+H]⁺=371.0.

2′-Chloro-4′-methylspiro[cyclohexane-1,7′-pyrrolo[3,4-b]pyridin]-5′(6′H)-one (Compound XXIX-7): A solution 2′-chloro-6′-(4-methoxybenzyl)-4′-methylspiro[cyclohexane-1,7′-pyrrolo[3,4-b]pyridin]-5′(6′H)-one (854.4 mg, 2.30 mmol) in TFA (15.0 mL) was stirred at 80° C. for 4 h. After the reaction was completed, the resulting mixture was concentrated under vacuum. The residue was purified by flash column chromatography with ethyl acetate/petroleum ether (80/20, v/v) to afford compound XXIX-7 (555.0 mg, 96%) as a light yellow solid. LCMS (ESI, m/z): [M+H]⁺=251.1.

2′-Chloro-4′-methyl-5′-oxo-5′,6′-dihydrospiro[cyclohexane-1,7′-pyrrolo[3,4-b]pyridine] 1′-oxide (Compound XXIX-8): To a mixture of 2′-chloro-4′-methylspiro[cyclohexane-1,7′-pyrrolo[3,4-b]pyridin]-5′(6′H)-one (141.1 mg, 0.56 mmol) in CH₂Cl₂ (5.0 mL) was added m-CPBA (971.1 mg, 5.63 mmol). The reaction mixture was stirred at room temperature for 16 h. After the reaction was completed, the resulting mixture was concentrated under vacuum. The residue was purified by flash column chromatography with ethyl acetate/petroleum ether (80/20, v/v) to afford compound XXIX-8 (52.1 mg, 35%) as a white solid. LCMS (ESI, m/z): [M+H]⁺=267.1.

2′-((6-Aminopyrimidin-4-yl)amino)-4′-methyl-5′-oxo-5′,6′-dihydrospiro [cyclohexane-1,7′-pyrrolo[3,4-b]pyridine] 1′-oxide (Compound XXIX): To a solution of 2′-chloro-4′-methyl-5′-oxo-5′,6′-dihydrospiro[cyclohexane-1,7′-pyrrolo[3,4-b]pyridine] 1′-oxide (52.1 mg, 0.20 mmol) in 1,4-dioxane (10.0 mL) was added tert-butyl N-(6-aminopyrimidin-4-yl)carbamate (61.4 mg, 0.29 mmol), XantPhos (45.1 mg, 0.08 mmol), Cs₂CO₃ (117.6 mg, 0.36 mmol) and Pd₂(dba)₃ (35.7 mg, 0.04 mmol). The resulting mixture was stirred at 120° C. for 16 h under N₂. After the reaction was completed, the resulting mixture was cooled to room temperature and filtered. The filtrate was concentrated under reduced pressure. The residue was purified by flash column chromatography with CH₂Cl₂/MeOH (85/15, v/v) and then purified by Prep-HPLC with the following conditions: Column: Xselect CSH OBD Column 30×150 mm, 5 um; Mobile Phase A: Water (10 mmol/L NH₄HCO₃), Mobile Phase B: ACN; Flow rate: 60 mL/min; Gradient: 20% B to 27% B in 7 min; 254/220 nm; to afford compound XXIX (4.1 mg, 10%) as a white solid. [M+H]⁺=341.0. ¹H NMR (300 MHz, DMSO-d₆): δ 9.34 (s, 1H), 8.50 (s, 1H), 8.25 (s, 1H), 6.77 (s, 2H), 6.31 (s, 1H), 2.89-2.81 (m, 2H), 2.57 (s, 3H), 1.71-1.64 (m, 4H), 1.30-1.24 (m, 4H).

Following the synthetic route described above for Example 18 and substituting the appropriate reagents, starting materials and purification methods known to those skilled in the art, the compounds listed below were synthesized.

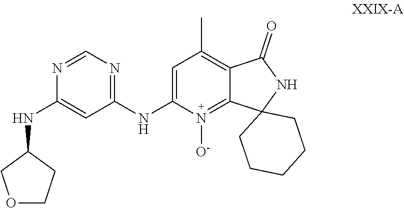

(S)-4′-methyl-5′-oxo-2′-((6-((tetrahydrofuran-3-yl)amino)pyrimidin-4-yl)amino)-5′,6′-dihydrospiro[cyclohexane-1,7′-pyrrolo[3,4-b]pyridine] 1′-oxide (Compound XXIX-A): LCMS (ESI, m/z): [M+H]⁺=411.3. ¹H NMR (300 MHz, DMSO-d₆): δ 10.20 (s, 1H), 9.33 (s, 1H), 8.51 (s, 1H), 8.33 (s, 1H), 7.54 (d, J=6.0 Hz, 1H), 6.43 (s, 1H), 4.33 (s, 1H), 3.90-3.71 (m, 3H), 3.57-3.53 (m, 1H), 2.88-2.72 (m, 2H), 2.57 (s, 3H), 2.28-2.08 (m, 1H), 1.98-1.80 (m, 1H), 1.79-1.67 (m, 5H), 1.30-1.19 (m, 3H).

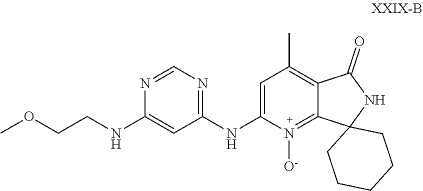

2′-((6-((2-methoxyethyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-B): LCMS (ESI, m/z): [M+H]⁺=399.4. ¹H NMR (300 MHz, DMSO-d₆): δ 10.14 (s, 1H), 9.33 (s, 1H), 8.49 (s, 1H), 8.31 (s, 1H), 7.34 (s, 1H), 6.43 (s, 1H), 3.46-3.37 (m, 4H), 3.28 (s, 3H), 2.91-2.81 (m, 2H), 2.57 (s, 3H), 1.70-1.64 (m, 5H), 1.30-1.26 (m, 3H).

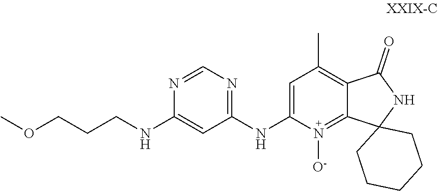

2′-((6-((3-methoxypropyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-C): LCMS (ESI, m/z): [M+H]⁺=413.3. ¹H NMR (400 MHz, DMSO-d₆): δ 10.15 (s, 1H), 9.32 (s, 1H), 8.51 (s, 1H), 8.29 (s, 1H), 7.31 (s, 1H), 6.40 (s, 1H), 3.40-3.38 (m, 2H), 3.24 (s, 3H), 2.87-2.84 (m, 2H), 2.57 (s, 3H), 1.77-1.70 (m, 7H), 1.25-1.20 (m, 3H).

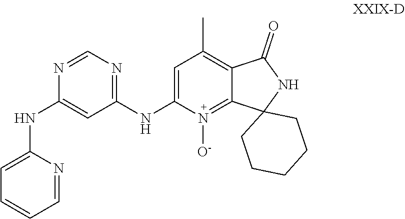

4′-methyl-5′-oxo-2′-((6-(pyridin-2-ylamino)pyrimidin-4-yl)amino)-5′,6′-dihydrospiro[cyclohexane-1,7′-pyrrolo[3,4-b]pyridine] 1′-oxide (Compound XXIX-D): LCMS (ESI, m/z): [M+H]⁺=418.1. ¹H NMR (300 MHz, DCl): δ 9.62 (s, 1H), 9.07-9.05 (m, 1H), 9.02-8.96 (m, 1H), 8.75 (s, 1H), 8.55 (s, 1H), 8.26-8.16 (m, 3H), 3.29 (s, 3H), 2.99-2.90 (m, 2H), 2.39-2.00 (m, 8H).

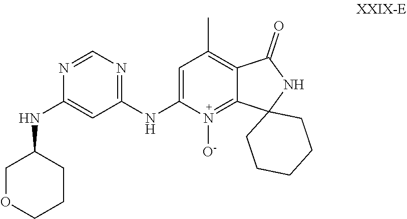

(S)-4′-methyl-5′-oxo-2′-((6-((tetrahydro-2H-pyran-3-yl)amino)pyrimidin-4-yl)amino)-5′,6′-dihydrospiro[cyclohexane-1,7′-pyrrolo[3,4-b]pyridine] 1′-oxide (Compound XXIX-E): LCMS (ESI, m/z): [M+H]⁺=425.3. ¹H NMR (400 MHz, DMSO-d₆): δ 10.16 (s, 1H), 9.33 (s, 1H), 8.49 (s, 1H), 8.32 (s, 1H), 7.26 (s, 1H), 6.43 (s, 1H), 3.87-3.73 (m, 3H), 3.16-3.11 (m, 1H), 2.91-2.83 (m, 2H), 2.68-2.57 (m, 4H), 1.97-1.95 (m, 1H), 1.71-1.53 (m, 8H), 1.32-1.19 (m, 3H).

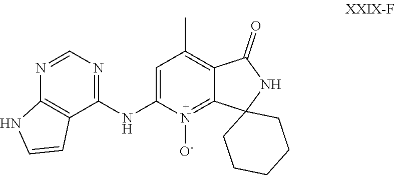

2′-((7H-pyrrolo[2,3-d]pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-F): LCMS (ESI, m/z): [M+H]⁺=365.3. ¹H NMR (300 MHz, DMSO-d₆): δ 12.22 (s, 1H), 10.42 (s, 1H), 9.45 (s, 1H), 8.81 (s, 1H), 8.59 (s, 1H), 7.51 (d, J=3.0 Hz, 1H), 6.82 (d, J=2.7 Hz, 1H), 2.92-2.84 (m, 2H), 2.67 (s, 3H), 1.74-1.66 (m, 5H), 1.35-1.25 (m, 3H).

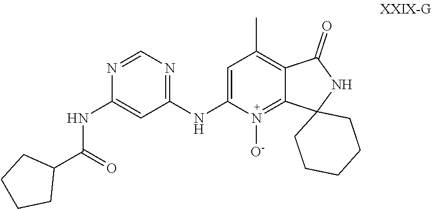

2′-((6-(cyclopentanecarboxamido)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-G): LCMS (ESI, m/z): [M+H]⁺=437.4. ¹H NMR (400 MHz, CDCl₃): δ 10.25 (s, 1H), 8.68-8.63 (m, 2H), 7.89-7.84 (m, 2H), 6.68 (s, 1H), 3.00-2.90 (m, 2H), 2.84-2.64 (m, 3H), 2.03-1.88 (m, 6H), 1.87-1.68 (m, 3H), 1.56-1.51 (m, 3H), 1.48-1.25 (m, 5H).

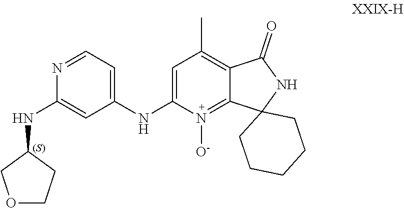

(S)-4′-methyl-5′-oxo-2′-((2-((tetrahydrofuran-3-yl)amino)pyridin-4-yl)amino)-5′,6′-dihydrospiro[cyclohexane-1,7′-pyrrolo[3,4-b]pyridine] 1′-oxide (Compound XXIX-H): LCMS (ESI, m/z): [M+H]⁺=410.3. ¹H NMR (300 MHz, DMSO-d₆): δ 9.62 (s, 1H), 9.29 (s, 1H), 7.93 (d, J=5.7 Hz, 1H), 7.23 (s, 1H), 6.76 (d, J=6.3 Hz, 1H), 6.61-6.60 (m, 1H) 6.51 (d, J=1.5 Hz, 1H), 4.36-4.30 (m, 1H), 3.90-3.81 (m, 2H), 3.76-3.70 (m, 1H), 3.56-3.52 (m, 1H), 2.91-2.85 (m, 2H), 2.55 (s, 3H), 2.28-2.14 (m, 1H), 1.84-1.69 (m, 7H), 1.40-1.27 (m, 2H).

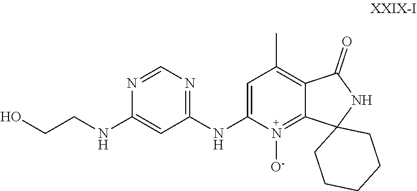

2′-((6-((2-hydroxyethyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-I): LCMS (ESI, m/z): [M+H]⁺=385.4. ¹H NMR (400 MHz, DMSO-d₆): δ 10.14 (s, 1H), 9.33 (s, 1H), 8.50 (s, 1H), 8.30 (s, 1H), 7.26 (s, 1H), 6.42 (s, 1H), 4.75-4.72 (m, 1H), 3.53-3.50 (m, 2H), 2.68-2.60 (m, 3H), 2.57 (s, 3H), 1.75-1.65 (m, 5H), 1.34-1.26 (m, 3H).

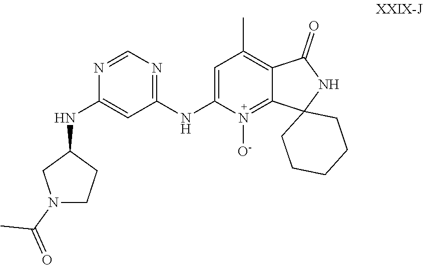

(S)-2′-((6-((1-acetylpyrrolidin-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-J): LCMS (ESI, m/z): [M+H]⁺=452.4. ¹H NMR (400 MHz, DMSO-d₆): δ 10.21 (s, 1H), 9.34 (s, 1H), 8.51 (s, 1H), 8.34 (d, J=2.8 Hz, 1H), 7.62-7.55 (m, 1H), 6.46 (s, 1H), 4.40-4.30 (m, 1H), 3.59-3.43 (m, 3H), 3.30-3.25 (m, 1H), 2.90-2.83 (m, 2H), 2.68-2.60 (m, 3H), 2.24-2.07 (m, 1H), 1.95-1.84 (m, 4H), 1.74-1.61 (m, 5H), 1.30-1.24 (m, 3H).

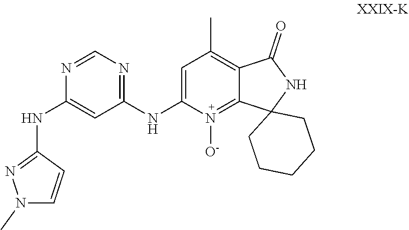

4′-methyl-2′-((6-((1-methyl-1H-pyrazol-3-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-K): LCMS (ESI, m/z): [M+H]⁺=421.3. ¹H NMR (400 MHz, DMSO-d₆): δ 10.25 (s, 1H), 9.74 (s, 1H), 9.35 (s, 1H), 8.52 (s, 1H), 8.45 (s, 1H), 7.59 (d, J=2.0 Hz, 1H), 7.15 (s, 1H), 6.24 (s, 1H), 3.80 (s, 3H), 2.91-2.83 (m, 2H), 2.68 (s, 3H), 1.71-1.65 (m, 5H), 1.30-1.25 (m, 3H).

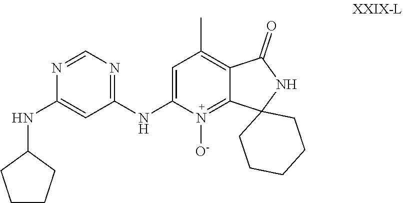

2′-((6-(cyclopentylamino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-L): LCMS (ESI, m/z): [M+H]⁺=409.3. ¹H NMR (400 MHz, CD₃OD): δ 8.60 (s, 1H), 8.31 (s, 1H), 8.21 (s, 1H), 6.25 (s, 1H), 4.20-4.10 (m, 1H), 3.01-2.94 (m, 2H), 2.68 (s, 3H), 2.09-2.01 (m, 2H), 1.88-1.85 (m, 2H), 1.84-1.77 (m, 2H), 1.75-1.70 (m, 4H), 1.69-1.51 (m, 2H) 1.50-1.37 (m, 3H).

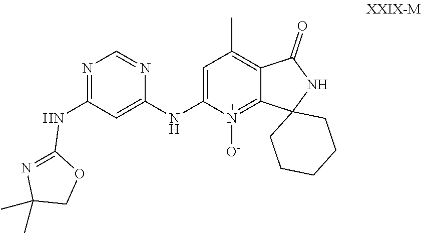

2′-((6-((4,4-dimethyl-4,5-dihydrooxazol-2-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-M): LCMS (ESI, m/z): [M+H]⁺=438.2. ¹H NMR (300 MHz, DMSO-d₆): δ 10.38 (s, 1H), 9.35 (s, 1H), 9.18 (s, 1H), 8.58 (d, J=5.4 Hz, 2H), 6.82 (s, 1H), 4.10 (s, 2H), 2.93-2.82 (m, 2H), 2.60 (s, 3H), 1.79-1.60 (m, 5H), 1.36-1.18 (m, 9H).

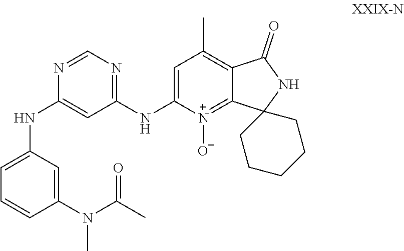 [SMILES: CC(=O)N(C)c1cccc(Nc2cc(Nc3cc(C)c4c([n+]3[O-])C3(CCCCC3)NC4=O)ncn2)c1]

4′-methyl-2′-((6-((3-(N-methylacetamido)phenyl)amino)pyrimidin-4-yl)amino)-5′-oxo-5′,6′-dihydrospiro[cyclohexane-1,7′-pyrrolo[3,4-b]pyridine] 1′-oxide (Compound XXIX-N): LCMS (ESI, m/z) [M+H]⁺=488.2. ¹H NMR (300 MHz, DMSO-d₆): δ 10.46 (s, 1H), 9.66 (s, 1H), 9.36 (s, 1H), 8.54 (d, J=5.4 Hz, 2H), 7.61-7.55 (m, 2H), 7.41-7.36 (m, 1H), 6.97 (d, J=6.9 Hz, 1H), 6.81 (s, 1H), 3.18 (s, 3H), 2.94-2.83 (m, 2H), 2.60 (s, 3H), 1.85 (s, 3H), 1.72-1.60 (m, 5H), 1.32-1.27 (m, 3H).

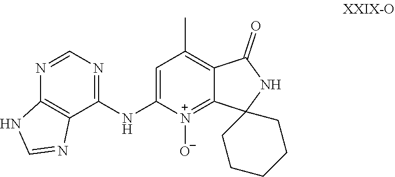 [SMILES: Cc1cc(Nc2ncnc3[nH]cnc23)[n+]([O-])c2c1C(=O)NC21CCCCC1]

2′-((9H-purin-6-yl)amino)-4′-methyl-5′-oxo-5′,6′-dihydrospiro[cyclohexane-1,7′-pyrrolo[3,4-b]pyridine] 1′-oxide (Compound XXIX-O): LCMS (ESI, m/z): [M+H]⁺=366.1. ¹H NMR (400 MHz, DMSO-d₆): δ 10.73 (s, 1H), 9.46 (s, 1H), 8.81 (s, 1H), 8.68 (s, 1H), 8.47 (s, 1H), 2.90-2.83 (m, 2H), 2.67 (s, 3H), 1.75-1.66 (m, 5H), 1.34-1.27 (m, 3H).

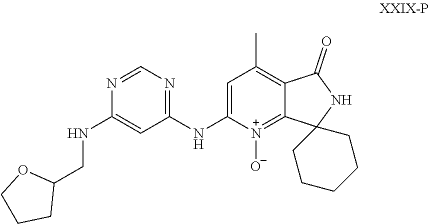 [SMILES: Cc1cc(Nc2cc(NCC3CCCO3)ncn2)[n+]([O-])c2c1C(=O)NC21CCCCC1]

4′-methyl-5′-oxo-2′-((6-(((tetrahydrofuran-2-yl)methyl)amino)pyrimidin-4-yl)amino)-5′,6′-dihydrospiro[cyclohexane-1,7′-pyrrolo[3,4-b]pyridine] 1′-oxide (Compound XXIX-P): LCMS (ESI, m/z): [M+H]⁺=425.3. ¹H NMR (400 MHz, DMSO-d₆): δ 10.15 (s, 1H), 9.32 (s, 1H), 8.49 (s, 1H), 8.30 (s, 1H), 7.37 (s, 1H), 6.45 (s, 1H), 4.01-3.94 (m, 1H), 3.81-3.76 (m, 1H), 3.66-3.61 (m, 1H), 3.60-3.44 (m, 2H), 2.91-2.83 (m, 2H), 2.57 (s, 3H), 1.97-1.65 (m, 8H), 1.58-1.52 (m, 1H), 1.29-1.23 (m, 3H).

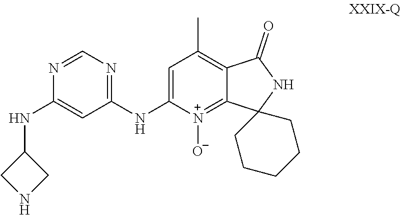 [SMILES: Cc1cc(Nc2cc(NC3CNC3)ncn2)[n+]([O-])c2c1C(=O)NC21CCCCC1]

2′-((6-(azetidin-3-ylamino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-Q): LCMS (ESI, m/z): [M+H]⁺=396.2. ¹H NMR (400 MHz, DMSO-d₆): δ 9.32 (s, 1H), 8.51 (s, 1H), 8.31 (s, 1H), 7.76 (s, 1H), 6.36 (s, 1H), 4.61-4.49 (m, 1H), 3.65-3.61 (m, 2H), 3.47-3.43 (m, 3H), 2.91-2.83 (m, 2H), 2.57 (s, 3H), 1.78-1.61 (m, 5H), 1.29-1.17 (m, 3H).

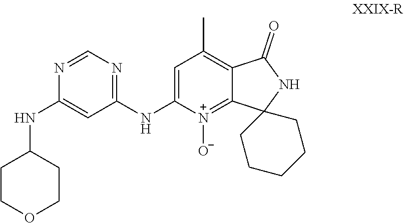 [SMILES: Cc1cc(Nc2cc(NC3CCOCC3)ncn2)[n+]([O-])c2c1C(=O)NC21CCCCC1]

4′-methyl-5′-oxo-2′-((6-((tetrahydro-2H-pyran-4-yl)amino)pyrimidin-4-yl)amino)-5′,6′-dihydrospiro[cyclohexane-1,7′-pyrrolo[3,4-b]pyridine] 1′-oxide (Compound XXIX-R): LCMS (ESI, m/z): [M+H]⁺=425.3. ¹HNMR (400 MHz, DMSO-d₆): δ 10.17 (s, 1H), 9.32 (s, 1H), 8.51 (s, 1H), 8.31 (s, 1H), 7.30 (s, 1H), 6.43 (s, 1H), 3.90-3.87 (m, 2H), 3.42-3.32 (m, 3H), 2.91-2.83 (m, 2H), 2.57 (s, 3H), 1.86-1.83 (m, 2H), 1.71-1.61 (m, 5H), 1.52-1.42 (m, 2H), 1.30-1.23 (m, 3H).

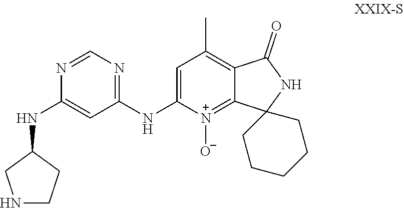 [SMILES: Cc1cc(Nc2cc(N[C@H]3CCNC3)ncn2)[n+]([O-])c2c1C(=O)NC21CCCCC1]

(S)-4′-methyl-5′-oxo-2′-((6-(pyrrolidin-3-ylamino)pyrimidin-4-yl)amino)-5′,6′-dihydrospiro[cyclohexane-1,7′-pyrrolo[3,4-b]pyridine] 1′-oxide (Compound XXIX-S): LCMS (ESI, m/z): [M+H]⁺=410.1. ¹HNMR (400 MHz, CD₃OD): δ 8.64 (s, 1H), 8.36 (s, 1H), 6.28 (s, 1H), 4.43-4.35 (m, 1H), 3.28-3.24 (m, 1H), 3.17-3.11 (m, 1H), 3.05-2.94 (m, 3H), 2.90-2.85 (m, 1H), 2.68 (s, 3H), 2.29-2.20 (m, 1H), 1.88-1.78 (m, 4H), 1.74-1.63 (m, 2H), 1.49-1.40 (m, 3H).

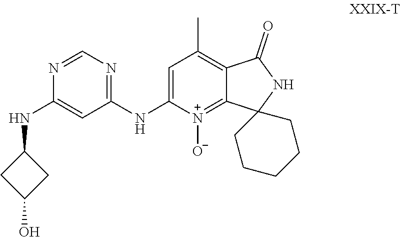 [SMILES: Cc1cc(Nc2cc(N[C@H]3C[C@H](O)C3)ncn2)[n+]([O-])c2c1C(=O)NC21CCCCC1]

trans-2′-((6-(((1r,3r)-3-hydroxycyclobutyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-T): LCMS (ESI, m/z): [M+H]⁺=411.3. ¹H NMR (400 MHz, DMSO-d₆): δ 10.21 (s, 1H), 9.33 (s, 1H), 8.53 (s, 1H), 8.29 (s, 1H), 7.57 (s, 1H), 6.33 (s, 1H), 5.04 (d, J=5.2 Hz, 1H), 4.32-4.27 (m, 2H), 2.91-2.83 (m, 2H), 2.68 (s, 3H), 2.20-2.17 (m, 4H), 1.71-1.64 (m, 5H), 1.29-1.20 (m, 3H).

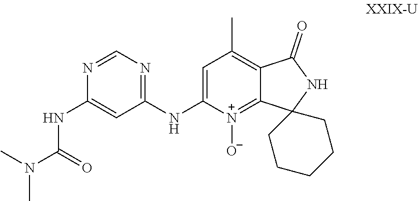 [SMILES: Cc1cc(Nc2cc(NC(=O)N(C)C)ncn2)[n+]([O-])c2c1C(=O)NC21CCCCC1]

2′-((6-(3,3-dimethylureido)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-U): LCMS (ESI, m/z): [M+H]⁺=412.3. ¹HNMR (400 MHz, DMSO-d₆): δ 10.39 (s, 1H), 9.38 (s, 1H), 9.28 (s, 1H), 8.57-8.54 (m, 2H), 7.71 (s, 1H), 2.96 (s, 6H), 2.90-2.84 (m, 2H), 2.60 (s, 3H), 1.78-1.61 (m, 5H), 1.30-1.23 (m, 3H).

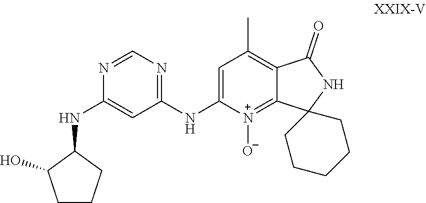 [SMILES: Cc1cc(Nc2cc(N[C@H]3CCC[C@@H]3O)ncn2)[n+]([O-])c2c1C(=O)NC21CCCCC1]

2′-((6-(((1S,2S)-2-hydroxycyclopentyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-V): LCMS (ESI, m/z): [M+H]⁺=425.3. ¹H NMR (400 MHz, DMSO-d₆): δ 10.14 (s, 1H), 9.33 (s, 1H), 8.51 (s, 1H), 8.30 (s, 1H), 7.25 (s, 1H), 6.41 (s, 1H), 4.85 (d, J=4.0 Hz, 1H), 3.94-3.62 (m, 2H), 2.91-2.83 (m, 2H), 2.58 (s, 3H), 2.12-2.03 (m, 1H), 1.88-1.80 (m, 1H), 1.74-1.65 (m, 7H), 1.52-1.42 (m, 2H), 1.28-1.19 (m, 3H).

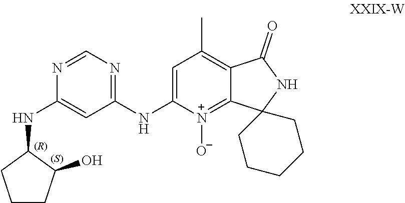

2′-((6-(((1R,2S)-2-hydroxycyclopentyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-W): LCMS (ESI, m/z): [M+H]+=425.3. 1H NMR (400 MHz, DMSO-d₆): δ 10.10 (s, 1H), 9.32 (s, 1H), 8.49 (s, 1H), 8.30 (s, 1H), 6.93 (s, 1H), 6.49 (s, 1H), 4.75 (s, 1H), 4.22-3.98 (m, 2H), 2.90-2.83 (m, 2H), 2.58 (s, 3H), 1.94-1.83 (m, 1H), 1.79-1.51 (m, 10H), 1.29-1.21 (m, 3H).

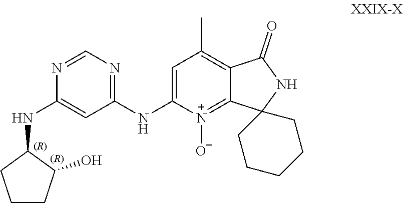

2′-((6-(((1R,2R)-2-hydroxycyclopentyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-X): LCMS (ESI, m/z): [M+H]⁺=425.2. ¹H NMR (400 MHz, DMSO-d₆): δ 10.19 (s, 1H), 9.33 (s, 1H), 8.51 (s, 1H), 8.29 (s, 1H), 7.26 (s, 1H), 6.41 (s, 1H), 4.85 (d, J=4.0 Hz, 1H), 3.92-3.85 (m, 2H), 2.92-2.81 (m, 2H), 2.67 (s, 3H), 2.11-2.03 (m, 1H), 1.87-1.80 (m, 1H), 1.79-1.65 (m, 7H), 1.52-1.46 (m, 2H), 1.30-1.22 (m, 3H).

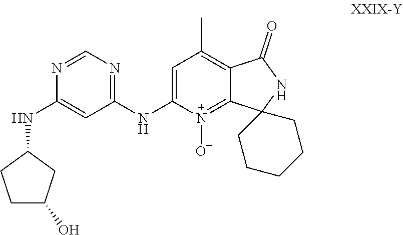 [SMILES: Cc1cc(Nc2cc(N[C@H]3CC[C@@H](O)C3)ncn2)[n+]([O-])c2c1C(=O)NC21CCCCC1]

2′-((6-((cis-3-hydroxycyclopentyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-Y): LCMS (ESI, m/z): [M+H]⁺=425.4. ¹H NMR (400 MHz, DMSO-d₆): δ 10.13 (s, 1H), 9.30 (s, 1H), 8.50 (s, 1H), 8.29 (s, 1H), 7.23 (s, 1H), 6.40 (s, 1H), 4.63 (d, J=3.6 Hz, 1H), 4.13-4.10 (m, 1H), 2.91-2.83 (m, 2H), 2.58 (s, 3H), 2.26-2.20 (m, 1H), 1.97-1.89 (m, 1H), 1.80-1.57 (m, 8H), 1.50-1.39 (m, 1H), 1.31-1.19 (m, 3H).

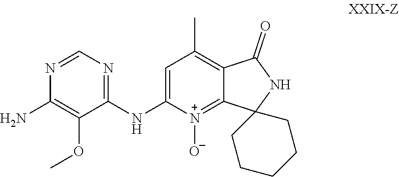 [SMILES: COc1c(N)ncnc1Nc1cc(C)c2c([n+]1[O-])C1(CCCCC1)NC2=O]

2′-((6-amino-5-methoxypyrimidin-4-yl)amino)-4′-methyl-5′-oxo-5′,6′-dihydrospiro[cyclohexane-1,7′-pyrrolo[3,4-b]pyridine] 1′-oxide (Compound XXIX-Z): LCMS (ESI, m/z): [M+H]⁺=371.3. ¹H NMR (400 MHz, DMSO-d₆): δ 10.23 (s, 1H), 9.38 (s, 1H), 8.56 (s, 1H), 8.08 (s, 1H), 6.90 (s, 2H), 3.76 (s, 3H), 2.88-2.80 (m, 2H), 2.60 (s, 3H), 1.70-1.64 (m, 5H), 1.30-1.27 (m, 3H).

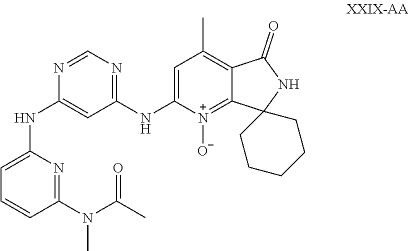 [SMILES: CC(=O)N(C)c1cccc(Nc2cc(Nc3cc(C)c4c([n+]3[O-])C3(CCCCC3)NC4=O)ncn2)n1]

4′-methyl-2′-((6-((6-(N-methylacetamido)pyridin-2-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AA): LCMS (ESI, m/z): [M+H]⁺=489.2. ¹H NMR (400 MHz, DMSO-d₆): δ 10.42 (s, 1H), 10.27 (s, 1H), 9.42 (s, 1H), 8.59 (s, 1H), 8.53 (s, 1H), 7.83-7.79 (m, 1H), 7.59 (s, 1H), 7.52 (d, J=8.0 Hz, 1H), 7.07 (d, J=7.2 Hz, 1H), 3.31 (s, 3H), 2.90-2.83 (m, 2H), 2.60 (s, 3H), 2.07 (s, 3H), 1.74-1.64 (m, 5H), 1.30-1.24 (m, 3H).

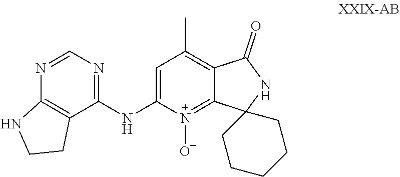 [SMILES: Cc1cc(Nc2ncnc3c2CCN3)[n+]([O-])c2c1C(=O)NC21CCCCC1]

2′-((6,7-dihydro-5H-pyrrolo[2,3-d]pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AB): LCMS (ESI, m/z): [M+H]⁺=367.3. ¹HNMR (400 MHz, DMSO-d₆): δ 9.79 (s, 1H), 9.40 (s, 1H), 8.32-8.28 (m, 2H), 7.97 (s, 1H), 3.72-3.68 (m, 2H), 3.12-3.07 (m, 2H), 2.83-2.76 (m, 2H), 2.57 (s, 3H), 1.73-1.64 (m, 3H), 1.30-1.24 (m, 5H).

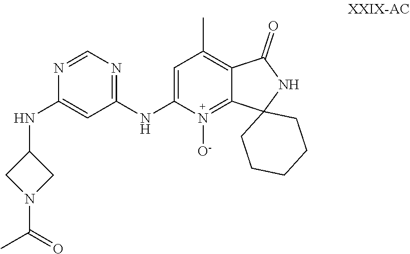

2′-((6-((1-acetylazetidin-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AC): LCMS (ESI, m/z): [M+H]⁺=438.3. ¹H NMR (400 MHz, DMSO-d₆): δ 10.29 (s, 1H), 9.34 (s, 1H), 8.53 (s, 1H), 8.36 (s, 1H), 7.95 (d, J=5.2 Hz, 1H), 6.44 (s, 1H), 4.50-4.41 (m, 2H), 4.17-4.13 (m, 1H), 3.98-3.95 (m, 1H), 3.77-3.73 (m, 1H), 2.91-2.83 (m, 2H), 2.57 (s, 3H), 1.77-1.61 (m, 8H), 1.30-1.18 (m, 3H).

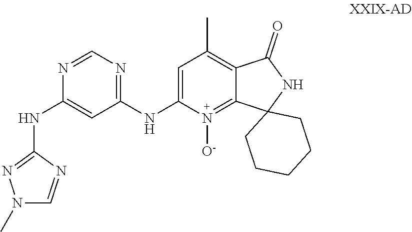

4′-methyl-2′-((6-((1-methyl-Ill-1,2,4-triazol-3-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AD): [M+H]⁺=422.2. ¹H NMR (400 MHz, DMSO-d₆): δ 10.30 (s, 1H), 10.21 (s, 1H), 9.38 (s, 1H), 8.54 (s, 1H), 8.50 (s, 1H), 8.35 (s, 1H), 7.64 (s, 1H), 3.86 (s, 3H), 2.89-2.84 (m, 2H), 2.61 (s, 3H), 1.79-1.67 (m, 5H), 1.38-1.23 (m, 3H).

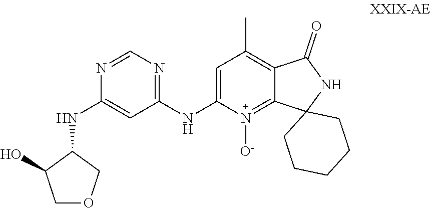

2′-((6-((trans-4-hydroxytetrahydrofuran-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AE): LCMS (ESI, m/z): [M+H]⁺=427.2. ¹H NMR (400 MHz, DMSO-d₆): δ 10.22 (s, 1H), 9.33 (s, 1H), 8.51 (s, 1H), 8.35 (s, 1H), 7.51 (s, 1H), 6.43 (s, 1H), 5.29 (d, J=3.6 Hz, 1H), 4.26-4.17 (m, 1H), 4.15-4.03 (m, 2H), 4.02-3.89 (m, 1H), 3.70-3.64 (m, 1H), 3.62-3.56 (m, 1H), 2.90-2.83 (m, 2H), 2.58 (s, 3H), 1.81-1.65 (m, 5H), 1.30-1.16 (m, 3H).

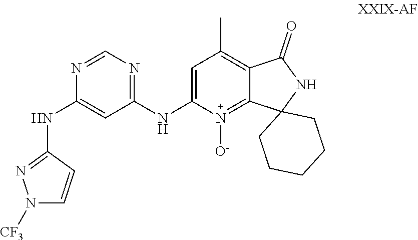

4′-methyl-5′-oxo-2′-[(6-{[1-(trifluoromethyl)pyrazol-3-yl]amino}pyrimidin-4-yl)amino]-6′H-spiro[cyclohexane-1,7′-pyrrolo[3,4-b]pyridin]-1′-ium-1′-olate (Compound XXIX-AF): LCMS (ESI, m/z): [M+H]⁺=475.1. ¹H NMR (400 MHz, DMSO-d₆): δ 10.45 (s, 1H), 10.39 (s, 1H), 9.38 (s, 1H), 8.55-8.52 (m, 2H), 8.37 (d, J=2.8 Hz, 1H), 7.20 (s, 1H), 6.78 (d, J=2.4 Hz, 1H), 2.91-2.84 (m, 2H), 2.60 (s, 3H), 1.72-1.62 (m, 5H), 1.35-1.20 (m, 3H).

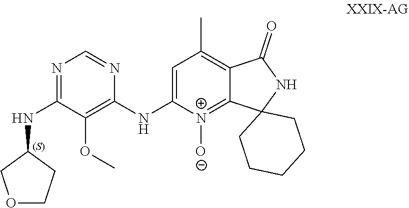

(S)-2′-((5-methoxy-6-((tetrahydrofuran-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AG): LCMS (ESI, m/z): [M+H]⁺=441.2. ¹H NMR (400 MHz, DMSO-d₆): δ 10.23 (s, 1H), 9.41 (s, 1H), 8.55 (s, 1H), 8.20 (s, 1H), 7.32 (d, J=6.4 Hz, 1H), 4.61-4.57 (m, 1H), 3.92-3.86 (m, 2H), 3.75-3.70 (m, 4H), 3.62-3.59 (m, 1H), 2.88-2.80 (m, 2H), 2.61 (s, 3H), 2.22-2.13 (m, 1H), 2.04-1.96 (m, 1H), 1.70-1.61 (m, 5H), 1.31-1.24 (m, 3H).

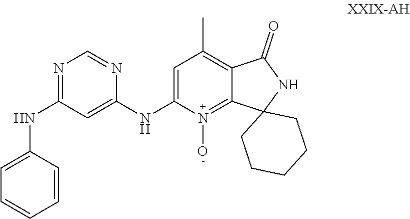

4′-methyl-5′-oxo-2′-((6-(phenyl amino) pyrimidin-4-yl) amino)-5′,6′-dihydrospiro[cyclohexane-1,7′-pyrrolo[3,4-b] pyridine] 1′-oxide (Compound XXIX-AH): LCMS (ESI, m/z): [M+H]⁺=417.3 ¹H NMR (400 MHz, DMSO-d₆): δ 10.42 (s, 1H), 9.51 (s, 1H), 9.35 (s, 1H), 8.55-8.37 (m, 2H), 7.69-7.59 (m, 2H), 7.33-7.23 (m, 2H), 7.04 (s, 1H), 6.81 (s, 1H), 2.93-2.83 (m, 2H), 2.66 (s, 3H), 1.78-1.70 (m, 5H), 1.29-1.19 (m, 3H).

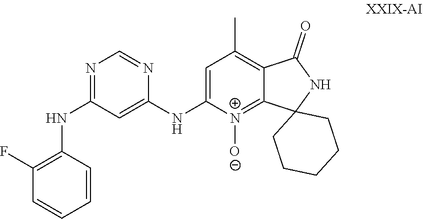 [SMILES: Cc1cc(Nc2cc(Nc3ccccc3F)ncn2)[n+]([O-])c2c1C(=O)NC21CCCCC1]

2′-((6-((2-fluorophenyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AI): LCMS (ESI, m/z): [M+H]⁺=435.2. ¹H NMR (400 MHz, DMSO-d₆): δ 10.37 (s, 1H), 9.36 (s, 1H), 9.26 (s, 1H), 8.54 (s, 1H), 8.44 (s, 1H), 7.73-7.68 (m, 1H), 7.31-7.19 (m, 3H), 6.73 (s, 1H), 2.90-2.83 (m, 2H), 2.59 (s, 3H), 1.74-1.65 (m, 5H), 1.30-1.21 (m, 3H).

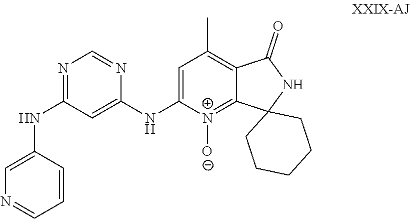 [SMILES: Cc1cc(Nc2cc(Nc3cccnc3)ncn2)[n+]([O-])c2c1C(=O)NC21CCCCC1]

4′-methyl-5′-oxo-2′-((6-(pyridin-3-ylamino)pyrimidin-4-yl)amino)-5′,6′-dihydrospiro[cyclohexane-1,7′-pyrrolo[3,4-b]pyridine] 1′-oxide (Compound XXIX-AJ): LCMS (ESI, m/z): [M+H]⁺=418.3. ¹H NMR (400 MHz, DMSO-d₆): δ 10.52 (s, 1H), 9.73 (s, 1H), 9.39 (s, 1H), 8.78 (d, J=2.4 Hz, 1H), 8.57-8.54 (m, 2H), 8.23-8.22 (m, 1H), 8.12-8.09 (m, 1H), 7.37-7.34 (m, 1H), 6.86 (s, 1H), 2.92-2.85 (m, 2H), 2.60 (s, 3H), 1.75-1.60 (m, 5H), 1.31-1.21 (m, 3H).

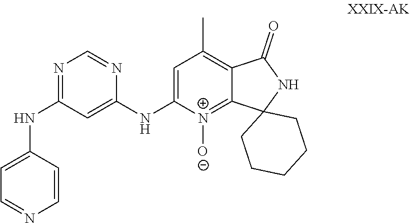 [SMILES: Cc1cc(Nc2cc(Nc3ccncc3)ncn2)[n+]([O-])c2c1C(=O)NC21CCCCC1]

4′-methyl-5′-oxo-2′-{[6-(pyridin-4-ylamino)pyrimidin-4-yl]amino}-6′H-Spiro[cyclohexane-1,7′-pyrrolo[3,4-b]pyridin]-1′-ium-1′-olate (Compound XXIX-AK): LCMS (ESI, m/z): [M+H]⁺=418.2. ¹H NMR (400 MHz, DMSO-d₆): δ 10.61 (s, 1H), 10.01 (s, 1H), 9.41 (s, 1H), 8.63-8.57 (m, 2H), 8.38 (d, J=6.0 Hz, 2H), 7.67 (d, J=6.4 Hz, 2H), 6.94 (s, 1H), 2.92-2.84 (m, 2H), 2.60 (s, 3H), 1.74-1.62 (m, 5H), 1.31-1.21 (m, 3H).

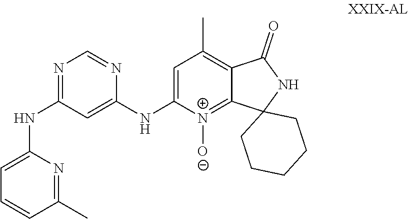 [SMILES: Cc1cccc(Nc2cc(Nc3cc(C)c4c([n+]3[O-])C3(CCCCC3)NC4=O)ncn2)n1]

4′-methyl-2′-((6-((6-methylpyridin-2-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AL): LCMS (ESI, m/z): [M+H]⁺=432.2. ¹HNMR (400 MHz, DMSO-d₆): δ 10.33 (s, 1H), 10.05 (s, 1H), 9.38 (s, 1H), 8.55-8.51 (m, 2H), 7.78 (s, 1H), 7.63-7.59 (m, 1H), 7.38 (d, J=8.0 Hz, 1H), 6.86 (d, J=7.2 Hz, 1H), 2.92-2.84 (m, 2H), 2.61 (s, 3H), 2.48 (s, 3H), 1.72-1.66 (m, 5H), 1.31-1.21 (m, 3H).

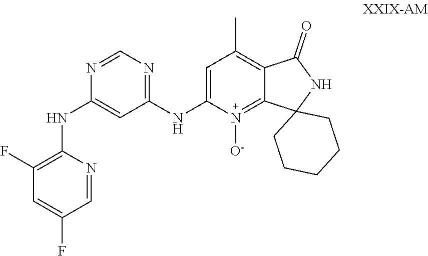

2′-((6-((3,5-difluoropyridin-2-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AM): LCMS (ESI, m/z): [M+H]⁺=454.3. ¹H NMR (400 MHz, DMSO-d₆): δ 10.45 (s, 1H), 9.80 (s, 1H), 9.37 (s, 1H), 8.58 (s, 1H), 8.52 (s, 1H), 8.31 (s, 1H), 8.03-7.99 (m, 1H), 7.48 (s, 1H), 2.90-2.85 (m, 2H), 2.68 (s, 3H), 1.71-1.55 (m, 5H), 1.28-1.18 (m, 3H).

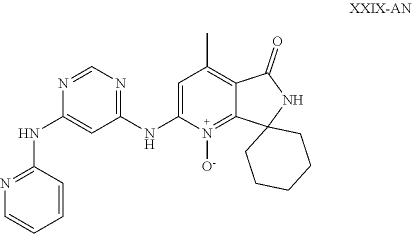

4′-methyl-2′-((5-methyl-6-(pyridin-2-ylamino)pyrimidin-4-yl)amino)-5′-oxo-5′,6′-dihydrospiro[cyclohexane-1,7′-pyrrolo[3,4-b]pyridine] 1′-oxide (Compound XXIX-AN): LCMS (ESI, m/z): [M+H]⁺=432.3. ¹H NMR (400 MHz, DMSO-d₆): δ 10.25 (s, 1H), 9.41 (s, 1H), 9.10 (s, 1H), 8.59 (s, 1H), 8.51 (s, 1H), 8.32-8.31 (m, 1H), 7.99 (d, J=8.4 Hz, 1H), 7.78-7.74 (m, 1H), 7.06-7.03 (m, 1H), 2.89-2.82 (m, 2H), 2.63 (s, 3H), 2.28 (s, 3H), 1.72-1.61 (m, 5H), 1.32-1.23 (m, 3H).

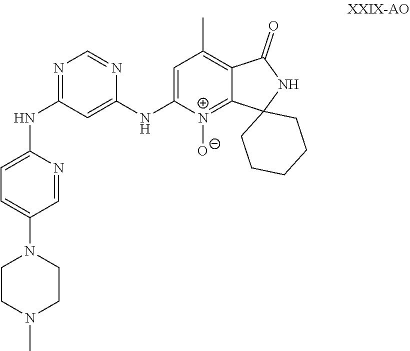 [SMILES: Cc1cc(Nc2cc(Nc3ccc(N4CCN(C)CC4)cn3)ncn2)[n+]([O-])c2c1C(=O)NC21CCCCC1]

4′-methyl-2′-[(6-{[5-(4-methylpiperazin-1-yl)pyridin-2-yl]amino}pyrimidin-4-yl)amino]-5′-oxo-5′,6′-dihydrospiro[cyclohexane-1,7′-pyrrolo[3,4-b]pyridin]-1′-ium-1′-olate (Compound XXIX-AO): LCMS (ESI, m/z): [M+H]⁺=516.4. ¹H NMR (400 MHz, DMSO-d₆): δ 10.26 (s, 1H), 9.87 (s, 1H), 9.37 (s, 1H), 8.53-8.50 (m, 2H), 8.02 (s, 1H), 7.61 (s, 1H), 7.44 (s, 2H), 3.15-3.11 (m, 4H), 2.91-2.83 (m, 2H), 2.60 (s, 3H), 2.49-2.46 (m, 4H), 2.23 (s, 3H), 1.75-1.61 (m, 5H), 1.31-1.21 (m, 3H).

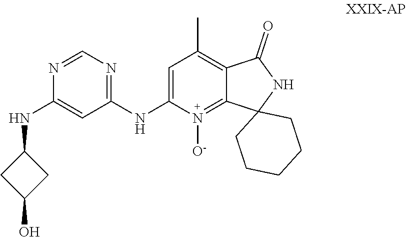

2′-((6-(((cis)-3-hydroxycyclobutyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AP): LCMS (ESI, m/z): [M+H]⁺=411.2. ¹H NMR (400 MHz, DMSO-d₆): δ 10.19 (s, 1H), 9.38 (s, 1H), 8.52 (s, 1H), 8.28 (s, 1H), 7.52 (d, J=5.6 Hz, 1H), 6.34 (s, 1H), 5.08 (s, 1H), 3.87-3.83 (m, 1H), 2.91-2.83 (m, 2H), 2.73-2.64 (m, 2H), 2.57 (s, 3H), 1.81-1.61 (m, 8H), 1.29-1.22 (m, 3H).

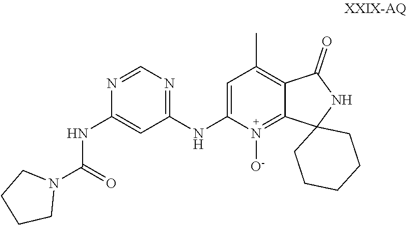

4′-methyl-5′-oxo-2′-((6-(pyrrolidine-1-carboxamido)pyrimidin-4-yl)amino)-5′,6′-dihydrospiro[cyclohexane-1,7′-pyrrolo[3,4-b]pyridine] 1′-oxide (Compound XXIX-AQ): LCMS (ESI, m/z): [M+H]⁺=438.4. ¹H NMR (400 MHz, DMSO-d₆): δ 10.38 (s, 1H), 9.38 (s, 1H), 9.10 (s, 1H), 8.58 (s, 1H), 8.54 (s, 1H), 7.79 (s, 1H), 3.43-3.33 (m, 4H), 2.87-2.84 (m, 2H), 2.60 (s, 3H), 1.87-1.82 (m, 4H), 1.78-1.65 (m, 5H), 1.30-1.24 (m, 3H).

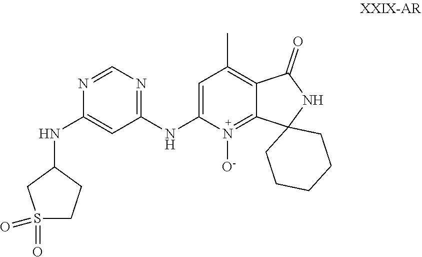

2′-((6-((1,1-dioxidotetrahydrothiophen-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AR): LCMS (ESI, m/z): [M+H]⁺=459.2. ¹H NMR (400 MHz, DMSO-d₆): δ 10.28 (s, 1H), 9.34 (s, 1H), 8.53 (s, 1H), 8.38 (s, 1H), 7.73 (d, J=6.8 Hz, 1H), 6.51 (s, 1H), 4.62 (s, 1H), 3.60-3.55 (m, 1H), 3.40-3.35 (m, 2H), 3.22-3.15 (m, 1H), 2.98-2.83 (m, 3H), 2.58 (s, 3H), 2.19-2.13 (m, 1H), 1.71-1.61 (m, 5H), 1.30-1.21 (m, 3H).

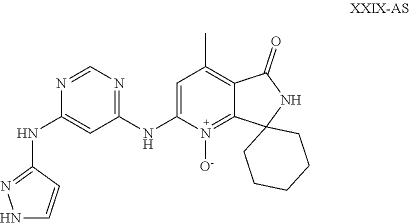

2′-((6-((1H-pyrazol-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AS): [M+H]⁺=407.3. ¹H NMR (400 MHz, DMSO-d₆): δ 12.32 (s, 1H), 10.24 (s, 1H), 9.75 (s, 1H), 9.35 (s, 1H), 8.73-8.45 (m, 2H), 7.64 (s, 1H), 7.12 (s, 1H), 6.31 (s, 1H), 2.92-2.80 (m, 2H), 2.58 (s, 3H), 1.70-1.55 (m, 5H), 1.30-1.22 (m, 3H).

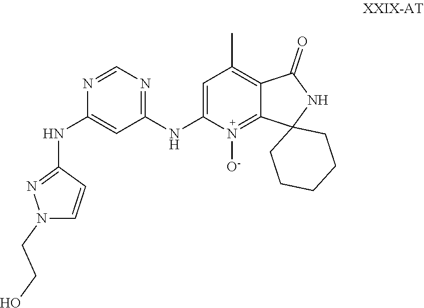

2′-((6-((1-(2-hydroxyethyl)-1H-pyrazol-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AT): LCMS (ESI, m/z): [M+H]⁺=451.2. ¹H NMR (400 MHz, DMSO-d₆): δ 10.26 (s, 1H), 9.78 (s, 1H), 9.35 (s, 1H), 8.51 (s, 1H), 8.45 (s, 1H), 7.61 (s, 1H), 7.15 (s, 1H), 6.25 (s, 1H), 4.88-4.85 (m, 1H), 4.10-4.07 (m, 2H), 3.78-3.74 (m, 2H), 2.91-2.83 (m, 2H), 2.60 (s, 3H), 1.71-1.60 (m, 5H), 1.30-1.23 (m, 3H).

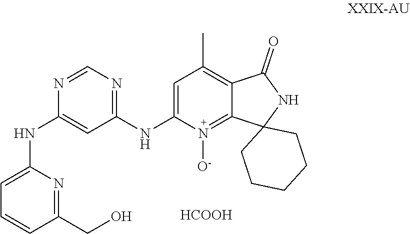

2′-((6-((6-(hydroxymethyl)pyridin-2-yl)amino)pyrimidin-4-yl)amino)-4′-methyl-5′-oxo-5′,6′-dihydrospiro[cyclohexane-1,7′-pyrrolo[3,4-b]pyridine] 1′-oxide formate salt (Compound XXIX-AU): LCMS (ESI, m/z): [M+H]⁺=448.3. ¹H NMR (400 MHz, DMSO-d₆): δ 10.39 (s, 1H), 10.09 (s, 1H), 9.37 (s, 1H), 8.55 (d, J=5.6 Hz, 2H), 8.26 (s, 1H), 7.90 (s, 1H), 7.73-7.69 (m, 1H), 7.35 (d, J=8.4 Hz, 1H), 7.05 (d, J=7.2 Hz, 1H), 5.38 (s, 1H), 4.57 (d, J=10.0 Hz, 2H), 2.92-2.85 (m, 2H), 2.61 (s, 3H), 1.78-1.62 (m, 5H), 1.31-1.25 (m, 3H).

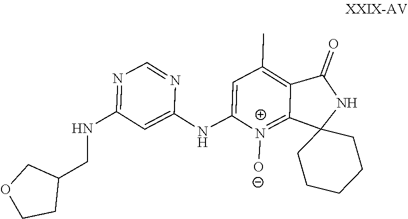 [SMILES: Cc1cc(Nc2cc(NCC3CCOC3)ncn2)[n+]([O-])c2c1C(=O)NC21CCCCC1]

4′-methyl-5′-oxo-2′-((6-(((tetrahydrofuran-3-yl)methyl)amino)pyrimidin-4-yl)amino)-5′,6′-dihydrospiro[cyclohexane-1,7′-pyrrolo[3,4-b]pyridine] 1′-oxide (Compound XXIX-AV): LCMS (ESI, m/z): [M+H]⁺=425.2. ¹H NMR (400 MHz, DMSO-d₆): δ 10.17 (s, 1H), 9.32 (s, 1H), 8.52 (s, 1H), 8.31 (s, 1H), 7.45 (s, 1H), 6.44 (s, 1H), 3.78-3.69 (m, 2H), 3.65-3.60 (m, 1H), 3.47-3.44 (m, 1H), 3.30-3.22 (m, 2H), 2.91-2.83 (m, 2H), 2.57 (s, 3H), 2.01-1.92 (m, 1H), 1.78-1.52 (m, 6H), 1.33-1.20 (m, 3H).

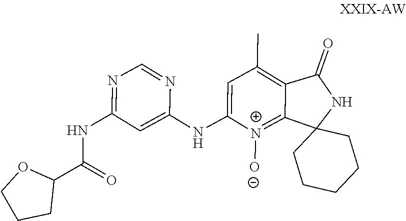 [SMILES: Cc1cc(Nc2cc(NC(=O)C3CCCO3)ncn2)[n+]([O-])c2c1C(=O)NC21CCCCC1]

4′-methyl-5′-oxo-2′-((6-(tetrahydrofuran-2-carboxamido)pyrimidin-4-yl)amino)-5′,6′-dihydrospiro[cyclohexane-1,7′-pyrrolo[3,4-b]pyridine] 1′-oxide (Compound XXIX-AW): LCMS (ESI, m/z): [M+H]⁺=439.3. ¹H NMR (400 MHz, DMSO-d₆): δ 10.66 (s, 1H), 10.11 (s, 1H), 9.40 (s, 1H), 8.67 (s, 1H), 8.56 (s, 1H), 8.07 (s, 1H), 4.54-4.50 (m, 1H), 4.01-3.95 (m, 1H), 3.87-3.82 (m, 1H), 2.90-2.83 (m, 2H), 2.60 (s, 3H), 2.27-2.18 (m, 1H), 2.02-1.83 (m, 3H), 1.74-1.62 (m, 5H), 1.30-1.21 (m, 3H).

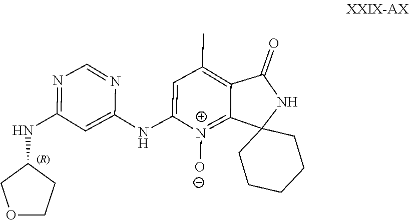

(R)-4′-methyl-5′-oxo-2′-((6-((tetrahydrofuran-3-yl)amino)pyrimidin-4-yl)amino)-5′,6′-dihydrospiro[cyclohexane-1,7′-pyrrolo[3,4-b]pyridine] 1′-oxide (Compound XXIX-AX): LCMS (ESI, m/z): [M+H]⁺=411.1. ¹H NMR (400 MHz, DMSO-d₆): δ 10.20 (s, 1H), 9.32 (s, 1H), 8.51 (s, 1H), 8.33 (s, 1H), 7.65-7.43 (m, 1H), 6.43 (s, 1H), 4.34 (s, 1H), 3.90-3.81 (m, 2H), 3.75-3.64 (m, 1H), 3.56-3.54 (m, 1H), 3.10-2.88 (m, 2H), 2.58 (s, 3H), 2.21-2.17 (m, 1H), 1.86-1.51 (m, 6H), 1.33-1.21 (m, 3H).

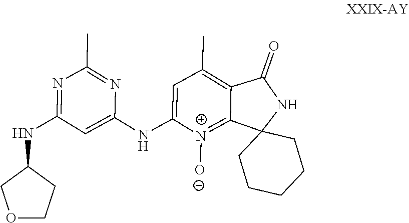 [SMILES: Cc1nc(Nc2cc(C)c3c([n+]2[O-])C2(CCCCC2)NC3=O)cc(N[C@H]2CCOC2)n1]

(S)-4′-methyl-2′-((2-methyl-6-((tetrahydrofuran-3-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AY): LCMS (ESI, m/z): [M+H]⁺=425.3. ¹H NMR (400 MHz, DMSO-d₆): δ 10.12 (s, 1H), 9.32 (s, 1H), 8.58 (s, 1H), 7.41 (d, J=6.0 Hz, 1H), 6.26 (s, 1H), 4.31 (s, 1H), 3.90-3.80 (m, 2H), 3.75-3.70 (m, 1H), 3.54-3.51 (m, 1H), 2.91-2.83 (m, 2H), 2.58 (s, 3H), 2.38 (s, 3H), 2.22-2.14 (m, 1H), 1.87-1.78 (m, 1H), 1.77-1.58 (m, 5H), 1.34-1.20 (m, 3H).

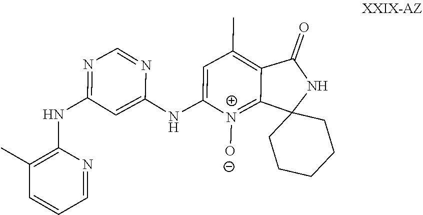 [SMILES: Cc1cccnc1Nc1cc(Nc2cc(C)c3c([n+]2[O-])C2(CCCCC2)NC3=O)ncn1]

4′-methyl-2′-((6-((3-methylpyridin-2-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AZ): LCMS (ESI, m/z): [M+H]⁺=432.2. ¹H NMR (400 MHz, DMSO-d₆): δ 10.38 (s, 1H), 9.36 (s, 1H), 9.01 (s, 1H), 8.57 (s, 1H), 8.53 (s, 1H), 8.25 (d, J=3.6 Hz, 1H), 7.69 (s, 1H), 7.64 (d, J=7.2 Hz, 1H), 7.08-7.05 (m, 1H), 2.91-2.83 (m, 2H), 2.60 (s, 3H), 2.31 (s, 3H), 1.75-1.61 (m, 5H), 1.30-1.21 (m, 3H).

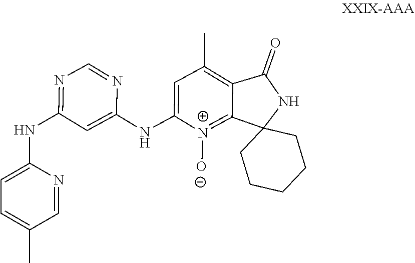 [SMILES: Cc1ccc(Nc2cc(Nc3cc(C)c4c([n+]3[O-])C3(CCCCC3)NC4=O)ncn2)nc1]

4′-methyl-2′-((6-((5-methylpyridin-2-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AAA): LCMS (ESI, m/z): [M+H]⁺=432.3. ¹H NMR (400 MHz, DMSO-d₆): δ 10.32 (s, 1H), 10.02 (s, 1H), 9.37 (s, 1H), 8.54 (d, J=3.2 Hz, 2H), 8.17 (s, 1H), 7.75 (s, 1H), 7.57 (d, J=8.0 Hz, 1H), 7.46 (d, J=8.4 Hz, 1H), 2.90-2.85 (m, 2H), 2.61 (s, 3H), 2.25 (s, 3H), 1.75-1.61 (m, 5H), 1.31-1.24 (m, 3H).

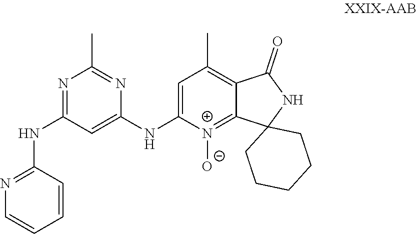 [SMILES: Cc1nc(Nc2ccccn2)cc(Nc2cc(C)c3c([n+]2[O-])C2(CCCCC2)NC3=O)n1]

4′-methyl-2′-((2-methyl-6-(pyridin-2-ylamino)pyrimidin-4-yl)amino)-5′-oxo-5′,6′-dihydrospiro[cyclohexane-1,7′-pyrrolo[3,4-b]pyridine] 1′-oxide (Compound XXIX-AAB): LCMS (ESI, m/z): [M+H]⁺=432.3. ¹H NMR (400 MHz, DMSO-d₆): δ 10.08 (s, 1H), 9.91 (s, 1H), 9.20 (s, 1H), 8.53 (s, 1H), 8.31 (d, J=4.8 Hz, 1H), 7.74-7.67 (m, 2H), 7.50-7.45 (m, 1H), 6.98-6.94 (m, 1H), 6.75-6.55 (m, 1H), 3.94-3.90 (m, 1H), 2.93-2.84 (m, 2H), 2.62 (s, 3H), 1.79-1.57 (m, 5H), 1.32-1.20 (m, 3H).

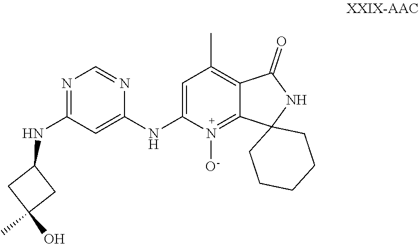

4′-methyl-5′-oxo-2′-[(6-{[trans-3-hydroxy-3-methylcyclobutyl]amino}pyrimidin-4-yl)amino]-6′H-spiro[cyclohexane-1,7′-pyrrolo[3,4-b]pyridin]-1′-ium-1′-olate (Compound XXIX-AAC): LCMS (ESI, m/z): [M+H]⁺=425.3. ¹H NMR (400 MHz, DMSO-d₆): δ 10.20 (s, 1H), 9.33 (s, 1H), 8.53 (s, 1H), 8.32-8.29 (m, 1H), 7.52-7.45 (m, 1H), 6.39 (s, 1H), 4.98 (s, 1H), 4.02-3.88 (m, 1H), 2.91-2.83 (m, 2H), 2.57 (s, 3H), 2.39-2.32 (m, 2H), 1.99-1.94 (m, 2H), 1.74-1.62 (m, 4H), 1.29-1.20 (m, 5H).

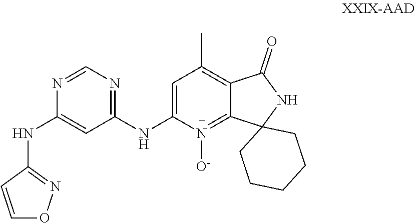

2′-((6-(isoxazol-3-ylamino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AAD): LCMS (ESI, m/z): [M+H]⁺=408.2.41 NMR (400 MHz, DMSO-d₆): δ 10.42 (s, 1H), 9.38 (s, 1H), 8.76 (d, J=2.0 Hz, 1H), 8.56 (d, J=9.2 Hz, 2H), 7.34 (s, 1H), 6.70 (d, J=1.6 Hz, 1H), 2.91-2.83 (m, 2H), 2.60 (s, 3H), 1.71-1.62 (m, 5H), 1.30-1.22 (m, 3H).

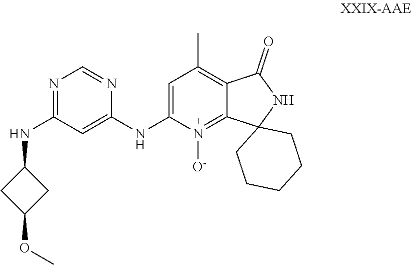

2′-((6-((cis-3-methoxycyclobutyl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AAE): LCMS (ESI, m/z): [M+H]⁺=425.2. ¹H NMR (400 MHz, DMSO-d₆): δ 10.20 (s, 1H), 9.33 (s, 1H), 8.52 (s, 1H), 8.30 (s, 1H), 7.56 (d, J=6.4 Hz, 1H), 6.37 (s, 1H), 3.65-3.58 (m, 2H), 3.31 (s, 3H), 2.97-2.83 (m, 2H), 2.73-2.62 (m, 2H), 2.57 (s, 3H), 1.93-1.63 (m, 7H), 1.37-1.19 (m, 3H).

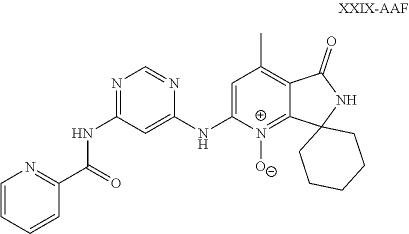 [SMILES: Cc1cc(Nc2cc(NC(=O)c3ccccn3)ncn2)[n+]([O-])c2c1C(=O)NC21CCCCC1]

4′-methyl-5′-oxo-2′-((6-(picolinamido)pyrimidin-4-yl)amino)-5′,6′-dihydrospiro[cyclohexane-1,7′-pyrrolo[3,4-b]pyridine] 1′-oxide (Compound XXIX-AAF): LCMS (ESI, m/z): [M+H]⁺=446.1. ¹H NMR (400 MHz, DMSO-d₆): δ 10.81 (s, 1H), 10.47 (s, 1H), 9.42 (s, 1H), 8.80-8.78 (m, 1H), 8.74 (s, 1H), 8.60 (s, 1H), 8.29-8.24 (m, 2H), 8.17-8.13 (m, 1H), 7.79-7.76 (m, 1H), 2.93-2.85 (m, 2H), 2.62 (s, 3H), 1.72-1.66 (m, 5H), 1.31-1.24 (m, 3H).

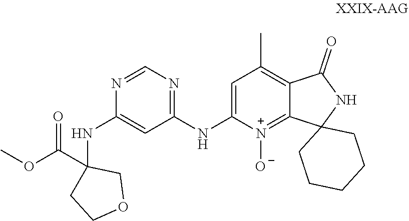 [SMILES: COC(=O)C1(Nc2cc(Nc3cc(C)c4c([n+]3[O-])C3(CCCCC3)NC4=O)ncn2)CCOC1]

2′-((6-((3-(methoxycarbonyl)tetrahydrofuran-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AAG): LCMS (ESI, m/z): [M+H]⁺=469.2. ¹H NMR (400 MHz, DMSO-d₆): δ 8.37 (s, 1H), 8.24 (s, 1H), 6.37 (s, 1H), 4.25 (d, J=9.6 Hz, 1H), 3.87-3.81 (m, 3H), 3.56 (s, 3H), 2.80-2.73 (m, 2H), 2.53 (s, 3H), 2.42-2.35 (m, 1H), 2.23-2.14 (m, 1H), 1.73-1.68 (m, 3H), 1.61-1.46 (m, 2H), 1.25-1.19 (m, 3H).

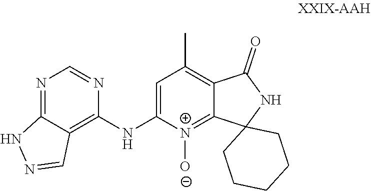 [SMILES: Cc1cc(Nc2ncnc3[nH]ncc23)[n+]([O-])c2c1C(=O)NC21CCCCC1]

2′-((1H-pyrazolo[3,4-d]pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AAH): LCMS (ESI, m/z): [M+H]⁺=366.1. ¹H NMR (400 MHz, DMSO-d₆): δ 9.48 (s, 1H), 8.75-8.63 (m, 3H), 2.93-2.83 (m, 2H), 2.66 (s, 3H), 1.76-1.61 (m, 5H), 1.33-1.27 (m, 3H).

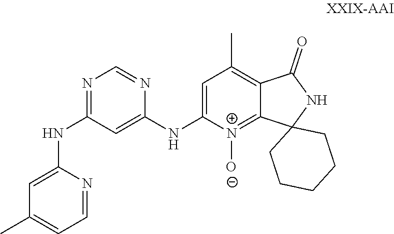 [SMILES: Cc1ccnc(Nc2cc(Nc3cc(C)c4c([n+]3[O-])C3(CCCCC3)NC4=O)ncn2)c1]

4′-methyl-2′-((6-((4-methylpyridin-2-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AAI): LCMS (ESI, m/z): [M+H]⁺=432.3. ¹HNMR (400 MHz, DMSO-d₆): δ 10.35 (s, 1H), 10.07 (s, 1H), 9.38 (s, 1H), 8.56-8.51 (m, 2H), 8.20 (d, J=5.2 Hz, 1H), 7.83 (s, 1H), 7.33 (s, 1H), 6.86 (d, J=5.2 Hz, 1H), 2.91-2.83 (m, 2H), 2.61 (s, 3H), 2.31 (s, 3H), 1.72-1.62 (m, 5H), 1.31-1.24 (m, 3H).

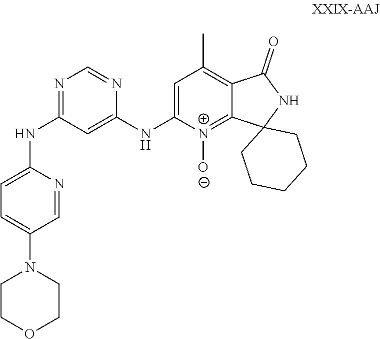 [SMILES: Cc1cc(Nc2cc(Nc3ccc(N4CCOCC4)cn3)ncn2)[n+]([O-])c2c1C(=O)NC21CCCCC1]

4′-methyl-2′-((6-((5-morpholinopyridin-2-yl)amino)pyrimidin-4-yl)amino)-5′-oxo-5′,6′-dihydrospiro[cyclohexane-1,7′-pyrrolo[3,4-b]pyridine] 1′-oxide (Compound XXIX-AAJ): LCMS (ESI, m/z): [M+H]⁺=503.3. ¹H NMR (400 MHz, DMSO-d₆): δ 10.27 (s, 1H), 9.89 (s, 1H), 9.37 (s, 1H), 8.53-8.51 (m, 2H), 8.03-8.02 (m, 1H), 7.62 (s, 1H), 7.46 (s, 2H), 3.78-3.75 (m, 4H), 3.11-3.09 (m, 4H), 2.92-2.85 (m, 2H), 2.60 (s, 3H), 1.78-1.63 (m, 5H), 1.31-1.21 (m, 3H).

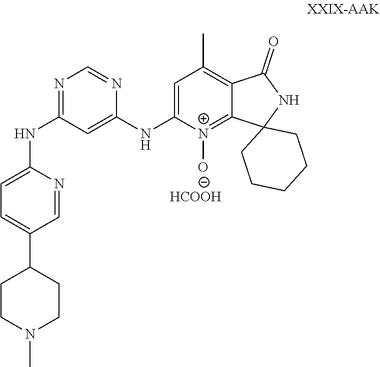 [SMILES: Cc1cc(Nc2cc(Nc3ccc(C4CCN(C)CC4)cn3)ncn2)[n+](O)c2c1C(=O)NC21CCCCC1 ; "HCOOH"]

4′-methyl-2′-((6-((5-(1-methylpiperidin-4-yl)pyridin-2-yl)amino)pyrimidin-4-yl)amino)-5′-oxo-5′,6′-dihydrospiro[cyclohexane-1,7′-pyrrolo[3,4-b]pyridine] 1′-oxide formate (Compound XXIX-AAK): LCMS (ESI, m/z): [M+H]⁺=515.3. ¹H NMR (400 MHz, DMSO-d₆): δ 10.33 (s, 1H), 10.05 (s, 1H), 9.37 (s, 1H), 8.54 (d, J=1.2 Hz, 2H), 8.22-8.20 (m, 2H), 7.80 (s, 1H), 7.66-7.63 (m, 1H), 7.46 (d, J=8.4 Hz, 1H), 2.97-2.81 (m, 5H), 2.61 (s, 3H), 2.26 (s, 3H), 2.10-2.04 (m, 2H), 1.81-1.62 (m, 9H), 1.33-1.25 (m, 3H).

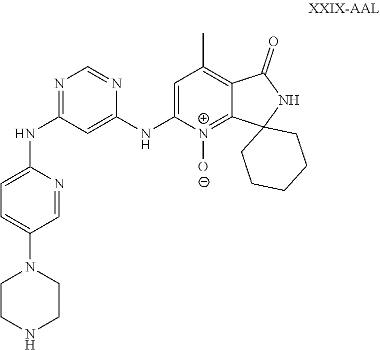 [SMILES: Cc1cc(Nc2cc(Nc3ccc(N4CCNCC4)cn3)ncn2)[n+]([O-])c2c1C(=O)NC21CCCCC1]

4′-methyl-5′-oxo-2′-[(6-{[5-(piperazin-1-yl)pyridin-2-yl]amino}pyrimidin-4-yl)amino]-6′H-spiro[cyclohexane-1,7′-pyrrolo[3,4-b]pyridin]-1′-ium-1′-olate (Compound XXIX-AAL): LCMS (ESI, m/z): [M+H]⁺=502.4. ¹H NMR (400 MHz, DMSO-d₆): δ 10.29 (s, 1H), 9.93 (s, 1H), 9.40 (s, 1H), 8.54-8.51 (m, 2H), 8.19 (s, 1H), 8.03 (d, J=1.6 Hz, 1H), 7.64 (s, 1H), 7.46 (s, 2H), 3.17-3.14 (m, 4H), 3.04-3.02 (m, 4H), 2.90-2.82 (m, 2H), 2.60 (s, 3H), 1.71-1.62 (m, 5H), 1.31-1.21 (m, 3H).

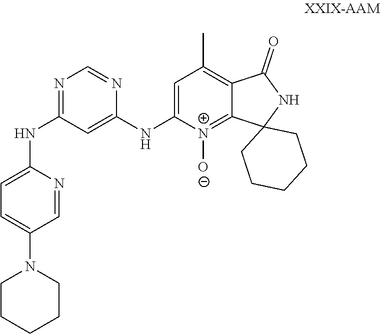 [SMILES: Cc1cc(Nc2cc(Nc3ccc(N4CCCCC4)cn3)ncn2)[n+]([O-])c2c1C(=O)NC21CCCCC1]

4′-methyl-5′-oxo-2′-((6-((5-(piperidin-1-yl)pyridin-2-yl)amino)pyrimidin-4-yl)amino)-5′,6′-dihydrospiro[cyclohexane-1,7′-pyrrolo[3,4-b]pyridine] 1′-oxide (Compound XXIX-AAM): LCMS (ESI, m/z): [M+H]⁺=501.2. ¹H NMR (400 MHz, DMSO-d₆): δ 8.51-8.48 (m, 2H), 7.99 (s, 1H), 7.56 (s, 1H), 7.41 (s, 2H), 3.13-3.08 (m, 4H), 2.87-2.82 (m, 2H), 2.59 (s, 3H), 1.70-1.52 (m, 12H), 1.32-1.21 (m, 4H).

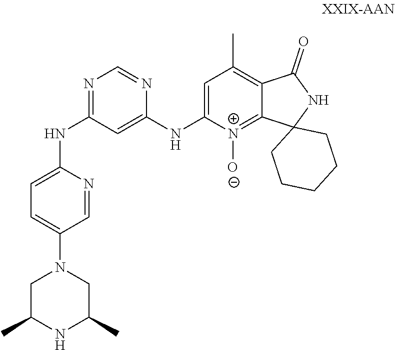 [SMILES: Cc1cc(Nc2cc(Nc3ccc(N4C[C@@H](C)N[C@@H](C)C4)cn3)ncn2)[n+]([O-])c2c1C(=O)NC21CCCCC1]

2′-((6-((5-(cis-3,5-dimethylpiperazin-1-yl)pyridin-2-yl)amino)pyrimidin-4-yl)amino)-4′-methyl-5′-oxo-5′,6′-dihydrospiro[cyclohexane-1,7′-pyrrolo[3,4-b]pyridine]1′-oxide (Compound XXIX-AAN): LCMS (ESI, m/z): [M+H]⁺=530.3. ¹H NMR (400 MHz, DMSO-d₆): δ 10.26 (s, 1H), 9.87 (s, 1H), 9.38 (s, 1H), 8.53-8.50 (m, 2H), 8.00 (s, 1H), 7.58 (s, 1H), 7.46-7.40 (m, 2H), 3.50-3.48 (m, 2H), 2.92-2.83 (m, 4H), 2.60 (s, 3H), 2.18-2.12 (m, 2H), 1.71-1.62 (m, 5H), 1.31-1.24 (m, 4H), 1.04 (d, J=6.0 Hz, 6H).

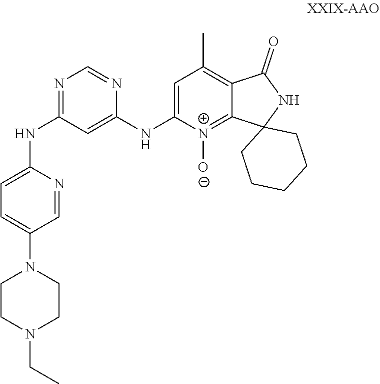 [SMILES: CCN1CCN(c2ccc(Nc3cc(Nc4cc(C)c5c([n+]4[O-])C4(CCCCC4)NC5=O)ncn3)nc2)CC1]

2′-[(6-{[5-(4-ethylpiperazin-1-yl)pyridin-2-yl]amino}pyrimidin-4-yl)amino]-4′-methyl-5′-oxo-6′H-spiro[cyclohexane-1,7′-pyrrolo[3,4-b]pyridin]-1′-ium-1′-olate (Compound XXIX-AAO): LCMS (ESI, m/z): [M+H]⁺=530.4. ¹H NMR (400 MHz, DMSO-d₆): δ 10.27 (s, 1H), 9.87 (s, 1H), 9.36 (s, 1H), 8.53-8.50 (m, 2H), 8.02 (s, 1H), 7.62 (s, 1H), 7.44 (s, 2H), 3.14-3.11 (m, 4H), 2.91-2.83 (m, 2H), 2.60 (s, 3H), 2.41-2.36 (m, 2H), 1.75-1.65 (m, 5H), 1.31-1.25 (m, 3H), 1.06-1.03 (m, 3H).

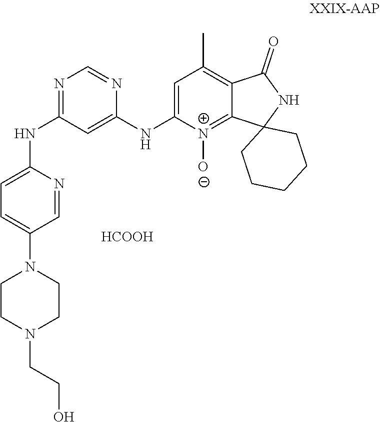 [SMILES: Cc1cc(Nc2cc(Nc3ccc(N4CCN(CCO)CC4)cn3)ncn2)[n+]([O-])c2c1C(=O)NC21CCCCC1 ; "HCOOH"]

2′-((6-((5-(4-(2-hydroxyethyl)piperazin-1-yl)pyridin-2-yl)amino)pyrimidin-4-yl)amino)-4′-methyl-5′-oxo-5′,6′-dihydrospiro[cyclohexane-1,7′-pyrrolo[3,4-b]pyridine]1′-oxide formate salt (Compound XXIX-AAP): LCMS (ESI, m/z): [M+H]⁺=546.4. ¹HNMR (400 MHz, DMSO-d₆+D₂O): δ 8.50-8.48 (m, 2H), 8.20 (s, 1H), 7.99 (s, 1H), 7.53 (s, 1H), 7.42 (s, 2H), 3.57-3.54 (m, 2H), 3.14-3.11 (m, 4H), 2.89-2.79 (m, 2H), 2.69-2.66 (m, 3H), 2.58-2.50 (m, 7H), 1.78-1.62 (m, 5H), 1.31-1.21 (m, 3H).

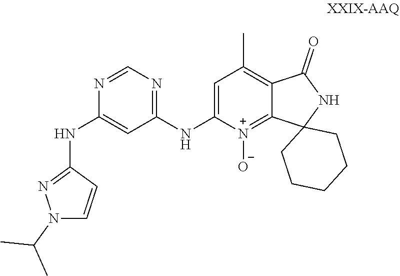 [SMILES: Cc1cc(Nc2cc(Nc3ccn(C(C)C)n3)ncn2)[n+]([O-])c2c1C(=O)NC21CCCCC1]

2′-((6-((1-isopropyl-1H-pyrazol-3-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AAQ): LCMS (ESI, m/z): [M+H]⁺=449.2. ¹H NMR (400 MHz, DMSO-d₆): δ 10.25 (s, 1H), 9.78 (s, 1H), 9.35 (s, 1H), 8.50 (s, 1H), 8.44 (s, 1H), 7.66 (d, J=2.0 Hz, 1H), 7.09 (s, 1H), 6.28 (s, 1H), 4.45-4.41 (m, 1H), 2.91-2.83 (m, 2H), 2.59 (s, 3H), 1.71-1.61 (m, 5H), 1.43 (d, J=6.8 Hz, 6H), 1.30-1.21 (m, 3H).

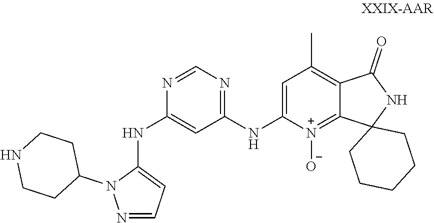 [SMILES: Cc1cc(Nc2cc(Nc3ccnn3C3CCNCC3)ncn2)[n+]([O-])c2c1C(=O)NC21CCCCC1]

4′-methyl-5′-oxo-2′-((6-((1-(piperidin-4-yl)-1H-pyrazol-5-yl)amino)pyrimidin-4-yl)amino)-5′,6′-dihydrospiro[cyclohexane-1,7′-pyrrolo[3,4-b]pyridine] 1′-oxide (Compound XXIX-AAR): LCMS (ESI, m/z): [M+H]⁺=490.3. ¹H NMR (400 MHz, DMSO-d₆): δ 10.23 (s, 1H), 9.77 (s, 1H), 9.35 (s, 1H), 8.51 (s, 1H), 8.44 (s, 1H), 7.66 (d, J=2.0 Hz, 1H), 7.11 (s, 1H), 6.26 (s, 1H), 4.13-4.08 (m, 1H), 3.08-3.00 (m, 2H), 2.91-2.83 (m, 2H), 2.59-2.50 (m, 5H), 1.97-1.94 (m, 2H), 1.84-1.63 (m, 7H), 1.32-1.22 (m, 4H).

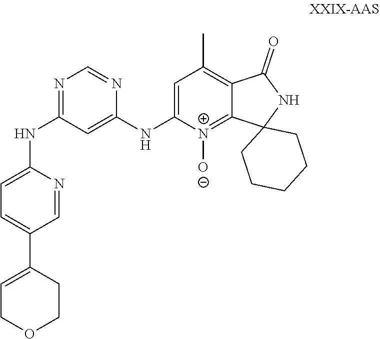 [SMILES: Cc1cc(Nc2cc(Nc3ccc(C4=CCOCC4)cn3)ncn2)[n+]([O-])c2c1C(=O)NC21CCCCC1]

2′-((6-((5-(3,6-dihydro-2H-pyran-4-yl)pyridin-2-yl)amino)pyrimidin-4-yl)amino)-4′-methyl-5′-oxo-5′,6′-dihydrospiro[cyclohexane-1,7′-pyrrolo[3,4-b]pyridine] 1′-oxide (Compound XXIX-AAS): LCMS (ESI, m/z): [M+H]⁺=500.2. ¹H NMR (400 MHz, DMSO-d₆): δ 10.36 (s, 1H), 10.20 (s, 1H), 9.38 (s, 1H), 8.55 (d, J=9.6 Hz, 2H), 8.41 (d, J=2.4 Hz, 1H), 7.87-7.83 (m, 2H), 7.53 (d, J=8.8 Hz, 1H), 6.28 (s, 1H), 4.24 (d, J=2.4 Hz, 2H), 3.86-3.83 (m, 2H), 2.91-2.83 (m, 2H), 2.61 (s, 3H), 2.49-2.47 (m, 2H), 1.72-1.65 (m, 5H), 1.31-1.24 (m, 3H).

Following the synthetic route described above for Example 18 and substituting the appropriate reagents, starting materials and purification methods known to those skilled in the art, the compounds listed below may be synthesized.

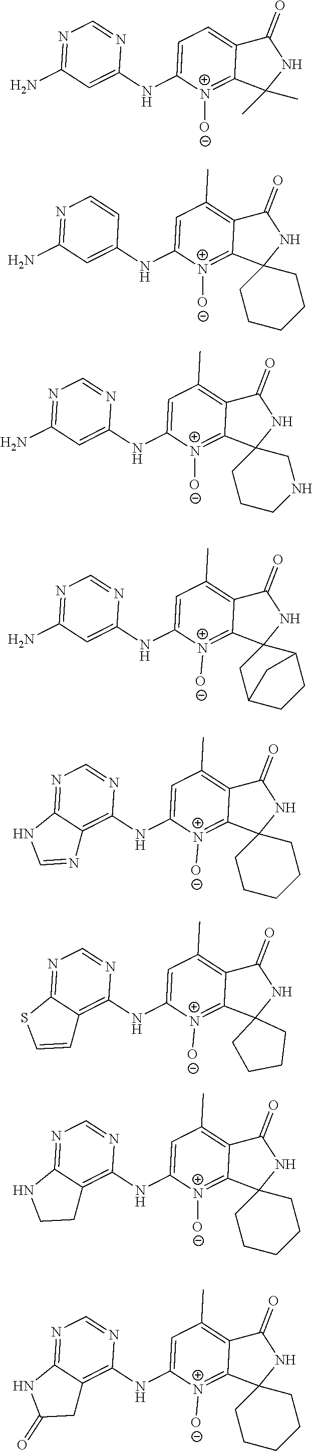

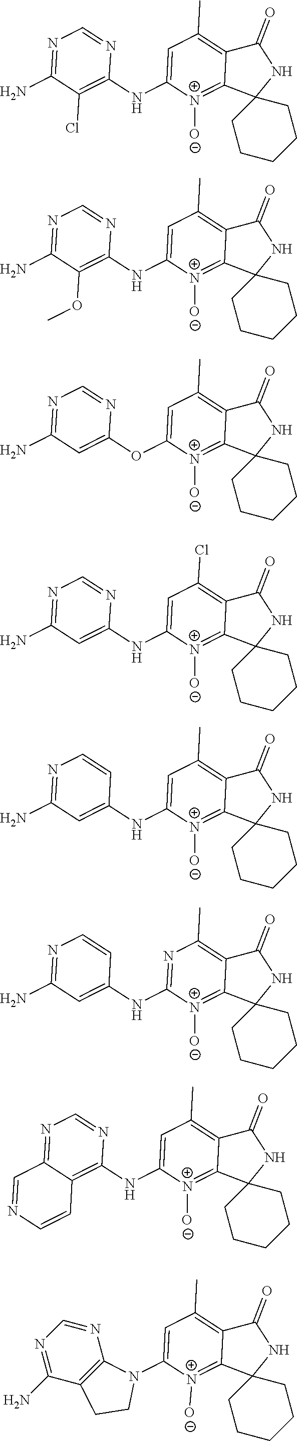

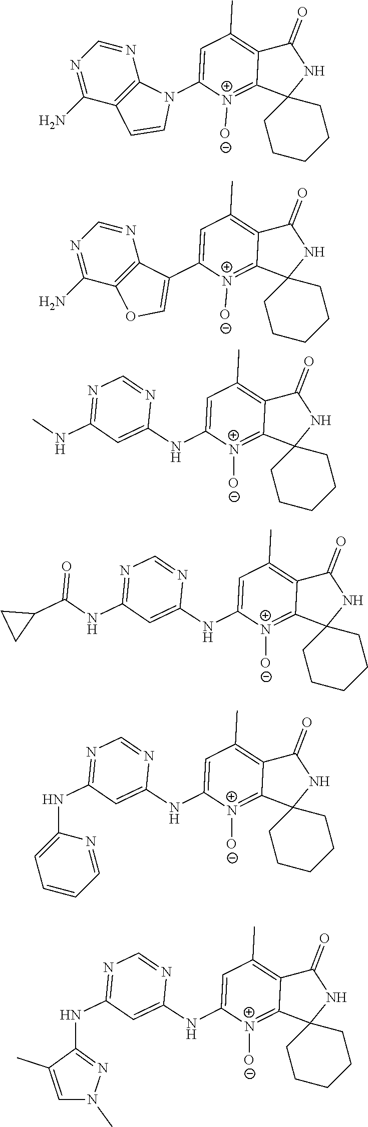

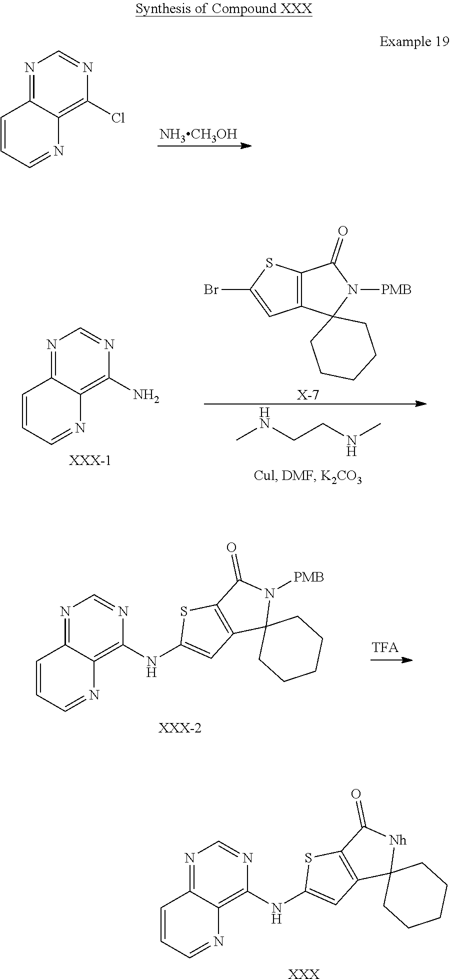

pyrido[3,2-d]pyrimidin-4-amine (Compound XXX-1): A solution of 4-chloropyrido[3,2-d]pyrimidine (1.0 g, 6.04 mmol) in NH₃/MeOH (10.0 mL, 7.0 mol/L) was stirred at room temperature for 16 h. After the reaction was completed, the resulting mixture was concentrated under reduced pressure. The residue was purified by flash column chromatography with CH₂Cl₂/MeOH (90/10, v/v) to afford the title compound (793.4 mg, 89%) as a white solid. LCMS (ESI, m/z): [M+H]⁺=147.1.

5′-(4-methoxybenzyl)-2′-(pyrido[3,2-d]pyrimidin-4-ylamino)spiro[cyclohexane-1,4′-thieno[2,3-c]pyrrol]-6′(5′H)-one (Compound XXX-2): To a solution of pyrido[3,2-d]pyrimidin-4-amine (200.0 mg, 1.37 mmol) in DMF (10.0 mL) was added 2′-bromo-5′-(4-methoxybenzyl)spiro[cyclohexane-1,4′-thieno[2,3-c]pyrrol]-6′(5′H)-one (556.1 mg, 1.37 mmol), methyl[2-(methylamino)ethyl]amine (12.1 mg, 0.14 mmol), CuI (26.1 mg, 0.14 mmol) and K₂CO₃ (283.7 mg, 2.05 mmol) at room temperature. The resulting mixture was stirred at 100° C. for 16 h under N₂. After the reaction was completed, the resulting mixture was cooled to room temperature and filtered. The filtrate was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (83/17, v/v) to afford the title compound (73.7 mg, 11%) as a yellow oil. LCMS (ESI, m/z): [M+H]⁺=472.2.

2′-(pyrido[3,2-d]pyrimidin-4-ylamino)spiro[cyclohexane-1,4′-thieno[2,3-c]pyrrol]-6′(5′H)-one (Compound XXX): A solution of 5′-(4-methoxybenzyl)-2′-(pyrido[3,2-d]pyrimidin-4-ylamino)spiro[cyclohexane-1,4′-thieno[2,3-c]pyrrol]-6′(5′H)-one (53.8 mg, 0.11 mmol) in TFA (1.0 mL) was stirred at 60° C. for 16 h. After the reaction was completed, the resulting mixture was cooled to room temperature. The pH value of the mixture was adjusted to 7.0 with saturated NaHCO₃ (aq.). The mixture was extracted with ethyl acetate. The combined organic layer was washed with brine, dried over anhydrous sodium sulfate and filtered. The filtrate was concentrated under vacuum. The residue was purified by Prep-HPLC with the following conditions Column (Xselect CSH OBD Column 30×150 mm, 5 um; Mobile Phase A: Water (0.1% FA), Mobile Phase B: ACN; Flow rate: 60 mL/min; Gradient: 28% to 38% in 8 min) to afford the title compound (9.3 mg, 23%) as a yellow solid. LCMS (ESI, m/z): [M+H]⁺=352.0. ¹H NMR (300 MHz, DMSO-d₆): δ 12.06 (s, 1H), 9.02-9.00 (m, 1H), 8.86 (s, 1H), 8.55 (s, 1H), 8.32-8.28 (m, 1H), 8.00-7.96 (m, 1H), 7.40 (s, 1H), 1.74-1.68 (m, 6H), 1.67-1.53 (m, 4H).

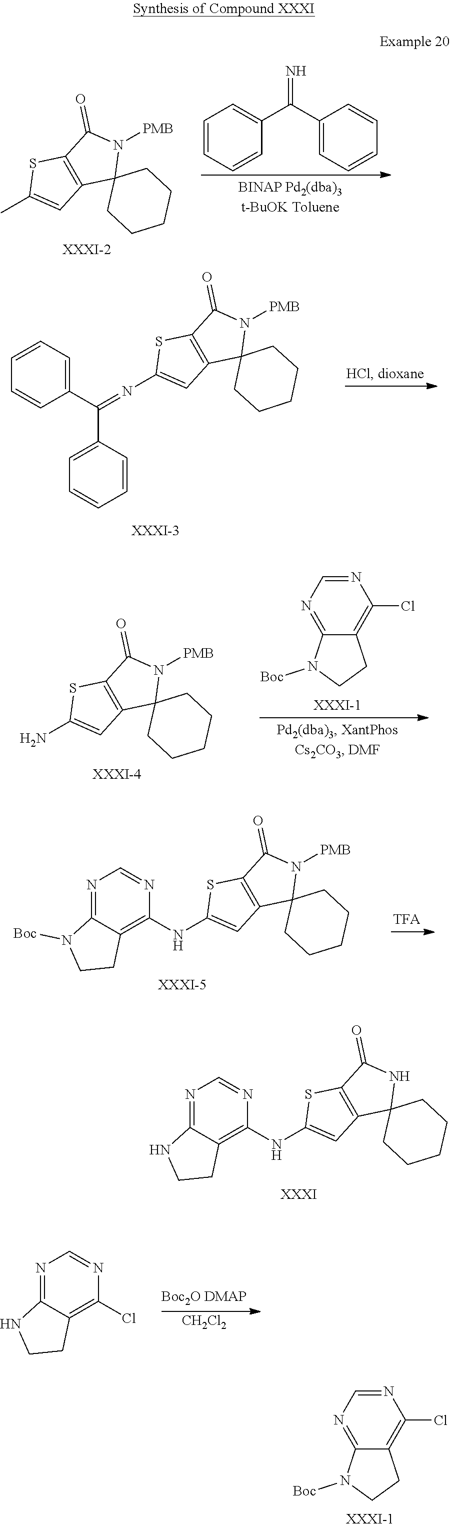

tert-butyl 4-chloro-5,6-dihydro-7H-pyrrolo[2,3-d]pyrimidine-7-carboxylate (Compound XXXI-1): To a solution of 4-chloro-6,7-dihydro-5H-pyrrolo[2,3-d]pyrimidine (200.0 mg, 1.29 mmol) in CH₂Cl₂ (6.0 mL) was added Boc₂O (336.7 mg, 1.54 mmol) and DMAP (15.7 mg, 0.13 mmol). The resulting mixture was stirring at room temperature for 16 h. After the reaction was completed, the resulting mixture was concentrated under reduced pressure. The residue was purified by flash column chromatography with petroleum ether/ethyl acetate (50/50, v/v) to afford the title compound (298.0 mg, 90%) as a white solid. LCMS (ESI, m/z): [M+H]⁺=256.1.

2′-((diphenylmethylene)amino)-5′-(4-methoxybenzyl)spiro[cyclohexane-1,4′-thieno[2,3-c]pyrrol]-6′(5′H)-one (Compound XXXI-2): To a solution of 2′-bromo-5′-(4-methoxybenzyl)spiro[cyclohexane-1,4′-thieno[2,3-c]pyrrol]-6′(5′H)-one (500.0 mg, 1.23 mmol) in toluene (5.0 mL) was added diphenylmethanimine (446.0 mg, 2.46 mmol), BINAP (306.5 mg, 0.49 mmol), Pd₂(dba)₃ (225.4 mg, 0.25 mmol) and t-BuOK (414.2 mg, 3.69 mmol) at room temperature. The resulting mixture was stirred at 100° C. for 16 h under N₂. After the reaction was completed, the resulting mixture was concentrated under reduced pressure. The residue was purified by flash column chromatography with petroleum ether/ethyl acetate (50/50, v/v) to afford the title compound (406.0 mg, 19%) as a white solid. LCMS (ESI, m/z): [M+H]⁺=507.2.

2′-amino-5′-(4-methoxybenzyl)spiro[cyclohexane-1,4′-thieno[2,3-c]pyrrol]-6′(5′H)-one (Compound XXXI-3): A solution of 2′-((diphenylmethylene)amino)-5′-(4-methoxybenzyl)spiro[cyclohexane-1,4′-thieno[2,3-c]pyrrol]-6′(5′H)-one (350.0 mg, 0.69 mmol) in HCl/1,4-dioxane (17.5 mL, 4.0 mol/L) was stirred at room temperature for 16 h.

After the reaction was completed, the mixture was evaporated in vacuo. The pH value of the mixture was adjusted to 7.0 with saturated NaHCO₃ (aq.). The mixture was extracted with ethyl acetate. The combined organic layer was washed with brine, dried over anhydrous sodium sulfate and filtered. The filtrate was concentrated under vacuum. The residue was purified by flash column chromatography with petroleum ether/ethyl acetate (48/52, v/v) to afford the title compound (105.0 mg, 77%) as an off-white solid. LCMS (ESI, m/z): [M+H]⁺=343.1.

tert-butyl 4-((5′-(4-methoxybenzyl)-6′-oxo-5′,6′-dihydrospiro[cyclohexane-1,4′-thieno[2,3-c]pyrrol]-2′-yl)amino)-5,6-dihydro-7H-pyrrolo[2,3-d]pyrimidine-7-carboxylate (Compound XXXI-4): To a solution of 2′-amino-5′-(4-methoxybenzyl)spiro [cyclohexane-1,4′-thieno[2,3-c]pyrrol]-6′(5′H)-one (100.0 mg, 0.29 mmol) in DMF (5.0 mL) was added tert-butyl 4-chloro-5,6-dihydro-7H-pyrrolo[2,3-d]pyrimidine-7-carboxylate (112.0 mg, 0.44 mmol), Pd₂(dba)₃ (53.5 mg, 0.06 mmol), XantPhos (67.6 mg, 0.12 mmol) and Cs₂CO₃ (142.7 mg, 0.44 mmol) at room temperature. The final reaction mixture was irradiated with microwave radiation at 120° C. for 2 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (84/16, v/v) to afford the title compound (157.6 mg, 91%) as a yellow solid. LCMS (ESI, m/z): [M+H]⁺=562.2.

2′-((6,7-dihydro-5H-pyrrolo[2,3-d]pyrimidin-4-yl)amino)spiro[cyclohexane-1,4′-thieno[2,3-c]pyrrol]-6′(5′H)-one (Compound XXXI): A solution of tert-butyl 4-((5′-(4-methoxybenzyl)-6′-oxo-5′,6′-dihydrospiro[cyclohexane-1,4′-thieno[2,3-c]pyrrol]-2′-yl)amino)-5,6-dihydro-7H-pyrrolo[2,3-d]pyrimidine-7-carboxylate (137.6 mg, 0.25 mmol) in TFA (2.0 mL) was stirred at 60° C. for 5 h. After the reaction was completed, the resulting mixture was cooled to room temperature. The pH value of the mixture was adjusted to 8.0 with saturated NaHCO₃ (aq.). The mixture was extracted with CH₂Cl₂. The combined organic layer was washed with brine, dried over anhydrous sodium sulfate and filtered. The filtrate was concentrated under vacuum. The residue was purified by Prep-HPLC with the following conditions (Column: Xselect CSH OBD Column 30×150 mm, 5 um; Mobile Phase A: Water (0.1% FA), Mobile Phase B: ACN; Flow rate: 60 mL/min; Gradient: 7% B to 21% B in 10 min; 254/220 nm) to afford the title compound (2.9 mg, 3%) as a white solid. LCMS (ESI, m/z): [M+H]⁺=342.0. ¹H NMR (400 MHz, DMSO-d₆): δ 10.01 (s, 1H), 8.31 (s, 1H), 8.07 (s, 1H), 6.94 (s, 1H), 6.71 (s, 1H), 3.54-3.42 (m, 2H), 2.97-2.92 (m, 2H), 1.69-1.64 (m, 6H), 1.51-1.45 (m, 4H).

Following the procedure described above for Example 20 and substituting the appropriate reagents, starting materials and purification methods known to those skilled in the art, the compounds listed below were synthesized.

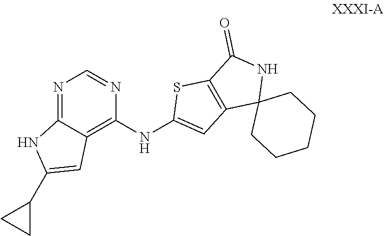 [SMILES: O=C1NC2(CCCCC2)c2cc(Nc3ncnc4[nH]c(C5CC5)cc34)sc21]

2′-((6-cyclopropyl-7H-pyrrolo[2,3-d]pyrimidin-4-yl)amino)spiro[cyclohexane-1,4′-thieno[2,3-c]pyrrol]-6′(5′H)-one (Compound XXXI-A): LCMS (ESI, m/z): [M+H]⁺=380.3. ¹H NMR (400 MHz, DMSO-d₆): δ 11.71 (s, 1H), 10.74 (s, 1H), 8.30-8.27 (m, 2H), 7.15 (s, 1H), 6.77 (s, 1H), 2.01-1.94 (m, 1H), 1.67-1.60 (m, 6H), 1.48-1.32 (m, 4H), 1.03-0.98 (m, 2H), 0.82-0.78 (m, 2H).

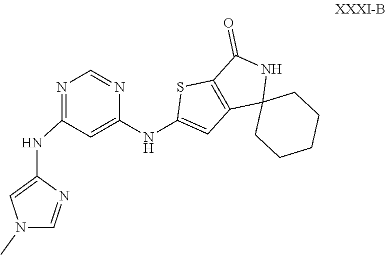 [SMILES: Cn1cnc(Nc2cc(Nc3cc4c(s3)C(=O)NC43CCCCC3)ncn2)c1]

2′-((6-((1-methyl-1H-imidazol-4-yl)amino)pyrimidin-4-yl)amino)spiro[cyclohexa ne-1,4′-thieno[2,3-c]pyrrol]-6′(5′H)-one (Compound XXXI-B): The title compound was synthesized by using 6-bromo-N-(1-methyl-1H-imidazol-4-yl)pyrimidin-4-amine as the starting material of which the synthesis was shown below. LCMS (ESI, m/z): [M+H]⁺=396.1. ¹H NMR (400 MHz, DMSO-d₆): δ 9.48 (s, 1H), 8.35 (s, 2H), 7.40 (s, 1H), 7.12 (s, 1H), 6.59 (s, 1H), 6.37 (s, 1H), 3.63 (s, 3H), 1.72-1.64 (m, 6H), 1.60-1.48 (m, 4H).

The synthesis of 6-bromo-N-(1-methyl-1H-imidazol-4-yl)pyrimidin-4-amine: To a solution of 1-methyl-1H-imidazol-4-amine (300.0 mg, 3.09 mmol) in dioxane (50.0 mL) was added 4,6-dibromopyrimidine (1469.6 mg, 6.18 mmol), Pd₂(dba)₃ (565.7 mg, 0.62 mmol), XantPhos (714.9 mg, 1.24 mmol) and Cs₂CO₃ (2012.8 mg, 6.17 mmol) at room temperature. The resulting mixture was stirred at 100° C. for 16 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with CH₂Cl₂/MeOH (92/8, v/v) to afford 6-bromo-N-(1-methyl-1H-imidazol-4-yl)pyrimidin-4-amine (130.0 mg, 17%) as a white solid. LCMS (ESI, m/z): [M+H]⁺=254.1.

### Example 21: Biological Activity

### Assay Protocols

MNK, a Ser/Thr kinase, is the only kinase known to phosphorylate eIF4E at serine 209. Aberrant expression and phosphorylation of eIF4E promotes tumorigenesis and has been implicated in cancer development and progression.

MNK Biochemical Enzymatic Assay. This protocol establishes the binding assays for MNK1 and MNK2 using ADP-Glo assay. MNK phosphorylates the substrate and converts ATP to ADP, which was detected by Envision and used to reflect the reminding activity of MNK. Reagents and equipment used in the assay are listed below, followed by the protocol.

| Number | Name | Vendor | Cat# |
|---|---|---|---|
| 1 | HEPES | Life Technologies | 15630-080 |
| 2 | NaCl | Sigma | S5886-1Kg |
| 3 | MgCl2 | Sigma | M1028 |
| 4 | BSA | Sigma | B2064-50G |
| 5 | Tween-20 | Bio-RAD | 170-6531 |
| 6 | ADP-Glo Kinase Assay | Promega | V9101 |
| 7 | MNK1 | Carna | |
| 8 | MNK2 | Carna | |
| 9 | ATP | Promega | V915B |
| 10 | Substrate peptide | NJ peptide | |
| 11 | Topseal A | PerkinElmer | E5341 |
| 12 | OptiPlate-384 | PerkinElmer | 6007290 |
| 13 | Envision | Perkin Elmer | 2104 |
| 14 | Centrifuge | Eppendorf | 5810R |
| 15 | Echo 550 Liquid Handler | Labcyte | Echo 550 |

a) Add 50 μL compound to 384-well dilution plate
b) Dilute compound 1:3 in succession in DMSO for each column for 10+0 pts (refer to dilution plate map)
c) Transfer 0.1 μL diluted compound solution in each row to 384 assay plate using Echo, each column containing 2 replicates (refer to assay plate map)
d) Add 5 μL enzyme working solution to 384-well assay plate, centrifuge 1000 RPM for 1 min
e) Incubate at 25° C. for 15 min
f) Add 5 μL substrate working solution to initiate reaction
g) Incubate at 25° C. for 60 min
h) Add 10 μL ADP Glo reagent, centrifuge 1000 RPM for 1 min
i) Incubate at 25° C. for 60 min
j) Add 20 μL kinase detection reagent, centrifuge 1000 RPM for 1 min
k) Incubate at 25° C. for 60 min
l) Read on Envision for US LUM as RLU
m) Data analysis: IC50s were determined based on a non-linear regression analysis of data collected.

p-eIF4E Signaling Cellular Assay. Phosphorylated eIF4E is assayed using the CisBio p-Eif4E HTRF assay kit. Reagents and equipment used in the assay are listed below, followed by the protocol.

| Number | Name | Vendor | Cat# |
|---|---|---|---|
| 1 | FBS | Gbico | 10099-141 |
| 2 | Penicillin- | Gbico | 15140-122 |
| | Streptomycin(10000 U/ml, 100 ml) | | |
| 3 | RPMI1640 | Invitrogen | A10491-0 |
| 4 | EIF4E phospho-S209 kit | Cisbio | 64EF4PEG |
| 5 | CulturPlate-384, White Opaque | PerkinElmer | 6007680-50 |
| | 384-well Microplate | | |
| 6 | 384-Well Polypropylene | Labcyte | P-05525-BC |
| | microplate, Clear, Flatt | | |
| | Bottom, Bar Code | | |
| 7 | Biological Safety Cabinet(Class | Thermo | 1389 |
| | II | Scientific | |
| 8 | CO2 Incubator | Thermo | 371 |
| | | Scientific | |
| 9 | Cell Counter | Invitrogen | Countess ® |
| | | | Automated Cell |
| | | | Counter |
| 10 | Plate shaker | Thermo | 4625- |
| | | Scientific | 1CECN/THZQ |
| 11 | Centrifuge | Eppendorf | 5810R |
| 12 | Echo 550 | Labcyte | 550 |
| 13 | Envision 2105 multilabel Reader | Perkin Elmer | Envision 2105 |
| 14 | Votexer | IKA | MS3 digital |

Cell medium: RPMI 1640+10% FBS+1*PS, TMD-8 were cultured as recommended and assayed in exponential growth phase.
a) Plate 30 μL of cells in 384 white assay plate in appropriate medium and proper cell density (20 k/well) based on Optimization at 37° C. under 5% CO2 atmosphere
b) Dispense 90 nL of compounds diluted in DMSO and treat for 40 min at 37° C. under 5% CO2 atmosphere
c) Remove cell supernatant by flicking the plate
d) Add 16 μL of supplemented lysis buffer (1×) for 30 minutes at RT under shaking
e) Add 4 μL of premixed antibody solutions prepared in the detection buffer. Cover the plate with a plate sealer. Incubate 0/N at RT
f) Read the fluorescence emission at 665 nm and 620 nm on Envision
g) Data analysis
(1) Ratio 665/615 signal is calculated for each well
(2) % Inhibition is calculated as follow:

% inhibition=100−(Signalcmpd-SignalAve_PC)/(SignalAve_VC-SignalAve_PC)×100.
(3) Calculate IC50 and Plot effect-dose curve of cmpds:

Calculate IC50 by fitting % Inhibition values and log of compound concentrations to nonlinear regression (dose response−variable slope) with Graphpad 8.0.

Y=Bottom+(Top−Bottom)/(1+10\hat{ }((Log IC50−X)*Hill Slope))
X: log of Inhibitor concentration; Y: % Inhibition.

### Biological Data

Compounds of the present technology as described herein were or are tested according to the protocol above and show or are expected to show IC₅₀ values equal to or below 1 uM in one or more of the above assays. Certain compounds exhibit or are expected to exhibit IC₅₀s of 100 nM or less, and others exhibit or are expected to exhibit IC₅₀s of 10 nM or less in one or more of the above binding assays. Exemplary results are shown in Table 2 for selected compounds.

TABLE 2

| | MNK1 binding | MNK2 binding | TMD-8 cell p- |
|---|---|---|---|
| | assay | assay | IF4E |
| Compound | IC50 (nM) | IC50 (nM) | IC50 (nM) |
| Compound I | D | D | ND |
| Compound IIa | A | A | ND |
| Compound IIb | A | A | ND |
| Compound III | D | D | ND |
| Compound VII | ND | D | ND |
| Compound VIII-A | ND | B | ND |
| Compound X | ND | B | ND |
| Compound X-A | ND | B | ND |
| Compound X-B | ND | C | ND |
| Compound X-C | ND | A | B |
| Compound X-D | ND | B | ND |
| Compound X-E | ND | B | ND |
| Compound XI | ND | D | ND |
| Compound XVII | ND | A | C |
| Compound XVII-A | ND | A | B |
| Compound XVII-B | ND | A | C |
| Compound XVII-C | ND | A | B |
| Compound XVIII | ND | D | ND |
| Compound XIX | ND | C | ND |
| Compound XX | ND | B | ND |
| Compound XX-7 | ND | C | ND |
| Compound XX-A | ND | B | ND |
| Compound XX-B | ND | C | ND |
| Compound XX-C | ND | C | ND |
| Compound XX-D | ND | A | C |
| Compound XX-E | ND | B | ND |
| Compound XXI | ND | C | ND |
| Compound XXI-A | ND | A | B |
| Compound XXII | ND | B | ND |
| Compound XXIII | ND | B | ND |
| Compound XXIII-A | ND | C | ND |
| Compound XXIII-B | ND | B | ND |
| Compound XXIII-C | ND | A | B |
| Compound XXIII-D | ND | D | ND |
| Compound XXIII-E | ND | D | ND |
| Compound XXIII-F | ND | B | ND |
| Compound XXIII-G | ND | B | ND |
| Compound XXIII-H | ND | D | ND |
| Compound XXIII-I | ND | C | ND |
| Compound XXIII-J | ND | A | C |
| Compound XXIII-K | ND | D | ND |
| Compound XXIII-L | ND | D | ND |
| Compound XXIII-M | ND | D | ND |
| Compound XXIII-N | ND | B | B |
| Compound XXIII-O | ND | B | ND |
| Compound XXIII-P | ND | A | B |
| Compound XXIII-Q | ND | B | ND |
| Compound XXIII-R | ND | C | ND |
| Compound XXIII-S | ND | C | ND |
| Compound XXIII-T | ND | A | B |
| Compound XXIII-U | ND | A | B |
| Compound XXIII-V | ND | B | ND |
| Compound XXIII-W | ND | B | ND |
| Compound XXIII-X | ND | B | ND |
| Compound XXIII-Y | ND | A | B |
| Compound XXIII-Z | ND | B | ND |
| Compound XXIV | ND | B | ND |
| Compound XXIV-A | ND | A | C |
| Compound XXIV-B | ND | A | B |
| Compound XXIV-C | ND | A | B |
| Compound XXIV-D | ND | C | ND |
| Compound XXIV-E | ND | B | ND |
| Compound XXIV-F | ND | B | ND |
| Compound XXIV-G | ND | A | C |
| Compound XXIV-H | ND | B | B |
| Compound XXIV-I | ND | A | C |
| Compound XXIV-J | ND | B | ND |
| Compound XXIV-K | ND | A | C |
| Compound XXIV-L | ND | C | ND |
| Compound XXIV-M | ND | B | ND |
| Compound XXV | ND | B | C |
| Compound XXV-5 | ND | C | ND |
| Compound XXV-6 | ND | B | ND |
| Compound XXV-A | ND | B | ND |
| Compound XXV-B | ND | B | ND |
| Compound XXV-C | ND | B | C |
| Compound XXV-D | ND | B | ND |
| Compound XXVIII | ND | A | B |
| Compound XXVIII-A | ND | A | A |
| Compound XXVIII-B | ND | A | B |
| Compound XXVIII-C | ND | A | B |
| Compound XXVIII-D | ND | B | ND |
| Compound XXVIII-E | ND | D | ND |
| Compound XXVIII-F | ND | D | ND |
| Compound XXVIII-G | ND | A | B |
| Compound XXVIII-H | ND | A | B |
| Compound XXVIII-I | ND | B | ND |
| Compound XXVIII-J | ND | B | ND |
| Compound XXVIII-K | ND | A | B |
| Compound XXVIII-L | ND | A | C |
| Compound XXVIII-M | ND | A | A |
| Compound XXVIII-N | ND | B | ND |
| Compound XXVIII-O | ND | A | C |
| Compound XXVIII-P | ND | A | B |
| Compound XXVIII-Q | ND | A | A |
| Compound XXVIII-R | ND | A | A |
| Coupound XXIX | ND | A | B |
| Coupound XXIX-A | ND | A | A |
| Coupound XXIX-B | ND | B | ND |
| Coupound XXIX-C | ND | B | ND |
| Coupound XXIX-D | ND | A | A |
| Coupound XXIX-E | ND | B | ND |
| Coupound XXIX-F | ND | A | ND |
| Coupound XXIX-G | ND | A | ND |
| Coupound XXIX-H | ND | C | ND |
| Coupound XXIX-I | ND | B | ND |
| Coupound XXIX-J | ND | C | ND |
| Coupound XXIX-K | ND | A | A |
| Coupound XXIX-L | ND | B | ND |
| Coupound XXIX-M | ND | C | ND |
| Coupound XXIX-N | ND | B | ND |
| Coupound XXIX-O | ND | B | ND |
| Coupound XXIX-P | ND | B | ND |
| Coupound XXIX-Q | ND | B | ND |
| Coupound XXIX-R | ND | C | ND |
| Coupound XXIX-S | ND | B | ND |
| Coupound XXIX-T | ND | B | ND |
| Coupound XXIX-U | ND | B | ND |
| Coupound XXIX-V | ND | B | ND |
| Coupound XXIX-W | ND | C | ND |
| Coupound XXIX-X | ND | B | ND |
| Coupound XXIX-Y | ND | B | ND |
| Coupound XXIX-Z | ND | A | A |
| Coupound XXIX-AA | ND | A | A |
| Coupound XXIX-AB | ND | B | ND |
| Coupound XXIX-AC | ND | B | ND |
| Coupound XXIX-AD | ND | A | B |
| Coupound XXIX-AE | ND | A | B |
| Coupound XXIX-AF | ND | A | A |
| Coupound XXIX-AG | ND | C | ND |
| Coupound XXIX-AH | ND | A | A |
| Coupound XXIX-AI | ND | B | ND |
| Coupound XXIX-AJ | ND | B | ND |
| Coupound XXIX-AK | ND | B | ND |
| Coupound XXIX-AL | ND | A | B |
| Coupound XXIX-AM | ND | A | B |
| Coupound XXIX-AN | ND | D | ND |
| Coupound XXIX-AO | ND | A | A |
| Coupound XXIX-AP | ND | A | B |
| Coupound XXIX-AQ | ND | B | ND |
| Coupound XXIX-AR | ND | C | ND |
| Coupound XXIX-AS | ND | A | A |
| Coupound XXIX-AT | ND | A | A |
| Coupound XXIX-AU | ND | A | A |
| Coupound XXIX-AV | ND | B | ND |
| Coupound XXIX-AW | ND | A | B |
| Coupound XXIX-AX | ND | B | ND |
| Coupound XXIX-AY | ND | B | ND |
| Coupound XXIX-AZ | ND | B | ND |
| Coupound XXIX-AAA | ND | A | A |
| Coupound XXIX-AAB | ND | C | ND |
| Coupound XXIX-AAC | ND | A | B |
| Coupound XXIX-AAD | ND | A | A |
| Coupound XXIX-AAE | ND | A | A |
| Coupound XXIX-AAF | ND | B | ND |
| Coupound XXIX-AAG | ND | C | ND |
| Coupound XXIX-AAH | ND | B | ND |
| Coupound XXIX-AAI | ND | A | A |
| Coupound XXIX-AAJ | ND | A | A |
| Coupound XXIX-AAK | ND | A | A |
| Coupound XXIX-AAL | ND | A | A |
| Coupound XXIX-AAM | ND | A | A |
| Coupound XXIX-AAN | ND | A | A |
| Coupound XXIX-AAO | ND | A | A |
| Coupound XXIX-AAP | ND | A | A |
| Coupound XXIX-AAQ | ND | A | A |
| Coupound XXIX-AAR | ND | A | ND |
| Coupound XXIX-AAS | ND | A | ND |
| Compound XXX | ND | B | ND |
| Compound XXXI | ND | A | C |
| Compound XXXI-A | ND | A | A |
| Compound XXXI-B | ND | B | C |

A: 0.1-10 nM, B: >10 nM-100 nM, C: >100 nM-1 μM, D: >1 μM, ND: not determined

### Illustrative Embodiments

Paragraph 1: A compound having a structure of Formula I or Formula II:

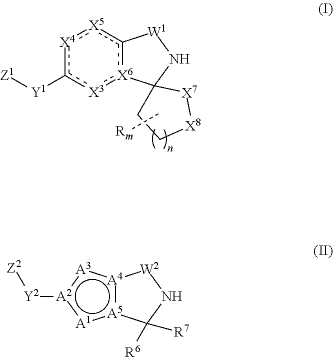

stereoisomers, tautomers, and/or pharmaceutically acceptable salts thereof; wherein W¹ and W² are independently C(═NR⁹), C(═O), C(═S), S(═O), or S(═O)₂, Y¹ and Y² are independently absent, NH NR¹⁰, O, CHR¹⁴, C(═O), S(═O), S(O)₂, cyclopropyl, or a 5-member heteroarylene ring; Z¹ and Z² are independently a heteroaryl moiety selected from Formulas III, IV, V. VI, VII, VIII, IX, X, or XI:

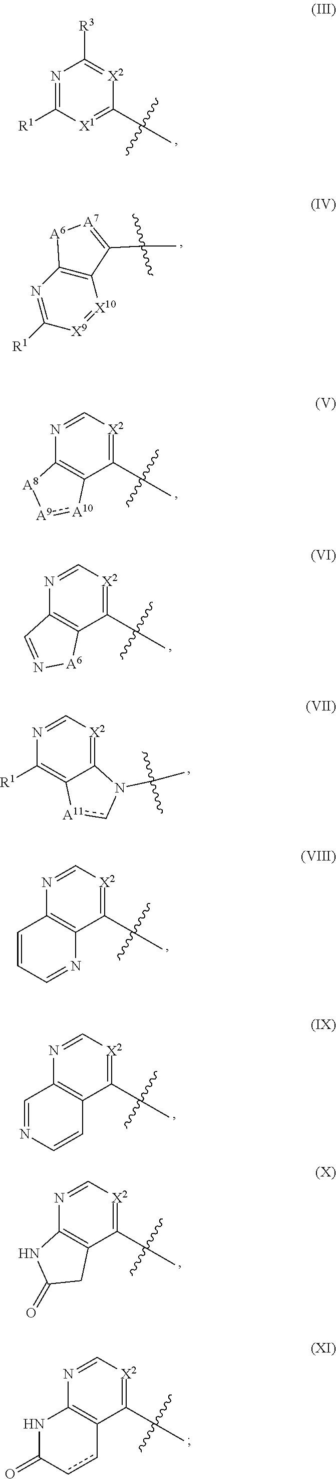

wherein the dotted lines in Formulas V, VII, and XI indicate a single or double bond;
A¹ is CR^{2e}, N, NR⁸, O or S, provided that the ring of which it is a member is a heteroaryl ring; A² is C or N, provided that the ring of which it is a member is a heteroaryl ring; A³ is CR^{2f}, N, NR⁸, O or S, provided that the ring of which it is a member is a heteroaryl ring; A⁴ and A⁵ are each C or one is C and the other N, such that the ring of which they are members is a heteroaryl ring; A⁶ is NR⁸, O, S, or S(═O); A⁷ is CR^{2g} g or N; A⁸ is NR⁸, NHC(O), O, S, S(═O); A⁹ is CH, CH₂, C(O), CR¹⁵, CR¹⁸, or N, provided that when A⁹ is CR^{IB} A¹⁰ is CR¹⁶; A¹⁰ is CH, CH₂, CR¹⁶, CR¹⁹, N, NH, or S, provided that when A¹⁰ is CR¹⁶, A⁹ is CR¹⁵; A¹¹ is CH, CH₂ or N; X¹ is N or CR^{2a}; X² is N or CR^{2b}; X³ is N, N(O), C(═O) or CR^{2c}; X⁴ is N or CR⁴; X⁵ is N or CR⁵; X⁶ is C or N, wherein when X⁶ is C, the dotted lines in Formula I indicate aromatic bonds, and when X⁶ is N, then X³ is C(═O) and the dotted lines in Formula I indicate single or double bonds; X⁷ and X⁸ are independently O, NH, N(O), NR¹⁰, NC(O)R¹¹, NC(O)OR¹¹, S, S(═O), S(═O)₂, CHR¹³, and C(═O), provided that X⁷ and X⁸ are not both O; X⁹ and X¹⁰ are independently N or CR^{2d}; R is independently at each occurrence halo, NO₂, NR⁸R¹⁰, OR¹¹, SR¹², CN, COOR¹³, or a substituted or unsubstituted C₁₋₆ alkyl, C₃₋₇ cycloalkyl, or C₂₋₆ alkenyl group; or when m is at least 2, the two R moieties together form a C₁₋₄ alkylene bridge between non-adjacent ring members; R¹, R^{2a}, R^{2b}, R^{2c}, R^{2d}, R^{2e}, R^{2f}, R^{2g}, and R³ are independently at each occurrence H, halo, NO₂, NR⁸R¹⁰, OR¹¹, SR¹², CN, COOR¹³, or a substituted or unsubstituted C₁₋₆ alkyl, C₃₋₇ cycloalkyl, or, C₂₋₆ alkenyl group; R⁴ and R⁵ are independently H, halo, CN, OH, SR¹², NO₂, NR⁸R¹⁰, or a substituted or unsubstituted C₁₋₆ alkyl, C₁₋₆ alkoxy, or C₂₋₆ alkene; or R⁴ and R⁵ when present, together with the carbon atoms to which they are attached, form a fused phenyl or a 5- or 6-membered cycloalkenyl, heterocyclyl or heteroaryl ring; R⁶ and R⁷ are independently H, NHR¹⁰, or a substituted or unsubstituted C₁₋₈-alkyl, C₂₋₈-alkenyl, cycloalkyl, cycloalkylalkyl, aryl, aralkyl, heterocyclyl, heterocyclylalkyl, heteroaryl, or heteroarylalkyl group; or R⁶ and R⁷ together with the carbon to which they are attached form a substituted or unsubstituted cycloalkyl or heterocyclyl ring; R⁸ and R¹⁰ are independently at each occurrence H, an amino protecting group, or a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group; or R⁸ and R¹⁰ together with the nitrogen to which they are attached form a substituted or unsubstituted heterocyclyl ring;
R⁹ is at independently each occurrence H or substituted or unsubstituted alkyl group; R¹¹ is independently at each occurrence H, a hydroxyl protecting group, or a substituted or unsubstituted alkyl, cycloalkyl, alkenyl, heterocyclyl, heteroaryl, aryl or aralkyl group;
R¹² is independently at each occurrence H, a thiol protecting group, or a substituted or unsubstituted alkyl, cycloalkyl, alkenyl, aryl or aralkyl group; R¹³ is independently at each occurrence H or a substituted or unsubstituted alkyl, cycloalkyl, alkenyl, aryl or aralkyl group; and R¹⁴ is H, OH, or a substituted or unsubstituted alkyl group; R¹⁵ and R¹⁶, together with the carbons to which they are attached, form a cyclohexenyl ring, optionally substituted with C(O)R¹⁷, C(O)OR¹⁷, or C(O)NR⁸R¹⁰; R¹⁷ is independently at each occurrence H or a substituted or unsubstituted alkyl, cycloalkyl, or alkenyl group; R¹⁸ and R¹⁹ are independently selected from CN, C(O)R¹⁷, C(O)OR¹⁷, C(O)NR⁸R¹⁰, or a substituted or unsubstituted alkyl, cycloalkyl, or alkenyl group; m is 0, 1, 2 or 3; and n is 1 or 2; provided that when X⁴ is CH, at least one of X⁷ and X⁸ is a heteroatom, or n is at least 2 and the two R moieties together form a C₁₋₄ alkylene bridge between non-adjacent ring members, or W¹ is S(═O)₂, or X³ is N(O). In some embodiments, when X⁴ is CH, then X³ is N(O).

Paragraph 2: The compound of paragraph 1, wherein Y¹ or Y² is NH.

Paragraph 3: The compound of paragraph 1, wherein Y¹ or Y² is an oxazole, isoxazole, thiazole, imidazole, oxadiazole, dioxazole, or isothiazole.

Paragraph 4: The compound of any one of paragraphs 1-3, wherein Z¹ or Z² is a moiety of Formula III.

Paragraph 5: The compound of any one of paragraphs 1-3, wherein Z¹ or Z² is a moiety of Formula IV.

Paragraph 6: The compound of any one of paragraphs 1-3, wherein Z¹ or Z² is a moiety of Formula V. In some embodiments, the compound is formula II. In some embodiments, A⁸ is NH, A⁹ is CH, and A¹⁰ is CH.

Paragraph 7: The compound of any one of paragraphs 1-3, wherein Z¹ or Z² is a moiety of Formula VI.

Paragraph 8: The compound of any one of paragraphs 1-3, wherein Z¹ or Z² is a moiety of Formula VII.

Paragraph 9: The compound of any one of paragraphs 1-3, wherein Z¹ or Z² is a moiety of Formula VIII.

Paragraph 10: The compound of any one of paragraphs 1-3, wherein Z¹ or Z² is a moiety of Formula IX.

Paragraph 11: The compound of any one of paragraphs 1-3, wherein Z¹ or Z² is a moiety of Formula X.

Paragraph 12: The compound of any one of paragraphs 1-3, wherein Z¹ or Z² is a moiety of Formula XI.

Paragraph 13: The compound of any one of paragraphs 1-12, wherein X¹ is CH.

Paragraph 14: The compound of any one of paragraphs 1-13, wherein X² is CH.

Paragraph 15: The compound of any one of paragraphs 1-13, wherein X² is N.

Paragraph 16: The compound of any one of paragraphs 1-15, wherein R¹ is NR⁸R¹⁰.

Paragraph 17: The compound of any one of paragraphs 1-15, wherein A⁶ is NR⁸.

Paragraph 18: The compound of any one of paragraphs 1-17, wherein A⁷ is CH.

Paragraph 19: The compound of any one of paragraphs 1-18, wherein X⁹ is N.

Paragraph 20: The compound of any one of paragraphs 1-19, wherein A⁸ is NR⁸.

Paragraph 21: The compound of any one of paragraphs 1-19, wherein A⁸ is S.

Paragraph 22: The compound of any one of paragraphs 1-21, wherein A⁹ is N.

Paragraph 23: The compound of any one of paragraphs 1-21, wherein A⁹ is CH or CR¹⁸.

Paragraph 24: The compound of any one of paragraphs 1-23, wherein R¹⁸ is a substituted or unsubstituted alkyl or cycloalkyl group.

Paragraph 25: The compound of any one of paragraphs 1-24, wherein A¹⁰ is N or NH.

Paragraph 26: The compound of any one of paragraphs 1-24, wherein A¹⁰ is CH or CH₂.

Paragraph 27: The compound of any one of paragraphs 1-21, wherein A⁹ is R¹⁵ and A¹⁰ is R¹⁶ and R¹⁵ and R¹⁶, together with the carbons to which they are attached, form a cyclohexenyl ring, optionally substituted with C(O)R¹⁷, C(O)OR¹⁷, or C(O)NR⁸R¹⁰.

Paragraph 28: The compound of paragraph 27, wherein the cyclohexenyl ring is substituted with C(O)NR⁸R¹⁰.

Paragraph 29: The compound of any one of paragraphs 1-28, wherein R⁸ is H.

Paragraph 30: The compound of any one of paragraphs 1-28, wherein R⁸ is CH₃.

Paragraph 31: The compound of any one of paragraphs 1-30, wherein R¹⁰ is H.

Paragraph 32: The compound of any one of paragraphs 1-30, wherein R¹⁰ is an amino protecting group, or a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group.

Paragraph 33: The compound of paragraph 32, wherein R¹⁰ is a substituted or unsubstituted alkyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group.

Paragraph 34: The compound of paragraph 33, wherein R¹⁰ is selected from the group consisting of:

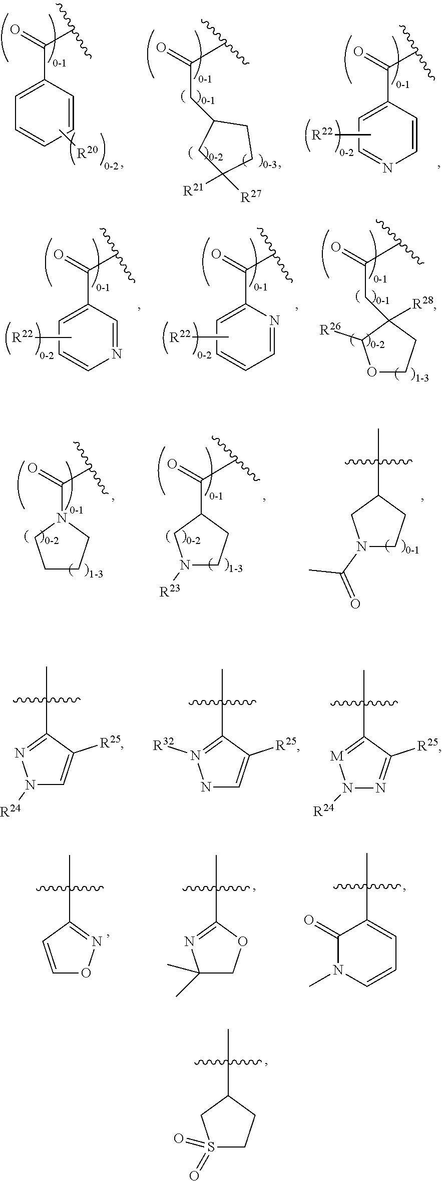

—CH₃, —(CH₂)₁₋₃OH, —(CH₂)₁₋₃OCH₃, —C(O)N(CH₃)₂, and —C(O)NHCH₃,
wherein R²⁰ is halo, or N(CH₃)C(O)CH₃; R²¹ is H, OH, or OCH₃; R²² is CH₃, CH₂OH, —C(O)NHCH₃, —N(CH₃)C(O)CH₃,

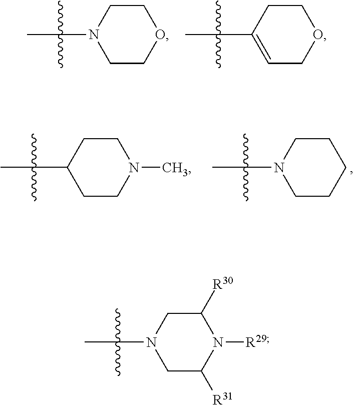

R²³ is H or C(O)CH₃; R²⁴ is H, CH₃, CF₃, (CH₂)₁₋₂OH, or CH(CH₃)₂; R²⁵ is H or CH₃; R²⁶ at each location is independently H or OH; R²⁷ is H or CH₃; R²⁸ is H or C(O)OCH₃; R²⁹ is H, CH₃, CH₂CH₃, or CH₂CH₂OH; R³⁰ and R³¹ are each independently H or CH₃; R³² is H or

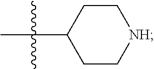 [SMILES: *C1CCNCC1]

and M is CH or N. In some embodiment, R¹⁰ is selected from the group consisting of: hydrogen,

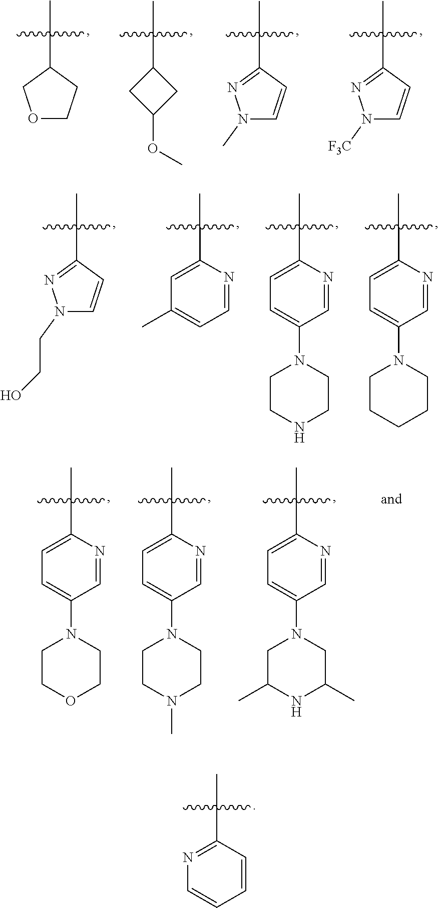

In some embodiments, Z¹ and Z² are formula III.

Paragraph 35: The compound of any one of paragraphs 1-28, wherein R⁸ and R¹⁰ together with the nitrogen to which they are attached form a substituted or unsubstituted heterocyclyl ring.

Paragraph 36: The compound of paragraph 35, wherein R⁸ and R¹⁰ together with the nitrogen to which they are attached are selected from the group consisting of:

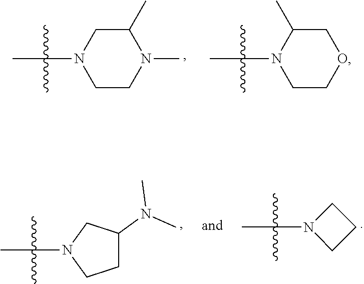

Paragraph 37: The compound of any preceding paragraph having the structure of Formula I.

Paragraph 38: The compound of paragraph 37, wherein X³ is CR^{2c}.

Paragraph 39: The compound of paragraph 37, wherein X³ is CH.

Paragraph 40: The compound of paragraphs 37, wherein X³ is C(O) and X⁶ is N.

Paragraph 41: The compound of any one of paragraphs 37 to 40, wherein X⁴ is CR⁴.

Paragraph 42: The compound of paragraph 41, wherein X⁴ is CH.

Paragraph 43: The compound of any one of paragraphs 37-42, wherein X⁵ is CR⁵.

Paragraph 44: The compound of paragraph 43, wherein R⁵ is C₁-C₄ alkyl or C₂-C₃ alkenyl.

Paragraph 45: The compound of any one of paragraphs 37-44, wherein X⁴ is CR⁴, X⁵ is CR⁵, and R⁴ and R⁵ together with the carbon atoms to which they are attached, form a fused phenyl, pyrrolinyl, or pyrrolyl ring.

Paragraph 46: The compound of any one of paragraphs 37-45, wherein X⁷ is O or NH.

Paragraph 47: The compound of any one of paragraphs 37-45, wherein X⁷ is S, SO, or SO₂.

Paragraph 48: The compound of any one of paragraphs 37-47, wherein X⁸ is CH₂ or C(═O).

Paragraph 49: The compound of any one of paragraphs 37-47, wherein X⁸ is NH.

Paragraph 50: The compound of any one of paragraphs 37-49, wherein m is 0.

Paragraph 51: The compound of any one of paragraphs 37-49, wherein m is 2 and the two R moieties together form a C₁₋₄ alkylene bridge between non-adjacent ring members.

Paragraph 52: The compound of paragraph 51, wherein the two R moieties form a methylene or ethylene bridge.

Paragraph 53: The compound of any one of paragraphs 37-52, wherein W¹ is C(═O).

Paragraph 54: The compound of any one of paragraphs 37-53, wherein n is 2.

Paragraph 55: The compound of any one of paragraphs 37-54, wherein the compound of Formula I is a compound of Formula IA or IB:

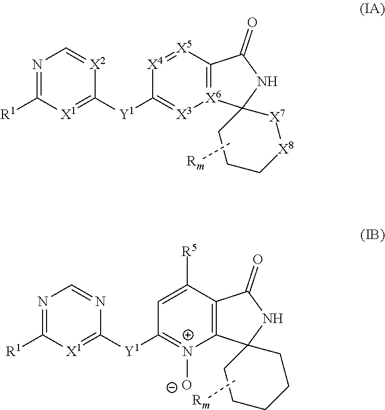

or a stereoisomer, tautomer, and/or pharmaceutically acceptable salt thereof. The compound of Formula IB may be the compound of formula IB-1:

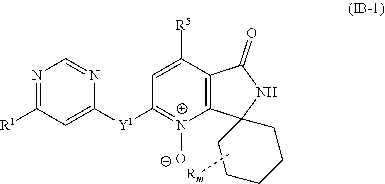

or a stereoisomer, tautomer, and/or pharmaceutically acceptable salt thereof.

Paragraph 56: The compound of any one of paragraphs 1-36, having the structure of Formula II.

Paragraph 57: The compound of paragraph 56, wherein A¹ is CH.

Paragraph 58: The compound of paragraph 56, wherein A¹ is N or NH.

Paragraph 59: The compound of paragraph 56, wherein A¹ is O.

Paragraph 60: The compound of paragraph 56, wherein A¹ is S.

Paragraph 61: The compound of any one of paragraphs 56-60, wherein A² is C.

Paragraph 62: The compound of any one of paragraphs 56-60, wherein A² is N.

Paragraph 63: The compound of any one of paragraphs 56-62, wherein A³ is S.

Paragraph 64: The compound of any one of paragraphs 56-62, wherein A³ is N or NH.

Paragraph 65: The compound of any one of paragraphs 56-64, wherein A³ is CH.

Paragraph 66: The compound of any one of paragraphs 56-64, wherein A³ is O.

Paragraph 67: The compound of any one of paragraphs 56-66, wherein A⁴ is C.

Paragraph 68: The compound of any one of paragraphs 56-66, wherein A⁴ is N.

Paragraph 69: The compound of any one of paragraphs 56-68, wherein A⁵ is C.

Paragraph 70: The compound of any one of paragraphs 56-68, wherein A⁵ is N.

Paragraph 71: The compound of any one of paragraphs 56-70, wherein Y² is absent.

Paragraph 72: The compound of any one of paragraphs 56-71, wherein R⁶ and R⁷ together form a cyclopentyl, cyclohexyl, bicyclo[2.2.1]heptanyl, pyranyl, piperidinyl, or tetrahydrothiopyran or oxides thereof.

Paragraph 73: The compound of any one of paragraphs 56-72, wherein W² is C(═O).

Paragraph 74: The compound of paragraph 73 having a structure selected from the group consisting of Formula IIA, Formula IIB, Formula IIC, Formula IID, Formula IIE, and Formula IIF:

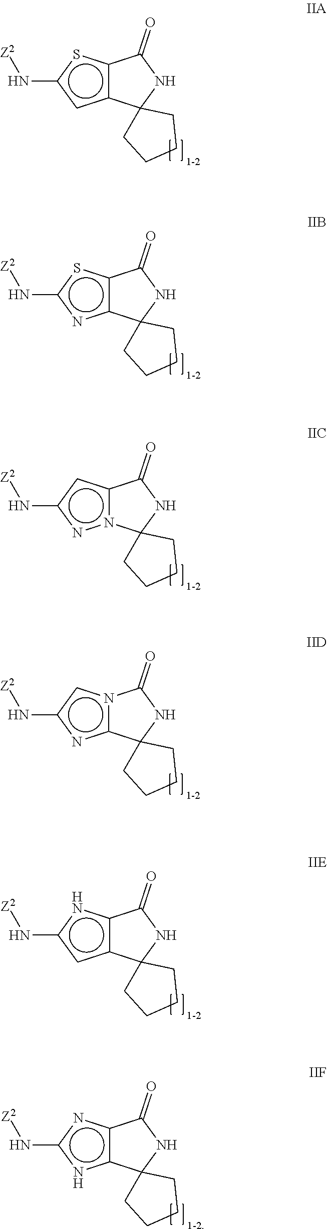

Paragraph 73: The compound of any one of paragraphs 1-74, wherein the compound is selected from the group consisting of compounds XXIX-A, XXIX-AAE, XXIX-Z, XXIX-AF, XXIX-AT, XXIX-AAI, XXIX-AAL, XXIX-AAM, XXIX-AAJ, XXIX-AO, XXIX-AN, XXVIII, XXVIII-A, XXVIII-M, XXVIII-R, XXVII-A, and XXIII-C.

Paragraph 76: A composition comprising the compound of any one of paragraphs 1-75 and a pharmaceutically acceptable carrier.

Paragraph 77: A pharmaceutical composition comprising an effective amount of the compound of any one of paragraphs 1-75 for treating an MNK-mediated disorder or condition.

Paragraph 78: The pharmaceutical composition of paragraph 77 wherein the MNK-mediated disorder or condition is selected from the group consisting of colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome.

Paragraph 79: A method of treatment comprising administering an effective amount of a compound of any one of paragraphs 1-74, or administering a pharmaceutical composition comprising an effective amount of a compound of any one of paragraphs 1-75, to a subject suffering from an MNK-mediated disorder or condition.

Paragraph 80: The method of paragraph 79, wherein the disorder or condition is selected from the group consisting of colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome.

Paragraph 80: A method for inhibiting the activity of Mnk in at least one cell overexpressing Mnk, comprising contacting the at least one cell with an effective amount of the compound according to any one of paragraphs 1-75.

### EQUIVALENTS

While certain embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. A compound having a structure of Formula I or Formula II: 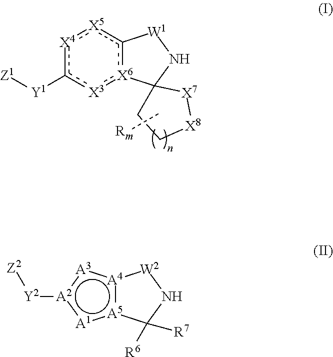
stereoisomers, tautomers, and/or pharmaceutically acceptable salts thereof; wherein
W¹ and W² are independently C(═NR⁹), C(═O), C(═S), S(═O), or S(═O)₂,
Y¹ and Y² are independently absent, NH, NR¹⁰, O, CHR¹⁴, C(═O), S(═O), S(O)₂, cyclopropyl, or a 5-member heteroarylene ring;
Z¹ and Z² are independently a heteroaryl moiety selected from Formulas III, IV, V, VI, VII, VIII, IX, X, or XI: 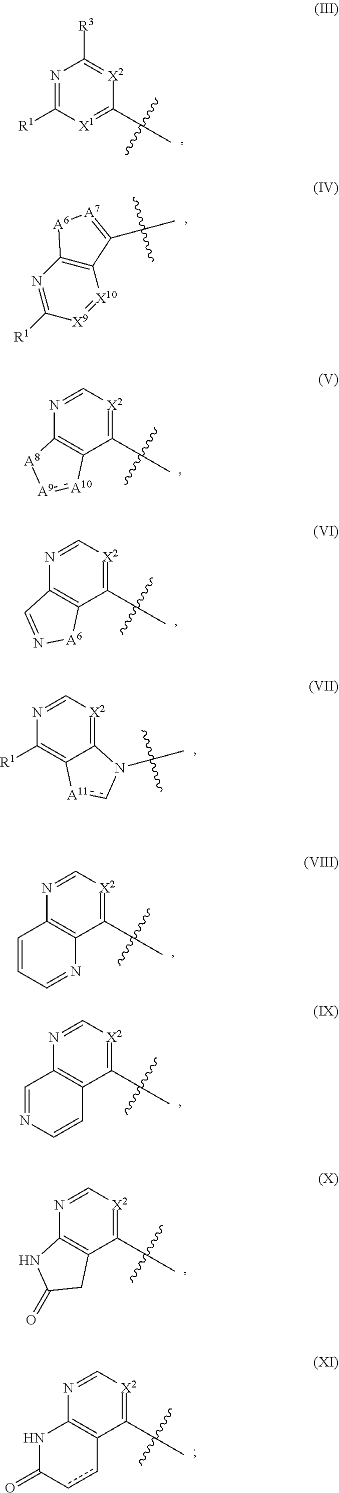
wherein the dotted lines in Formulas V, VII, and XI indicate a single or double bond;
A¹ is CR^{2e}, N, NR⁸, O or S, provided that the ring of which it is a member is a heteroaryl ring;
A² is C or N, provided that the ring of which it is a member is a heteroaryl ring;
A³ is CR^{2f}, N, NR⁸, O or S, provided that the ring of which it is a member is a heteroaryl ring;
A⁴ and A⁵ are each C or one is C and the other N, such that the ring of which they are members is a heteroaryl ring;
A⁶ is NR⁸, O, S, or S(═O);
A⁷ is CR^{2g} or N;
A⁸ is NR⁸, NHC(O), O, S, S(═O);
A⁹ is CH, CH₂, C(O), CR¹⁵, CR¹⁸, or N, provided that when A⁹ is CR¹⁵, A¹⁰ is CR¹⁶;
A¹⁰ is CH, CH₂, CR¹⁶, CR¹⁹, N, NH, or S, provided that when A¹⁰ is CR¹⁶, A⁹ is CR¹⁵;
A¹¹ is CH, CH₂ or N;
X¹ is N or CR^{2a};
X² is N or CR^{2b};
X³ is N, N(O), C(═O) or CR^{2c};
X⁴ is N or CR⁴;
X⁵ is N or CR⁵;
X⁶ is C or N, wherein when X⁶ is C, the dotted lines in Formula I indicate aromatic bonds, and when X⁶ is N, then X³ is C(═O) and the dotted lines in Formula I indicate single or double bonds;
X⁷ and X⁸ are independently O, NH N(O) NR¹⁰, NC(O)R¹¹, NC(O)OR¹¹, S, S(═O), S(═O)₂, CHR¹³, and C(═O), provided that X⁷ and X⁸ are not both O;
X⁹ and X¹⁰ are independently N or CR^{2d};
R is independently at each occurrence halo, NO₂, NR⁸R¹⁰, OR¹¹, SR₁₂, CN COOR¹³, or a substituted or unsubstituted C₁₋₆ alkyl, C₃₋₇ cycloalkyl, or C₂₋₆ alkenyl group; or when m is at least 2, the two R moieties together form a C₁₋₄ alkylene bridge between non adjacent ring members;
R¹, R^{2a}, R^{2b}, R^{2c}, R^{2d}, R^{2e}, R^{2f}, R^{2g}, and R³ are independently at each occurrence H, halo, NO₂, NR⁸R¹⁰, OR¹¹, SR¹², CN, COOR¹³, or a substituted or unsubstituted C₁₋₆ alkyl, C₃₋₇ cycloalkyl, or, C₂₋₆ alkenyl group;
R⁴ and R⁵ are independently H, halo, CN, OH, SR¹², NO₂, NR⁸R¹⁰, or a substituted or unsubstituted C₁₋₆ alkyl, C₁₋₆ alkoxy, or C₂₋₆ alkene; or R⁴ and R⁵ when present, together with the carbon atoms to which they are attached, form a fused phenyl or a 5- or 6-membered cycloalkenyl, heterocyclyl or heteroaryl ring;
R⁶ and R⁷ are independently H, NHR¹⁰, or a substituted or unsubstituted C₁₋₈-alkyl, C₂₋₈-alkenyl, cycloalkyl, cycloalkylalkyl, aryl, aralkyl, heterocyclyl, heterocyclylalkyl, heteroaryl, or heteroarylalkyl group; or R⁶ and R⁷ together with the carbon to which they are attached form a substituted or unsubstituted cycloalkyl or heterocyclyl ring;
R⁸ and R¹⁰ are independently at each occurrence H, an amino protecting group, or a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group; or R⁸ and R¹⁰ together with the nitrogen to which they are attached form a substituted or unsubstituted heterocyclyl ring;
R⁹ is independently at each occurrence H or substituted or unsubstituted alkyl group;
R¹¹ is independently at each occurrence H, a hydroxyl protecting group, or a substituted or unsubstituted alkyl, cycloalkyl, alkenyl, heterocyclyl, heteroaryl, aryl or aralkyl group;
R¹² is independently at each occurrence H, a thiol protecting group, or a substituted or unsubstituted alkyl, cycloalkyl, alkenyl, aryl or aralkyl group;
R¹³ is independently at each occurrence H or a substituted or unsubstituted alkyl, cycloalkyl, alkenyl, aryl or aralkyl group; and
R¹⁴ is H, OH, or a substituted or unsubstituted alkyl group;
R¹⁵ and R¹⁶, together with the carbons to which they are attached, form a cyclohexenyl ring, optionally substituted with C(O)R¹⁷, C(O)OR¹⁷, or C(O)NR⁸R¹⁰;
R¹⁷ is independently at each occurrence H or a substituted or unsubstituted alkyl, cycloalkyl, or alkenyl group;
R¹⁸ and R¹⁹ are independently selected from CN, C(O)R¹⁷, C(O)OR¹⁷, C(O)NR⁸R¹⁰, or a substituted or unsubstituted alkyl, cycloalkyl, or alkenyl group;
m is 0, 1, 2 or 3; and
n is 1 or 2;
provided that when X⁴ is CH, at least one of X⁷ and X⁸ is a heteroatom, or n is at least 2 and the two R moieties together form a C₁₋₄ alkylene bridge between non-adjacent ring members, or W¹ is S(═O)₂, or X³ is N(O).

2. The compound of claim 1 having the structure of Formula I.

3. The compound of claim 1, wherein the compound of Formula I is a compound of Formula IB: 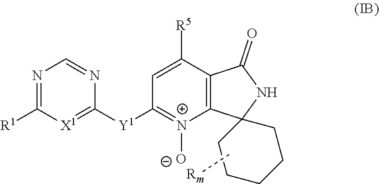
or a stereoisomer, tautomer, and/or pharmaceutically acceptable salt thereof.

4. The compound of claim 1, wherein the compound of Formula I is a compound of Formula IB-1: 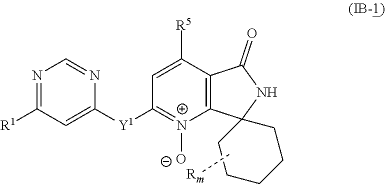
or a stereoisomer, tautomer, and/or pharmaceutically acceptable salt thereof.

5. The compound of claim 1, wherein the compound of Formula I is a compound of Formula IA: 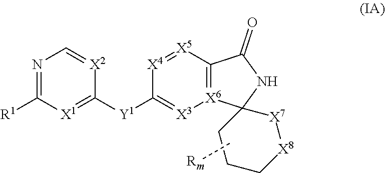
or a stereoisomer, tautomer, and/or pharmaceutically acceptable salt thereof.

6. The compound of claim 1, wherein Y¹ or Y² is NH.

7. The compound of claim 1, wherein Y¹ or Y² is an oxazole, isoxazole, thiazole, imidazole, oxadiazole, dioxazole, or isothiazole.

8. The compound of claim 1, wherein Z¹ or Z² is a moiety of Formula III.

9. The compound of claim 1, wherein Z¹ or Z² is a moiety of Formula IV.

10. The compound of claim 1, wherein Z¹ or Z² is a moiety of Formula V.

11. The compound of claim 1, wherein Z¹ or Z² is a moiety of Formula VI.

12. The compound of claim 1, wherein Z¹ or Z² is a moiety of Formula VII.

13. The compound of claim 1, wherein Z¹ or Z² is a moiety of Formula VIII.

14. The compound of claim 1, wherein Z¹ or Z² is a moiety of Formula IX.

15. The compound of claim 1, wherein Z¹ or Z² is a moiety of Formula X.

16. The compound of claim 1, wherein Z¹ or Z² is a moiety of Formula XI.

17. The compound of claim 1, wherein R¹ is NR⁸R¹⁰.

18. The compound of claim 1, wherein R⁸ is H.

19. The compound of claim 1, wherein R¹⁰ is an amino protecting group, or a substituted or unsubstituted alkyl, alkenyl, C(O)-alkyl, C(O)-cycloalkyl, C(O)-aryl, C(O)-heteroaryl, C(O)-heterocyclyl, C(O)NH-alkyl, C₁-C₄ alkyl-OH, C₁-C₄ alkylene-O—C₁-C₄ alkyl, cycloalkyl, cycloalkylalkyl, aryl, aralkyl, heterocyclylalkyl, heterocyclyl, heteroaryl, or heteroarylene-heterocyclyl group.

20. The compound of claim 19, wherein R¹⁰ is selected from the group consisting of: 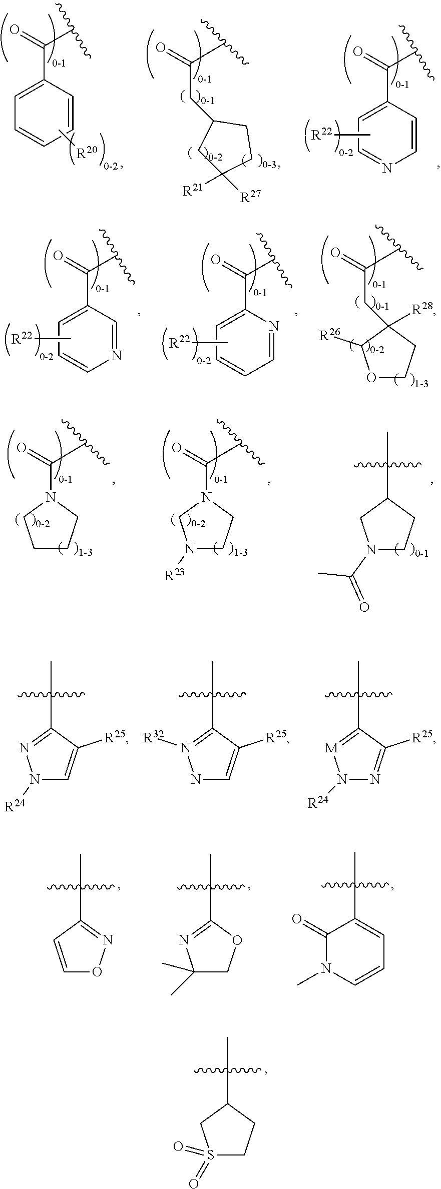
CH₃, —(CH₂)₁₋₃OH, —(CH₂)₁₋₃OCH₃, —C(O)N(CH₃)₂, and —C(O)NHCH₃,
wherein R²⁰ is halo, or N(CH₃)C(O)CH₃; R²¹ is H, OH, or OCH₃; R²² is CH₃, CH₂OH, —C(O)NHCH₃, —N(CH₃)C(O)CH₃, 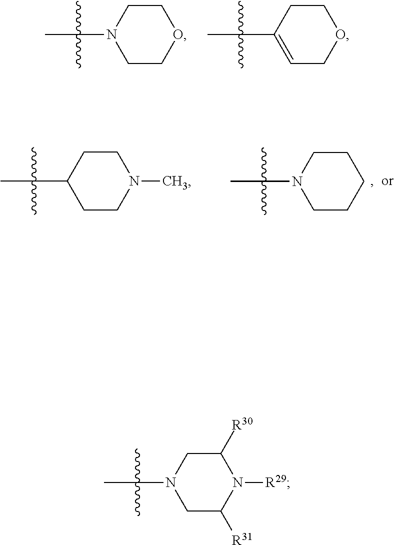
R²³ is H or C(O)CH₃; R²⁴ is H, CH₃, CF₃, (CH₂)₁₋₂OH, or CH(CH₃)₂; R²⁵ is H or CH₃; R²⁶ at each location is independently H or OH; R²⁷ is H or CH₃; R²⁸ is H or C(O)OCH₃; R²⁹ is H, CH₃, CH₂CH₃, or CH₂CH₂OH; R³⁰ and R³¹ are each independently H or CH₃; R³² is H or R³² is H or 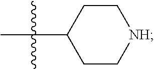 [SMILES: *C1CCNCC1]
and M is CH or N.

21. The compound of claim 1, wherein R⁸ and R¹⁰ together with the nitrogen to which they are attached form a substituted or unsubstituted heterocyclyl ring.

22. The compound of claim 21, wherein R⁸ and R¹⁰ together with the nitrogen to which they are attached are selected from the group consisting of:

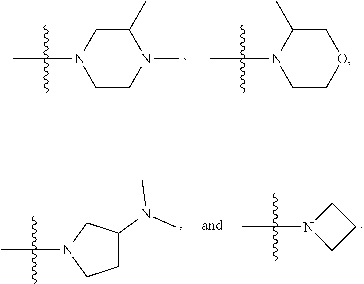

23. The compound of claim 1, having the structure of Formula II.

24. The compound of claim 23 having a structure selected from the group consisting of Formula IIA, Formula IIB, Formula IIC, Formula IID, Formula IIE, and Formula IIF:

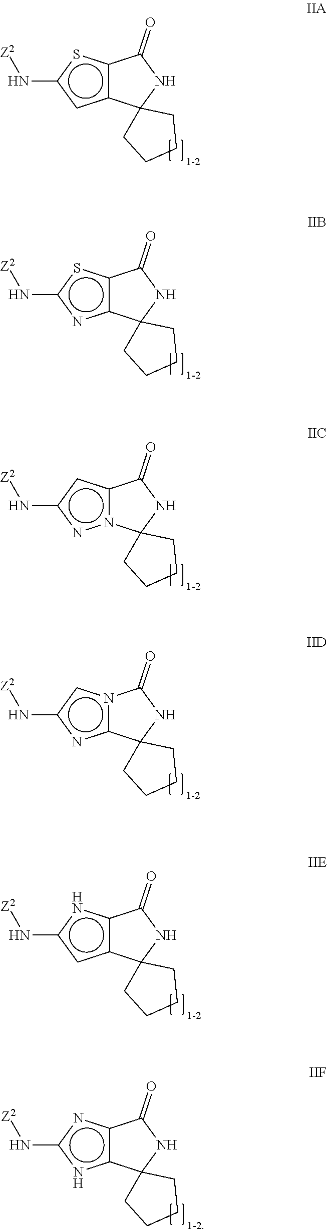

25. A composition comprising the compound of claim 1 and a pharmaceutically acceptable carrier.

26. A pharmaceutical composition comprising an effective amount of the compound of claim 1 for treating an MNK-mediated disorder or condition.

27. The pharmaceutical composition of claim 26 wherein the MNK-mediated disorder or condition is selected from the group consisting of colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome.

28. A method of treatment comprising administering an effective amount of a compound of claim 1, or administering a pharmaceutical composition comprising an effective amount of a compound of claim 1, to a subject suffering from an MNK-mediated disorder or condition.

29. The method of claim 28, wherein the disorder or condition is selected from the group consisting of colorectal cancer, bladder cancer, gastric cancer, esophageal cancer, head and neck cancer, CNS cancer, malignant glioma, glioblastoma, hepatocellular cancers, thyroid cancer, liver cancer, lung cancer, non-small cell cancer, small cell lung cancer, melanoma, myeloma, pancreatic cancer, pancreatic carcinoma, renal cell carcinoma, cervical cancer, urothelial cancer, prostate cancer, castration-resistant prostate cancer, ovarian cancer, breast cancer, triple-negative breast cancer, leukemia, Hodgkin's lymphoma, non-Hodgkin's lymphoma, B-cell lymphoma, T-cell lymphoma, hairy cell lymphoma, diffuse large B-cell lymphoma, Burkitt's lymphoma, multiple myeloma, and myelodysplastic syndrome.

30. A method for inhibiting the activity of Mnk in at least one cell overexpressing Mnk, comprising contacting the at least one cell with an effective amount of the compound according to claim 1.
